(19) United States
(12) Patent Application Publication
(10) Pub. No.: US 2011/0038876 A1
(43) Pub. Date: Feb. 17, 2011

(54) HETEROCYCLIC COMPOUNDS AND USE THEREOF AS ERK INHIBITORS

(76) Inventors: Robert Sun, Natick, MA (US); Alan B. Cooper, West Caldwell, NJ (US); Yongqi Deng, Newton, MA (US); Tong Wang, New Vernon, NJ (US); Yang Nan, Malden, MA (US); Hugh Y. Zhu, Warren, NJ (US); Sobhana Babu Boga, Scotch Plains, NJ (US); Xiaolei Gao, Bridgewater, NJ (US); Joseph M. Kelly, Parlin, NJ (US); Sunil Paliwal, Monroe Township, NJ (US); Hon-Chung Tsui, East Brunswick, NJ (US); Ronald J. Doll, Convent Station, NJ (US); Neng-Yang Shih, Lexington, MA (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/601,476

(22) PCT Filed: Jun. 17, 2008
(86) PCT No.: PCT/US08/07509

§ 371 Date: Sep. 16, 2010

Related U.S. Application Data
(60) Provisional application No. 60/936,188, filed on Jun. 18, 2007.

(51) Int. Cl. A61K 31/519; C07D 487/04; C07D 487/08; A61K 31/496; A61K 39/395; A61P 35/00; A61P 35/02

(52) U.S. Cl. 424/158.1; 544/251; 514/267; 514/252.16; 424/172.1

(57) ABSTRACT

Disclosed are the ERK inhibitors of formula 1.0: [Formula (1.0)] and the pharmaceutically acceptable salts, esters and solvates thereof. Q is a piperidine or piperazine ring that can have a bridge or a fused ring. The piperidine ring can have a double bond in the ring. All other substitutents are as defined herein. Also disclosed are methods of treating cancer using the compounds of formula 1.0.

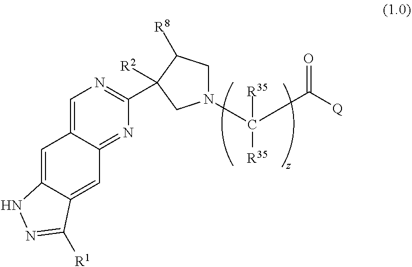

### REFERENCE TO RELATED APPLICATION

This application claims the benefit of U.S. Provisional Application No. 60/936,188 filed Jun. 18, 2007.

### BACKGROUND

The processes involved in tumor growth, progression, and metastasis are mediated by signaling pathways that are activated in cancer cells. The ERK pathway plays a central role in regulating mammalian cell growth by relaying extracellular signals from ligand-bound cell surface tyrosine kinase receptors such as erbB family, PDGF, FGF, and VEGF receptor tyrosine kinase. Activation of the ERK pathway is via a cascade of phosphorylation events that begins with activation of Ras. Activation of Ras leads to the recruitment and activation of Raf, a serine-threonine kinase. Activated Raf then phosphorylates and activates MEK1/2, which then phosphorylates and activates ERK1/2. When activated, ERK1/2 phosphorylates several downstream targets involved in a multitude of cellular events including cytoskeletal changes and transcriptional activation. The ERK/MAPK pathway is one of the most important for cell proliferation, and it is believed that the ERK/MAPK pathway is frequently activated in many tumors. Ras genes, which are upstream of ERK1/2, are mutated in several cancers including colorectal, melanoma, breast and pancreatic tumors. The high Ras activity is accompanied by elevated ERK activity in many human tumors. In addition, mutations of BRAF, a serine-threonine kinase of the Raf family, are associated with increased kinase activity. Mutations in BRAF have been identified in melanomas (60%), thyroid cancers (greater than 40%) and colorectal cancers. These observations indicate that the ERK1/2 signalling pathway is an attractive pathway for anticancer therapies in a broad spectrum of human tumours.

Therefore, a welcome contribution to the art would be small-molecules (i.e., compounds) that inhibit ERK activity (i.e., ERK1 and ERK2 activity), which small-molecules would be useful for treating a broad spectrum of cancers, such as, for example, melanoma, pancreatic cancer, thyroid cancer, colorectal cancer, lung cancer, breast cancer, and ovarian cancer. Such a contribution is provided by this invention.

### SUMMARY OF THE INVENTION

This invention provides compounds that inhibit the activity of ERK1 and/or the activity of ERK2.

The compounds of this invention also inhibit the phosphorylation of ERK1 and ERK2.

Thus, this invention provides compounds that are ERK inhibitors (i.e., ERK1 inhibitors and/or ERK2 inhibitors), said compounds being of the formula 1.0:

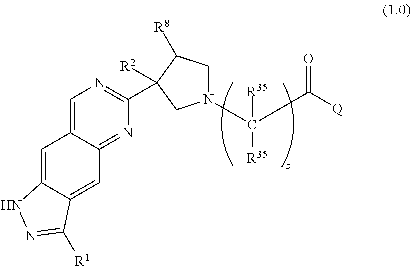

or the pharmaceutically acceptable salts, esters and solvates thereof, wherein:

Q is selected from the group consisting of: piperidinyl, piperazinyl, tetrahydropyridinyl (e.g., 1,2,3,6-tetrahydropyridinyl), bridged piperazinyl, bridged piperidinyl, bridged tetrahydropyridinyl, substituted piperidinyl, substituted piperazinyl, substituted tetrahydropyridinyl (e.g., a substituted 1,2,3,6-tetrahydro-pyridinyl), bridged substituted piperazinyl, bridged substituted piperidinyl, and bridged substituted tetrahydropyridinyl;

z is 1 to 3 (and preferably 1); and

R¹, R², R⁸, and R³⁵ are as defined below.

This invention provides compounds of formula 1.0 (for example, as described in any one of Embodiment Nos. 1 to 92) in pure or isolated form.

This invention provides compounds of formula 1.0 (for example, as described in any one of Embodiment Nos. 1 to 92) in pure form.

This invention provides compounds of formula 1.0 (for example, as described in any one of Embodiment Nos. 1 to 92) in isolated form.

This invention provides compounds of formula 1.0.

This invention provides pharmaceutically acceptable salts of the compounds of formula 1.0.

This invention provides pharmaceutically acceptable esters of the compounds of formula 1.0.

This invention provides solvates of the compounds of formula 1.0.

This invention provides the final compounds of Examples 1 to 12.

This invention also provides a pharmaceutical composition comprising an effective amount of at least one (e.g., 1, 2 or 3, 1 or 2, and usually 1) compound of formula 1.0 (for example, as described in any one of Embodiment Nos. 1 to 93) and a pharmaceutically acceptable carrier.

This invention also provides a pharmaceutical composition comprising an effective amount of at least one (e.g., 1, 2 or 3, 1 or 2, and usually 1) compound of formula 1.0 (for example, as described in any one of Embodiment Nos. 1 to 93) and an effective amount of at least one other (e.g., 1, 2 or 3, 1 or 2, and usually 1) pharmaceutically active ingredient (such as, for example, a chemotherapeutic agent), and a pharmaceutically acceptable carrier.

This invention also provides a method of inhibiting ERK (i.e., inhibiting the activity of ERK) in a patient in need of such treatment comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method of inhibiting ERK1 (i.e., inhibiting the activity of ERK1) in a patient in need of such treatment comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method of inhibiting ERK2 (i.e., inhibiting the activity of ERK2) in a patient in need of such treatment comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method of inhibiting ERK1 and ERK2 (i.e., inhibiting the activity of ERK1 and ERK2) in a patient in need of such treatment comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method of treating cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93) in combination with at least one (e.g., 1, 2 or 3, 1 or 2, and usually 1) signal transduction inhibitor.

This invention also provides a method of treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93) in combination with at least one (e.g., 1, 2 or 3, 1 or 2, and usually 1) signal transduction inhibitor.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma, in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating lung cancer, pancreatic cancer, colon cancer (e.g., colorectal cancer), myeloid leukemias (e.g., AML, CML, and CMML), thyroid cancer, myelodysplastic syndrome (MDS), bladder carcinoma, epidermal carcinoma, melanoma, breast cancer, prostate cancer, head and neck cancers (e.g., squamous cell cancer of the head and neck), ovarian cancer, brain cancers (e.g., gliomas, such as glioma blastoma multiforme), cancers of mesenchymal origin (e.g., fibrosarcomas and rhabdomyosarcomas), sarcomas, tetracarcinomas, nuroblastomas, kidney carcinomas, hepatomas, non-Hodgkin's lymphoma, multiple myeloma, or anaplastic thyroid carcinoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), wherein said cancer is selected from the group consisting of: melanoma, pancreatic cancer, thyroid cancer, colorectal cancer, lung cancer, breast cancer, and ovarian cancer.

This invention also provides a method for treating cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent wherein said cancer is selected from the group consisting of: melanoma, pancreatic cancer, thyroid cancer, colorectal cancer, lung cancer, breast cancer, and ovarian cancer.

This invention also provides a method for treating melanoma in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating melanoma in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating melanoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating melanoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating pancreatic cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating pancreatic cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating pancreatic cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating pancreatic cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating thyroid cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating thyroid cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating thyroid cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating thyroid cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g.; 1, 2 or 3, 1 or 2, or 1) chemotherapeutic agent.

This invention also provides a method for treating colorectal cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating colorectal cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating colorectal cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating colorectal cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating lung cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating lung cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating lung canter patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating lung cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating breast cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating breast cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating breast cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating breast cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating ovarian cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating ovarian cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating ovarian cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating ovarian cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides methods of treating breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 (for example, as described in any one of Embodiment Nos. 1 to 93) in combination with hormonal therapies (i.e., antihormonal agents).

This invention also provides methods of treating breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93) in combination with hormonal therapies (i.e., antihormonal agents).

This invention also provides methods of treating breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 (for example, as described in any one of Embodiment Nos. 1 to 93) in combination with hormonal therapies (i.e., antihormonal agents), and in combination with an effective amount of at least one (e.g., 1, 2 or 3, 1 or 2, or 1) chemotherapeutic agent.

This invention also provides methods of treating breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93) in combination with hormonal therapies (i.e., antihormonal agents), and in combination with an effective amount of at least one (e.g., 1, 2 or 3, 1 or 2, or 1) chemotherapeutic agent.

The methods of treating breast cancer described herein include the treatment of hormone-dependent metastatic and advanced breast cancer, adjuvant therapy for hormone-dependent primary and early breast cancer, the treatment of ductal carcinoma in situ, and the treatment of inflammatory breast cancer in situ.

The methods of treating hormone-dependent breast cancer can also be used to prevent breast cancer in patients having a high risk of developing breast cancer.

Thus, this invention also provides methods of preventing breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 (for example, as described in any one of Embodiment Nos. 1 to 93) in combination with hormonal therapies (i.e., antihormonal agents).

This invention also provides methods of preventing breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93) in combination with hormonal therapies (i.e., antihormonal agents).

This invention also provides methods of preventing breast cancer (i.e., post-menopausal and premenopausal breast cancer, e.g., hormone-dependent breast cancer) in a patient in need of such treatment, said treatment comprising the administration of an effective amount of at least one (e.g., 1, 2 or 3, 1 or 2, and usually 1) compound of formula 1.0 (for example, as described in any one of Embodiment Nos. 1 to 93) in combination with hormonal therapies (i.e., antihormonal agents), and in combination with an effective amount of at least one (e.g., 1, 2 or 3, 1 or 2, or 1) chemotherapeutic agent.

This invention also provides methods of preventing breast cancer (i.e., post-menopausal and premenopausal breast cancer, e.g., hormone-dependent breast cancer) in a patient in need of such treatment, said treatment comprising the administration of an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93) in combination with hormonal therapies (i.e., antihormonal agents), and in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) a in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating brain cancer (e.g., glioma, such as glioma blastoma multiforme) in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating brain cancer (e.g., glioma, such as glioma blastoma multiforme) in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of a chemotherapeutic agent wherein said chemotherapeutic agent is temozolomide.

This invention also provides a method for treating brain cancer (e.g., glioma, such as glioma blastoma multiforme) in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of a chemotherapeutic agent, wherein said chemotherapeutic agent is temozolomide.

This invention also provides a method for treating prostate cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating prostate cancer in a patient in need of such treatment, said method comprising administering to said patient an effective amount of at least one (e.g., 1, 2 or 3, 1 or 2, and usually 1) compound of formula 1.0 (for example; as described in any one of Embodiment Nos. 1 to 93), in combination with an effective amount of at least one (e.g., 1, 2 or 3, 1 or 2, or 1) chemotherapeutic agent.

This invention also provides a method for treating prostate cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating prostate cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating myelodysplastic syndrome in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating myelodysplastic syndrome in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating myelodysplastic syndrome in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating myelodysplastic syndrome in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating myeloid leukemias in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating acute myelogenous leukemia (AML) in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating acute myelogenous leukemia (AML) in a patient in need of such treatment, said method comprising administering to said patient an effective amount of at least one (e.g., 1, 2 or 3, 1 or 2; and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating acute myelogenous leukemia (AML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating acute myelogenous leukemia (AML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating chronic myelomonocytic leukemia (CMML) in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating chronic myelomonocytic leukemia (CMML) in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating chronic myelomonocytic leukemia (CMML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating chronic myelomonocytic leukemia (CMML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating chronic myelogenous leukemia (chronic myeloid leukemia, CML) in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating chronic myelogenous leukemia (chronic myeloid leukemia, CML) in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating chronic myelogenous leukemia (chronic myeloid leukemia, CML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating chronic myelogenous leukemia (chronic myeloid leukemia, CML) in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating myeloid leukemias in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating myeloid leukemias in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating bladder cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating bladder cancer in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating bladder cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating bladder cancer in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating non-Hodgkin's lymphoma in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93).

This invention also provides a method for treating non-Hodgkin's lymphoma in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating non-Hodgkin's lymphoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating non-Hodgkin's lymphoma in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating multiple myeloma in a patient in need of such treatment, said method comprising administering to said patient an effective amount of at least one (e.g., 1; 2 or 3, 1 or 2; and usually 1) compound of formula 1.0 (for example, as described in any one of Embodiment Nos. 1 to 93).

This invention also provides a method for treating multiple myeloma in a patient in need of such treatment, said method comprising administering to said patient an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

This invention also provides a method for treating multiple myeloma in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93).

This invention also provides a method for treating multiple myeloma in a patient in need of such treatment, said method comprising administering to said patient an effective amount of a pharmaceutical composition comprising an effective amount of at least one (e.g., 1, 2 or 3, 1 or 2, and usually 1) compound of formula 1.0 (for example, as described in any one of Embodiment Nos. 1 to 93), in combination with an effective amount of at least one (e.g., 1, 2 or 3, 1 or 2, or 1) chemotherapeutic agent.

In the methods of this invention the compounds of this invention can be administered concurrently or sequentially (i.e., consecutively) with the chemotherapeutic agents or the signal transduction inhibitor.

The methods of treating cancers described herein can optionally include the administration of an effective amount of radiation (i.e., the methods of treating cancers described herein optionally include the administration of radiation therapy).

### DETAILED DESCRIPTION OF THE INVENTION

As described herein, unless otherwise indicated, the use of a drug or compound in a specified period is per treatment cycle. For example, once a day means once per day of each day of the treatment cycle. Twice a day means twice per day each day of the treatment cycle. Once a week means one time per week during the treatment cycle. Once every three weeks means once per three weeks during the treatment cycle.

The following abbreviations have the following meanings unless defined otherwise:
ACN Acetonitrile
AcOH Acetic acid
DAST (diethylamino)sulfur trifluoride
DCC Dicyclohexylcarbodiimide
DCU Dicyclohexylurea
DCM Dichloromethane
DI Deionized water
DIAD Diisopropylazodicarboxylate
DIEA Diisopropylethylamine
DMAP 4-Dimethylaminopyridine
DME Dimethoxyethane
DMF Dimethylformamide
DMFDMA N,N-Dimethylformamide dimethylacetal
DMSO Dimethyl sulfoxide
DTT Dithiothreitol
EDCI 1-(3-dimethylamino-propyl)-3-ethylcarbodiimide hydrochloride
EtOAc Ethyl acetate
EtOH Ethanol
HATU N,N,N′,N′-Tetramethyl-O-(7-Azabenzotriazol-1-yl)Uronium hexafluorophosphate
Hex hexanes
HOBt 1-Hydroxylbenzotriazole
HPLC High pressure liquid chromatography
LCMS Liquid chromatography mass spectrometry
LDA Lithium diisopropylamide
mCPBA meta-Chloroperoxybenzoic acid
MeOH Methanol
MTT (3-[4,5-dimethyl-thiazol-2-yl]-2,5-diphenyltetrazolium bromide, Thiazolyl blue)
NMR Nuclear magnetic resonance
PFP Pentafluorophenol
PMB p-methoxybenzyl
Pyr Pyridine
Rb Round bottom flask
Rbt Round bottom flask
RT Room temperature
SEMCI 2-(Trimethylsily)ethoxy methyl chloride
TEA Triethylamine
Tr Triphenyl methane
Trt Triphenyl methane
TrCI Triphenyl methane chloride
TFA Trifluoroacetic acid
THF Tetrahydrofuran
TLC Thin layer chromatography
TMS Trimethylsilyl

As used herein, unless otherwise specified, the following terms have the following meanings:

“anti-cancer agent” means a drug (medicament or pharmaceutically active ingredient) for treating cancer;

“antineoplastic agent” means a drug (medicament or pharmaceutically active ingredient) for treating cancer (i.e., a chemotherapeutic agent);

“at least one”, as used in reference to the number of compounds of this invention means for example 1-6, generally 1-4, more generally 1, 2 or 3, and usually one or two, and more usually one;

“at least one”, as used in reference to the number of chemotherapeutic agents used, means for example 1-6, generally 1-4, more generally 1, 2 or 3, and usually one or two, or one;

“chemotherapeutic agent” means a drug (medicament or pharmaceutically active ingredient) for treating cancer (i.e., and antineeoplastic agent);

“compound” with reference to the antineoplastic agents, includes the agents that are antibodies;

“concurrently” means (1) simultaneously in time (e.g., at the same time); or (2) at different times during the course of a common treatment schedule;

“consecutively” means one following the other;

“different” as used in the phrase “different antineoplastic agents” means that the agents are not the same compound or structure; preferably, “different” as used in the phrase “different antineoplastic agents” means not from the same class of antineoplastic agents; for example, one antineoplastic agent is a taxane, and another antineoplastic agent is a platinum coordinator compound;

“effective amount” or “therapeutically effective amount” is meant to describe an amount of compound or a composition of the present invention, or an amount of radiation, effective in treating or inhibiting the diseases or conditions described herein, and thus producing the desired therapeutic, ameliorative, inhibitory or preventative effect; thus, for example, in the methods of treating cancer described herein “effective amount” (or “therapeutically effective amount”) means, for example, the amount of the compound (or drug), or radiation, that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor; for example, in the treatment of lung cancer (e.g., non small cell lung cancer) a therapeutically effective amount is that amount that alleviates or eliminates cough, shortness of breath and/or pain; also, for example, an effective amount, or a therapeutically effective amount of the ERK inhibitor (i.e., a compound of this invention) is that amount which results in the reduction in ERK (ERK1 and/or ERK2) activity and phosphorylation; the reduction in ERK activity may be determined by the analysis of pharmacodynamic markers such as phosphorylated RSK1,2 and phosphorylated ERK1,2, using techniques well known in the art;

“Ex” in the tables represents “Example”;

“one or more” has the same meaning as “at least one”;

“patient” means an animal, such as a mammal (e.g., a human being, and preferably a human being);

“prodrug” means compounds that are rapidly transformed, for example, by hydrolysis in blood, in vivo to the parent compound, i.e., to the compounds of formula 1.0 or to a salt and/or to a solvate thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this invention includes Prodrugs of the novel compounds of this invention;

sequentially-represents (1) administration of one component of the method ((a) compound of the invention, or (b) chemotherapeutic agent, signal transduction inhibitor and/or radiation therapy) followed by administration of the other component or components; after adminsitration of one component, the next component can be administered substantially immediately after the first component, or the next component can be administered after an effective time period after the first component; the effective time period is the amount of time given for realization of maximum benefit from the administration of the first component; and

“solvate” means a physical association of a compound of this invention with one or more solvent molecules; this physical association involves varying degrees of ionic and covalent bonding, including hydrogen bonding; in certain instances the solvate will be capable of isolation, for example when one or more solvent molecules are incorporated in the crystal lattice of the crystalline solid; “solvate” encompasses both solution-phase and isolatable solvates; non-limiting examples of suitable solvates include ethanolates, methanolates, and the like; “hydrate” is a solvate wherein the solvent molecule is H₂O.

As used herein, unless otherwise specified, the following terms have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl, and the like):
“acyl” means an H—C(O)—, alkyl-C(O)—, alkenyl-C(O)—, Alkynyl-C(O)—, cycloalkyl-C(O)—, cycloalkenyl-C(O)—, or cycloalkynyl-C(O)— group in which the various groups are as defined below (and as defined below, the alkyl, alkenyl, alkynyl, cycloalkyl, cycloalkenyl and cycloalkynyl moieties can be substituted); the bond to the parent moiety is through the carbonyl; preferred acyls contain a lower alkyl; Non-limiting examples of suitable acyl groups include formyl, acetyl, propanoyl, 2-methylpropanoyl, butanoyl and cyclohexanoyl;
“alkenyl” means an aliphatic hydrocarbon group (chain) comprising at least one carbon to carbon double bond, wherein the chain can be straight or branched, and wherein said group comprises about 2 to about 15 carbon atoms; Preferred alkenyl groups comprise about 2 to about 12 carbon atoms in the chain; and more preferably about 2 to about 6 carbon atoms in the chain; branched means that one or more lower alkyl groups, such as methyl, ethyl or propyl, or alkenyl groups are attached to a linear alkenyl chain; “lower alkenyl” means an alkenyl group comprising about 2 to about 6 carbon atoms in the chain, and the chain can be straight or branched; the term “substituted alkenyl” means that the alkenyl group is substituted by one or more independently selected substituents, and each substituent is independently selected from the group consisting of: halo, alkyl, aryl, cycloalkyl, cyano, alkoxy and —S(alkyl); non-limiting examples of suitable alkenyl groups include ethenyl, propenyl, n-butenyl, 3-methylbut-2-enyl, n-pentenyl, octenyl and decenyl;
“alkoxy” means an alkyl-O— group (i.e., the bond to the parent moiety is through the ether oxygen) in which the alkyl group is unsubstituted or substituted as described below; non-limiting examples of suitable alkoxy groups include methoxy, ethoxy; n-propoxy, isopropoxy, n-butoxy and heptoxy;
“alkoxycarbonyl” means an alkyl-O—CO— group (i.e.; the bond to the parent moiety is through the carbonyl) wherein the alkyl group is unsubstituted or substituted as previously defined; non-limiting examples of suitable alkoxycarbonyl groups include methoxycarbonyl and ethoxycarbonyl;
“alkyl” (including the alkyl portions of other moieties, such as trifluoroalkyl and alkyloxy) means an aliphatic hydrocarbon group (chain) that can be straight or branched wherein said group comprises about 1 to about 20 carbon atoms in the chain; preferred alkyl groups comprise about 1 to about 12 carbon atoms in the chain; more preferred alkyl groups comprise about 1 to about 6 carbon atoms in the chain; branched means that one or more lower alkyl groups, such as methyl, ethyl or propyl, are attached to a linear alkyl chain; “lower alkyl” means a group comprising about 1 to about 6 carbon atoms in the chain, and said chain can be straight or branched; the term “substituted alkyl” means that the alkyl group is substituted by one or more independently selected substituents, and wherein each substituent is independently selected from the group consisting of: halo, aryl, cycloalkyl, cyano, hydroxy, alkoxy, alkylthio, amino, —NH(alkyl), —NH(cycloalkyl), —N(alkyl)₂, carboxy, —C(O)O-alkyl and —S(alkyl); non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl;
“alkylaryl” (or alkaryl) means an alkyl-aryl-group (i.e., the bond to the parent moiety is through the aryl group) wherein the alkyl group is unsubstituted or substituted as defined above, and the aryl group is unsubstituted or substituted as defined below; preferred alkylaryls comprise a lower alkyl group; non-limiting examples of suitable alkylaryl groups include o-tolyl, p-tolyl and xylyl;
“alkylheteroaryl” means an alkyl-heteroaryl-group (i.e., the bond to the parent moiety is through the heteroaryl group) wherein the alkyl is unsubstituted or substituted as defined above and the heteroaryl group is unsubstituted or substituted as defined below;
“alkylsulfinyl” means an alkyl-S(O)— group (i.e., the bond to the parent moiety is through the sulfinyl) wherein the alkyl group is unsubstituted or substituted as previously defined; preferred groups are those in which the alkyl group is lower alkyl;
“alkylsulfonyl” means an alkyl-S(O₂)— group (i.e., the bond to the parent moiety is through the sulfonyl) wherein the alkyl group is unsubstituted or substituted as previously defined; preferred groups are those in which the alkyl group is lower alkyl;
“alkylthio” means an alkyl-S— group (i.e., the bond to the parent moiety is through the sulfur) wherein the alkyl group is unsubstituted or substituted as previously described; non-limiting examples of suitable alkylthio groups include methylthio, ethylthio, i-propylthio and heptylthio;
“alkynyl” means an aliphatic hydrocarbon group (chain) comprising at least one carbon to carbon triple bond, wherein the chain can be straight or branched, and wherein the group comprises about 2 to about 15 carbon atoms in the; preferred alkynyl groups comprise about 2 to about 12 carbon atoms in the chain; and more preferably about 2 to about 4 carbon atoms in the chain; Branched means that one or more lower alkyl groups, such as methyl, ethyl or propyl, are attached to a linear alkynyl chain; “lower alkynyl” means an alkynyl group comprising about 2 to about 6 carbon atoms in the chain, and the chain can be straight or branched; non-limiting examples of suitable alkynyl groups include ethynyl, propynyl, 2-butynyl, 3-methylbutynyl, n-pentynyl, and decynyl; the term “substituted alkynyl” means that the alkynyl group is substituted by one or more independently selected, and each substituent is independently selected from the group consisting of alkyl; aryl and cycloalkyl;
“amino means a —NH₂ group;
“aralkenyl” (or arylalkenyl) means an aryl-alkenyl-group the bond to the parent moiety is through the alkenyl group) wherein the aryl group is unsubstituted or substituted as defined below, and the alkenyl group is unsubstituted or substituted as defined above; preferred aralkenyls contain a lower alkenyl group; non-limiting examples of suitable aralkenyl groups include 2-phenethenyl and 2-naphthylethenyl;
“aralkyl” (or arylalkyl) means an aryl-alkyl-group (i.e., the bond to the parent moiety is through the alkyl group) wherein the aryl is unsubstituted or substituted as defined below and the alkyl is unsubstituted or substituted as defined above; preferred aralkyls comprise a lower alkyl group; non-limiting examples of suitable aralkyl groups include benzyl, 2-phenethyl and naphthalenylmethyl;
“aralkyloxy” (or arylalkyloxy) means an aralkyl-O— group (i.e., the bond to the parent moiety is through the ether oxygen) wherein the aralkyl group is unsubstituted or substituted as previously described; non-limiting examples of suitable aralkyloxy groups include'benzyloxy and 1- or 2-naphthalenemethoxy;
“aralkoxycarbonyl” means an aralkyl-O—C(O)— group (i.e., the bond to the parent moiety is through the carbonyl) wherein the aralkyl group is unsubstituted or substituted as previously defined; a non-limiting example of a suitable aralkoxycarbonyl group is benzyloxycarbonyl;
“aralkylthio” means an aralkyl-S— group (i.e., the bond to the parent moiety is through the sulfur) wherein the aralkyl group is unsubstituted or substituted as previously described; a non-limiting example of a suitable aralkylthio group is benzylthio;
“aroyl” means an aryl-C(O)— group (i.e., the bond to the parent moiety is through the carbonyl) wherein the aryl group is unsubstituted or substituted as defined below; non-limiting examples of suitable groups include benzoyl and 1- and 2-naphthoyl;
“aryl” (sometimes abbreviated “ar”) means an aromatic monocyclic or multicyclic ring system comprising about 6 to about 14 carbon atoms, preferably about 6 to about 10 carbon atoms; the aryl group can be optionally substituted with one or more independently selected “ring system substituents” (defined below). Non-limiting examples of suitable aryl groups include phenyl and naphthyl;
“arylalkynyl” means an aryl-alkynyl-group (i.e., the bond to the parent moiety is through the alkynyl group) wherein the aryl group is unsubstituted or substituted as defined above, and the alkynyl group is unsubstituted or substituted as defined above;
“arylaminoheteroaryl” means an aryl-amino-heteroaryl group (i.e., the bond to the parent moiety is through the heteroaryl group) wherein the aryl group is unsubstituted or substituted as defined above, the amino group is as defined above (i.e., a —NH— here), and the heteroaryl group is unsubstituted or substituted as defined below;
“arylheteroaryl” means an aryl-heteroaryl group-(i.e., the bond to the parent moiety is through the heteroaryl group) wherein the aryl group is unsubstituted or substituted as defined above, and the heteroaryl group is unsubstituted or substituted as defined below;
“aryloxy” means an aryl-O— group (i.e., the bond to the parent moiety is through the ether oxygen) wherein the aryl group is unsubstituted or substituted as defined above; non-limiting examples of suitable aryloxy groups include phenoxy and naphthoxy;
“aryloxycarbonyl” means an aryl-O—C(O)— group (i.e., the bond to the parent moiety is through the carbonyl) wherein the aryl group is unsubstituted or substituted as previously defined; non-limiting examples of suitable aryloxycarbonyl groups include phenoxycarbonyl and naphthoxycarbonyl;
“arylsulfinyl” means an aryl-S(O)— group (i.e., the bond to the parent moiety is through the sulfinyl) wherein aryl is unsubstituted or substituted as previously defined;
“arylsulfonyl” means an aryl-S(O₂)— group (i.e., the bond to the parent moiety is through the sulfonyl) wherein aryl is unsubstituted or substituted as previously defined;
“arylthio” means an aryl-S— group (i.e., the bond to the parent moiety is through the sulfur) wherein the aryl group is unsubstituted or substituted as previously described; non-limiting examples of suitable arylthio groups include phenylthio and naphthylthio;
“cycloalkenyl” means a non-aromatic mono or multicyclic ring system comprising about 3 to about 10 carbon atoms, preferably about 5 to about 10 carbon atoms that contains at least one carbon-carbon double bond; preferred cycloalkenyl rings contain about 5 to about 7 ring atoms; the cycloalkenyl can be optionally substituted with one or more independently selected “ring system substituents” (defined below); Non-limiting examples of suitable monocyclic cycloalkenyls include cyclopentenyl, cyclohexenyl, cycloheptenyl, and the like; a non-limiting example of a suitable multicyclic cycloalkenyl is norbornylenyl;
“cycloalkyl” means a non-aromatic mono- or multicyclic ring system comprising about 3 to about 7 carbon atoms, preferably about 3 to about 6 carbon atoms; the cycloalkyl can be optionally substituted with one or more independently selected “ring system substituents” (defined below); non-limiting examples of suitable monocyclic cycloalkyls include cyclopropyl, cyclopentyl, cyclohexyl, cycloheptyl and the like; non-limiting examples of suitable multicyclic cycloalkyls include 1-decalin, norbornyl, adamantyl and the like;
“cycloalkylalkyl” means a cycloalkyl-alkyl-group (i.e., the bond to the parent moiety is through the alkyl group) wherein the cycloalkyl moiety is unsubstituted or substituted as defined above, and the alkyl moiety is unsubstituted or substituted as defined above;
“halo” means fluoro, chloro; bromo, or iodo groups; preferred halos are fluoro, chloro or bromo, and more preferred are fluoro and chloro;
“halogen” means fluorine, chlorine, bromine, or iodine; preferred halogens are fluorine, chlorine and bromine;
“haloalkyl” means an alkyl, as defined above, wherein one or more hydrogen atoms on the alkyl is replaced by a halo group, as defined above;
“heteroaralkenyl” means a heteroaryl-alkenyl-group (i.e., the bond to the parent moiety is through the alkenyl group) wherein the heteroaryl group is unsubstituted or substituted as defined below, and the alkenyl group is unsubstituted or substituted as defined above;
“heteroaralkyl” (or heteroarylalkyl) means a heteroaryl-alkyl-group (i.e., the bond to the parent moiety is through the alkyl group) in which the heteroaryl is unsubstituted or substituted as defined below, and the alkyl group is unsubstituted or substituted as defined above; preferred heteroaralkyls comprise an alkyl group that is a lower alkyl group; non-limiting examples of suitable aralkyl groups include pyridylmethyl, 2-(furan-3-yl)ethyl and quinolin-3-ylmethyl;
“heteroaralkylthio” means a heteroaralkyl-S— group wherein the heteroaralkyl group is unsubstituted or substituted as defined above;
“heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination; preferred heteroaryls comprise about 5 to about 6 ring atoms; the “heteroaryl” can be optionally substituted by one or more independently selected “ring system substituents” (defined below); the prefix aza, oxa or thia before the heteroaryl root name means that at least a nitrogen, oxygen or sulfur atom, respectively, is present as a ring atom; a nitrogen atom of a heteroaryl can be optionally oxidized to the corresponding N-oxide; non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl, and furopyridine

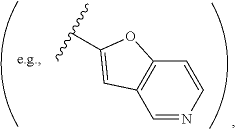

and the like;
“heteroarylalkynyl” (or heteroaralkynyl) means a heteroaryl-alkynyl-group (i.e., the bond to the parent moiety is through the alkynyl group) wherein the heteroaryl group is unsubstituted or substituted as defined above, and the alkynyl group is unsubstituted or substituted as defined above;
“heteroarylaryl” (or heteroararyl) means a heteroaryl-aryl-group (i.e., the bond to the parent moiety is through the aryl group) wherein the heteroaryl group is unsubstituted or substituted as defined above, and the aryl group is unsubstituted or substituted as defined above;
“heteroarylheteroarylaryl” means a heteroaryl-heteroaryl-group (i.e., the bond to the parent moiety is through the last heteroaryl group) wherein each heteroaryl group is independently unsubstituted or substituted as defined above;
“heteroarylsulfinyl” means a heteroaryl-SO— group wherein the heteroaryl group is unsubstituted or substituted as defined above;
“heteroarylsulfonyl” means a heteroaryl-SO₂— group wherein the heteroaryl group is unsubstituted or substituted as defined above;
“heteroarylthio” means a heteroaryl-S— group wherein the heteroaryl group is unsubstituted or substituted as defined above;
“heterocyclenyl” (or heterocycloalkenyl) means a non-aromatic monocyclic or multicyclic ring system comprising about 3 to about 10 ring atoms, preferably about to about 10 ring atoms, in which one or more of the atoms in the ring system is an element other than carbon (for example one or more heteroatoms independently selected from the group consisting of nitrogen, oxygen and sulfur atom), and which contains at least one carbon-carbon double bond or carbon-nitrogen double bond; there are no adjacent oxygen and/or sulfur atoms present in the ring system; Preferred heterocyclenyl rings contain about 5 to about 6 ring atoms; the prefix aza, oxa or thia before the heterocyclenyl root name means that at least a nitrogen, oxygen or sulfur atom, respectively, is present as a ring atom; the heterocyclenyl can be optionally substituted by one or more independently selected “Ring system substituents” (defined below); the nitrogen or sulfur atom of the heterocyclenyl can be optionally oxidized to the corresponding N-oxide, S-oxide or S,S-dioxide; non-limiting examples of suitable monocyclic azaheterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like; Non-limiting examples of suitable oxaheterocyclenyl groups include 3,4-dihydro-2H-pyran, dihydrofuranyl, fluorodihydrofuranyl, and the like; A non-limiting example of a suitable multicyclic oxaheterocyclenyl group is 7-oxabicyclo[2.2.1]heptenyl; non-limiting examples of suitable monocyclic thiaheterocyclenyl rings include dihydrothiophenyl, dihydrothiopyranyl, and the like;
“heterocycloalkylalkyl” (or heterocyclylalkyl) means a heterocycloalkyl-alkyl-group (i.e., the bond to the parent moiety is through the alkyl group) wherein the heterocycloalkyl group (i.e., the heterocyclyl group) is unsubstituted or substituted as defined below, and the alkyl group is unsubstituted or substituted as defined above;
“heterocyclyl” (or heterocycloalkyl) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination; there are no adjacent oxygen and/or sulfur atoms present in the ring system; preferred heterocyclyls contain about 5 to about 6 ring atoms; the prefix aza, oxa or thia before the heterocyclyl root name means that at least a nitrogen, oxygen or sulfur atom respectively is present as a ring atom; the heterocyclyl can be optionally substituted by one or more independently selected “ring system substituents” (defined below); the nitrogen or sulfur atom of the heterocyclyl can be optionally oxidized to the corresponding N-oxide, S-oxide or S,S-dioxide; non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like;
“hydroxyalkyl” means a HO-alkyl-group wherein the alkyl group is substituted or unsubstituted as defined above; preferred hydroxyalkyls comprise a lower alkyl; Non-limiting examples of suitable hydroxyalkyl groups include hydroxymethyl and 2-hydroxyethyl; and
“ring system substituent” means a substituent attached to an aromatic or non-aromatic ring system that, for example, replaces an available hydrogen on the Ting system; ring system substituents are each independently selected from the group consisting of: alkyl, aryl, heteroaryl, aralkyl, alkylaryl, aralkenyl, heteroalkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, R⁶⁰R⁶⁵N—, R⁶⁰R⁶⁵N-alkyl-, R⁶⁰R⁶⁵NC(O)— and R⁶⁰R⁶⁵NSO₂—, wherein R⁶⁰ and R⁶⁵ are each independently selected from the group consisting of: hydrogen, alkyl, aryl, and aralkyl; “Ring system substituent” also means a cyclic ring of 3 to 7 ring atoms, wherein 1-2 ring atoms can be heteroatoms, attached to an aryl, heteroaryl, heterocyclyl or heterocyclenyl ring by simultaneously substituting two ring hydrogen atoms on said aryl, heteroaryl, heterocyclyl or heterocyclenyl ring; Non-limiting examples include:

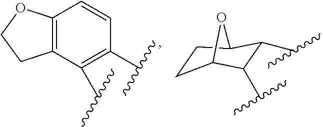

and the like

Lines drawn into a ring mean that the indicated bond may be attached to any of the substitutable ring carbon atoms.

Any carbon or heteroatom with unsatisfied valences in the text, schemes, examples, structural formulae, and any Tables herein is assumed to have the hydrogen atom or atoms to satisfy the valences.

One or more compounds of the invention may also exist as, or optionally converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy; show the presence of the solvent (or water) in the crystals as a solvate (dr hydrate).

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, capsules, pills and the like. Similarly, the herein-described methods of treating a patient by administering a pharmaceutical composition of the present invention is also intended to encompass the administration of the afore-said bulk composition and individual dosage units.

Prodrugs of the compounds of the invention are also contemplated herein. The term “prodrug”, as employed herein, denotes a compound that is a drug precursor which, upon administration to a subject, undergoes chemical conversion by metabolic or chemical processes to yield a compound of formula 1.0 or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

For example, if a compound of formula 1.0, or a pharmaceutically acceptable salt, hydrate or solvate of the compound, contains a carboxylic acid functional group, a prodrug can comprise an ester formed by the replacement of the hydrogen atom of the acid group with a group such as, for example, (C₁-C₈)alkyl, (C₂-C₁₂)alkanoyloxy-methyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxy-carbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl); carbamoyl-(C₁-C₂)alkyl, N,N-di (C₁-C₂)alkylcarbamoyl-(C1-C2)alkyl and piperidino-, pyrrolidino, or morpholino(C₂-C₃)alkyl, and the like.

Similarly, if a compound of formula 1.0 contains an alcohol functional group, a prodrug can be formed by the replacement of the hydrogen atom of the alcohol group with a group such as, for example, (C₁-C₆)alkanoyloxymethyl, 1-((C₁-C₆)alkanoyl-oxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of formula 1.0 incorporates an amine functional group, a prodrug can be formed by the replacement of a hydrogen atom in the amine group with a group such as, for example, R⁷⁰-carbonyl, R⁷⁰O-carbonyl, NR⁷OR⁷⁵-carbonyl where R⁷⁰ and R⁷⁵ are each independently (C₁-C₁₀)alkyl, (C₃-C₇) cycloalkyl, benzyl, or R⁷⁰-carbonyl is a natural α-aminoacyl or natural α-aminoacyl, —C(OH)C(O)OY⁸⁰ wherein Y⁸⁰ is H, (C₁-C₆)alkyl or benzyl, —C(OY⁸²)Y⁸⁴ wherein Y⁸² is (C₁-C₄) alkyl and Y⁸⁴ is (C₁-C₆)alkyl, carboxy (C₁-C₆)alkyl, amino(C₁-C₄)alkyl or mono-N- or di-N,N—(C₁-C₆)alkylaminoalkyl, —C(Y⁸⁶)Y⁸⁸ wherein Y⁸⁶ is H or methyl and Y⁸⁸ is mono-N- or di-N,N—(C₁-C₆)alkylamino morpholino, piperidin-1-yl or pyrrolidin-1-yl, and the like.

This invention also includes the compounds of this invention in isolated and purified form.

Polymorphic forms of the compounds of formula 1.0, and of the salts, solvates and prodrugs of the compounds of formula 1.0, are intended to be included in the present invention.

Certain compounds of the invention may exist in different isomeric (e.g., enantiomers, diastereoisomers, atropisomers) forms. The invention contemplates all such isomers both in pure form and in admixture, including racemic mixtures. Enol forms are also included.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons); rotameric forms, atropisomers; and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Diasteromeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diasteromeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

The compounds of formula 1.0 form salts that are also within the scope of this invention. Reference to a compound of formula 1.0 herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formula 1.0 contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable salts) are preferred. Salts of the compounds of the formula 1.0 may be formed, for example, by reacting a compound of formula 1.0 with an amount of acid or base, such as an equivalent amount, in a medium such as one in which the salt precipitates or in an aqueous medium followed by lyophilization. Acids (and bases) which aregenerally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Intl. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexyl-amine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid and base salts are intended to be pharmaceutically acceptable salts within the scope of the invention and all acid and base salts are considered equivalent to the free forms of the corresponding compounds for purposes of the invention.

Compounds of formula 1.0, and salts, solvates and prodrugs thereof, may exist in their tautomeric form (for example, as an amide or imino ether). All such tautomeric forms are contemplated herein as part of the present invention.

In hetero-atom containing ring systems of this invention, there are no hydroxyl groups on carbon atoms adjacent to a N, O or S, and there are no N or S groups on carbon adjacent to another heteroatom. Thus, for example, in the ring:

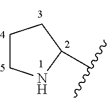

there is no —OH attached directly to carbons marked 2 and 5.

The compounds of formula 1.0 may exist in different tautomeric forms, and all such forms are embraced within the scope of the invention. Also, for example, all keto-enol and imine-enamine forms of the compounds are included in the invention.

Tautomeric forms such as, for example, the moieties:

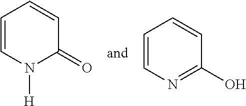

are considered equivalent in certain embodiments of this invention.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R³, etc.) occurs more than one time in any moiety or in any compound of formula 1.0, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of formula 1.0 (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of formula 1.0 can generally be prepared by following procedures analogous to those disclosed in the Schemes and/or in the Examples hereinbelow, by substituting an appropriate isotopically labelled reagent for a non-isotopically labelled reagent.

This invention provides compounds of formula 1.0:

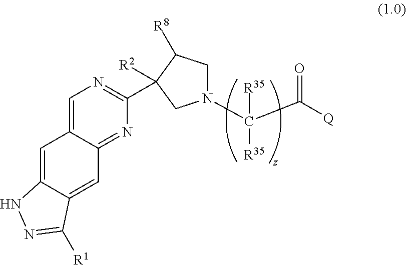

or the pharmaceutically acceptable salts, esters or solvates thereof, wherein:

z is 1 to 3 (i.e., 1, 2 or 3, and preferably 1);

Q is a substituent selected from the group consisting of:

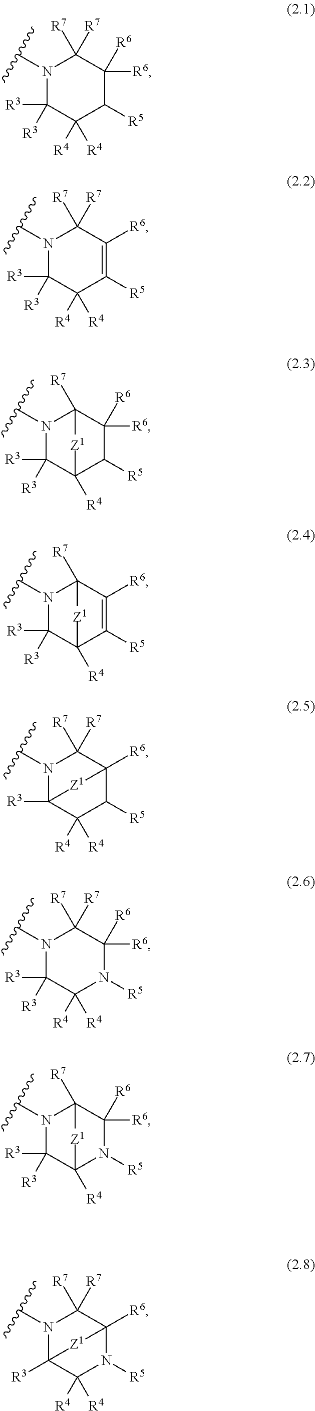

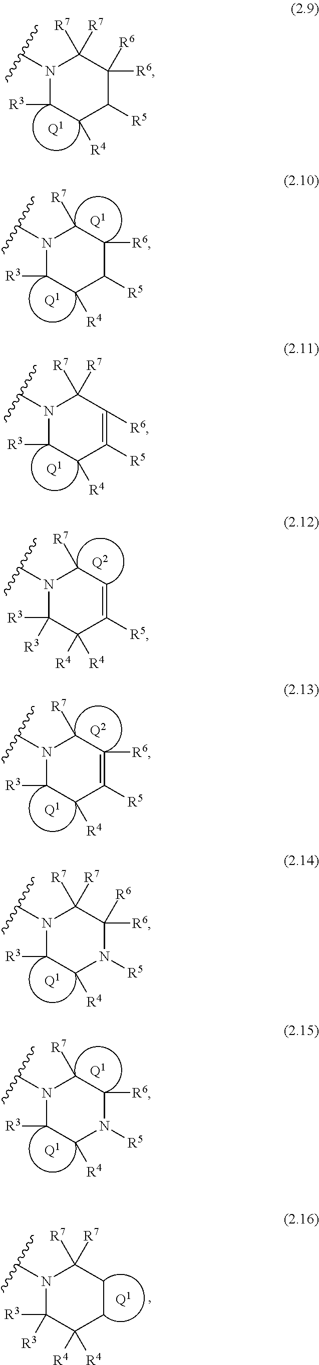

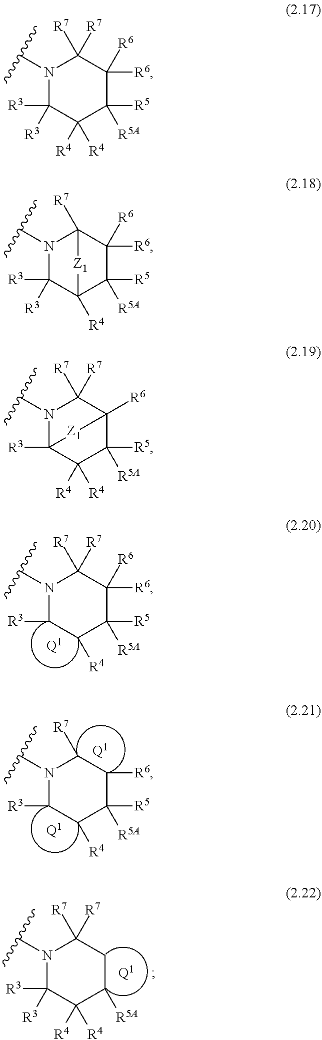

Each Q¹ represents a ring independently selected from the group consisting of: cycloalkyl, substituted cycloalkyl, heterocycloalkyl, substituted heterocycloalkyl, aryl, substituted aryl, heteroaryl, and substituted heteroaryl, wherein said substituted rings are substituted with 1 to 3 substituents independently selected from the group consisting of: halo (e.g., Cl, F, Br) and the R¹⁰ moieties; provided that when Q¹ is aryl, heteroaryl, substituted aryl or substituted heteroaryl then the carbon atoms at the ring junction (i.e., the two carbon atoms common to the fused rings) are not substituted;

Q² represents a ring selected from the group consisting of: cycloalkyl, substituted cycloalkyl, heterocycloalkyl, and substituted heterocycloalkyl, wherein said substituted rings are substituted with 1 to 3 substituents independently selected from the group consisting of: the R¹⁰ moieties;

Z¹ represents —(C(R²⁴)₂)_{w}— wherein each R²⁴ is independently selected from the group consisting of: H, alkyl (e.g., C₁ to C₆ alkyl, for example methyl) and F, and wherein w is 1, 2 or 3, and generally w is 1 or 2, and usually w is 1, and wherein in one example each R²⁴ is H, and in another example w is 1, and in another example each R²⁴ is H and w is 1, preferably w is 1 and each R²⁴ is H (i.e., preferably Z¹ is —CH₂—);

Z² is selected from the group consisting of: —N(R⁴⁴)—, —O— and —C(R⁴⁶)₂— (e.g., Z² is —NH—, —O— or —CH₂—);

m is 1 to 6;

n is 1 to 6;

p is 0 to 6;

t is 0, 1, or 2;

R¹ is selected from the group consisting of:
(1) —CN,
(2) —NO₂,
(3) —OR¹⁰,
(4) —SR¹⁰,
(5) —N(R¹⁰)₂,
(6) R¹⁰,
(7) —C(O)R¹⁰ (in one example R¹⁰ is a 4 to 6 membered heterocycloalkyl ring, in another example R¹⁰ is a 4 to 6 membered heterocycloalkyl ring comprising one nitrogen atom, and in another example R¹⁰ is a 4 to 6 membered heterocycloalkyl ring comprising one nitrogen atom wherein said ring is bound to the carbonyl moiety (—C(O)—) through the ring nitrogen),
(8) —(C(R³⁰)₂)ₙ—NR³²—C(O)—R¹⁰ (e.g., —(CH₂)ₙ—NH—C(O)—R¹⁰, for example wherein n is 1), wherein in one example n is 1, each R³⁰ is H, R³² is H, and R¹⁰ is selected from the group consisting of: cycloalkyl (e.g., cyclopropyl) and alkyl (e.g., methyl and i-propyl), and wherein in another example n is 1, each R³⁰ is H, R³² is H, and R¹⁰ is selected from the group consisting of: methyl, i-propyl and cyclopropyl,
(9)) —(C(R³⁰)₂)ₙ—NR³²—S(O)ᵣR¹⁰ (e.g., —(CH₂)ₙ—NH—S(O)ₜ—R¹⁰, for example wherein n is 1 and t is 2) wherein in one example n is 1, each R³⁰ is H, R³² is H, t is 2, and R¹⁰ is selected from the group consisting of: cycloalkyl (e.g., cyclopropyl) and alkyl (e.g., methyl and i-propyl), and wherein in another example n is 1, each R³⁰ is H, R³² is H, t is 2, R¹⁰ is selected from the group consisting of: methyl, i-propyl and cyclopropyl, and wherein in another example n is 1, each R³⁰ is H, R³² is H, t is 2, and R¹⁰ is methyl,
(10) —(C(R³⁰)₂)ₙ—NR³²—C(O)—N(R³²)—R¹⁰ (e.g., —(CH₂)ₙ—NH—C(O)—NH—R¹⁰, for example wherein n is 1) wherein in one example n is 1, each R³⁰ is H, each R³² is H, and R¹⁰ is alkyl (e.g., methyl and i-propyl), and wherein in another example n is 1, each R³⁰ is H, each R³² is H, and R¹⁰ is selected from the group consisting of: methyl and i-propyl,
(11)

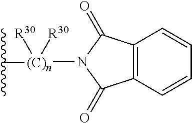

wherein in one example n is 1 and each R³⁰ is H, i.e., a moiety of the formula:

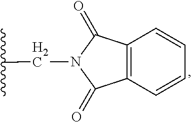

(12) —CF₃,
(13) —C(O)OR¹⁰ wherein in one example R¹⁰ is selected from the group consisting of: H, alkyl (e.g., methyl and ispropyl) and cyclopropyl (e.g., cyclopropyl), and wherein in another example R¹⁰ is selected from the group consisting of: H and alkyl, and wherein in another example R¹⁰ is selected from the group consisting of: H and methyl,
(14)) —(C(R³⁰)₂)ₙR¹³ (e.g., —(CH₂)ₙR¹³) wherein in one example n is 1, each R³⁰ is H, and R¹³ is selected from the group consisting of: —OH and —N(R¹⁰)₂, wherein each R¹⁰ is independently selected, and wherein in another example n is 1, each R³⁰ is H, and R¹³ is selected from the group consisting of: —OH and —N(R¹⁰)₂, and each R¹⁰, is H (i.e., R¹³ is —OH or —NH₂),
(15) alkenyl (e.g., —CH═CHCH₃),
(16) —NR³²—C(O)—R¹⁴ (e.g., —NH—C(O)—R¹⁴) wherein in one example R³² is H and R¹⁴ is selected from the group consisting of: cycloalkyl (e.g., cyclopropyl), alkyl (e.g., methyl and propyl), aryl (e.g., phenyl), amino (i.e., —NH₂), and heteroaryl (e.g., pyridyl, such as, for example 2-pyridyl, 3-pyridyl, 4-pyridyl, pyrazolyl and imidazolyl), and wherein in another example R³² is H and R¹⁴ is selected from the group consisting of: cyclopropyl, methyl, propyl, phenyl, and amino,
(17)

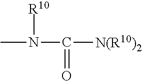

wherein each R¹⁰ is independently selected, for example:
   (a) in one example moiety (20) is:

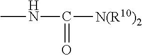

wherein each R¹⁰ is independently selected,
(b) in another example moiety (20) is:

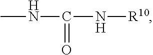

and
(c) in another example moiety (20) is:

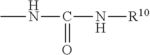

wherein R¹⁰ is selected from the group consisting of: aryl (e.g., phenyl) and alkyl (e.g., ethyl, and preferably R¹⁰ is phenyl or ethyl,
(18)

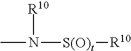

wherein each R¹⁰ is independently selected, and wherein in one example each R¹⁰ is independently selected and t is 2, and wherein in another example moiety (18) is —NH—S(O)ₜ—R¹⁰, and wherein in another example moiety (18) is —NH—S(O)ᵣR¹⁰ wherein t is 2, and wherein in another example moiety (18) is —NH—S(O)ₜ—R¹⁰. t is 2, and R¹⁰ is alkyl (e.g., methyl),
(19)

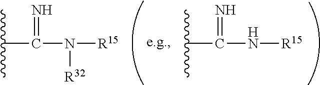

(also written as —C(NH)N(R¹⁵)R³² and —C(NH)NH(R¹⁵), respectively), wherein in one example R¹⁵ is —OH, and in another example R³² is H and R¹⁵ is —OH,
(20) —C(O) —NR³²—(C(R³⁰)₂)ₚ—OR¹⁰ (e.g., —C(O)—NH—(CH₂)ₚ—OR¹⁰, and, for example, —C(O)—NH—(CH₂)ₚ—OR¹⁰ wherein p is 2) wherein:
   (a) in one example p is 2,
   (b) in another example R³² is H,
   (c) in another example R¹⁰ is selected from the group consisting of: H and alkyl (e.g., methyl),
   (d) in another example R¹⁰ is selected from the group consisting of: H and alkyl (e.g., methyl), and R³² is H,
   (e) in another example R¹⁰ is selected from the group consisting of: H and alkyl (e.g., methyl), R³² is H, an p is 2,
   (f) in another example R³² is H, each R³⁰ is H, and R¹⁰ is alkyl,
   (g) in another example R³² is H, each R³⁰ is H, and R¹⁰ is methyl,
   (h) in another example R³² is H, each R³⁰ is H, p is 2 and R¹⁰ is alkyl, and
   (i) in another example R³² is H, each R³⁰ is H, p is 2 and R¹⁰ is methyl,
(21) —C(O)N(R¹⁰)₂ wherein each R¹⁰ is independently selected, and preferably each R¹⁰ is independently selected from the group consisting of: (a) H, (b) alkyl (e.g., methyl, butyl, and i-propyl), (c) heteroaryl (e.g., pyridyl), (d) aryl (e.g., phenyl), and (e) cycloalkyl (e.g., cyclopropyl), wherein for example, each R¹⁰ is selected from the group consisting of: H, methyl, butyl, i-propyl, pyridyl, phenyl and cyclopropyl, wherein, for example, said —C(O)N(R¹⁰)₂ moiety is selected from the group consisting of: —C(O)NH₂, —C(O)NH(CH₃), —C(O)NH(CH)(CH₃)₂ (i.e., —C(O)NH(i-propyl)), —C(O)NH(C₄H₉), —C(O)NH(C₆H₅) (i.e., —C(O)NH(phenyl)), —C(O)NH(C₃H₅) (i.e., —C(O)NH(cyclopropyl), and —C(O)NH(C₅H₄N) (i.e., —C(O)NH(pyridyl), such as

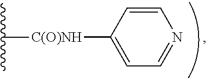

(22) —C(O)—NR³²—C(R¹⁸)₃ (e.g., —C(O)—NH—C(R¹⁸)₃) wherein each R¹⁸ is independently selected from the group consisting of: R¹⁰ and —C(O)OR¹⁹, and R¹⁹ is selected from the group consisting of:alkyl (e.g., methyl) and substituted arylalkyl (e.g., —CH₂C₆H₄OH (i.e., hydroxybenzyl) such as, for example, -p-CH₂C₆H₄OH (i.e., p-OHbenzyl), and wherein:
   (a) in one example R¹⁸ and R¹⁹ are as defined above with the proviso that at least one R¹⁸ substitutent is other than H (e.g., in one example one R¹⁸ is H and the remaining two R¹⁸ groups are other than H, and in another example two R¹⁸ substituents are H and the remaining R¹⁸ substituent is other than H),
   (b) in another example R¹⁸ is selected from the group consisting of: H, aryl (e.g., phenyl), substituted aryl (e.g., substituted phenyl, such as, for example halophenyl-, such as, for example, fluorophenyl (e.g., o-F-phenyl)), and —C(O)OR¹⁹,
   (c) in another example R¹⁸ is selected from the group consisting of: H, phenyl, fluorophenyl (e.g., o-F-phenyl), —C(O)OCH₃, —C(O)OCH₂C₆H₄OH (i.e., —C(O)O(OHbenzyl), such as, —C(O)O(p-OHbenzyl)),
   (d) in another example R¹⁸ is selected from the group consisting of: H, aryl (e.g., phenyl), substituted aryl (e.g., substituted phenyl, such as, for example halophenyl-, such as, for example, fluorophenyl (e.g., o-F-phenyl)), and —C(O)OR¹⁹, provided that at least one R¹⁸ substitutent is other than H (e.g., in one example one R¹⁸ is H and the remaining two R¹⁸ groups are other than H, and in another example two R¹⁸ substituents are H and the remaining R¹⁸ substituent is other than H),
   (e) in another example R¹⁸ is selected from the group consisting of: H, phenyl, fluorophenyl (e.g., o-F-phenyl), —C(O)OCH₃, —C(O)OCH₂C₆H₄OH (i.e., —C(O)O(OHbenzyl), such as, —C(O)O(p-OHbenzyl)), provided that at least one R¹⁸ substitutent is other than H (e.g., in one example one R¹⁸ is H and the remaining two R¹⁸ groups are other than H, and in another example two R¹⁸ substituents are H and the remaining R¹⁸ substituent is other than H),
   (f) in another example R³² is H, and each R¹⁸ is independently selected from the group consisting of: R¹⁰ and —C(O)OR¹⁹, and R¹⁹ is selected from the group consisting of:alkyl (e.g., methyl) and substituted arylalkyl (e.g., —CH₂C₆H₄OH (i.e., hydroxybenzyl) such as, for example, -p-CH₂C₆H₄OH (i.e., p-OHbenzyl),
   (g) in another example R³² is H and R¹⁸ and R¹⁹ are as defined in paragraph (a),
   (h) in another example R³² is H and R¹⁸ and R¹⁹ are as defined in paragraph (b),
   (i) in another example R³² is H and R¹⁸ and R¹⁹ are as defined in paragraph (c),
   (j) in another example R³² is H and R¹⁸ and R¹⁹ are as defined in paragraph (d),
   (k) in another example R³² is H and R¹⁸ and R¹⁹ are as defined in paragraph (e), and
   (l) in another example R³² is H and R¹⁸ and R¹⁹ are as defined in paragraph (f),
(23) —C(O)—NR³²—(C(R³⁰)₂)ₙ—C(O)—N(R¹⁰)₂ (e.g., —C(O)—NH—(CH₂)ₙ—C(O)—NH₂), and wherein:
   in one example R³² is H,
   in another example each R³⁰ is H,
   in another example n is 1,
   in another example n is 1 and R³² is H,
   in another example each R¹⁰ is H,
   in another example R³² is H and each R³⁰ is H,
   in another example R³² is H, each R³⁰ is H and n is 1,
   in another example R³² is H, each R³⁰ is H, n is 1, and each R¹⁰ is H,
   in another example R³² is H, n is 1, each R³⁰ is independently selected from the group consisting of: H and alkyl, and each R¹⁰ is independently selected from the group consisting of: H and alkyl, and
   in another example R³² is H, n is 1, and each R³⁰ is independently selected from the group consisting of: H, methyl, ethyl and i-propyl (or each R³⁰ is independently selected from the group consisting of H and i-propyl, or one R³⁰ is i-propyl and the other R³⁰ is H), and each R¹⁰ is independently selected from the group consisting of: H methyl, ethyl and i-propyl (or each R¹⁰ is H),
(24) heterocycloalkenyl, such as, for example:

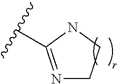

wherein r is 1 to 3, and wherein in one example r is 1, i.e., in one, example the heterocycloalkenyl is dihydroimidazolyl, such as, for example:

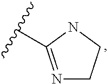

(25)

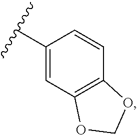

(26) arylalkenyl-(aralkenyl-), for example, aryl(C₂ to C₆)alkenyl-, such as for example, —CH═CH-phenyl, and
(27) halo (e.g., Br, CI, and F, and in one example, Br);

R² is selected from the group consisting of:
(1) H,
(2) —CN,
(3) halo (e.g., F),
(4) alkyl (e.g., C₁ to C₆ alkyl, such as, for example, methyl, ethyl and propyl),
(5) substituted alkyl (e.g., substituted C₁ to C₆ alkyl, such as, for example, substituted methyl and substituted ethyl) wherein said substituted alkyl is substituted with 1 to 3 substitutents (e.g., 1 substituent) selected from the group consisting of: (a) —OH, (b) —O-alkyl (e.g., —O—(C₁-C₃alkyl), such as, for example, —OCH₃), (c) —O-alkyl (e.g., —O—(C₁-C₃alkyl)) substituted with 1 to 3 F atoms (examples of said —O-substituted alkyl portion include, but are not limited to, —OCHF₂ and —OCF₃), and (d) —N(R⁴⁰)₂ wherein each R⁴⁰ is independently selected from the group consisting of: (i) H, (ii) C₁-C₃ alkyl (e.g., methyl), (iii) —CF₃, and (e) halo (for example F, Cl, and Br, and also for example F, examples of a halo substituted alky group include, but are not limited to, —CHF₂), (examples of said substituted alkyl groups described in (5) include but are not limited to —CH(OH)CH₃, —CH₂OH, and —CH₂OCH₃),
(6) alkynyl (e.g., ethynyl),
(7) alkenyl (e.g., —CH₂—CH═CH₂),
(8) —(CH₂)ₘR¹¹,
(9) —N(R²⁶)₂,
(10) —OR²³ (e.g., —OH, —OCH₃ and —O-phenyl),
(11) —N(R²⁶)C(O)R⁴² wherein in one example R²⁶ is H or C₁ to C₆ alkyl (e.g., methyl) and R⁴² is alkyl (e.g., methyl), and in another example —N(R²⁶)C(O)R⁴² is —NHC(O)CH₃,
(12) cycloalkyl (e.g., C₃ to C₆ cycloalkyl, such as, for example, cyclopropyl and cyclohexyl),
(13) cycloalkylalkyl (e.g., C₃ to C₆ cycloalkyl-(C₁ to C₃)alkyl-, such as, for example, cyclopropyl-CH₂— and cyclohexyl-CH₂—),
(14)

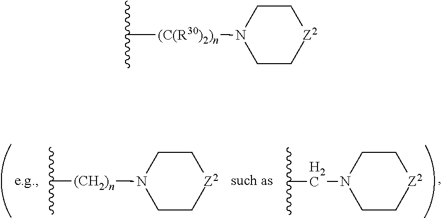

(15) —O-(substituted alkyl) wherein said substituted alkyl is substituted with 1 to 3 F atoms (examples of said —O-(substituted alkyl) moiety include, but are not limited to, —OCHF₂ and —OCF₃),
(16) —S(O)ₜ-alkyl, such as, for example, (a) —S-alkyl (i.e., t is 0) such as, for example, —S—CH₃, and (b) —S(O)₂-alkyl (i.e., t is 2) such as, for example, —S(O)₂CH₃,
(17) —C(O)-alkyl (e.g., —C(O)CH₃),
(18)

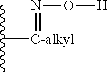

wherein methyl is an example of said alkyl moiety,
(19)

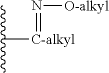

wherein each alkyl is independently selected, examples of this moiety include, but are not limited to:

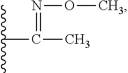

(20)

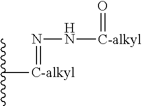

which each alkyl is independently selected, examples of this moiety include, but are not limited to,

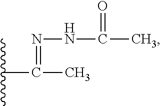

(21)

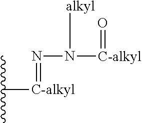

wherein each alkyl is independently selected,
(22) —N(R⁴⁸)—C(O)—R⁴⁸ wherein each R⁴⁸ is independently selected from the group consisting of: H and alkyl (e.g., C₁ to C₆ alkyl, such as, for example, methyl), and wherein examples of this moiety include, but are not limited to, —NH—C(O)—H, and —N(CH₃)—C(O)H, and
(23) —C(O)-alkyl, such as, for example, —C(O)—(C₁-C₆ alkyl), such as, for example, —C(O)CH₃; and

wherein:
(a) in one example said (14) moiety is

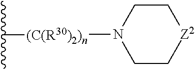

and n is 1,
(b) in another example said (14) moiety is

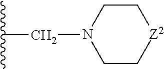

(i.e., n is 1, and each R³⁰ is H),
(c) in another example Z² is —NH— in (a),
(d) in another example Z² is —NH— in (b),
(e) in another example Z² is —O— in (a),
(f) in another example Z² is —O— in (b),
(g) in another example Z² is —CH₂— in (a),
(h) in another example Z² is —CH₂— in (b),
(i) in another example R² is —(CH₂)ₘR¹¹ and m is 1,
(j) in another example R² is —N(R²⁶)₂,
(k) in another example R² is —N(R²⁶)₂, and each R²⁶ is H (i.e., R² is —NH₂),
(l) in another example R² is —OR²³, and
(m) in another example R² is —OH (i.e., R²³ is H);

each R³, R⁴, R⁵, R⁶ and R⁷ is independently selected from the group consisting of:
(1) H,
(2) alkenyl (e.g., —CH₂CH═CH₂),
(3) substituted alkenyl,
(4) alkyl,
(5) substituted alkyl,
(6) cycloalkyl,
(7) substituted cycloalkyl,
(8) cycloalkylalkyl-,
(9) substituted cycloalkylalkyl-,
(10) heterocycloalkyl,
(11) substituted heterocycloalkyl,
(12) heterocycloalkylalkyl-,
(13) substituted heterocycloalkylalkyl-,
(14) —C(O)R¹⁰ wherein in one example R¹⁰ is selected from the group consisting of: alkyl (e.g., C₁ to C₆, e.g., methyl),
(15) arylheteroaryl- (e.g., phenylthiadiazolyl-),
(16) substituted arylheteroaryl- (e.g., substituted phenylthiadiazolyl-),
(17) heteroarylaryl-, such as, for example, pyrimidinylphenyl-, pyrazinylphenyl-, pyridinylphenyl- (i.e., pyridylphenyl-), furanylphenyl-, thienylphenyl-, thiazolylphenyl-, oxadiazolylphenyl-, and pyridazinylphenyl-,
(18) substituted heteroarylaryl-, such as, for example, substituted pyrimidinylphenyl-, substituted pyrazinylphenyl-, substituted pyridinylphenyl- (i.e., substituted pyridylphenyl-), substituted furanylphenyl-, substituted thienylphenyl-, substituted thiazolylphenyl-, substituted pyrimidinylphenyl, substituted oxadiazolylphenyl-, and substituted pyridazinylphenyl-,
(19) aryl (e.g., phenyl),
(20) substituted aryl (e.g., substituted phenyl),
(21) heteroaryl (e.g., thiazolyl, thienyl, pyridyl, and pyrimidinyl),
(22) substituted heteroaryl (e.g., substituted thiazolyl, substituted pyridyl and substituted pyrimidinyl), examples of substituted heteroaryl groups include, for example bromothiazolyl-, bromopyrimidinyl-, fluoropyrimidinyl-, and ethenylpyrimidinyl-,
(23) heteroarylheteroaryl- (e.g., pyrimidinylpyridyl-, pyrimidinylthiazolyl-, and pyrimidinylpyrazinyl-),
(24) substituted heteroarylheteroaryl- (e.g., substituted pyrimidinylpyridyl-, and substituted pyrimidinylpyrazinyl-),
(25) arylaminoheteroaryl- (e.g., phenyl-NH-oxadiazolyl-),
(26) substituted arylaminoheteroaryl- (e.g., substituted phenyl-NH-oxadiazolyl-),
(27) arylalkynyl- (e.g., aryl(C₂ to C₄)alkynyl such as, for example phenylethynyl-),
(28) substituted arylalkynyl- (e.g., substituted aryl(C₂ to C₄)alkynyl-, such as, for example, substituted phenylethynyl-),
(29) heteroarylalkynyl- (e.g., heteroaryl(C₂ to C₄)alkynyl-, such as, for example, pyrimidinylethynyl-),
(30) substituted heteroarylalkynyl- (e.g., substituted heteroaryl(C₂ to C₄)alkynyl-, such as, for example substituted pyrimidinylethynyl-),
(31) benzoheteroaryl (i.e., a fused phenyl and heteroaryl rings), such as, for example, benzothiazole and quinoxaline;

wherein said R³, R⁴, R⁵, R⁶ and R⁷ substituted groups (7), (9), (11), (13), (16), (18), (20), (22), (24), (26), (28) and (30) are substituted with 1 to 3 substituents independently selected from the group consisting of: —NH₂, —NHR²⁰ (e.g., —NHCH₂CH₃ and —NHCH₃), —N(R²⁰)₂ wherein each R²⁰ is independently selected, alkyl (e.g., C₁ to C₆ alkyl, e.g., methyl, ethyl, and i-propyl), alkenyl (e.g., C₂ to C₆ alkenyl, such as, for example —CH═CH₂), halo (e.g., F, Cl and Br, and in another example F), —C(O)—NH—R²⁸ (e.g., —C(O)—NH—CH₃), —C(O)OR²⁸ (e.g., —C(O)OC₂H₅), —C(O)R²⁸ (e.g., —C(O)CH₃), and —OR²⁰ (e.g., —OCH₃),

wherein said R³, R⁴, R⁵, R⁶ and R⁷ substituted groups (3) and (5) are substituted with 1 to 3 substituents independently selected from the group consisting of: —NH₂, halo (e.g., F, Cl and Br, and in another example F), —C(O)—NH—R²⁸ (e.g., —C(O)—NH—CH₃), —C(O)OR²⁸ (e.g., —C(O)OC₂H₅), and —C(O)R²⁸ (e.g., —C(O)CH₃), and

wherein:
in one example said substituted heteroarylaryl (moiety (18) above) is substituted with 1 to 3 substituents independently selected from the group consisting of: —NH₂, alkyl (e.g., C₁ to C₆ alkyl, e.g., methyl), halo (e.g., F, Cl and Br, such as, for example F),
in another example said substituted aryl (moiety (20) above) is substituted with 1 to 3 substituents independently selected from the group consisting of halo (e.g., F, Cl and Br), —C(O)—NH—R²⁶ (e.g., —C(O)—NH—CH₃), —C(O)OR²⁸ (e.g., —C(O)O—C₂H₅), and —C(O)R²⁸ (e.g., —C(O)CH₃), and in another example said substituted heteroaryl (moiety (22) above) is substituted with 1 to 3 substitutents selected from the group consisting of: halo (e.g., Br, F, and Cl), alkenyl (e.g., C₂ to C₆ alkenyl, such as, for example, —CH═CH₂);

R^{5A} is selected from the group consisting of: halo (for example, F, Cl, and Br, and in another example F), —OH, alkyl (e.g., C₁ to C₆ alkyl, such as, for example, —CH₃), —O-alkyl (such as, for example, —O—(C₁ to C₆ alkyl), also, for example, —O—(C₁ to C₃ alkyl), also for example, —O—(C₁ to C₂ alkyl), and in one example —O—CH₃);

R⁸ is selected from the group consisting of: H, —OH, —N(R¹⁹)₂ (e.g., —NH₂), —NR¹⁹C(O)R¹² (e.g., —NHC(O)CH₃), and alkyl (e.g., methyl);

each R⁹ is independently selected from the group consisting of:halogen, —CN, —NO₂, —OR¹⁹, —SR¹⁰, —N(R¹⁰)₂, and R¹⁰;

each R¹⁹ is independently selected from the group consisting of: H, alkyl, aryl, arylalkyl, heteroaryl, heteroarylalkyl, cycloalkyl, cycloalkylalkyl, heterocycloalkyl, heterocycloalkylalkyl, alkylheteroaryl-, alkylaryl-, substituted alkyl, substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl-, substituted alkylaryl-, heterocycloalkenyl

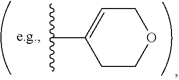

and substituted heterocycloalkenyl, and wherein:

said R¹⁰ substituted alkyl is substituted with 1 to 3 substituents independently selected from the group consisting of: —NH₂, —NHR²⁰, —NO₂, —CN, —OR²⁸, halo (e.g., F; Cl and Br, and in another example F), —C(O)—NH—R²⁶ (e.g., —C(O)—NH—CH₃, i.e., R²⁶ is alkyl, such as methyl), —C(O)OR²⁸ (e.g., —C(O)OC₂H₅, i.e., R²⁶ is alkyl, such as ethyl), and —C(O)R²⁶ (e.g., —C(O)CH₃, i.e., R²⁶ is alkyl, such as methyl), and

said R¹⁰ substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl- are substituted with 1 to 3 substituents independently selected from the group consisting of: (1) —NH₂, (2) —NO₂, (3) —CN, (4) —OH, (5) —OR²⁰, (6) —OCF₃, (7) alkyl (e.g., C₁ to C₆ alkyl) substituted with 1 to 3 independently selected halo atoms (e.g., F, Cl and Br), examples of the substituted alkyl include, but are not limited to, —CF₃, —CHF₂ and—CH₂F, (8) —C(O)R³⁸ (e.g., R³⁸ is H or alkyl (e.g., C₁ to C₆ alkyl, such as, for example, methyl or ethyl), for example, R³⁸ is alkyl (e.g., methyl), thus, an example of —C(O)R³⁸ is —C(O)CH₃), (9) alkyl (e.g., C₁ to C₆ alkyl, e.g., methyl, ethyl, and i-propyl), (10) alkenyl (e.g., C₂ to C₆ alkenyl, such as, for example —CH═CH₂), (11) halo (e.g., F, Cl and Br, and in another example F), (12) —C(O)—NH—R²⁶ (e.g., —C(O)—NH—CH₃), (13) —C(O)OR³⁸ (e.g., R³⁸ is H or alkyl (e.g., C₁ to C₆ alkyl, such as, for example, methyl or ethyl), for example, R³⁸ is alkyl (e.g., methyl or ethyl), thus, for example, —C(O)OR³⁸ is —C(O)OC₂H₅), (14) —C(O)—NR³²—(C(R³⁰)₂)ₙ—N(R³⁸)₂ (e.g., —C(O)—NH—(CH₂)ₙ—N(R³⁸)₂) (wherein (a) in one example R³² is H, (b) in another example each R³⁰ is H, (c) in another example n is 2, (d) in another example each R³⁸ is independently selected, (e) in another example each R³⁸ is independently selected from the group consisting of: H and alkyl (e.g., methyl), (f) in another example R³² is H, each R³⁰ is H, and each R³⁸ is independently selected, (g) in another example R³² is H, each R³⁰ is H, and each R³⁸ is independently selected from the group consisting of: H and alkyl (e.g., methyl), (15) —S(O)ₜR³⁸ (wherein in one example t is 2, and in another example R³⁸ is alkyl (e.g., methyl or isopropyl), and in another example t is 2 and R³⁸ is alkyl (e.g., methyl or isopropyl)), (16) —C(O)—NR³²—R³⁸ (e.g., —C(O)—NR³²—R³⁸) (wherein one example R³² is H, in another example R³⁸ is alkyl (e.g., propyl), and in another example R³² is H and R³⁸ is alkyl (e.g., propyl)), (17) —NR³²—C(O)—R³⁸ (e.g., —NH—C(O)—R³⁸) (wherein in one example R³² is H, in another example R³⁸ is alkyl (e.g., methyl), and in another example R³² is H and R³⁸ is alkyl (e.g., methyl)), (18)

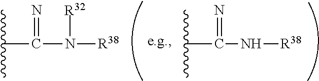

(wherein in one example R³² is H, in another example R³⁸ is H, and in another example R³² is H and R³⁸ is H), (19) —NHR²⁰ (e.g., —NHCH₃, —NHC₂H₅), (20) cycloalkyl (e.g., C₃ to C₆ cycloalkyl, such as, for example, cyclopropyl), (21) —O-alkyl-O—R²⁰ (e.g., —O—(C₁ to C₆)alkyl-OR²⁰, such as, for example, —O—CH₂CH₂—OCH₃), (22) hydroxyalkyl (e.g., hydroxy(C₁ to C₆)alkyl, such as, for example, —CH₂OH and —C(CH₃)₂OH), (23) —N(R²⁰)₂ wherein each R²⁰ is independently selected (e.g., —N(CH₃)₂), (24) -alkyl-OR²⁰ (e.g., —(C₁ to C₆)alkyl-OR²⁰, such as, for example, —CH₂OCH₃), (25) —O-alkyl-OH (e.g., —O—(C₁ to C₆)alkyl-OH, such as, for example, —O—CH₂—CH₂-OH), (26) —NH(hydroxyalkyl) (e.g., —NH(hydroxy(C₁ to C₆)alkyl, such as, for example, —NH(CH₂CH₂OH)), and (27) oxazolidinone, such as, for example,

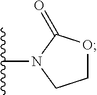

R¹¹ is selected from the group consisting of: F, —OH, —CN, —OR¹⁰, —NHNR¹R¹⁰, —SR¹⁰ and heteroaryl (e.g., triazolyl, such as, for example,

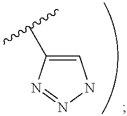

R¹² is selected from the group consisting of: alkyl, aryl, heteroaryl, cycloalkyl, cycloalkylalkyl, heterocycloalkyl and heterocycloalkylalkyl;

R¹⁴ is selected from the group consisting of: alkyl, aryl, heteroaryl, cycloalkyl, cycloalkylalkyl-, heterocycloalkyl, alkylheterocycloalkyl, heterocycloalkylalkyl-, alkylheteroaryl- and alkylaryl-;

R¹⁵ is selected from the group consisting of: H, —OH, alkyl, aryl, heteroaryl, cycloalkyl, cycloalkylalkyl-, heterocycloalkyl and heterocycloalkylalkyl-, alkylheteroaryl- and alkylaryl-;

R²⁰ represents alkyl (e.g., C₁ to C₆ alkyl, such as, for example, methyl, ethyl or isopropyl);

R²³ is selected from the group consisting of: H, alkyl (e.g., C₁ to C₆ alkyl, such as, for example, methyl and i-propyl), aryl (e.g., phenyl), cycloalkyl (e.g., C₃ to C₆ cycloalkyl, such as, for example, cyclopropyl and cyclohexyl), and cycloalkylalkyl-(e.g., C₃ to C₆ cycloalkylalkyl-, such as —(CH₂)ₙ-cycloalkyl, such as —(CH₂)ₙ—(C₃ to C₆)cycloalkyl, wherein each H of each —(CH₂)ₙ— moiety can independently be substituted with an alkyl group (e.g., C₁ to C₆ alkyl, such as, for example, methyl), and wherein in one example n is 1 and the —CH₂— moiety is not substituted, that is, —CH₂-cycloalkyl, such as, —CH₂-cyclopropyl, is an example of said cycloalkylalkyl- moiety);

each R²⁶ is independently selected from the group consisting of: H and alkyl (e.g., C₁ to C₆ alkyl, such as, for example, methyl and ethyl);

R²⁸ is alkyl (e.g., C₁ to C₆ alkyl, such as, for example, methyl or ethyl);

each R³⁰ is independently selected from the group consisting of: H, alkyl (e.g., C₁ to C₆ alkyl, such as, for example methyl, ethyl and i-propyl), and F, and wherein in one example each R³⁰ is H;

each R³² is independently selected from the group consisting of: H and alkyl (e.g., C₁ to C₆ alkyl, such as, for example methyl, ethyl and propyl), and wherein each R³² is generally H;

each R³⁵ is independently selected from the group consisting of: H and C₁ to C₆ alkyl (e.g., methyl, ethyl, i-propyl, and propyl), and wherein in one example both R³⁵ substitutents are the same or different alkyl groups (e.g., both R³⁵ groups are the same alkyl group, such as methyl), and in another example one R³⁵ group is H and the other R³⁵ group is alkyl, such as methyl), and in another example each R³⁵ is preferably H;

each R³⁸ is independently selected from the group consisting of: H, alkyl, aryl, arylalkyl, heteroaryl, heteroarylalkyl, cycloalkyl, cycloalkylalkyl, heterocycloalkyl, heterocycloalkylalkyl, alkylheteroaryl-, alkylaryl-, substituted alkyl, substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl-, and wherein:

said R³⁸ substituted alkyl is substituted with 1 to 3 substituents independently selected from the group consisting of: —NH₂, —NO₂, —CN, —OR²⁶, halo (e.g., F, Cl and Br, and in another example F), —C(O)—NH—R²⁸ (e.g., —C(O)—NH—CH₃), —C(O)OR²⁸ (e.g., —C(O)OC₂H₅), and —C(O)R²⁸ (e.g., —C(O)CH₃), and

said R³⁸ substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl- are substituted with 1 to 3 substituents independently selected from the group consisting of: (1) —NH₂, (2) —NO₂, (3) —CN, (4) —OH, (5) —OR²⁰, (6) —OCF₃, (7) —CF₃, (8) —C(O)R²⁶ (e.g., R²⁶ is H or C₁ to C₆ alkyl, such as, for example, methyl or ethyl, for example, R²⁶ is alkyl (e.g., methyl), thus, an example of —C(O)R²⁶ is —C(O)CH₃), (9) alkyl (e.g., C₁ to C₆ alkyl, e.g., methyl, ethyl, and i-propyl), (10) alkenyl (e.g., C₂ to C₆ alkenyl, such as, for example —CH═CH₂), (11) halo (e.g., F, Cl and Br, and in another example F), (12) —C(O)—NH—R²⁶ (e.g., —C(O)—NH—CH₃), (13) —C(O)OR²⁶ (e.g., R²⁶ is H or e.g., C₁ to C₆ alkyl, such as, for example, methyl or ethyl, for example, R²⁶ is alkyl (e.g., methyl or ethyl), thus, for example, —C(O)OR²⁶ is —C(O)OC₂H₅), (14) —C(O)—NR³²—(C(R³⁰)₂)ₙ—N(R²⁶)₂ (e.g., —C(O)—NH—(CH₂)ₙ—N(R²⁶)₂) (wherein (a) in one example R³² is H, (b) in another example each R³⁰ is H, (c) in another example n is 2, (d) in another example each R²⁶ is independently selected, (e) in another example each R²⁶ is independently selected from the group consisting of: H and methyl), (f) in another example R³² is H, each R³⁰ is H, and each R²⁶ is independently selected, (g) in another example R³² is H, each R³⁰ is H, and each R²⁶ is independently selected from the group consisting of: H and methyl), (15) —S(O)ₜR²⁶ (wherein in one example t is 2, and in another example R²⁶ is methyl, and in another example t is 2 and R²⁶ is methyl), (16) —C(O)N(R³²)(R²⁶) (wherein in one example R³² is H, in another example R²⁶ is alkyl (e.g., propyl), and in another example R³² is H and R²⁶ is alkyl (e.g., propyl)), (17) —NR³²C(O)R²⁶ (e.g., —NHC(O)R²⁶) (wherein in one example R³² is H, in another example R²⁶ is alkyl (e.g., methyl), and in another example R³² is H and R²⁶ is alkyl (e.g., methyl)), (18)

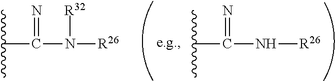

(wherein in one example R³² is H, in another example R²⁶ is H, and in another example R³² is H and R²⁶ is H); and (19) —NHR²⁰;

R⁴² is selected from the group consisting of: alkyl (e.g., C₁ to C₆ alkyl, such as, for example —CH₃), aryl (e.g., phenyl), heteroaryl (e.g., thiazolyl and pyridyl), and cycloalkyl (e.g., C₃ to C₆ cycloalkyl, such as, for example, cyclopropyl);

R⁴⁴ is selected from the group consisting of: H, C₁ to C₆ alkyl, such as, for example, C₁ to C₃ alkyl, such as, for example; methyl, ethyl and i-propyl), cycloalkyl (e.g., C₃ to C₆ cycloalkyl, such as, for example, cyclopropyl and cyclohexyl), and cycloalkylalkyl (e.g., (C₃ to C₆)cycloalkyl(C₁ to C₆)alkyl, such as, for example, (C₃ to C₆)cycloalkyl(C₁ to C₃)alkyl, such as, for example, (C₃ to C₆)cycloalkyl-methyl-, such as, for example, cyclopropyl-methyl- and cyclohexyl-methyl-), and in one example, R⁴⁴ is H; and

Each R⁴⁶ is independently selected from the group consisting of: H, alkyl (e.g., C₁ to C₆ alkyl, such as, for example, C₁ to C₃ alkyl, such as, for example, methyl, ethyl and i-propyl), cycloalkyl (e.g., C₃ to C₆ cycloalkyl, such as, for example, cyclopropyl and cyclohexyl), and cycloalkylalkyl (e.g., (C₃ to C₆)cycloalkyl(C₁ to C₆)alkyl, such as, for example, (C₃ to C₆)cycloalkyl(C₁ to C₃)alkyl, such as, for example, (C₃ to C₆)cycloalkyl-methyl-, such as, for example, cyclopropyl-methyl- and cyclohexyl-methyl-), and in one example, each R⁴⁶ is H.

When R¹ is a cycloalkyl group (i.e., R¹ is R¹⁰ wherein R¹⁰ is cycloalkyl), examples of said cycloalkyl group include, but are limited to, cyclopropyl and cyclobutyl.

When R¹ is a heterocycloalkyl group (i.e., R¹ is R¹⁰ wherein R¹⁰ is heterocycloalkyl), examples of said heterocycloalkyl group include, but are limited to, morpholinyl, pyrrolidinyl, piperidinyl and piperazinyl.

When R¹ is a heteroaryl group (i.e., R¹ is R¹⁰ and R¹⁰ is heteroaryl), examples of said heteroaryl group include, but are not limited to,

(a) unsubstituted heteroaryl,

(b) heteroaryl substituted with 1 to 3 substituents independently selected from the group consisting of: —C(O)R³⁸ (e.g., R³⁸ is alkyl such as methyl), —NHR²⁰ (e.g., —NHCH₃), —OR²⁰ (e.g., —OCH₃), cycloalkyl (e.g., cyclopropyl) and halo (e.g., Cl),

(c) heteroaryl selected from the group consisting of: pyrrolyl, pyrazolyl, imidazolyl, furanyl, thienyl, thiazolyl, pyridyl, pyridyl N—O, and pyrimidinyl,

(d) heteroaryl selected from the group consisting of: pyrrolyl, pyrazolyl, imidazolyl, furanyl, thienyl, thiazolyl, pyridyl, pyridyl N—O, and pyrimidinyl, wherein said heteroaryl is substituted with 1 to 3 substituents independently selected from the group consisting of: —C(O)R³⁸ (e.g., R³⁸ is alkyl such as methyl), —NHR²⁰ (e.g., —NHCH₃), —OR²⁰ (e.g., —OCH₃), cycloalkyl (e.g., cyclopropyl) and halo (e.g., Cl), and

(e) heteroaryl selected from the group consisting of: thienyl substituted with —C(O)R³⁸ (such as, for example, thienyl substituted with —C(O)CH₃), thiazolyl substituted with —NHR²⁰ such as, for example (thazolyl substituted with —NHCH₃), pyridyl substituted with halo (such as, for example, pyridyl substituted with —Cl), pyridyl substituted with —OR²⁰ (such as, for example, pyridyl substituted with methyl), and pyrimidinyl substituted with —OR²⁰ (such as, for example, pyrimidinyl substituted with —OCH₃).

When R¹ is a heteroarylalkyl group (i.e., R¹ is R¹⁰ and R¹⁰ is heteroarylalkyl), examples of said heteroarylalkyl group include, but are not limited to,

(a) unsubstituted heteroarylalkyl-

(b) heteroarylalkyl-substituted with 1 to 3 substituents independently selected from the group consisting of: —C(O)R³⁸ (e.g., R³⁸ is alkyl such as methyl), —NHR²⁰ (e.g., —NHCH₃), —OR²⁰ (e.g., —OCH₃), and halo (e.g., Cl),

(c) heteroarylalkyl-selected from the group consisting of: pyrrolylalkyl- (e.g., pyrrolylCH₂—), pyrazolylalkyl- (e.g., pyrazolylCH₂—), imidazolylalkyl- (e.g., imdazolyl-CH₂—), furanylalkyl- (e.g., furanylCH₂—), thienylalkyl- (e.g., thienylCH₂—), thiazolylalkyl-(e.g., thiazolylCH₂—), pyridylalkyl- (e.g., pyridylCH₂—), pyridyl N—O alkyl- (e.g., pyridyl(N—O)CH₂—), and pyrimidinylalkyl- (e.g., pyrimidinylCH₂—),

(d) heteroarylalkyl-selected from the group consisting of: pyrrolylalkyl- (e.g., pyrrolylCH₂—), pyrazolylalkyl- (e.g., pyrazolylCH₂—), imidazolylalkyl- (e.g., imdazolylCH₂—), furanylalkyl- (e.g., furanylCH₂—), thienylalkyl- (e.g., thienylCH₂—), thiazolylalkyl- (e.g., thiazolylCH₂—), pyridylalkyl- (e.g., pyridylCH₂—), pyridyl N—O alkyl-(e.g., pyridyl(N—O)CH₂—), and pyrimidinylalkyl- (e.g., pyrimidinylCH₂—), wherein said heteroaryl is substituted with 1 to 3 substituents independently selected from the group consisting of: —C(O)R³⁸ (e.g., R³⁸ is alkyl such as methyl), —NHR²⁶ (e.g., —NHCH₃), —OR²⁶ (e.g., —OCH₃), and halo (e.g., Cl), and

(e) heteroarylalkyl-selected from the group consisting of: thienylalkyl-substituted with a —C(O)R²⁰ group (such as, for example, thienylCH₂— substituted with —C(O)CH₃), thiazolylalkyl-substituted with—NHR²⁶ such as, for example (thazolylCH₂-substituted with-NHCH₃), pyridylalkyl-substituted with halo (such as, for example, pyridylCH₂-substituted with —Cl), pyridylalkyl-substituted with —OR²⁶ (such as, for example, pyridylCH₂— substituted with methyl), and pyrimidinylalky-substituted with —OR²⁶ (such as, for example, pyrimidinylCH₂— substituted with —OCH₃).

When R¹ is an aryl group (i.e., R¹ is R¹⁰ and R¹⁰ is aryl), examples of said aryl group include, but are not limited to, phenyl and naphthyl, and preferably phenyl.

When R is an arylalkyl R¹ is R¹⁰ and R¹⁰ is arylalkyl); examples said arylalkyl group include, but are not limited to, —(C(R³⁶)₂)ₙphenyl (e.g., —(CH₂)ₙphenyl), wherein in one example said arylalkyl- is —(C(R³⁰)₂)ₙphenyl wherein n is 1, and in another example said arylalkyl- is —(CH₂)ₙphenyl wherein n is 1 (i.e., said arylalkyl- is benzyl).

When R¹ is a substituted arylalkyl group (i.e., R¹ is R¹⁰ and R¹⁰ is a substituted arylalkyl), examples of said substituted arylalkyl group include, but are not limited to, —(C(R³⁶)₂)ₙ substituted phenyl (e.g., —(CH₂)ₙ substituted phenyl), wherein in one example said substituted arylalkyl- is —(C(R³⁰)₂)ₙ substituted phenyl wherein n is 1, and in another example said substituted arylalkyl- is —(CH₂)ₙ substituted phenyl wherein n is 1 (i.e., said substituted arylalkyl- is substituted benzyl), wherein the aryl moiety of said substituted arylalkyl is substituted with 1 to 3 substituents independently selected from the group consisiting of: halo (e.g., F, Cl and Br), —CF₃, and —OR²⁶ (e.g., —OCH₃).

Those skilled in the art will appreciate that when Q¹ is aryl, substituted aryl, heteroaryl or substituted heteroaryl the two carbon atoms common to the two fused rings are not substituted. Thus, there is no R³ and no R⁴ groups in 2.9 when Q¹ is aryl, substituted aryl, heteroaryl or substituted heteroaryl. There is no R³ and no R⁴ groups in 2.10 when Q¹ fused to the R³ and R⁴ positions is aryl, substituted aryl, heteroaryl or substituted heteroaryl. There is no R⁶ and no R⁷ groups in 2.10 when Q¹ fused to the R⁶ and R⁷ positions is aryl, substituted aryl, heteroaryl or substituted heteroaryl. There is no R³ and no R⁴ groups in 2.11 when Q¹ fused to the R³ and R⁴ positions is aryl, substituted aryl, heteroaryl or substituted heteroaryl. There is no R³ and no R⁴ groups in 2.13 when Q¹ fused to the R³ and R⁴ positions is aryl, substituted aryl, heteroaryl or substituted heteroaryl. There is no R³ and no R⁴ groups in 2.14 when Q¹ fused to the R³ and R⁴ positions is aryl, substituted aryl, heteroaryl or substituted heteroaryl. There is no R³ and no R⁴ groups in 2.15 when Q¹ fused to the R³ and R⁴ positions is aryl, substituted aryl, heteroaryl or substituted heteroaryl. There is no R⁶ and no R⁷ groups in 2.15 when Q¹ fused to the R³ and R⁴ positions is aryl, substituted aryl, heteroaryl or substituted heteroaryl.

In one embodiment of the compounds of formula 1.0, z is 1. Thus, in this embodiment the compounds of formula 1.0 have the formula 1.0A1:

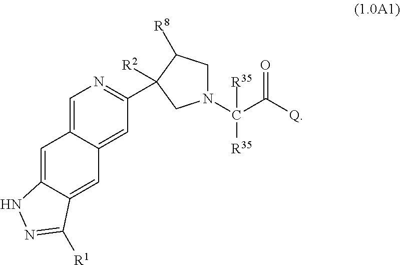

In another embodiment of the compounds of formula 1.0, z is 1, and each R³⁵ is independently selected from the group consisting of: H, methyl, ethyl, i-propyl and propyl (e.g., one R³⁵ is H and the other is methyl, or both R³⁵ substituents are methyl, or preferably both R³⁵ substitutents are H).

In another embodiment of the compounds of formula 1.0, each R³⁵ is H. Thus, in this embodiment the compounds of formula 1.0 have the formula 1.0B1:

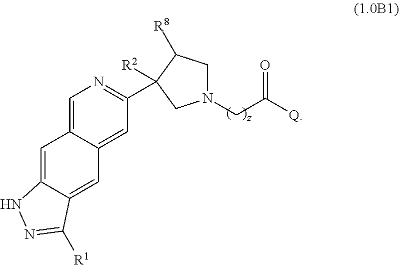

In another embodiment of the compounds of formula 1.0, z is preferably 1 and each R³⁵ is preferably H. Thus, in this embodiment the compounds of formula 1.0 have the formula 1.0C1:

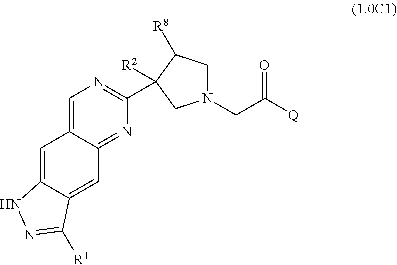

Another embodiment of this invention is directed to compounds of formula 1.0 having the formula 1.1A:

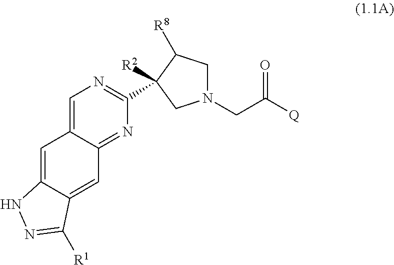

Examples of Q include, but are not limited to: moieties 2.1, 2.2, 2.3., 2.4, 2.5, 2.6, 2.7, 2.8, 2.9, 2.10, 2.11, 2.14, or 2.15 wherein each R³, R⁴, R⁶, and R⁷ is independently selected from the group consisting of: H and alkyl (e.g., C₁ to C₆ alkyl, such as, for example methyl).

Examples of Q also include, but are not limited to: moieties 2.1, 2.2, 2.3., 2.4, 2.5, 2.6, 2.7, 2.8, 2.9, 2.10, 2.11, 2.14, or 2.15 wherein each R³, R⁴, R⁶, and R⁷ is H.

Examples of Q also include, but are not limited to: moieties 2.17, 2.18, 2.19, 2.20 and 2.21 wherein each R³, R⁴, R⁶, and R⁷ is independently selected from the group consisting of: H and alkyl (e.g., C₁ to C₆ alkyl, such as, for example methyl).

Examples of Q also include, but are not limited to: moieties 2.17, 2.18, 2.19, 2.20 and 2.21 wherein each R³, R⁴, R⁶, and R⁷ is H.

Examples of Q include, but are not limited to: moieties 2.12, 2.13, or 2.16 wherein each R³, R⁴, and R⁷ is independently selected from the group consisting of: H and alkyl (e.g., C₁ to C₆ alkyl, such as, for example methyl).

Examples of Q also include, but are not limited to: moieties 2.12, 2.13, or 2.16 wherein each R³, R⁴, and R⁷ is H.

Examples of Q include, but are not limited to: moiety 2.22 wherein each R³, R⁴, and R⁷ is independently selected from the group consisting of: H and alkyl (e.g., C₁ to C₆ alkyl, such as, for example methyl).

Examples of Q also include, but are not limited to: moiety 2.22 wherein each R³, R⁴, and R⁷ is H.

Thus, in one example of Q, Q is moiety 2.1 wherein each R³, R⁴, R⁶, and R⁷ is independently selected from the group consisting of: H and alkyl (e.g., C₁ to C₆ alkyl, such as, for example methyl).

In another example of Q, Q is moiety 2.1 wherein each R³, R⁴, R⁶, and R⁷ is independently selected from the group consisting of: H and methyl.

In another example of Q, Q is moiety 2.1 wherein each R³, R⁴, R⁶, and R⁷ is H.

In another example of Q, Q is moiety 2.2 wherein each R³, R⁴, R⁶, and R⁷ is independently selected from the group consisting of H, and alkyl (e.g., C₁ to C₆ alkyl, such as, for example methyl).

In another example of Q, Q is moiety 2.2 wherein each R³, R⁴, R⁶, and R⁷ is independently selected from the group consisting of: H and methyl.

In another example of Q, Q is moiety 2.2 wherein each R³, R⁴, R⁶, and R⁷ is H.

In another example of Q, Q is moiety 2.3 wherein each R³, R⁴, R⁶, and R⁷ is independently selected from the group consisting of: H and alkyl (e.g., C₁ to C₆ alkyl, such as, for example methyl).

In another example of Q, Q is moiety 2.3 wherein each R³, R⁴, R⁶, and R⁷ is independently selected from the group consisting of: H and methyl.

In another example of Q, Q is moiety 2.3 wherein each R³, R⁴, R⁶, and R⁷ is H.

In another example of Q, Q is moiety 2.4 wherein each R³, R⁴, R⁶, and R⁷ is independently selected from the group consisting of: H and alkyl (e.g., C₁ to C₆ alkyl, such as, for example methyl).

In another example of Q, Q is moiety 2.4 wherein each R³, R⁴, R⁶, and R⁷ is independently selected from the group consisting of: H and methyl.

In another example of Q, Q is moiety 2.4 wherein each R³, R⁴, R⁶, and R⁷ is H.

In another example of Q, Q is moiety 2.5 wherein each R³, R⁴, R⁶, and R⁷ is independently selected from the group consisting of: H and alkyl (e.g., C₁ to C₆ alkyl, such as, for example methyl).

In another example of Q, Q is moiety 2.5 wherein each R³, R⁴, R⁶, and R⁷ is independently selected from the group consisting of: H and methyl.

In another example of Q, Q is moiety 2.5 wherein each R³, R⁴, R⁶, and R⁷ is H.

In another example of Q, Q is moiety 2.6 wherein each R³, R⁴, R⁶, and R⁷ is independently selected from the group consisting of: H and alkyl (e.g., C₁ to C₆ alkyl, such as, for example methyl).

In another example of Q, Q is moiety 2.6 wherein each R³, R⁴, R⁶, and R⁷ is independently selected from the group consisting of: H and methyl.

In another example of Q, Q is moiety 2.7 wherein each R³, R⁴, R⁶, and R⁷ is independently selected from the group consisting of: H and alkyl (e.g., C₁ to C₆ alkyl, such as, for example methyl).

In another example of Q, Q is moiety 2.7 wherein each R³, R⁴, R⁶, and R⁷ is independently selected from the group consisting of: H and methyl.

In another example of Q, Q is moiety 2.7 wherein each R³, R⁴, R⁶, and R⁷ is H.

In another example of Q, Q is moiety 2.8 wherein each R³, R⁴, R⁶, and R⁷ is independently selected from the group consisting of: H and alkyl (e.g., C₁ to C₆ alkyl, such as, for example methyl).

In another example of Q, Q is moiety 2.8 wherein each R³, R⁴, R⁶, and R⁷ is independently selected from the group consisting of: H and methyl.

In another example of Q, Q is moiety 2.8 wherein each R³, R⁴, R⁶, and R⁷ is H.

In another example of Q, Q is moiety 2.9 or 2.10 wherein each R³, R⁴, R⁶, and R⁷ is independently selected from the group consisting of: H and alkyl (e.g., C₁ to C₆ alkyl, such as, for example methyl).

In another example of Q, Q is moiety 2.9 or 2.10 wherein each R³, R⁴, R⁶, and R⁷ is independently selected from the group consisting of: H and methyl.

In another example of Q, Q is moiety 2.9 or 2.10 wherein each R³, R⁴, R⁶, and R⁷ is H.

In another example of Q, Q is moiety 2.11 wherein each R³, R⁴, R⁶, and R⁷ is independently selected from the group consisting of: H and alkyl (e.g., C₁ to C₆ alkyl, such as, for example methyl).

In another example of Q, Q is moiety 2.11 wherein each R³, R⁴, R⁶, and R⁷ is independently selected from the group consisting of: H and methyl.

In another example of Q, Q is moiety 2.11 wherein each R³, R⁴, R⁶, and R⁷ is H.

In another example of Q, Q is moiety 2.12 or 2.13 wherein each R³, R⁴, and R⁷ is independently selected from the group consisting of: H and alkyl (e.g., C₁ to C₆ alkyl, such as, for example methyl).

In another example of Q, Q is moiety 2.12 or 2.13 wherein each R³, R⁴, and R⁷ is independently selected from the group consisting of: H and methyl.

In another example of Q, Q is moiety 2.12 or 2.13 wherein each R³, R⁴, and R⁷ is H.

In another example of Q, Q is moiety 2.14 or 2.15 wherein each R³, R⁴, R⁶, and R⁷ is independently selected from the group consisting of: H and alkyl (e.g., C₁ to C₆ alkyl, such as, for example methyl).

In another example of Q, Q is moiety 2.14 or 2.15 wherein each R³, R⁴, R⁶, and R⁷ is independently selected from the group consisting of: H and methyl.

In another example of Q, Q is moiety 2.14 or 2.15 wherein each R³, R⁴, R⁶, and R⁷ is H.

In another example of Q, Q is moiety 2.16 wherein each R³, R⁴, and R⁷ is H.

In another example of Q, Q is moiety 2.17 wherein each R³, R⁴, R⁶, and R⁷ is independently selected from the group consisting of: H and alkyl (e.g., C₁ to C₆ alkyl, such as, for example methyl).

In another example of Q, Q is moiety 2.17 wherein each R³, R⁴, R⁶, and R⁷ is independently selected from the group consisting of: H and methyl.

In another example of Q, Q is moiety 2.17 wherein each R³, R⁴, R⁶, and R⁷ is H.

In another example of Q, Q is moiety 2.18 wherein each R³, R⁴, R⁶, and R⁷ is independently selected from the group consisting of: H and alkyl (e.g., C₁ to C₆ alkyl, such as, for example methyl).

In another example of Q, Q is moiety 2.18 wherein each R³, R⁴, R⁶, and R⁷ is independently selected from the group consisting of: H and methyl.

In another example of Q, Q is moiety 2.18 wherein each R³, R⁴, R⁶, and R⁷ is H.

In another example of Q, Q is moiety 2.19 wherein each R³, R⁴, R⁶, and R⁷ is independently selected from the group consisting of: H and alkyl (e.g., C₁ to C₆ alkyl, such as, for example methyl).

In another example of Q, Q is moiety 2.19 wherein each R³, R⁴, R⁶, and R⁷ is independently selected from the group consisting of: H and methyl.

In another example of Q, Q is moiety 2.19 wherein each R³, R⁴, R⁶, and R⁷ is H.

In another example of Q, Q is moiety 2.20 wherein each R³, R⁴, R⁶, and R⁷ is independently selected from the group consisting of: H and alkyl (e.g., C₁ to C₆ alkyl, such as, for example methyl).

In another example of Q, Q is moiety 2.20 wherein each R³, R⁴, R⁶, and R⁷ is independently selected from the group consisting of: H and methyl.

In another example of Q, Q is moiety 2.20 wherein each R³, R⁴, R⁶, and R⁷ is H.

In another example of Q, Q is moiety 2.21 wherein each R³, R⁴, R⁶, and R⁷ is independently selected from the group consisting of: H and alkyl (e.g., C₁ to C₆ alkyl, such as, for example methyl).

In another example of Q, Q is moiety 2.21 wherein each R³, R⁴, R⁶, and R⁷ is independently selected from the group consisting of: H and methyl.

In another example of Q, Q is moiety 2.21 wherein each R³, R⁴, R⁶, and R⁷ is H.

In another example of Q, Q is moiety 2.22 wherein each R³, R⁴, and R⁷ is independently selected from the group consisting of: H and alkyl (e.g., C₁ to C₆ alkyl, such as, for example methyl).

In another example of Q, Q is moiety 2.22 wherein each R³, R⁴, and R⁷ is independently selected from the group consisting of: H and methyl.

In another example of Q, Q is moiety 2.22 wherein each R³, R⁴, and R⁷ is H.

Another example of the Q substituent 2.3 is:

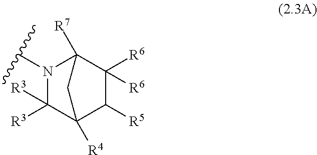

(i.e., each R²⁴ is H and w is 1).

Another example of the Q substituent 2.3 is:

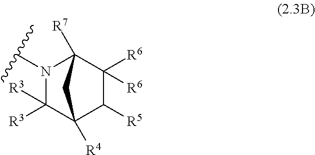

(i.e., each R²⁴ is H and w is 1).

Another example of the Q substitutent 2.3 is:

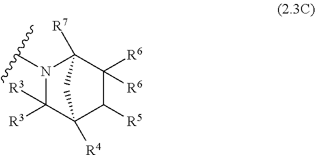

(i.e., each R²⁴ is H and w is 1).

An example of the Q substituent 2.4 is:

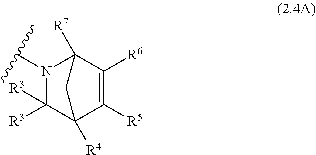

(i.e., each R²⁴ is H and w is 1).

Another example of the Q substituent 2.4 is:

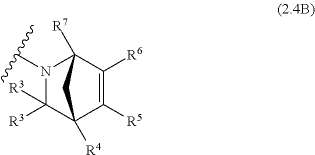

(i.e., each R²⁴ is H and w is 1).

Another example of the Q substituent 2.4 is:

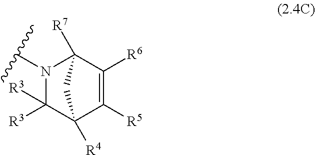

(i.e., each R²⁴ is H and w is 1).

An example of the Q substituent 2.5 is:

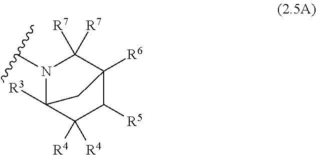

(i.e., each R²⁴ is H and w is 1).

Another example of the Q substituent 2.5 is:

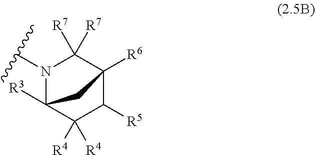

(i.e., each R²⁴ is H and w is 1).

Another example of the Q substituent 2.5 is:

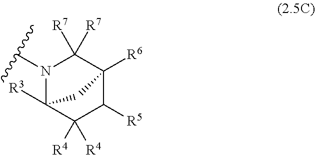

(i.e., each R²⁴ is H and w is 1).

An example of the Q substituent 2.6 is:

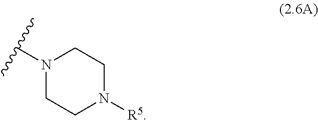

An example of the Q substituent 2.7 is:

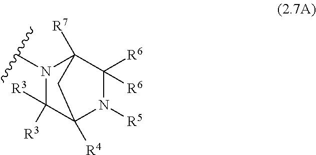

(i.e., each R²⁴ is H and w is 1).

An example of the Q substituent 2.7 is:

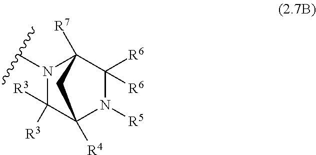

(i.e., each R²⁴ is H and w is 1).

An example of the Q substituent 2.7 is:

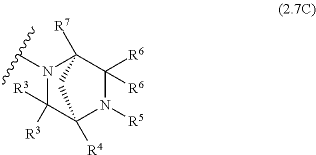

(i.e., each R²⁴ is H and w is 1).

An example of the Q substituent 2.8 is:

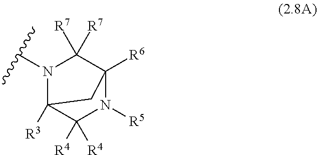

(i.e., each R²⁴ is H and w is 1).

Another example of the Q substituent 2.8 is:

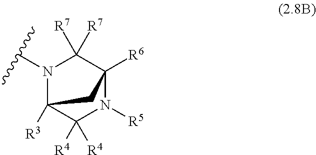

(i.e., each R²⁴ is H and w is 1).

Another example of the Q substituent 2.8 is:

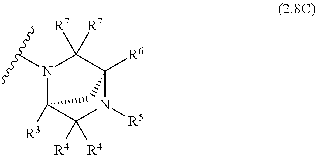

(i.e., each R²⁴ is H and w is 1).

Another example of the Q substituent 2.3 is:

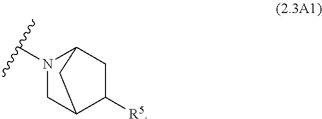

Another example of the Q substituent 2.3 is:

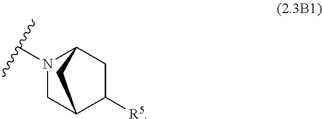

Another example of the Q substituent 2.3 is:

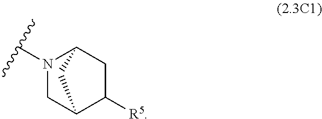

Another example of the Q substituent 2.4 is:

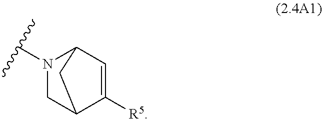

Another example of the Q substituent 2.4 is:

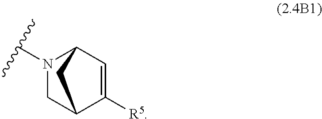

Another example of the Q substituent 2.4 is:

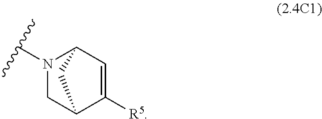

Another example of the Q substituent 2.5 is:

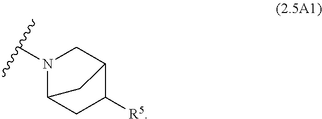

Another example of the Q substituent 2.5 is:

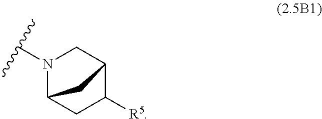

Another example of the Q substituent 2.5 is:

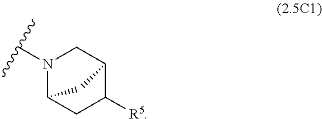

Another example of the Q substituent 2.7 is:

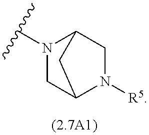

Another example of the Q substituent 2.7 is:

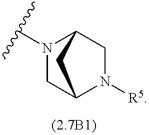

Another example of the Q substituent 2.7 is:

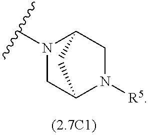

Another example of the Q substituent 2.8 is:

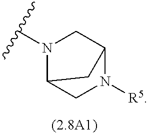

Another example of the Q substituent 2.8 is:

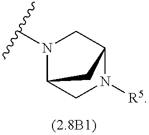

Another example of the Q substituent 2.8 is:

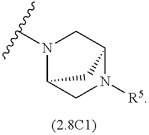

Another example of the Q substitutent is the piperazine ring:

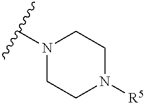

substituted with one or two substituents independently selected from the group consisting of R³ groups, provided that said one or two substitutents are not H. In one embodiment said substituents are selected from the group consisting of alkyl groups (e.g., C₁ to C₆ alkyl, e.g., methyl). In another embodiment there is one substituent on said piperazine ring. In another embodiment there is one substituent on said piperazine ring and said substituent is methyl.

Another example of the Q substituent is the piperazine ring:

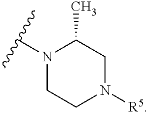

Another example of the Q substitutent is the piperidine ring:

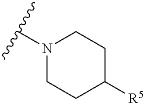

substituted with one or two substituents independently selected from the group consisting of R³ groups, provided that said one or two substitutents are not H. In one embodiment said substituents are selected from the group consisting of alkyl groups (e.g., C₁ to C₆ alkyl, e.g., methyl). In another embodiment there is one substituent on said piperidine ring. In another embodiment there is one substituent on said piperidine ring and said substituent is methyl.

In one example of the Q substituent 2.16

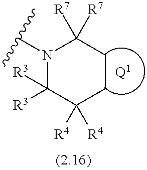

Q¹ is heteroaryl.

In another example of the Q substituent 2.16 Q¹ is aryl.

Thus, one example of the Q substituent 2.16 is 2.16A:

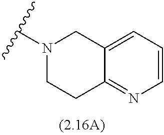

(i.e., Q¹ is pyridyl, and each R³, R⁴ and R⁷ is H).

In another example, the Q substituent 2.16 is 2.16A1:

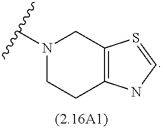

Another example of the Q substitutent 2.16 is 2.16B:

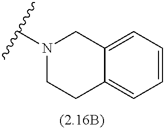

(i.e., Q¹ is phenyl, and each R³, R⁴ and R⁷ is H).

Another example of the Q substituent 2.16 is 2.16C

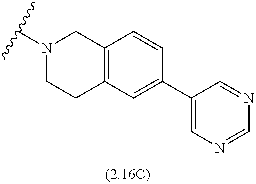

(i.e., Q¹ is substituted phenyl, and each R³, R⁴ and R⁷ is H).

Another example of the Q substituent 2.16 is 2.16D

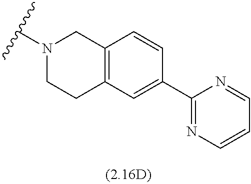

(i.e., Q¹ is substituted phenyl, and each R³, R⁴ and R⁷ is H).

Another example of the Q substituent 2.16 is 2.16E

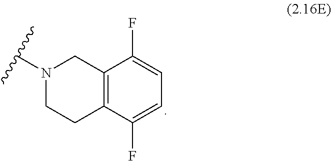

When the Q substitutent comprises two Q¹ rings, each Q¹ ring is independently selected. Generally, the Q¹ cycloalkyl rings and the Q¹ substituted cycloalkyl rings comprise 5 to 7 ring carbons. In general, the heterocycloalkyl Q¹ rings and the substituted heterocycloalkyl Q¹ rings comprise 5 to 7 ring carbons and comprise 1 to 3 (generally 1 or 2, or generally 1) ring heteroatoms selected from the group consisting of: O, N and S. In general, the heteroaryl Q¹ rings and the substituted heteroaryl Q¹ rings comprise 5 to 7 ring carbons and comprise 1 to 3 (generally 1 or 2, or generally 1) ring heteroatoms selected from the group consisting of: O, N and S. Examples of the Q¹ rings include, but are not limited to: piperidinyl, piperazinyl, pyranyl, pyrrolidinyl, morpholinyl, thiomorpholinyl, pyridyl, pyrimidinyl, pyrrolyl, pyrazolyl, furanyl, thienyl, thiazolyl, imidazolyl, cyclopentyl, cyclohexyl and cycloheptyl. Examples of the Q¹ rings also include, but are not limited to: substituted piperidinyl, substituted piperazinyl, substituted pyranyl, substituted pyrrolidinyl, substituted morpholinyl, substituted thiomorpholinyl, substituted pyridyl, substituted pyrimidinyl, substituted pyrrolyl, substituted pyrazolyl, substituted furanyl, substituted thienyl, substituted thiazolyl, substituted imidazolyl, substituted cyclopentyl, substituted cyclohexyl and substituted cycloheptyl wherein said substituted Q¹ rings are substituted with 1 to 3 substitutents selected from the R¹⁰ moieties.

Generally, the Q² cycloalkyl rings and the Q² substituted cycloalkyl rings comprise 5 to 7 ring carbons. In general, the heterocycloalkyl Q² rings and the substituted heterocycloalkyl Q¹ rings comprise 5 to 7 ring carbons and comprise 1 to 3 (generally 1 or 2, or generally 1) ring heteroatoms selected from the group consisting of: O, N and S.

Examples of the Q² rings include, but are not limited to: piperidinyl, piperazinyl, pyranyl, pyrrolidinyl, cyclopentyl, cyclohexyl and cycloheptyl. Examples of the Q² rings also include, but are not limited to: substituted piperidinyl, substituted piperazinyl, substituted pyranyl, substituted pyrrolidinyl, substituted morpholinyl, substituted thiomorpholinyl, substituted cyclopentyl, substituted cyclohexyl and substituted cycloheptyl wherein said substituted Q¹ rings are substituted with 1 to 3 substitutents selected from the R¹⁰ moieties.

In one example the Q substituent 2.17 is:

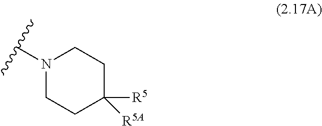

wherein R^{5A} is halo.

Another example of the Q substituent 2.17 is:

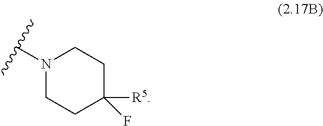

Another example of the Q substituent 2.17 is:

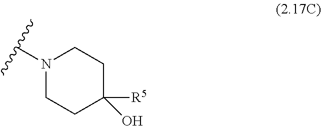

Another example of the Q substituent 2.17 is:

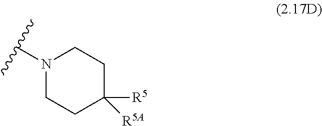

wherein R^{5A} is alkoxy, i.e., —O—(C₁ to C₆)alkyl, such as, for example, —O—(C₁ to C₃)alkyl, or —O—(C₁ to C₂)alkyl.

Another example of the Q substituent 2.17 is:

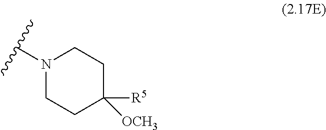

Another example of the Q substituent 2.17 is:

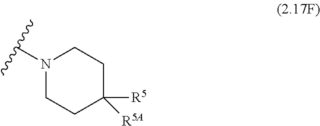

wherein R^{5A} is alkyl (e.g., —(C₁ to C₆)alkyl, such as, for example, —(C₁ to C₃)alkyl, or —(C₁ to C₂)alkyl).

Thus, another example of the Q substituent 2.17 is:

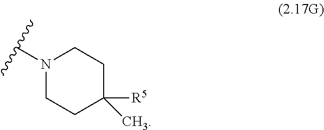

Another example of the Q substituent 2.17 is:

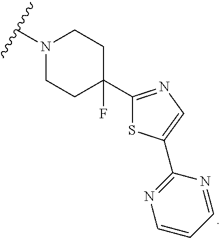

Another example of the Q substituent 2.17 is:

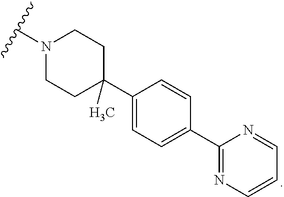

Another example of the Q substituent 2.17 is:

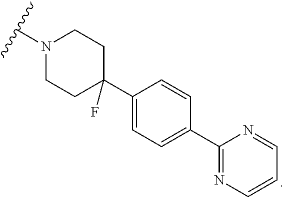

In another embodiment of this invention Q is:

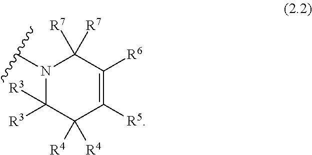

Thus, another example of Q is:

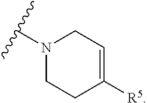

Another example of the Q substituent 2.2 is:

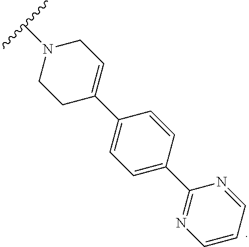

Another example of the Q substituent 2.2 is:

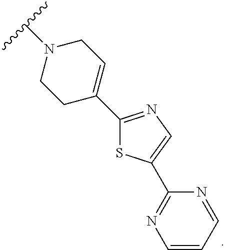

Another example of the Q substituent 2.2 is:

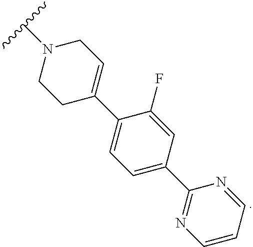

Another example of the Q substituent 2.2 is:

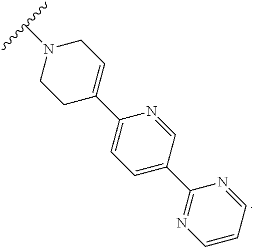

Another example of the Q substituent 2.2 is:

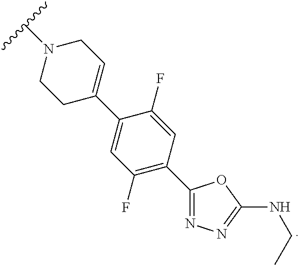

Another example of the Q substituent 2.2 is:

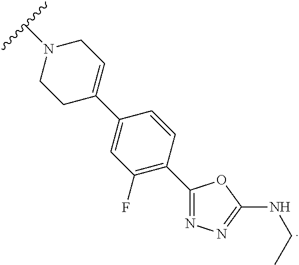

Another example of the Q substituent 2.2 is:

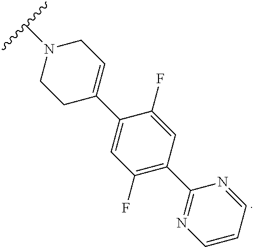

Another example of the Q substituent 2.2 is:

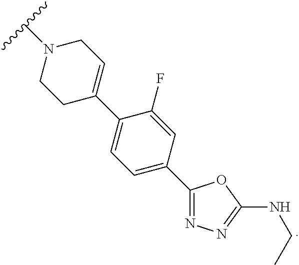

Another example of the Q substituent 2.6 is:

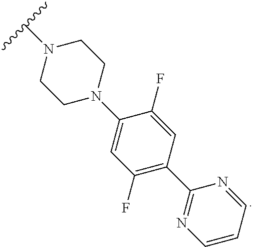

Another example of the Q substituent 2.6 is:

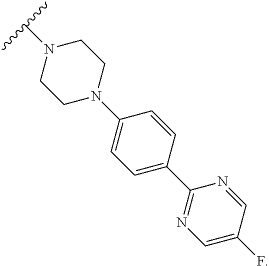

Another example of the Q substituent 2.6 is:

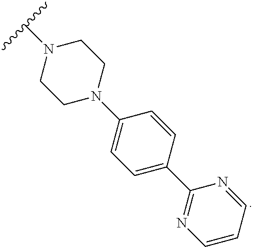

Another example of the Q substituent 2.6 is:

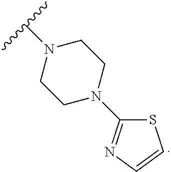

In another embodiment Q is:

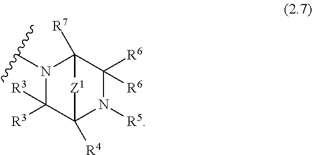

In another embodiment of this invention Q is:

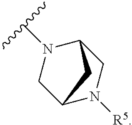

An example of the Q substituent 2.7 is:

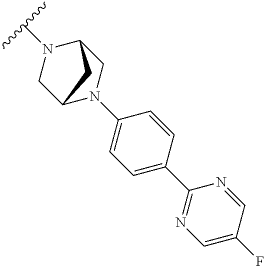

Examples of R¹ for the compounds of this invention (e.g., compounds of formulas 1.0, 1.0A1, 1.0B1, 1.0C1, and 1.1A) include, but are not limited to Br,

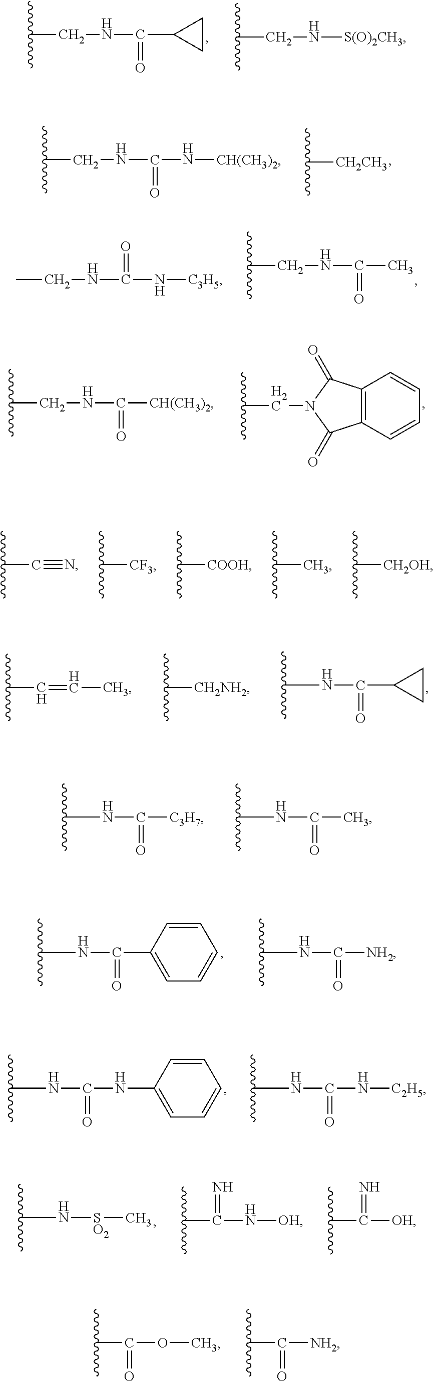

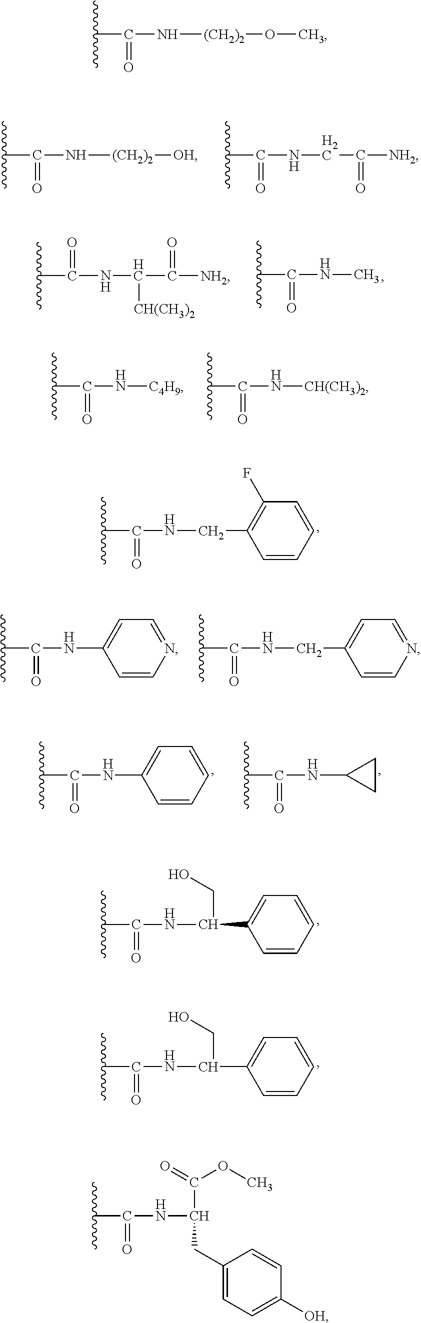

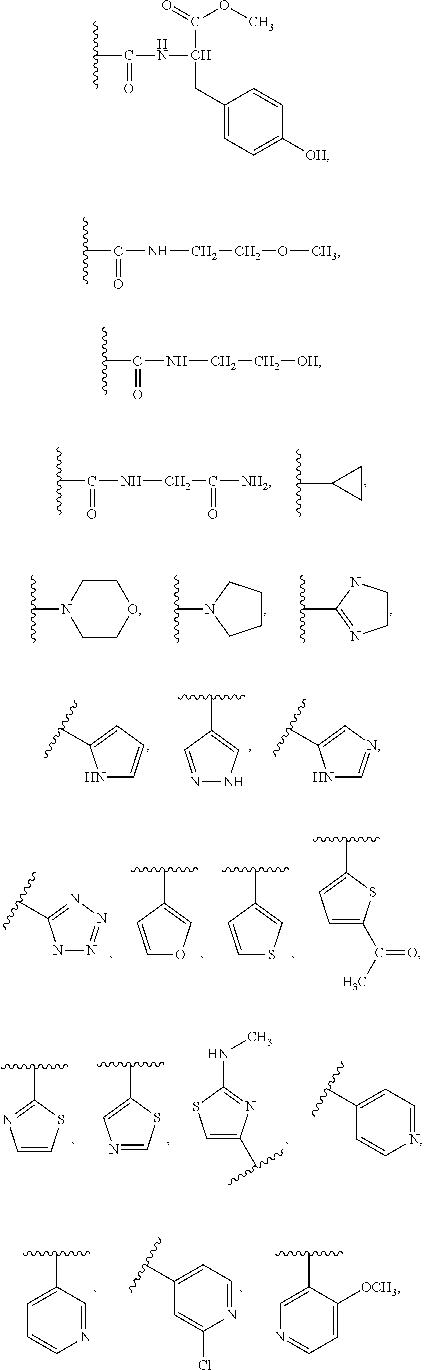

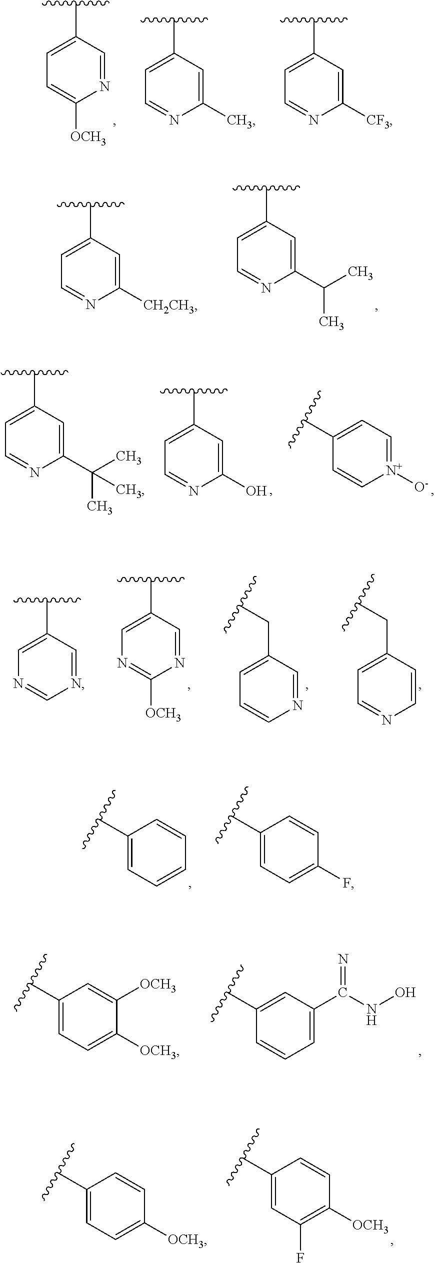

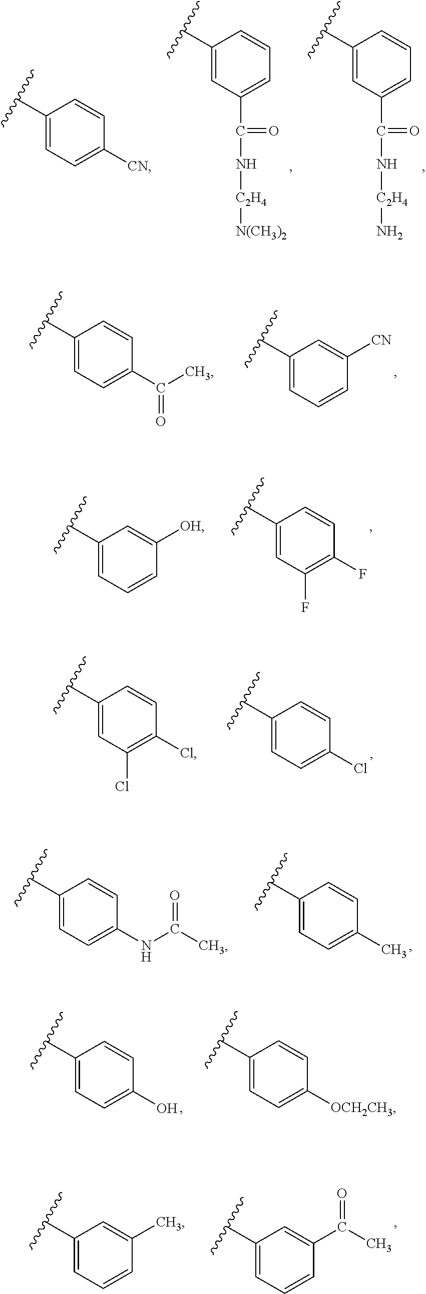

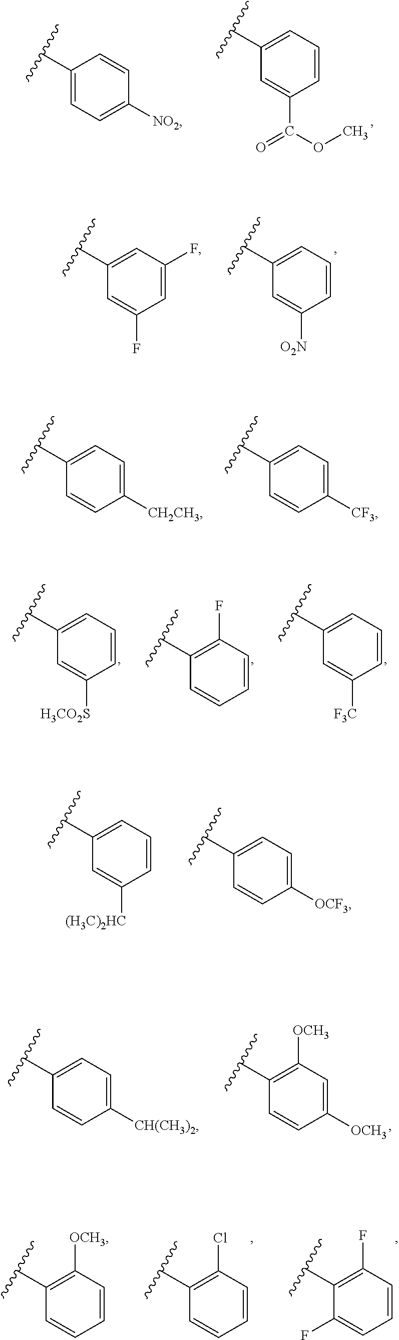

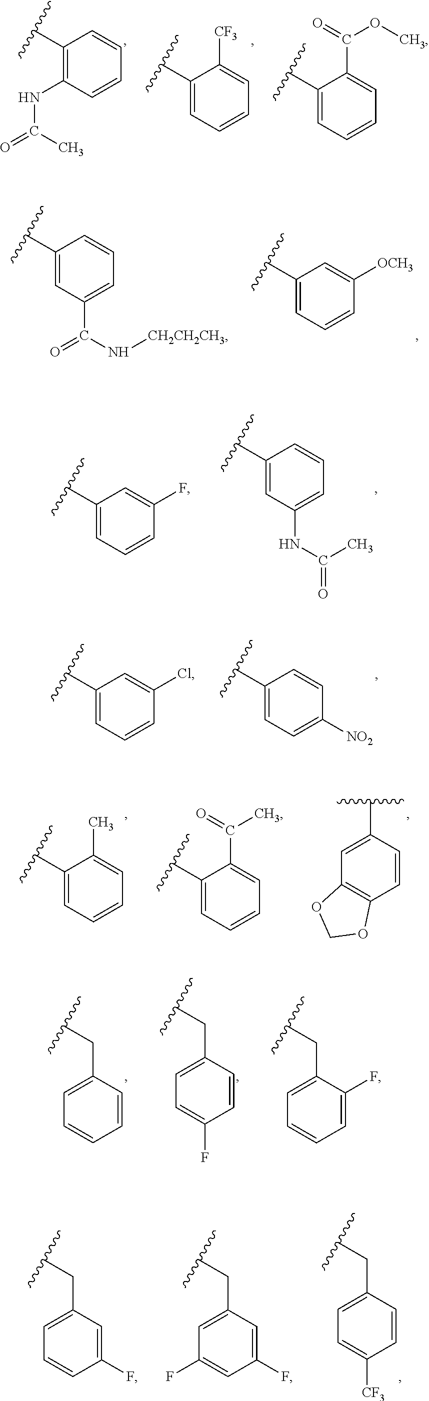

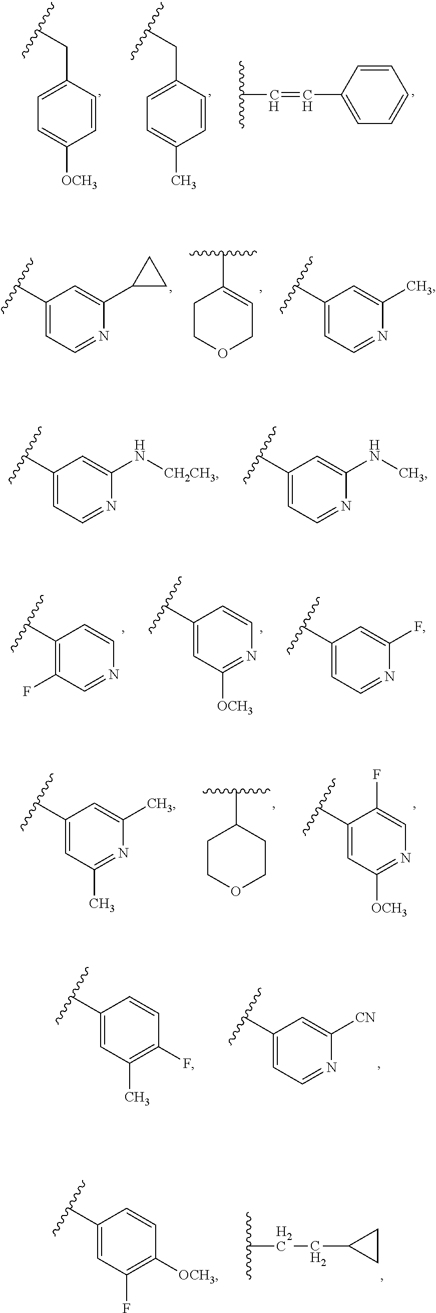

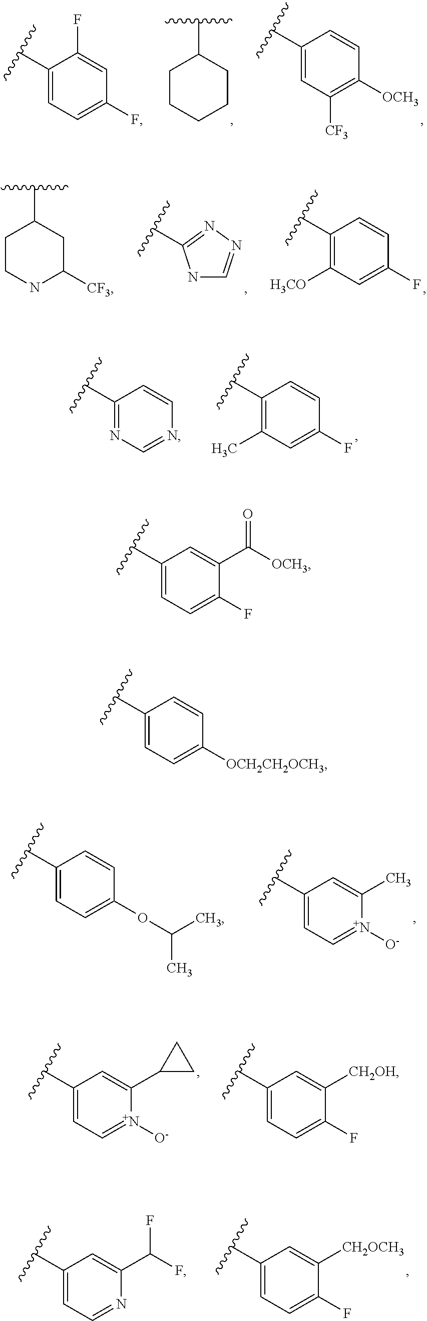

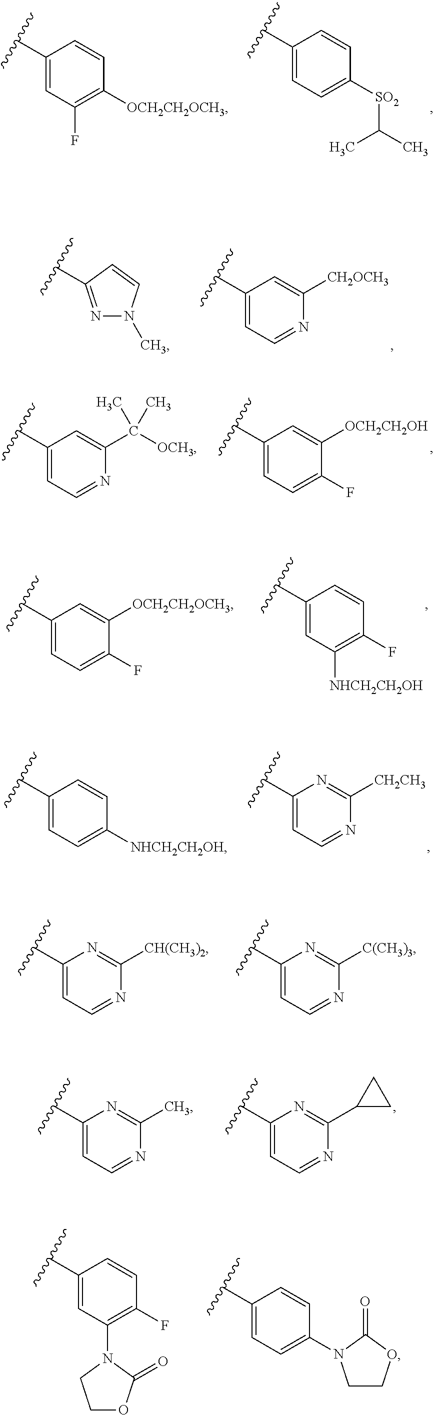

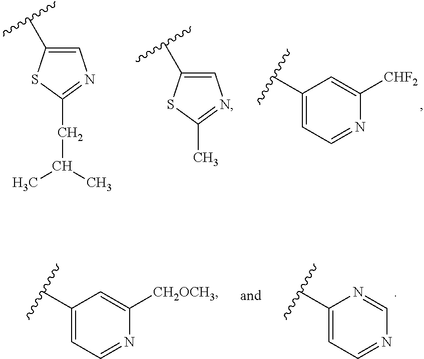

In one embodiment of this invention, R¹ is selected from the group consisting of:

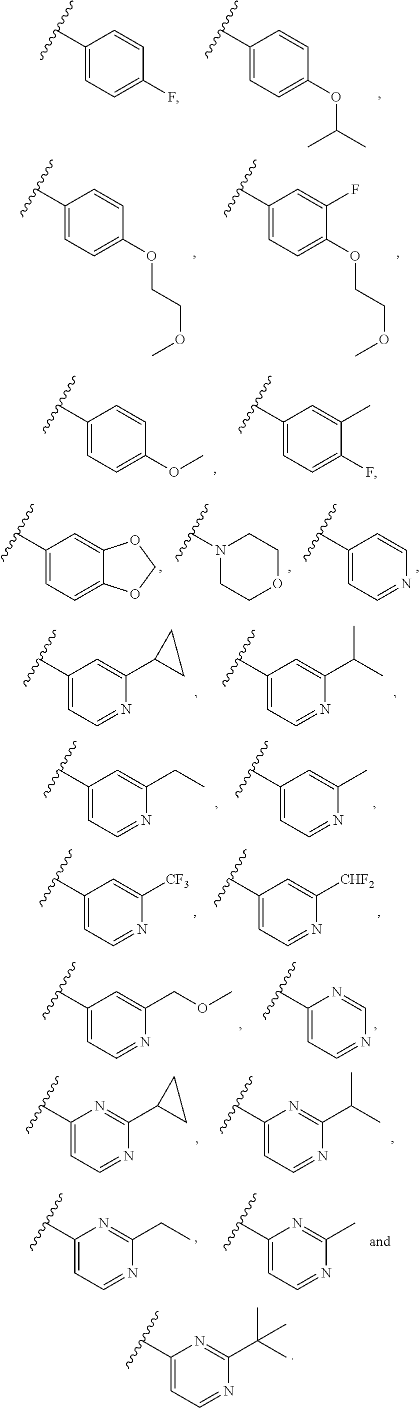

In another embodiment of this invention R¹ is selected from the group consisting of:

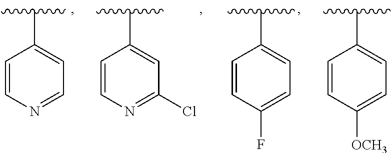

### and Br.

In another embodiment of this invention R¹ is selected from the group consisting of:

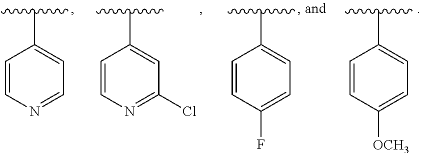

R¹, in one embodiment of this invention, is aryl (e.g., phenyl).

R¹, in one embodiment of this invention is substituted aryl, such as,

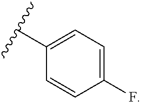

R¹, in another embodiment of this invention, is heteroaryl (e.g., in one embodiment R¹ is pyridyl N-oxide, and in another embodiment R¹ is pyridyl, such as

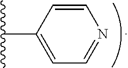

R¹, in one embodiment of this invention, is substituted heteroaryl (e.g., substituted pyridyl).

R¹, in one embodiment of this invention, is substituted heteroaryl (e.g., substituted pyridyl), such as, for example:

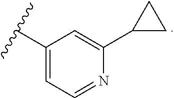

In another embodiment of this invention R¹ is:

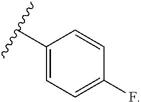

In another embodiment of this invention R¹ is:

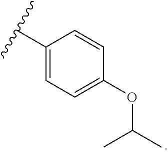

In another embodiment of this invention R¹ is:

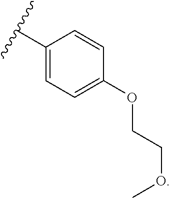

In another embodiment of this invention R¹ is:

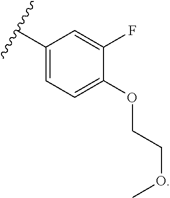

In another embodiment of this invention R¹ is:

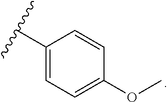

In another embodiment of this invention R¹ is:

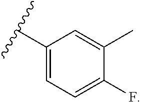

In another embodiment of this invention R¹ is:

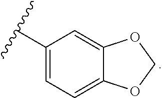

In another embodiment of this invention R¹ is:

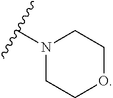

In another embodiment of this invention R¹ is:

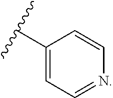

In another embodiment of this invention R¹ is:

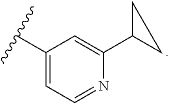

In another embodiment of this invention R¹ is:

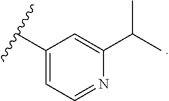

In another embodiment of this invention R¹ is:

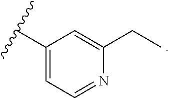

In another embodiment of this invention R¹ is:

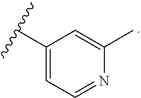

In another embodiment of this invention R¹ is:

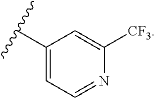

In another embodiment of this invention R¹ is:

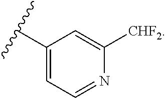

In another embodiment of this invention R¹ is:

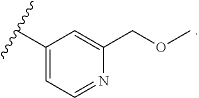

In another embodiment of this invention R¹ is:

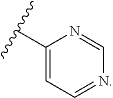

In another embodiment of this invention R¹ is:

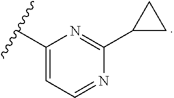

In another embodiment of this invention R¹ is:

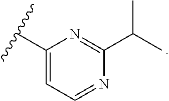

In another embodiment of this invention R¹ is:

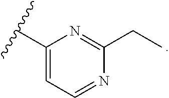

In another embodiment of this invention R¹ is:

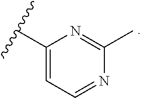

In another embodiment of this invention R¹ is:

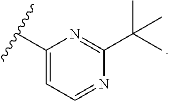

In another embodiment of this invention R¹ is:

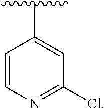

In another embodiment of this invention R¹ is:

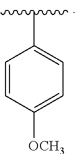

In another embodiment of this invention R¹ is Br.

Examples of R⁵ for the compounds of this invention (e.g., compounds of formulas 1.0, 1.0A1, 1.0B1, 1.0C1, and 1.1A) include but are not limited to:

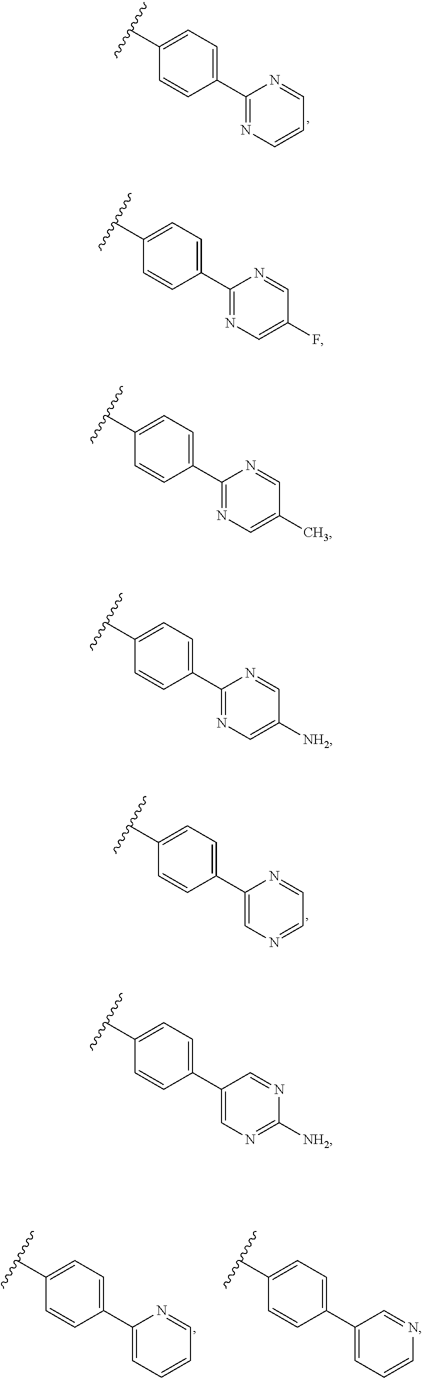

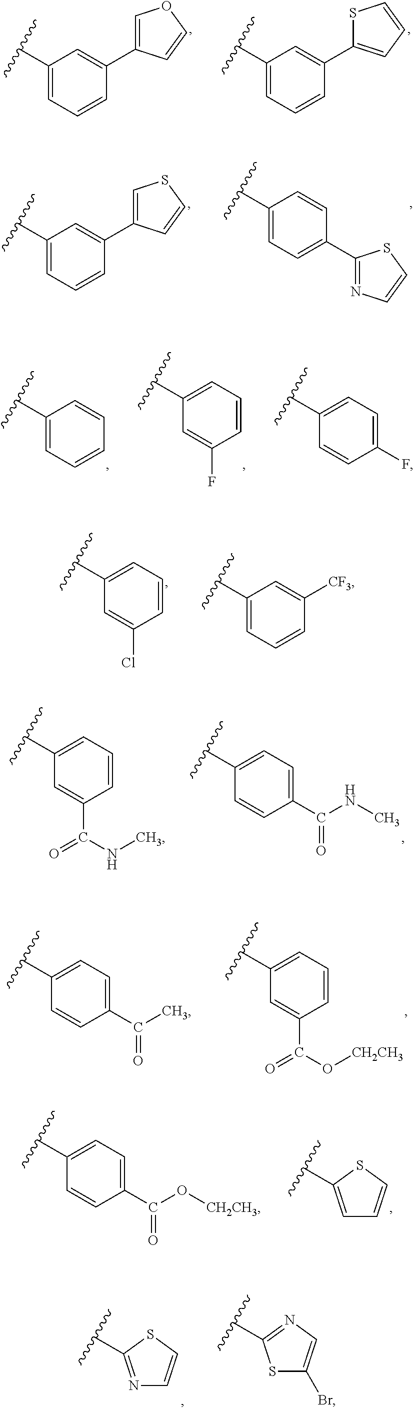

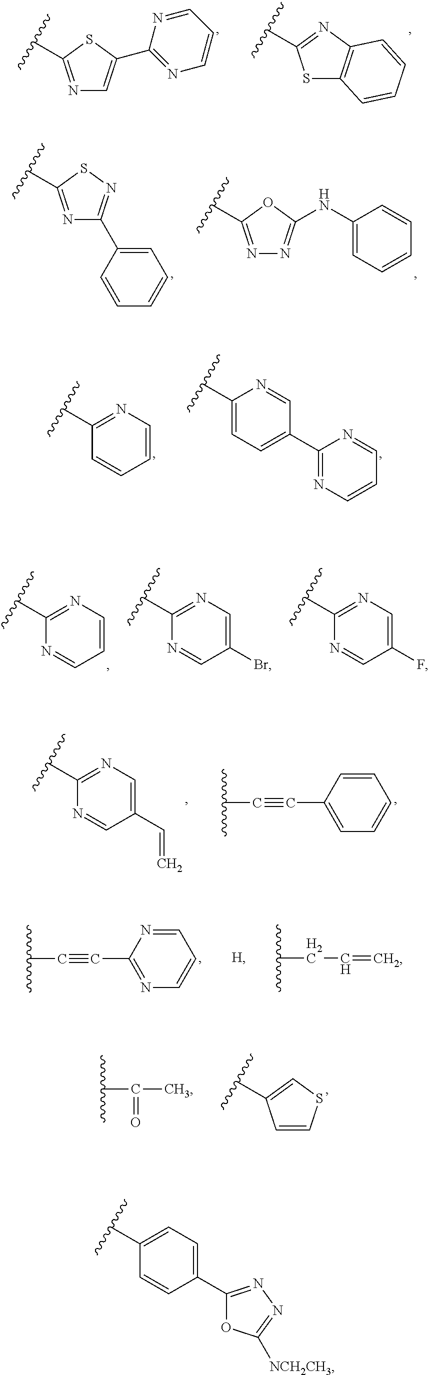

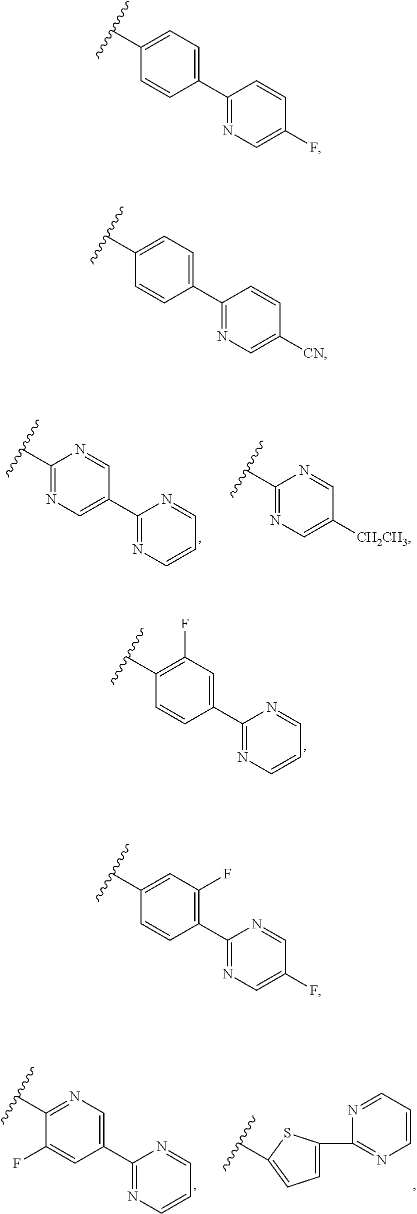

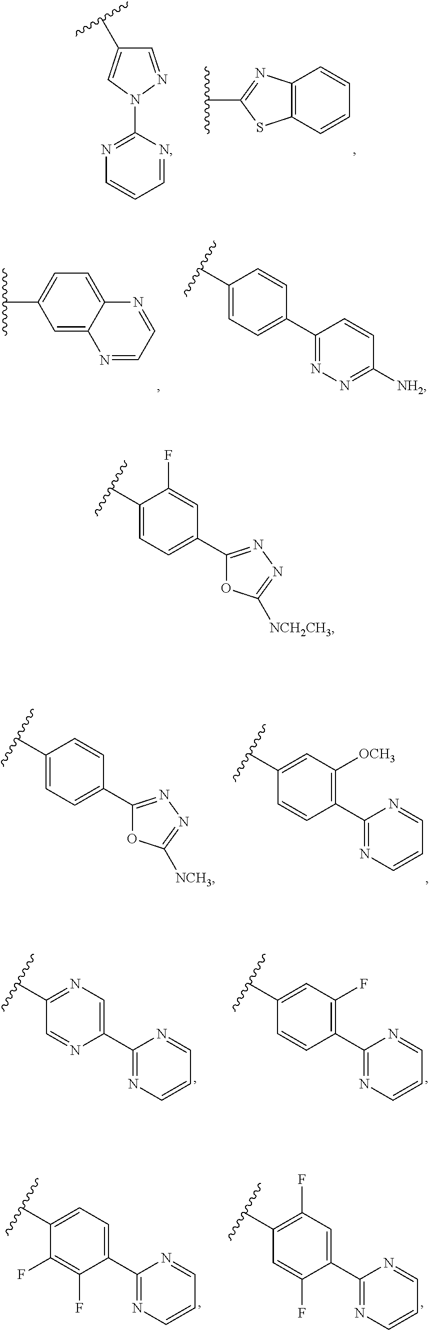

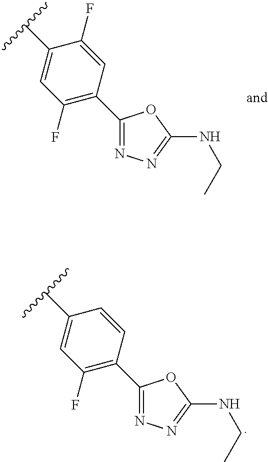

In another embodiment of this invention, R⁵ is selected from the group consisting of:

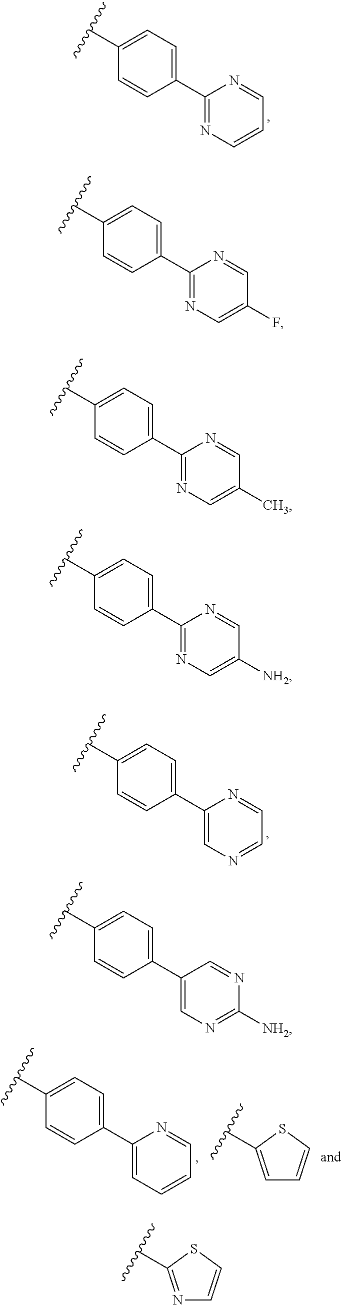

In another embodiment of this invention, R⁵ is selected from the group consisting of:

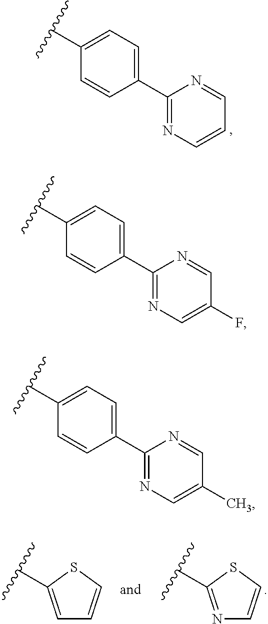

In another embodiment of this invention, R⁵ is selected from the group consisting of:

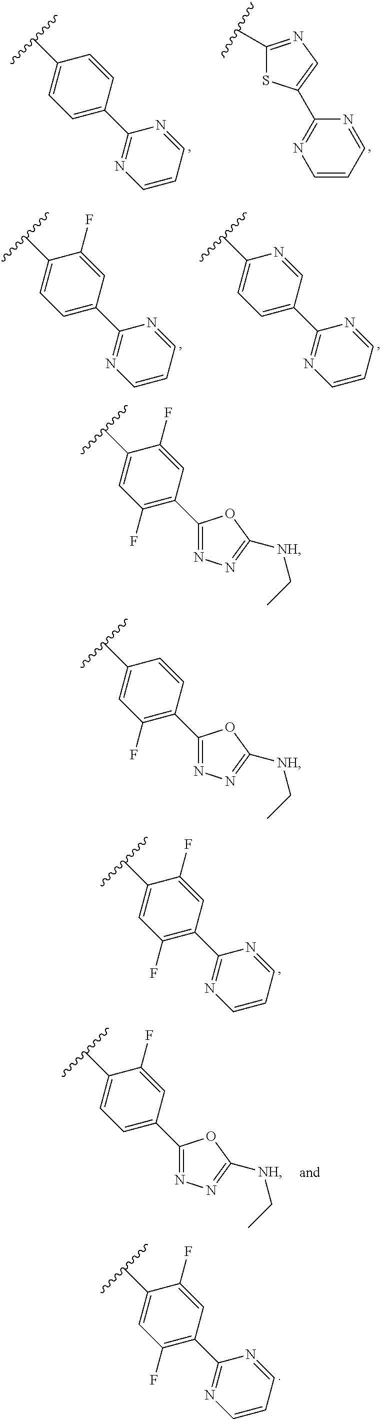

In another embodiment of this invention, R⁵ is selected from the group consisting of:

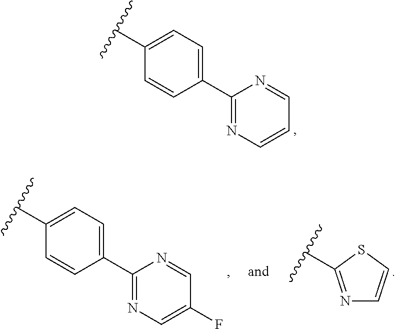

In another embodiment of this invention, R⁵ is:

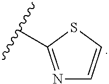

In another embodiment of this invention, R⁵ is:

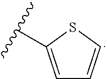

In another embodiment of this invention, R⁵ is:

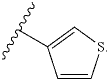

In another embodiment of this invention, R⁵ is:

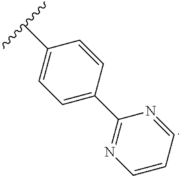

In another embodiment of this invention, R⁵ is:

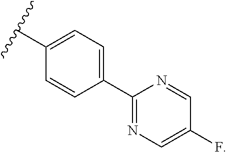

In another embodiment of this invention, R⁵ is:

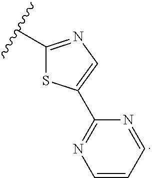

In another embodiment of this invention, R⁵ is:

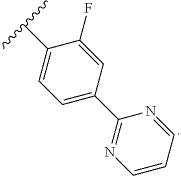

In another embodiment of this invention, R⁵ is:

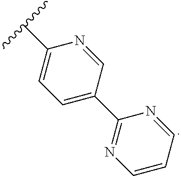

In another embodiment of this invention, R⁵ is:

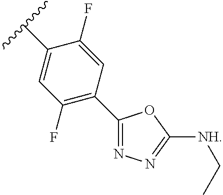

In another embodiment of this invention, R⁵ is:

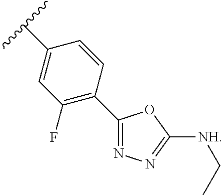

In another embodiment of this invention, R⁵ is:

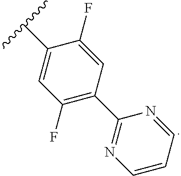

In another embodiment of this invention, R⁵ is:

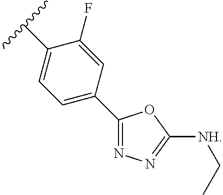

In another embodiment of this invention, R⁵ is:

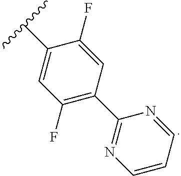

R², in one embodiment of this invention, is —(CH₂)ₘR¹¹, wherein R¹¹ is —OR¹⁰.

R², in another embodiment of this invention, is —(CH₂)ₘR¹¹, wherein R¹¹ is —OR¹⁰, and R¹⁰ is H or alkyl.

R², in another embodiment of this invention, is —(CH₂)ₘR¹¹, wherein R¹¹ is —OR¹⁰, and R¹⁰ alkyl (e.g., methyl).

R², in another embodiment of this invention, is —(CH₂)ₘR¹¹, wherein m is 1 and R¹¹ is —OR¹⁰.

R², in another embodiment of this invention, is —(CH₂)ₘR¹¹, wherein m is 1, R¹¹ is —OR¹⁰, and R¹⁰ is H or alkyl.

R², in another embodiment of this invention, is —(CH₂)ₘR¹¹, wherein m is 1, R¹¹ is —OR¹⁰, and R¹⁰ alkyl.

R², in another embodiment of this invention, is —(CH₂)ₘR¹¹, wherein m is 1, R¹¹ is —OR¹⁰, and R¹⁰ methyl (i.e., R² is —CH₂OCH₃).

R², in another embodiment of this invention, is —OR²³ wherein R²³ is alkyl, and said alkyl is methyl (i.e., R² is —OCH₃).

R², in another embodiment of this invention, is alkynyl. An example of an alkynyl group is ethynyl:

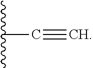

Another example of an alkynyl group is propynyl:

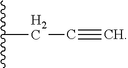

R², in another embodiment of this invention, is alkenyl. An example of an alkenyl group is —CH₂—CH═CH₂.

R², in another embodiment of this invention, is —OCH₃.

R², in another embodiment of this invention, is —S(O)ₜ-alkyl.

R², in another embodiment of this invention, is —S-alkyl (i.e., t is 0) such as, for example, —S—CH₃.

R², in another embodiment of this invention, is —S(O)₂-alkyl (i.e., t is 2) such as, for example, —S(O)₂CH₃.

R², in another embodiment of this invention, is —SCH₃.

R², in another embodiment of this invention, is —S(O)₂CH₃.

R², in another embodiment of this invention, is ethynyl

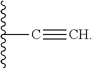

R², in another embodiment of this invention, is —CH₂OCH₃.

Preferably R² is selected from the group consisting of: ethynyl, —OCH₃, and —CH₂OCH₃.

Additional examples of the R²—(CH₂)ₘR¹¹ group include, but are not limited to —CH₂OH, —CH₂CN, —CH₂OC₂H₅, —(CH₂)₃OCH₃, —CH₂F and —CH₂-triazolyl, such as,

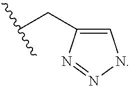

Additional examples of R² include, but are not limited to, H, —CH₂-morpholinyl, —SCH₃, —OC₂H₅, —OCH(CH₃)₂, —CH₂N(CH₃)₂, —CN, —CH(OH)CH₃, —C(O)CH₃, —CH₂C≡CH₃, —CH(CH₃)₂, —C(CH₃)═CH₂, —C(CH₃)═NOCH₃, —C(CH₃)═NOH, —C(CH₃)═NNHC(O)CH₃, —NH₂, —NHC(O)H, —NHCH₃, —CH₂—O—CH₂-cyclopropyl, —CH₂—O—CHF₂, —OCHF₂, —CHF₂, —CH₂C(CH₃)═CH₃, —CH₂CH₂CH₃, —N(CH₃)₂, —CH₂CH₃, —CF₃, —CH═CH₂, and —C(OH)(CH₃)₂.

R³, in one embodiment of this invention, is independently selected from the group consisting of: H and alkyl.

R³, in another embodiment of this invention, is independently selected from the group consisting of: H and methyl.

R³, in another embodiment of this invention, is H.

R⁴, in one embodiment of this invention, is H.

R⁴, in another embodiment of this invention, is selected from the group consisting of: H and alkyl.

R⁴, in another embodiment of this invention, is selected from the group consisting of: H and methyl.

R⁶, in one embodiment of this invention, is R⁶ H.

R⁷, in one embodiment of this invention, is independently selected from the group consisting of: H and alkyl.

R⁷, in another embodiment of this invention, is independently selected from the group consisting of: H and methyl.

R⁷, in one embodiment of this invention, is H.

R⁸, in one embodiment of this invention, is H.

One embodiment of this invention is directed to a compound of formula 1.0, preferably a compound of formula 1.0C1 (e.g., 1.1A) wherein substituent Q is 2.16, and each R³, R⁴, and R⁷ is independently selected from the group consisting of: H and methyl.

One embodiment of this invention is directed to a compound of formula 1.0, preferably a compound of formula 1.0C1 and more preferably a compound of formula, (e.g., 1.1A) wherein substituent Q is 2.16A, and each R³, R⁴, and R⁷ is independently selected from the group consisting of: H and methyl.

One embodiment of this invention is directed to a compound of formula 1.0, preferably a compound of formula 1.0C1 and more preferably a compound of formula 1.0C, (e.g., 1.1A) wherein substituent Q is 2.16B, and each R³, R⁴, and R⁷ is independently selected from the group consisting of: H and methyl.

One embodiment of this invention is directed to a compound of formula 1.0, preferably a compound of formula 1.0C1 (e.g., 1.1A) wherein substituent Q is 2.16, and each R³, R⁴, and R⁷ is H.

One embodiment of this invention is directed to a compound of formula 1.0, preferably a compound of formula 1.0C1 (e.g., 1.1A) wherein substituent Q is 2.16A, and each R³, R⁴, and R⁷ is H.

One embodiment of this invention is directed to a compound of formula 1.0, preferably a compound of formula 1.0C1 (e.g., 1.1A) wherein substituent Q is 2.16B, and each R³, R⁴, and R⁷ is H.

The compounds of this invention inhibit the activity of ERK1 and ERK2 Thus, this invention further provides a method of inhibiting ERK in mammals, especially humans, by the administration of an effective amount (e.g., a therapeutically effective amount) of one or more (e.g., one) compounds of this invention. The administration of the compounds of this invention to patients, to inhibit ERK1 and/or ERK2, is useful in the treatment of cancer.

In any of the methods of treating cancer described herein, unless stated otherwise, the methods can optionally include the administration of an effective amount of one or more (e.g., 1, 2 or 3, or 1 or 2, or 1) chemotherapeutic agents. The chemotherapeutic agents can be administered currently or sequentially with the compounds of this invention.

The methods of treating cancer described herein include methods wherein a combination of drugs (i.e., compounds, or pharmaceutically active ingredients, or pharmaceutical compositions) are used (i.e., the methods of treating cancer of this invention include combination therapies). Those skilled in the art will appreciate that the drugs are generally administered individually as a pharmaceutical composition. The use of a pharmaceutical composition comprising more than one drug is within the scope of this invention.

In any of the methods of treating cancer described herein, unless stated otherwise, the methods can optionally include the administration of an effective amount of radiation therapy. For radiation therapy, γ-radiation is preferred.

Examples of cancers which may be treated by the methods of this invention include, but are not limited to: (A) lung cancer (e.g., lung adenocarcinoma and non small cell lung cancer), (B) pancreatic cancers (e.g., pancreatic carcinoma such as, for example, exocrine pancreatic carcinoma), (C) colon cancers (e.g., colorectal carcinomas, such as, for example, colon adenocarcinoma and colon adenoma), (D) myeloid leukemias (for example, acute myelogenous leukemia (AML), CML, and CMML), (E) thyroid cancer, (F) myelodysplastic syndrome (MDS), (G) bladder carcinoma, (H) epidermal carcinoma, (I) melanoma, (J) breast cancer, (K) prostate cancer, (L) head and neck cancers (e.g., squamous cell cancer of the head and neck), (M) ovarian cancer, (N) brain cancers (e.g., gliomas, such as glioma blastoma multiforme), (O) cancers of mesenchymal origin (e.g., fibrosarcomas and rhabdomyosarcomas), (P) sarcomas, (Q) tetracarcinomas, (R) nuroblastomas, (S) kidney carcinomas, (T) hepatomas, (U) non-Hodgkin's lymphoma, (V) multiple myeloma, and (W) anaplastic thyroid carcinoma.

Chemotherapeutic agents (antineoplastic agent) include but are not limited microtubule affecting agents, alkylating agents, antimetabolites, natural products and their derivatives, hormones and steroids (including synthetic analogs), and synthetics.

Examples of alkylating agents (including nitrogen mustards, ethylenimine derivatives, alkyl sulfonates, nitrosoureas and triazenes) include: Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Examples of antimetabolites (including folic acid antagonists, pyrimidine analogs, purine analogs and adenosine deaminase inhibitors) include: Methotrexate, 5-Fluorouracil, Floxuridine, Cytarabine, 6-Mercaptopurine, 6-Thioguanine, Fludarabine phosphate, Pentostatine, and Gemcitabine.

Examples of natural products and their derivatives (including vinca alkaloids, antitumor antibiotics, enzymes, lymphokines and epipodophyllotoxins) include: Vinblastine, Vincristine, Vindesine, Bleomycin, Dactinomycin, Daunorubicin, Doxorubicin, Epirubicin, Idarubicin, Paclitaxel (paclitaxel is a microtubule affecting agent and is commercially available as Taxol®), Paclitaxel derivatives (e.g. taxotere), Mithramycin, Deoxyco-formycin, Mitomycin-C, L-Asparaginase, Interferons (especially IFN-a), Etoposide, and Teniposide.

Examples of hormones and steroids (including synthetic analogs) include: 17α-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, and Zoladex.

Examples of synthetics (including inorganic complexes such as platinum coordination complexes): Cisplatin, Carboplatin, Hydroxyurea, Amsacrine, Procarbazine, Mitotane, Mitoxantrone, Levamisole, and Hexamethylmelamine.

Examples of other chemotherapeutics include: Navelbene, CPT-11, Anastrazole, Letrazole, Capecitabinbe, Reloxafine, and Droloxafine.

A microtubule affecting agent (e.g., paclitaxel, a paclitaxel derivative or a paclitaxel-like compound), as used herein, is a compound that interferes with cellular mitosis, i.e., having an anti-mitotic effect, by affecting microtubule formation and/or action. Such agents can be, for instance, microtubule stabilizing agents or agent's which disrupt microtubule formation.

Microtubule affecting agents, useful in the methods of this invention, are well known to those skilled in the art and include, but are not limited to: Allocolchicine (NSC 406042), Halichondrin B (NSC 609395), Colchicine (NSC 757), Colchicine derivatives (e.g., NSC 33410), Dolastatin 10 (NSC 376128), Maytansine (NSC 153858), Rhizoxin (NSC 332598), Paclitaxel (Taxol®, NSC 125973), Paclitaxel derivatives (e.g., Taxotere, NSC 608832), Thiocolchicine (NSC 361792), Trityl Cysteine (NSC 83265), Vinblastine Sulfate (NSC 49842), Vincristine Sulfate (NSC 67574), Epothilone A, Epothilone, Discodermolide (see Service, (1996) Science, 274:2009), Estramustine, Nocodazole, MAP4, and the like. Examples of such agents are described in, for example, Bulinski (1997) J. Cell Sci. 110:3055-3064, Panda (1997) Proc. Natl. Acad. Sci. USA 94:10560-10564, Muhlradt (1997) Cancer Res. 57:3344-3346, Nicolaou (1997) Nature 387:268-272, Vasquez (1997) Mol. Biol. Cell. 8:973-985, and Panda (1996) J. Biol. Chem. 271:29807-29812.

Chemotherapeutic agents with paclitaxel-like activity include, but are not limited to, paclitaxel and paclitaxel derivatives (paclitaxel-like compounds) and analogues. Paclitaxel and its derivatives (e.g. Taxol and Taxotere) are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

More specifically, the term “paclitaxel” as used herein refers to the drug commercially available as Taxol® (NSC number: 125973). Taxol® inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

Additional microtubule affecting agents can be assessed using one of many such assays known-in the art, e.g., a semiautomated assay which measures the tubulin-polymerizing activity of paclitaxel analogs in combination with a cellular assay to measure the potential of these compounds to block cells in mitosis (see Lopes (1997) Cancer Chemother. Pharmacol. 41:37-47).

Generally, activity of a test compound is determined by contacting a cell with that compound and determining whether or not the cell cycle is disrupted, in particular, through the inhibition of a mitotic event. Such inhibition may be mediated by disruption of the mitotic apparatus, e.g., disruption of normal spindle formation. Cells in which mitosis is interrupted may be characterized by altered morphology (e.g., microtubule compaction, increased chromosome number, etc.).

Compounds with possible tubulin polymerization activity can be screened in vitro. For example, the compounds are screened against cultured WR21 cells (derived from line 69-2 wap-ras mice) for inhibition of proliferation and/or for altered cellular morphology, in particular for microtubule compaction. In vivo screening of positive-testing compounds can then be performed using nude mice bearing the WR21 tumor cells. Detailed protocols for this screening method are described by Porter (1995) Lab. Anim. Sci., 45(2):145-150.

Other methods of screening compounds for desired activity are well known to those of skill in the art. Typically such assays involve assays for inhibition of microtubule assembly and/or disassembly. Assays for microtubule assembly are described, for example, by Gaskin et al. (1974) J. Molec. Biol., 89: 737-758. U.S. Pat. No. 5,569,720 also provides in vitro and in vivo assays for compounds with paclitaxel-like activity.

Thus, in the methods of this invention wherein at least one chemotherapeutic agent is used, examples of said chemotherapeutic agents include those selected from the group consisting of: microtubule affecting agents, alkylating agents, antimetabolites, natural products and their derivatives, hormones and steroids (including synthetic analogs), and synthetics.

In the methods of this invention wherein at least one chemotherapeutic agent is used, examples of said chemotherapeutic agents also include: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

In the methods of this invention wherein at least one chemotherapeutic agent is used, examples of such chemotherapeutic agents include:

(1) taxanes such as paclitaxel (TAXOL®) and/or docetaxel (Taxotere®);

(2) platinum coordinator compounds, such as, for example, carboplatin, cisplatin and oxaliplatin (e.g. Eloxatin);

(3) EGF inhibitors that are antibodies, such as: HER2 antibodies (such as, for example trastuzumab (Herceptin®), Genentech, Inc.), Cetuximab (Erbitux, IMC-C225, ImClone Systems), EMD 72000 (Merck KGaA), anti-EFGR monoclonal antibody ABX (Abgenix), TheraClM-h-R3 (Center of Molecular Immunology), monoclonal antibody 425 (Merck KGaA), monoclonal antibody ICR-62 (ICR, Sutton, England); Herzyme (Elan Pharmaceutical Technologies and Ribozyme Pharmaceuticals), PKI 166 (Novartis), EKB 569 (Wyeth-Ayerst), GW 572016 (GlaxoSmithKline), CI 1033 (Pfizer Global Research and Development), trastuzmab-maytansinoid conjugate (Genentech, Inc.), mitumomab (Imclone Systems and Merck KGaA) and Melvax II (Imclone Systems and Merck KgaA);

(4) EGF inhibitors that are small molecules, such as, Tarceva (TM) (OSI-774, OSI Pharmaceuticals, Inc.), and Iressa (ZD 1839, Astra Zeneca);

(5) VEGF inhibitors that are antibodies such as: bevacizumab (Genentech, Inc.), and IMC-1C11 (ImClone Systems), DC 101 (a KDR VEGF Receptor 2 from ImClone Systems);

(6) VEGF kinase inhibitors that are small molecules such as SU 5416 (from Sugen, Inc), SU 6688 (from Sugen, Inc.), Bay 43-9006 (a dual VEGF and bRAF inhibitor from Bayer Pharmaceuticals and Onyx Pharmaceuticals);

(7) estrogen receptor antagonists or selective estrogen receptor modulators (SERMs), such as tamoxifen, idoxifene, raloxifene, trans-2,3-dihydroraloxifene, levormeloxifene, droloxifene, MDL 103,323, and acolbifene (Schering Corp.);

(8) anti-tumor nucleoside derivatives such as 5-fluorouracil, gemcitabine, capecitabine, cytarabine (Ara-C), fludarabine (F-Ara-A), decitabine, and chlorodeoxyadenosine (Cda, 2-Cda);

(9) epothilones such as BMS-247550 (Bristol-Myers Squibb), and EP0906 (Novartis Pharmaceuticals);

(10) topoisomerase inhibitors such as topotecan (Glaxo SmithKline), and Camptosar (Pharmacia);

(11) vinca alkaloids, such as, navelbine (Anvar and Fabre, France), vincristine and vinblastine;

(12) antibodies that are inhibitors of αVβ3 integrins, such as, LM-609 (see, Clinical Cancer Research, Vol. 6, page 3056-3061, August 2000, the disclosure of which is incorporated herein by reference thereto);

(13) folate antagonists, such as Methotrexate (MTX), and Premetrexed (Alimta);

(14) ribonucleotide reductase inhibitors, such as Hydroxyurea (HU);

(15) anthracyclines, such as Daunorubicin, Doxorubicin (Adriamycin), and Idarubicin;

(16) biologics, such as interferon (e.g., Intron-A and Roferon), pegylated interferon (e.g., Peg-Intron and Pegasys), and Rituximab (Rituxan, antibody used for the treatment of non-Hodgkin's lymphoma);

(17) thalidomide (or related imid);

(18) Bcr/abl kinase inhibitors, such as, for example Gleevec (STI-571), AMN-17, ONO12380, SU11248 (Sunitinib) and BMS-354825

(19) MEK1 and/or MEK2 inhibitors, such as PD0325901 and Arry-142886 (AZD6244);

(20) IGF-1 and IGF-2 inhibitors that are small molecules, such as, for example, NVP-AEW541;

(21) small molecule inhibitors of RAF and BRAF kinases, such as, for example, BAY 43-9006 (Sorafenib);

(22) small molecule inhibitors of cell cycle dependent kinases such as CDK1, CDK2, CDK4 and CDK6, such as, for example, CYC202, BMS387032, and Flavopiridol;

(23) alkylating agents, such as, for example, Temodar® brand of temozolomide;

(24) farnesyl protein transferase inhibitors, such as, for example:
(a) Sarasar® brand of lonifarnib (i.e., 4-[2-[4-(3,10-dibromo-8-chloro-6,11-dihydro-5H-benzo[5,6]cyclohepta[1,2-b]byridin-11-yl)-1-piperidinyl)-2-oxoethyl]-1-piperidinecarboxamide, see for example, U.S. Pat. No. 5,874,442 issued Feb. 23, 1999, and U.S. Pat. No. 6,632,455 issued Oct. 14, 2003 the disclosures of each being incorporated herein by reference thereto),
(b) Zarnestra® brand of tipifarnib (i.e., (R)-6-amino[(4-chlorophenyl)(1-methyl-1H-imidazol-5-yl)methyl]-4-(3-chlorophenyl)-1-methyl-2(1H)-quinolinone, see for example, WO 97/16443 published May 9, 1997 and U.S. Pat. No. 5,968,952 issued Oct. 19, 1999, the disclosures of each being incorporated herein by reference thereto), and
(c) Bristol-Myers Squibb 214662:

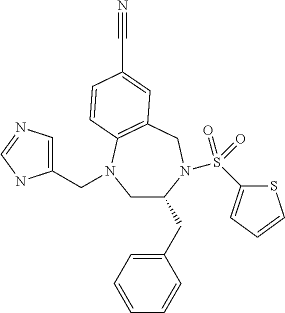

(see WO97/30992 published Aug. 28, 1997, U.S. Pat. No. 6,011,029 issued Jan. 4, 2000, and U.S. Pat. No. 6,455,523, the disclosures of each being incorporated herein by reference thereto).

The Bcr/abl kinase inhibitors, EGF receptor inhibitors, and HER-2 antibodies (EGF receptor inhibitors that are antibodies) described above are also known as signal transduction inhibitors. Therefore, chemotherapeutic agents, as used herein, include signal transduction inhibitors.

Typical signal transduction inhibitors, that are chemotherapeutic agents, include but are not limited to: (i) Bcr/abl kinase inhibitors such as, for example, STI 571 (Gleevec), (ii) Epidermal growth factor (EGF) receptor inhibitor such as, for example, Kinase inhibitors (Iressa, OSI-774) and antibodies (Imclone: C225 [Goldstein et al. (1995), Clin Cancer Res. 1:1311-1318], and Abgenix: ABX-EGF) and (iii) HER-2/neu receptor inhibitors such as, for example, Herceptin® (trastuzumab).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA), the Physician's Desk Reference, 56^{th} Edition, 2002 (published by Medical Economics company, Inc. Montvale, N.J. 07645-1742), and the Physician's Desk Reference, 57^{th} Edition, 2003 (published by Thompson PDR, Montvale, N.J. 07645-1742); the disclosures of which is incorporated herein by reference thereto.

For example, the compound of formula 1.0 (e.g., a pharmaceutical composition comprising the compound of formula 1.0); can be administered orally (e.g., as a capsule), and the chemotherapeutic agents can be administered intravenously, usually as an IV solution. The use of a pharmaceutical composition comprising more than one drug is within the scope of this invention.

The compound of formula 1.0 and the chemotherapeutic agents are administered in therapeutically effective dosages to obtain clinically acceptable results, e.g., reduction or elimination of symptoms or of the tumor. Thus, the compound of formula 1.0 and chemotherapeutic agents can be administered concurrently or consecutively in a treatment protocol. The administration of the chemotherapeutic agents can be made according to treatment protocols already known in the art.

In general when more than one chemotherapeutic agent is used in the methods of this invention, the chemotherapeutic agents are administered on the same day either concurrently or consecutively in their standard dosage form. For example, the chemotherapeutic agents are usually administered intravenously, preferably by an IV drip using IV solutions well known in the art (e.g., isotonic saline'(0.9% NaCl) or dextrose solution (e.g., 5% dextrose)).

When two or more chemotherapeutic agents are used, the chemotherapeutic agents are generally administered on the same day; however, those skilled in the art will appreciate that the chemotherapeutic agents can be administered on different days and in different weeks. The skilled clinician can administer the chemotherapeutic agents according to their recommended dosage schedule from the manufacturer of the agent and can adjust the schedule according to the needs of the patient, e.g., based on the patient's response to the treatment. For example, when gemcitabine is used in combination with a platinum coordinator compound, such as, for example, cisplatin, to treat lung cancer, both the gemcitabine and the cisplatin are given on the same day on day one of the treatment cycle, and then gemcitabine is given alone on day 8 and given alone again on day 15

The compounds of this invention and chemotherapeutic agents can be administered in a treatment protocol that usually lasts one to seven weeks, and is repeated typically from 6 to 12 times. Generally the treatment protocol can last one to four weeks. Treatment protocols of one to three weeks can also be used. A treatment protocol of one to two weeks can also be used. During this treatment protocol or cycle the compounds of this invention can be administered daily while the chemotherapeutic agents can be administered one or more times a week. Generally, a compound of this invention can be administered daily (i.e., once per day), and in one embodiment twice per day, and the chemotherapeutic agent is administered once a week or once every three weeks. For example, the taxanes (e.g., Paclitaxel (e.g., Taxol®) or Docetaxel (e.g., Taxotere®)) can be administered once a week or once every three weeks.

However, those skilled in the art will appreciate that treatment protocols can be varied according to the needs of the patient. Thus, the combination of compounds (drugs) used in the methods of this invention can be administered in variations of the protocols described above. For example, the compounds of this invention can be administered discontinuously rather than continuously during the treatment cycle. Thus, for example, during the treatment cycle the compounds of this invention can be administered daily for a week and then discontinued for a week, with this administration repeating during the treatment cycle. Or the compounds of this invention can be administered daily for two weeks and discontinued for a week, with This administration repeating during the treatment cycle. Thus, the compounds of this invention can be administered daily for one or more weeks during the cycle and discontinued for one or more weeks during the cycle, with this pattern of administration repeating during the treatment cycle. This discontinuous treatment can also be based upon numbers of days rather than a full week. For example, daily dosing for 1 to 6 days, no dosing for 1 to 6 days with this pattern repeating during the treatment protocol. The number of days (or weeks) wherein the compounds of this invention are not dosed do not have to equal the number of days (or weeks) wherein the compounds of this invention are dosed. Usually, if a discontinuous dosing protocol is used, the number of days or weeks that the compounds of this invention are dosed is at least equal or greater than the number of days or weeks that the compounds of this invention are not dosed.

The chemotherapeutic agent could be given by bolus or continuous infusion. The chemotherapeutic agent could be given daily to once every week, or once every two weeks, or once every three weeks, or once every four weeks during the treatment cycle. If administered daily during a treatment cycle, this daily dosing can be discontinuous over the number of weeks of the treatment cycle. For example, dosed for a week (or a number of days), no dosing for a week (or a number of days, with the pattern repeating during the treatment cycle.

The compounds of this invention can be administered orally, preferably as a solid dosage form, and in one embodiment as a capsule, and while the total therapeutically effective daily dose can be administered in one to four, or one to two divided doses per day, generally, the therapeutically effective dose is given once or twice a day, and in one embodiment twice a day. The compounds of this invention can be administered in an amount of about 50 to about 400 mg once per day, and can be administered in an amount of about 50 to about 300 mg once per day. The compounds of this invention are generally administered in an amount of about 50 to about 350 mg twice a day, usually 50 mg to about 200 mg twice a day, and in one embodiment about 75 mg to about 125 mg administered twice a day, and in another embodiment about 100 mg administered twice a day.

If the patient is responding, or is stable, after completion of the therapy cycle, the therapy cycle can be repeated according to the judgment of the skilled clinician. Upon completion of the therapy cycles, the patient can be continued on the compounds of this invention at the same dose that was administered in the treatment protocol, or, if the dose was less than 200 mg twice a day, the dose can be raised to 200 mg twice a day. This: maintenance dose can be continued until the patient progresses or can no longer tolerate the dose (in which case the dose can be reduced and the patient can be continued on the reduced dose).

The chemotherapeutic agents, used with the compounds of this invention, are administered in their normally prescribed dosages during the treatment cycle (i.e., the chemotherapeutic agents are administered according to the standard of practice for the administration of these drugs). For example: (a) about 30 to about 300 mg/m² for the taxanes; (b) about 30 to about 100 mg/m² for Cisplatin; (c) AUC of about 2 to about 8 for Carboplatin; (d) about 2 to about 4 mg/m² for EGF inhibitors that are antibodies; (e) about 50 to about 500 mg/m² for EGF inhibitors that are small molecules; (f) about 1 to about 10 mg/m² for VEGF kinase inhibitors that are antibodies; (g) about 50 to about 2400 mg/m² for VEGF inhibitors that are small molecules; (h) about 1 to about 20 mg for SERMs; (i) about 500 to about 1250 mg/m² for the anti-tumor nucleosides 5-Fluorouracil, Gemcitabine and Capecitabine; (j) for the anti-tumor nucleoside Cytarabine (Ara-C) 100-200 mg/m²/day for 7 to 10 days every 3 to 4 weeks, and high doses for refractory leukemia and lymphoma, i.e., 1 to 3 gm/m² for one hour every 12 hours for 4-8 doses every 3 to four weeks; (k) for the anti-tumor nucleoside Fludarabine (F-ara-A) 10-25 mg/m²/day every 3 to 4 weeks; (l) for the anti-tumor nucleoside Decitabine 30 to 75 mg/m² for three days every 6 weeks for a maximum of 8 cycles; (m) for the anti-tumor nucleoside Chlorodeoxyadenosine (CdA, 2-CdA) 0.05-0.1 mg/kg/day as continuous infusion for up to 7 days every 3 to 4 weeks; (n) about 1 to about 100 mg/m² for epothilones; (o) about 1 to about 350 mg/m² for topoisomerase inhibitors; (p) about 1 to about 50 mg/m² for vinca alkaloids; (q) for the folate antagonist Methotrexate (MTX) 20-60 mg/m² by oral, IV or IM every 3 to 4 weeks, the intermediate dose regimen is 80-250 mg/m² IV over 60 minutes every 3 to 4 weeks, and the high dose regimen is 250-1000 mg/m² IV given with leucovorin every 3 to 4 weeks; (r) for the folate antagonist Premetrexed (Alimta) 300-600 mg/m² (10 minutes IV infusion day 1) every 3 weeks; (s) for the ribonucleotide reductase inhibitor Hydroxyurea (HU) 20-50 mg/kg/day (as needed to bring blood cell counts down); (t) the platinum coordinator compound Oxaliplatin (Eloxatin) 50-100 mg/m² every 3 to 4 weeks (preferably used for solid tumors such as non-small cell lung cancer, colorectal cancer and ovarian cancer); (u) for the anthracycline daunorubicin 10-50 mg/m²/day IV for 3-5 days every 3 to 4 weeks; (v) for the anthracycline Doxorubicin (Adriamycin) 50-100 mg/m² IV continuous infusion over 1-4 days every 3 to 4 weeks, or 10-40 mg/m² IV weekly; (w) for the anthracycline Idarubicin 10-30 mg/m² daily for 1-3 days as a slow IV infusion over 10-20 minutes every 3 to 4 weeks; (x) for the biologic interferon (Intron-A, Roferon) 5 to 20 million IU three times per week; (y) for the biologic pegylated interferon (Peg-intron, Pegasys) 3 to 4 micrograms/kg/day chronic sub cutaneous (until relapse or loss of activity); (z) for the biologic Rituximab (Rituxan) (antibody used for non-Hodgkin's lymphoma) 200-400 mg/m² IV weekly over 4-8 weeks for 6 months; (aa) for the alkylating agent temozolomide 75 mg/m² to 250 mg/m², for example, 150 mg/m², or for example, 200 mg/m², such as 200 mg/m² for 5 days; and (bb) for the MEK1 and/or MEK2 inhibitor PD0325901, 15 mg to 30 mg, for example, 15 mg daily for 21 days every 4 weeks.

Gleevec can be used orally in an amount of about 200 to about 800 mg/day.

Thalidomide (and related imids) can be used orally in amounts of about 200 to about 800 mg/day, and can be contiuously dosed or used until releapse or toxicity. See for example Mitsiades et al., “Apoptotic signaling induced by immunomodulatory thalidomide analogs in human multiple myeloma cells; therapeutic implications”, Blood, 99(12):4525-30, Jun. 15, 2002, the disclosure of which is incorporated herein by reference thereto.

The FPT inhibitor Sarasar® (brand of Ionifarnib) can be administered orally (e.g., capsule) in amounts of about 50 to about 200 mg given twice a day, or in amounts of about 75 to about 125 mg given twice a day, or in amounts of about 100 to about 200 mg given twice a day, or in an amount of about 100 mg given twice a day.

Paclitaxel (e.g., Taxol®), for example, can be administered once per week in an amount of about 50 to about 100 mg/m² and in another example about 60 to about 80 mg/m². In another example Paclitaxel (e.g., Taxol®) can be administered once every three weeks in an amount of about 150 to about 250 mg/m² and in another example about 175 to about 225 mg/m².

In another example, Docetaxel (e.g., Taxotere®) can be administered once per week in an amount of about 10 to about 45 mg/m². In another example Docetaxel (e.g., Taxotere®) can be administered once every three weeks in an amount of about 50 to about 100 mg/m².

In another example Cisplatin can be administered once per week in an amount of about 20 to about 40 mg/m². In another example Cisplatin can be administered once every three weeks in an amount of about 60 to about 100 mg/m².

In another example Carboplatin can be administered once per week in an amount to provide an AUC of about 2 to about 3. In another example Carboplatin can be administered once every three weeks in an amount to provide an AUC of about 5 to about 8.

Other embodiments of this invention are described below. The embodiments have been numbered for the purpose of making it easier to refer to the embodiments. The term “in any one of Embodiment Nos.” or the term “of any of Embodiment Nos.”, as used below, means that the particular embodiment using that term is intended to cover any one of the embodiments referred to as if any one of the referred to embodiments had been individually described. “Nos.” is an abbreviation for Numbers.

Embodiment No. 1 is directed to a compound of formula 1.0, preferably a compound of formula 1.0C1, wherein Q is selected from the group consisting of substituents 2.1, 2.2, 2.3, 2.3A, 2.3B, 2.3C, 2.4A, 2.4B, 2.4C, 2.5A, 2.5B, 2.5C, 2.6A, 2.7A, 2.7B, 2.7C, 2.8A, 2.8B, 2.8C, 2.9 to 2.14, 2.15, 2.16 (e.g., 2.16A or 2.16B), 2.17, 2.17A, 2.17B, 2.17C, 2.17D, 2.17E, 2.18, 2.19, 2.20, 2.21 and 2.22.

Embodiment No. 2 is directed to a compound of formula 1.0, preferably a compound of formula 1.0C1 (e.g., 1.1A) wherein substituent Q is 2.1.

Embodiment No. 2 is directed to a compound of formula 1.0, preferably a compound of formula 1.0C1 (e.g., 1.1A) wherein substituent Q is 2.2.

Embodiment No. 3 is directed to a compound of formula 1.0 (e.g., 1.1A) wherein substituent Q is 2.3 (e.g., 2.3A, 2.3B or 2.3C).

Embodiment No. 4 is directed to a compound of formula 1.0 (e.g., 1.1A) wherein substituent Q is 2.4 (e.g., 2.4A, 2.4B or 2.4C).

Embodiment No. 5 is directed to a compound of formula 1.0, preferably a compound of formula 1.0C1 (e.g., 1.1A) wherein substituent Q is 2.5 (e.g., 2.5A, 2.5B or 2.5C).

Embodiment No. 6 is directed to any of compounds of formulas to a compound of formula 1.0, preferably a compound of formula 1.0C1 (e.g., 1.1A) wherein substituent Q is 2.6 (e.g., 2.6A).

Embodiment No. 7 is directed to a compound of formula 1.0, preferably a compound of formula 1.0C1 (e.g., 1.1A) wherein, substituent Q is 2.7.

Embodiment No. 8 is directed to a compound of formula 1.0, preferably a compound of formula 1.0C1 (e.g., 1.1A) wherein substituent Q is 2.8.

Embodiment No. 9 is directed to a compound of formula 1.0, preferably a compound of formula 1.0C1 (e.g., 1.1A) wherein substituent Q is 2.9.

Embodiment No. 10 is directed to a compound of formula 1.0, preferably a compound of formula 1.0C1 (e.g., 1.1A) wherein substituent Q is 2.10.

Embodiment No. 11 is directed to a compound of formula 1.0, preferably a compound of formula 1.0C1 (e.g., 1.1A) wherein substituent Q is 2.11.

Embodiment No. 12 is directed to a compound of formula 1.0, preferably a compound of formula 1.0C1 (e.g., 1.1A) wherein substituent Q is 2.12.

Embodiment No. 13 is directed to a compound of formula 1.0, preferably a compound of formula 1.0C1 (e.g., 1.1A) wherein substituent Q is 2.13.

Embodiment No. 14 is directed to a compound of formula 1.0, preferably a compound of formula 1.0C1 (e.g., 1.1A) wherein substituent Q is 2.14.

Embodiment No. 15 is directed to a compound of formula 1.0, preferably a compound of formula 1.0C1 (e.g., 1.1A) wherein substituent Q is 2.15.

Embodiment No. 16 is directed to a compound of formula 1.0, preferably a compound of formula 1.0C1 (e.g., 1.1A) wherein substituent Q is 2.16.

Embodiment No. 17 is directed to a compound of formula 1.0, preferably a compound of formula 1.0C1 (e.g., 1.1A) wherein substituent Q is 2.17 (e.g., 2.17A, 2.17B, 2.17C, 2.17D, or 2.17E).

Embodiment No. 18 is directed to a compound of formula 1.0, preferably a compound of formula 1.0C1 (e.g., 1.1A) wherein substituent Q is 2.18.

Embodiment No. 19 is directed to a compound of formula 1.0, preferably a compound of formula 1.0C1 (e.g., 1.1A) wherein substituent Q is 2.19.

Embodiment No. 20 is directed to a compound of formula 1.0, preferably a compound of formula 1.0C1 (e.g., 1.1A) wherein substituent Q is 2.20.

Embodiment No. 21 is directed to a compound of formula 1.0, preferably a compound of formula 1.0C1 (e.g., 1.1A) wherein substituent Q is 2.21.

Embodiment No. 22 is directed to a compound of formula 1.0, preferably a compound of formula 1.0C1 (e.g., 1.1A) wherein substituent Q is 2.22.

Embodiment No. 23 is directed to a compound of formula⁻1:0, preferably a compound of formula 1.0C1 (e.g., 1.1A) wherein each R³, R⁴, R⁶, and R⁷ is independently selected from the group consisting of: H and alkyl:

Embodiment No. 24 is directed to a compound of formula 1.0, preferably a compound of formula 1.0C1 (e.g., 1.1A) wherein each R³, R⁴, R⁶, and R⁷ is independently selected from the group consisting of: H and methyl.

Embodiment No. 25 is directed to a compound of formula 1.0, preferably a compound of formula 1.0C1 (e.g., 1.1A) wherein each R³, R⁴, R⁶, and R⁷ is H.

Embodiment No. 26 is directed to a compound of formula 1.0, preferably a compound of formula 1.0C1 (e.g., 1.1A) wherein substituent Q is selected from the group consisting of: moieties 2.1, 2.2, 2.3A, 2.3B, and 2.3C.

Embodiment No. 27 is directed to a compound of formula 1.0, preferably a compound of formula 1.0C1 (e.g., 1.1A) wherein substituent Q is selected from the group consisting of: moieties 2.1, 2.2, 2.3A, 2.3B, and 2.3C, and each R³, R⁴, R⁶, and R⁷ is independently selected from the group consisting of: H and alkyl.

Embodiment No. 28 is directed to a compound of formula 1.0, preferably a compound of formula 1.0C1 (e.g., 1.1A) wherein substituent Q is selected from the group consisting of: moieties 2.1, 2.2, 2.3A, 2.3B, and 2.3C, and each R³, R⁴, R⁶, and R⁷ is independently selected from the group consisting of: H and methyl.

Embodiment No. 29 is directed to a compound of formula 1.0, preferably a compound of formula 1.0C1 (e.g., 1.1A) wherein substituent Q is selected from the group consisting of: moieties 2.1, 2.2, 2.3A, 2.3B, and 2.3C, and each R³, R⁴, R⁶, and R⁷ is H.

Embodiment No. 30 is directed to a compound of formula 1.0, preferably a compound of formula 1.0C1 (e.g., 1.1A) wherein substituent Q is selected from the group consisting of: moiety 2.17.

Embodiment No. 31 is directed to a compound of formula 1.0, preferably a compound of formula 1.0C1 (e.g., 1.1A) wherein substituent Q is selected from the group consisting of: moiety 2.17, and each R³, R⁴, R⁶, and R⁷ is independently selected from the group consisting of: H and alkyl.

Embodiment No. 32 is directed to a compound of formula 1.0, preferably a compound of formula 1.0C1 (e.g., 1.1A) wherein substituent Q is selected from the group consisting of: moiety 2.17, and each R³, R⁴, R⁶, and R⁷ is independently selected from the group consisting of: H and methyl.

Embodiment No. 33 is directed to a compound of formula 1.0, preferably a compound of formula 1.0C1 (e.g., 1.1A) wherein substituent Q is selected from the group consisting of: moiety 2.17, and each R³, R⁴, R⁶, and R⁷ is H.

Embodiment No. 34 is directed to a compound of formula 1.0, preferably a compound of formula 1.0C1 (e.g., 1.1A) wherein substituent Q is selected from the group consisting of: moieties 2.1, and: (1) each R³, R⁴, R⁶, and R⁷ is independently selected from the group consisting of: H and alkyl, or (2) each R³, R⁴, R⁶, and R⁷ is independently selected from the group consisting of: H and methyl, or (3) each R³, R⁴, R⁶, and R⁷ is H.

Embodiment No. 35 is directed to a compound of formula 1.0, preferably a compound of formula 1.0C1 (e.g., 1.1A) wherein substituent Q is selected from the group consisting of: moieties 2.2, and: (1) each R³, R⁴, R⁶, and R⁷ is independently selected from the group consisting of: H and alkyl, or (2) each R³, R⁴, R⁶, and R⁷ is independently selected from the group consisting of: H and methyl, or (3) each R³, R⁴, R⁶, and R⁷ is H.

Embodiment No. 36 is directed to a compound of formula 1.0, preferably a compound of formula 1.0C1 (e.g., 1.1A) wherein substituent Q is selected from the group consisting of: moieties 2.3A, 2.3B, 2.3C, and: (1) each R³, R⁴, R⁶, and R⁷ is independently selected from the group consisting of: H and alkyl, or (2) each R³, R⁴, R⁶, and R⁷ is independently selected from the group consisting of: H and methyl, or (3) each R³, R⁴, R⁶, and R⁷ is H.

Embodiment No. 37 is directed to a compound of formula 1.0, preferably a compound of formula 1.0C1 (e.g., 1.1A) wherein substituent Q is selected from the group consisting of: moieties 2.6, 2.7A, 2.7B and 2.7C.

Embodiment No. 38 is directed to a compound of formula 1.0, preferably a compound of formula 1.0C1 (e.g., 1.1A) wherein substituent Q is selected from the group consisting of: moieties 2.6, 2.7A, 2.7B and 2.7C, and each R³, R⁴, R⁶, and R⁷ is independently selected from the group consisting of: H and alkyl.

Embodiment No. 39 is directed to a compound of formula 1.0, preferably a compound of formula 1.0C1 (e.g., 1.1A) wherein substituent Q is selected from the group consisting of: moieties 2.6, 2.7A, 2.7B and 2.7C, and each R³, R⁴, R⁶, and R⁷ is independently selected from the group consisting of: H and methyl.

Embodiment No. 40 is directed to a compound of formula 1.0, preferably a compound of formula 1.0C1 (e.g., 1.1A) wherein substituent Q is selected from the group consisting of: moieties 2.6, 2.7A, 2.7B and 2.7C, and each R³, R⁴; R⁶, and R⁷ is H.

Embodiment No. 41 is directed to a compound of formula 1.0, preferably a compound of formula 1.0C1 (e.g., 1.1A) wherein substituent Q is selected from the group consisting of: moiety 2.6, and: (1) each R³, R⁴, R⁶, and R⁷ is independently selected from the group consisting of: H and alkyl, or (2) each R³, R⁴, R⁶, and R⁷ is independently selected from the group consisting of: H and methyl, or (3) each R³, R⁴, R⁶, and R⁷ is H.

Embodiment No. 42 is directed to a compound of formula 1.0, preferably a compound of formula 1.0C1 (e.g., 1.1A) wherein substituent Q is selected from the group consisting of: moiety 2.7A, and: (1) each R³, R⁴, R⁶, and R⁷ is independently selected from the group consisting of: H and alkyl, or (2) each R³, R⁴, R⁶, and R⁷ is independently selected from the group consisting of: H and methyl, or (3) each R³, R⁴, R⁶, and R⁷ is H.

Embodiment No. 43 is directed to a compound of formula 1.0, preferably a compound of formula 1.0C1 (e.g., 1.1A) wherein substituent Q is selected from the group consisting of: moieties 2.7B and 2.7C, and: (1) each R³, R⁴, R⁶, and R⁷ is independently selected from the group consisting of: H and alkyl, or (2) each R³, R⁴, R⁶, and R⁷ is independently selected from the group consisting of: H and methyl, or (3) each R³, R⁴, R⁶, and R⁷ is H.

Embodiment No. 44 is directed to a compound of any one of Embodiment Nos. 1 to 43, wherein R¹ is selected from the group consisting of:

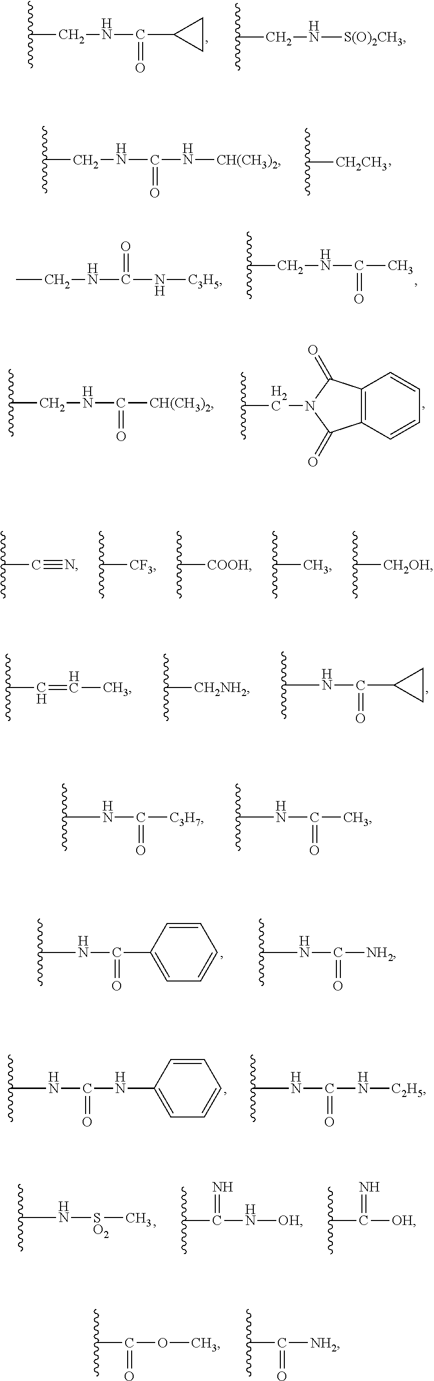

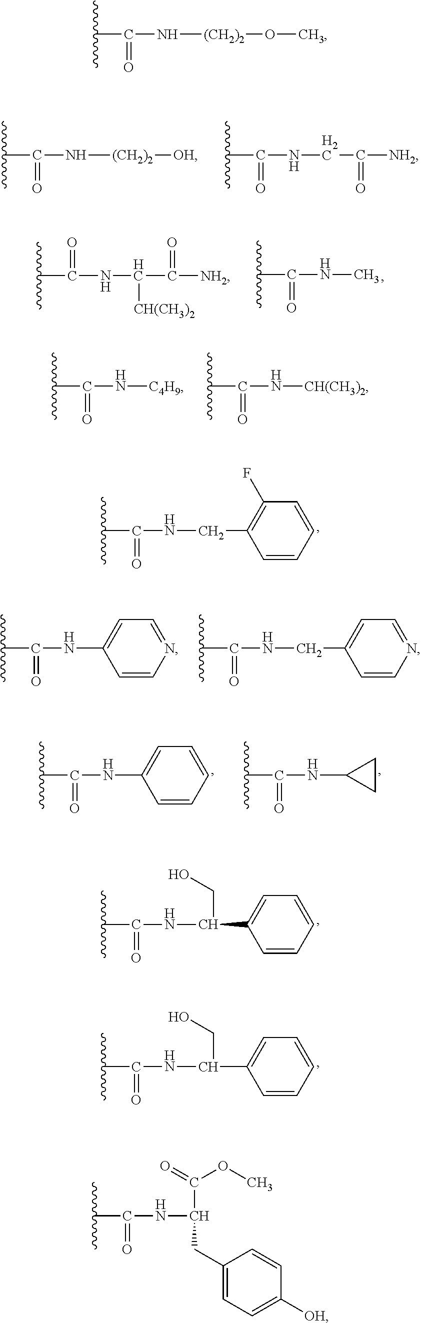

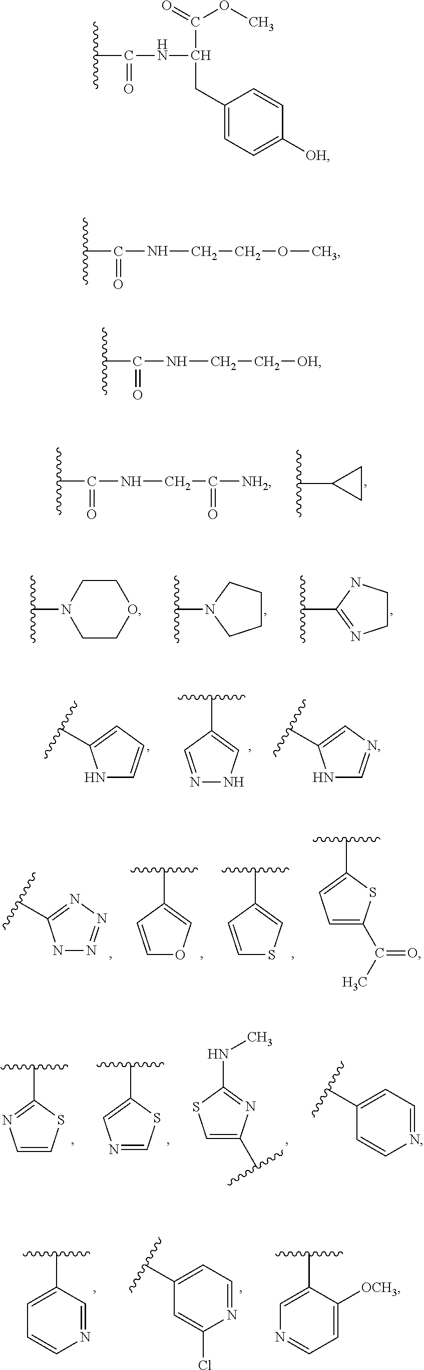

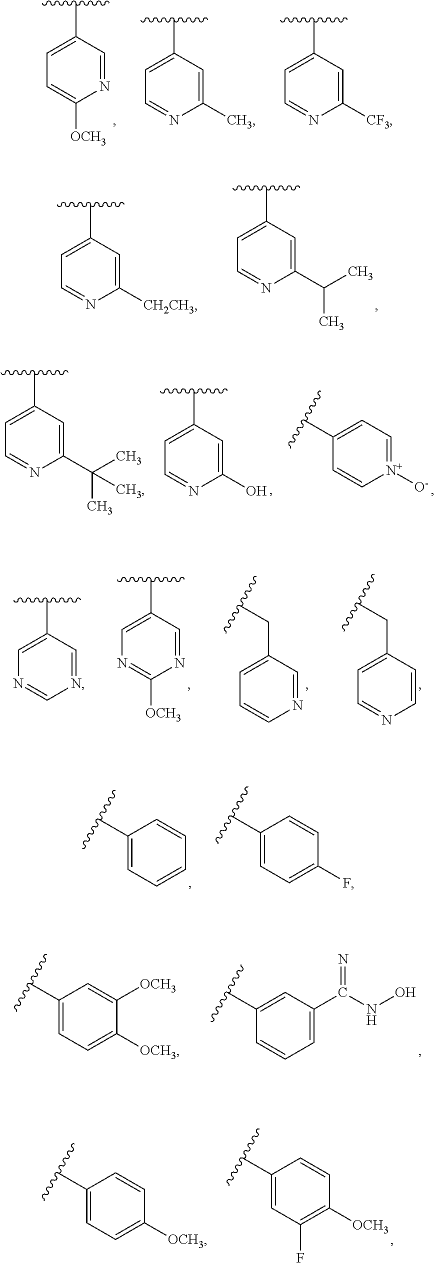

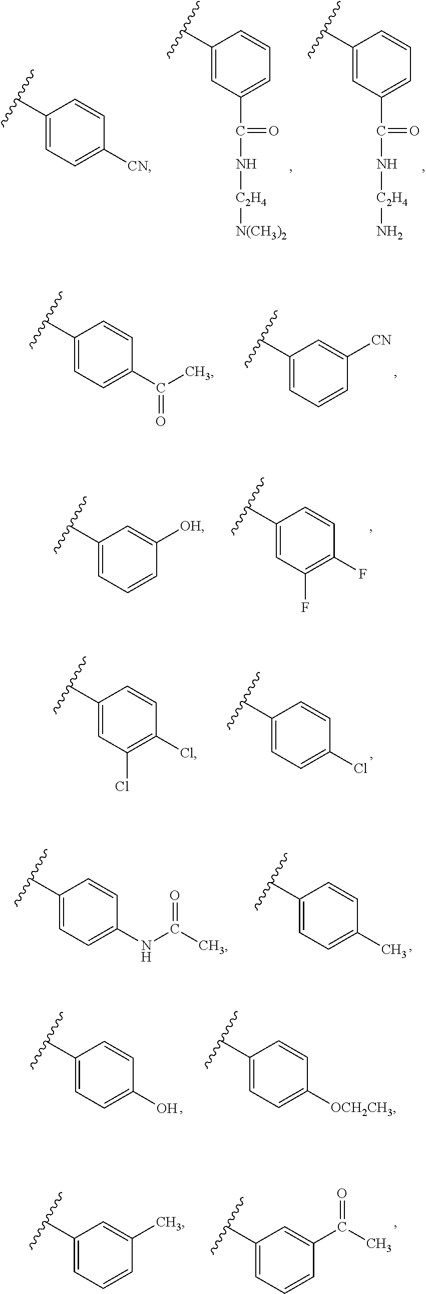

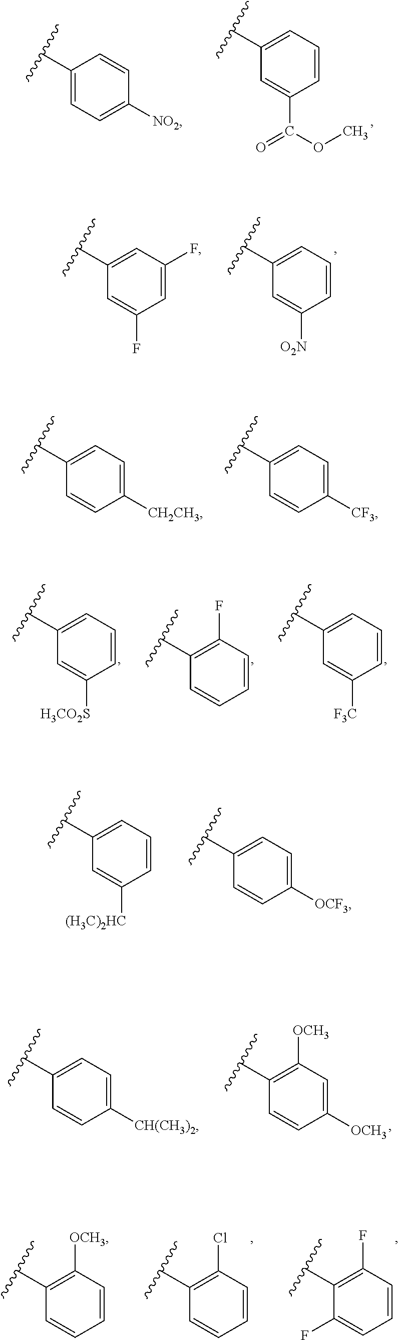

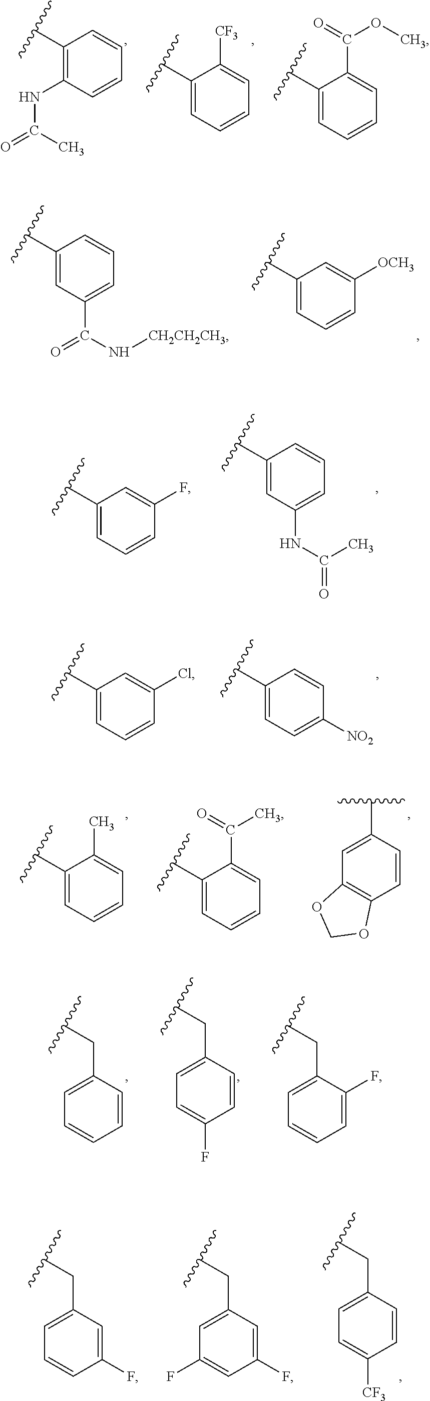

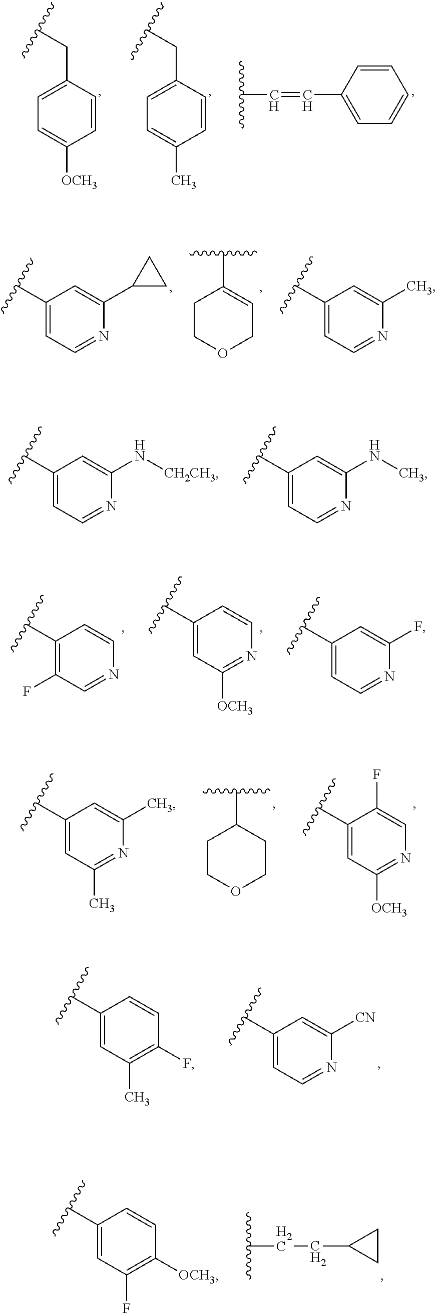

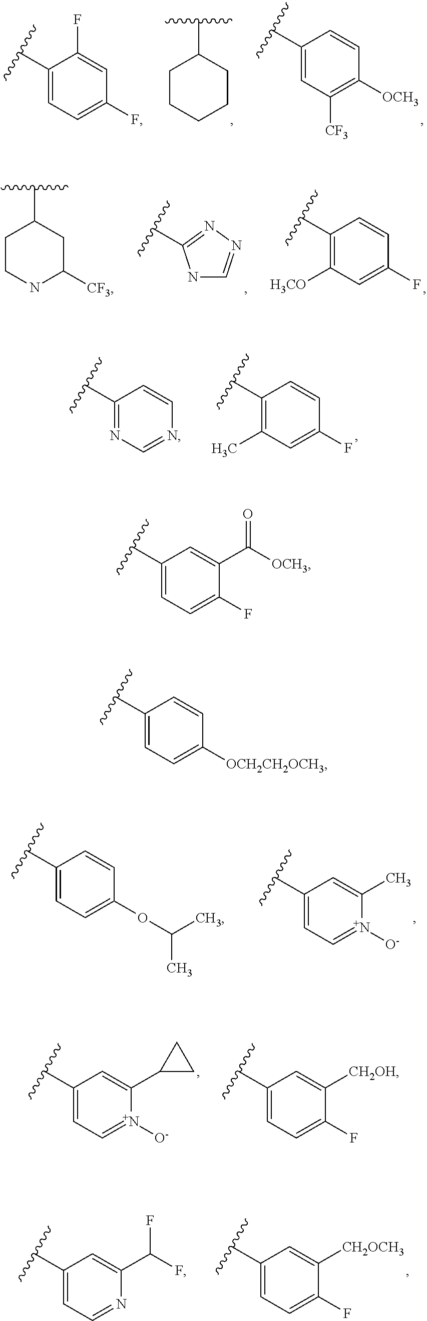

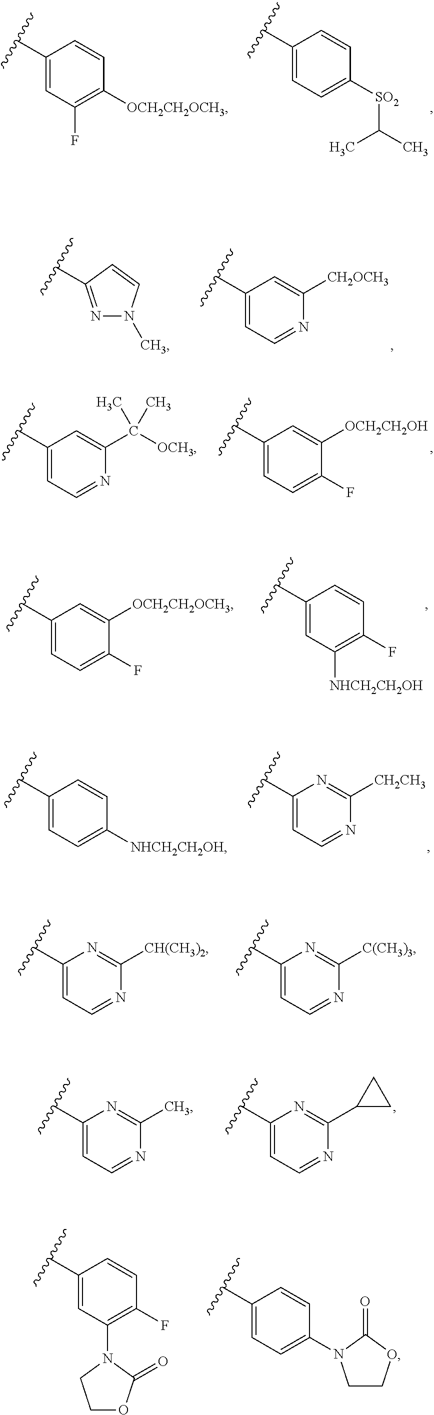

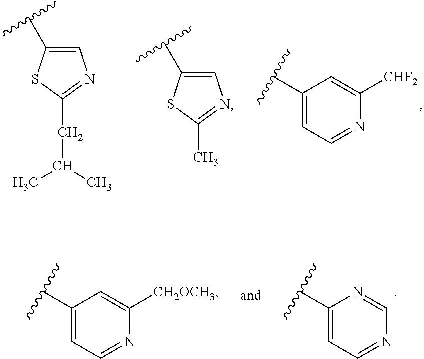

Embodiment No. 45 is directed to a compound of any one of Embodiment Nos. 1 to 43, wherein R¹ is aryl (e.g., phenyl).

Embodiment No. 46 is directed to a compound of any one of Embodiment Nos. 1 to 43, wherein R¹ is substituted aryl (e.g., substituted phenyl).

Embodiment No. 47 is directed to a compound of any one of Embodiment Nos. 1 to 43, wherein R¹ is heteroaryl (e.g., pyridyl, such as

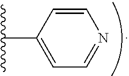

Embodiment No. 48 is directed to a compound of any one of Embodiment Nos. 1 to 43, wherein R¹ is substituted heteroaryl (e.g., substituted pyridyl).

Embodiment No. 49 is directed to a compound of any one of Embodiment Nos. 1 to 43, wherein R¹ is pyridyl substituted with cycloalkyl (e.g., cyclopropyl).

Embodiment No. 50 is directed to a compound of any one of Embodiment Nos. 1 to 43, wherein R¹ is pyridyl substituted with cyclopropyl.

Embodiment No. 51 is directed to a compound of any one of Embodiment Nos. 1 to 43, wherein R¹ is:

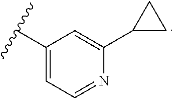

Embodiment No. 52 is directed to a compound of any one of Embodiment Nos. 1 to 43, wherein R¹ is phenyl substituted with halo.

Embodiment No. 53 is directed to a compound of any one of Embodiment Nos. 1 to 43, wherein R¹ is phenyl substituted with F.

Embodiment No. 54 is directed to a compound of any one of Embodiment Nos. 1 to 43, wherein R¹ is p-F-phenyl.

Embodiment No. 55 is directed to a compound of any one of Embodiment Nos. 1 to 43, wherein R¹ is pyridyl substituted with —CF₃.

Embodiment No. 56 is directed to a compound of any one of Embodiment Nos. 1 to 43, wherein R¹ is:

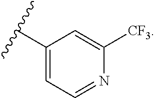

Embodiment No. 57 is directed to a compound of any one of Embodiment Nos. 1 to 43, wherein R¹ is pyridyl substituted with alkyl.

Embodiment No. 58 is directed to a compound of any one of Embodiment Nos. 1 to 43, wherein R¹ is pyridyl substituted with methyl.

Embodiment No. 59 is directed to a compound of any one of Embodiment Nos. 1 to 43, wherein R¹ is:

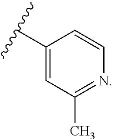

Embodiment No. 60 is directed to a compound of any one of Embodiment Nos. 1 to 43, wherein R¹ is p-CH₃O-phenyl.

Embodiment No. 61 is directed to a compound of any one of Embodiment Nos. 1 to 43, wherein R¹ is

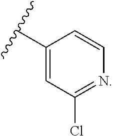

Embodiment No. 62 is directed to a compound of any one of Embodiment Nos. 1 to 43, wherein R¹ is pyridyl.

Embodiment No. 63 is directed to a compound of any one of Embodiment Nos. 1 to 43 wherein R⁵ is selected from the group consisting of:

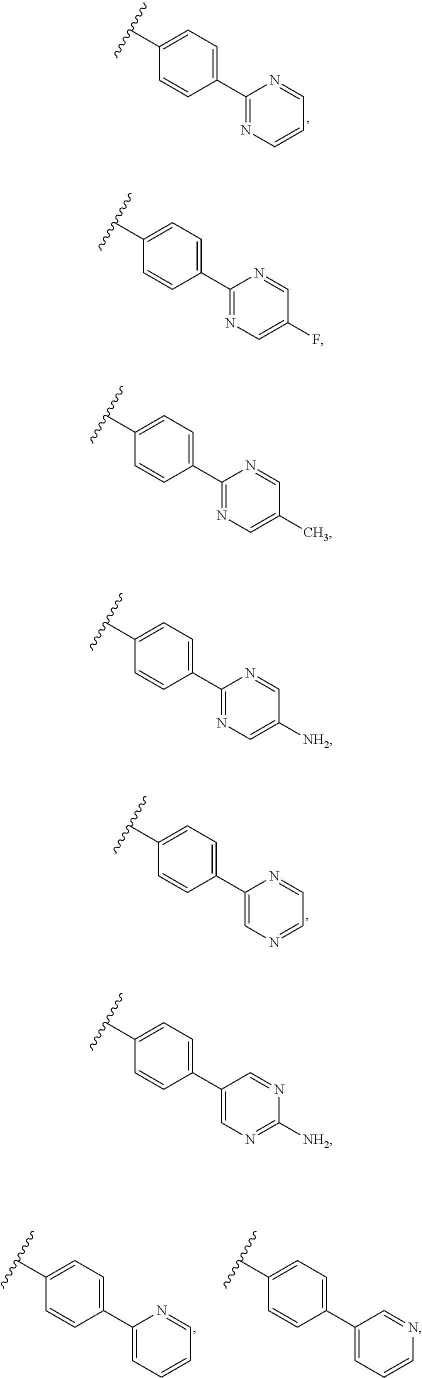

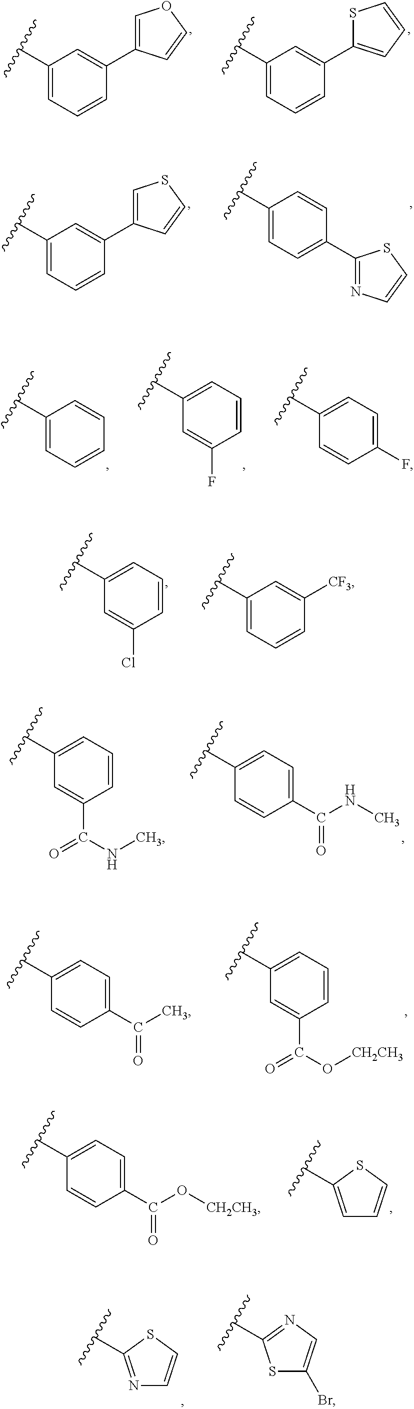

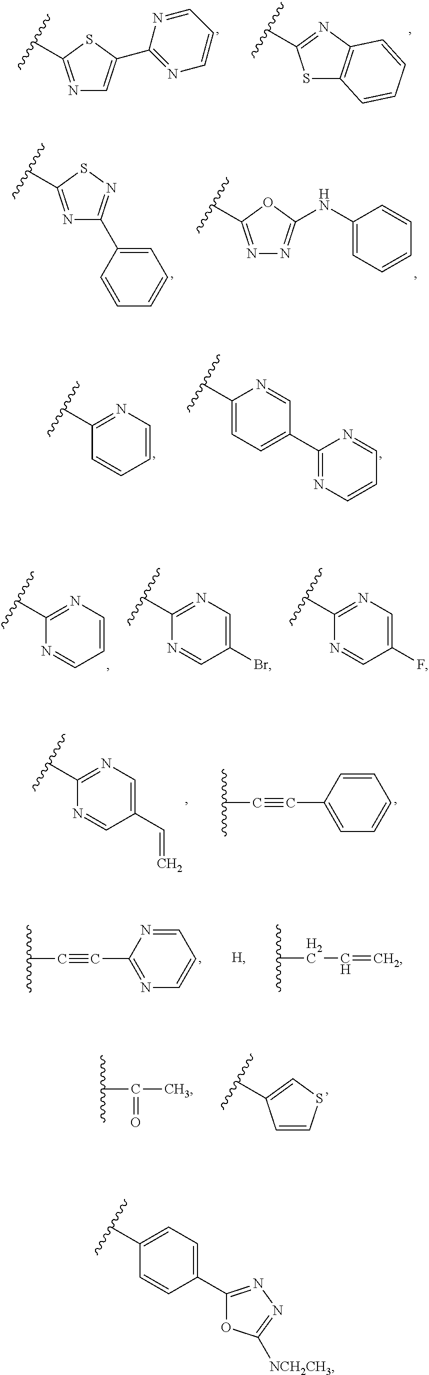

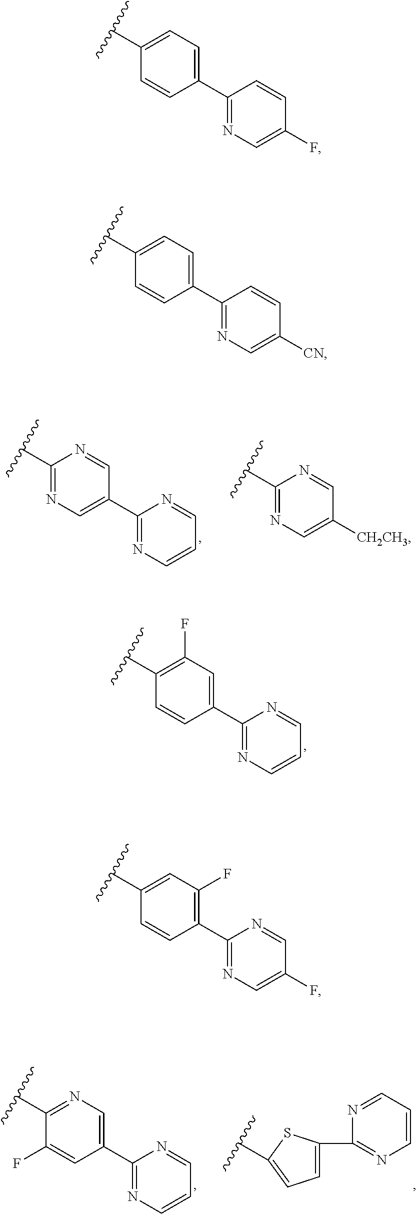

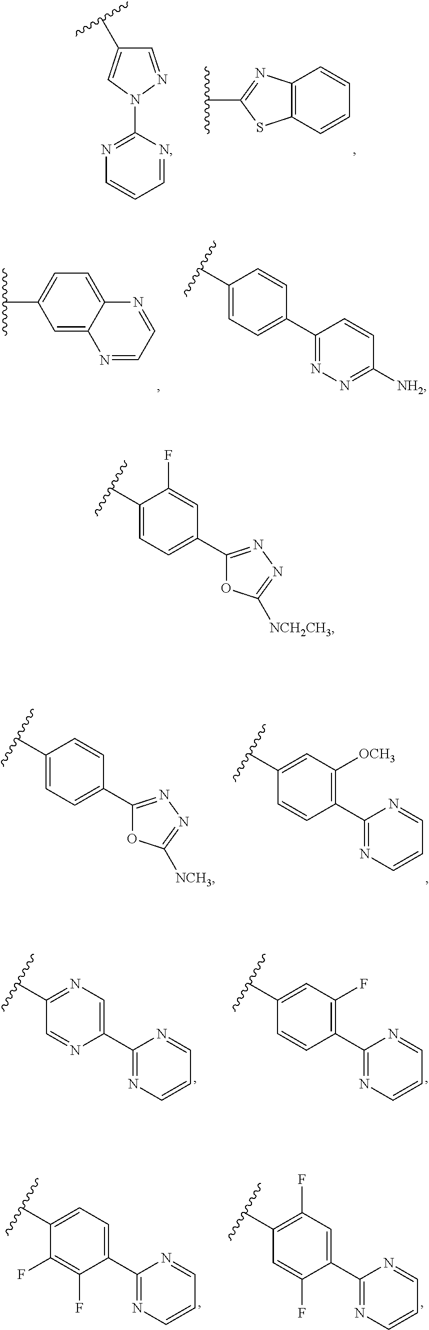

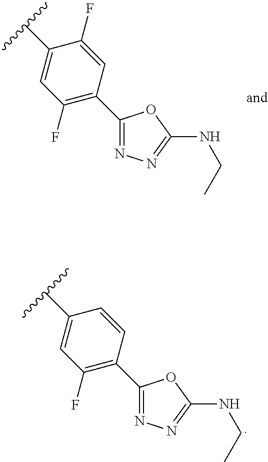

Embodiment No. 64 is directed to a compound of any one of Embodiment Nos. 1 to 43 wherein R⁵ is selected from the group consisting of:

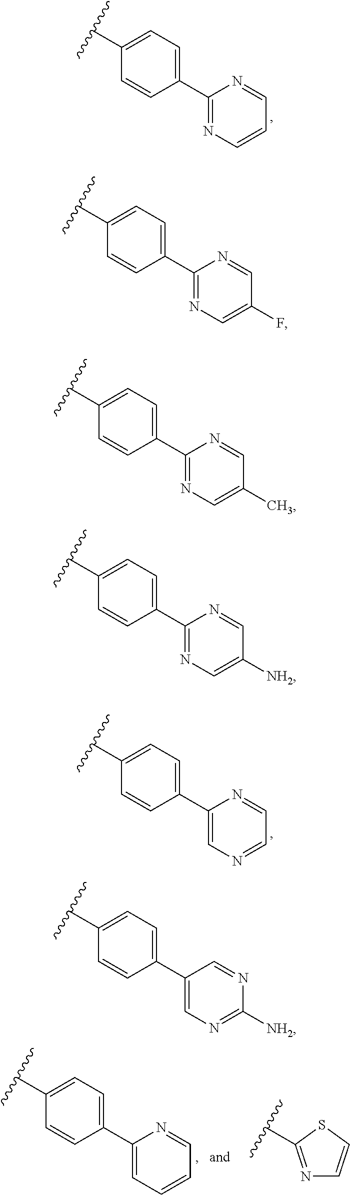

Embodiment No. 65 is directed to a compound of any one of Embodiment Nos. 1 to 43 wherein R⁵ is selected from the group consisting of:

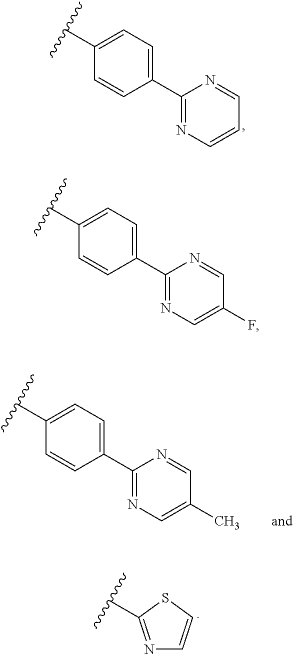

Embodiment No. 66 is directed to a compound of any one of Embodiment Nos. 1 to 43 wherein R⁵ is selected from the group consisting of:

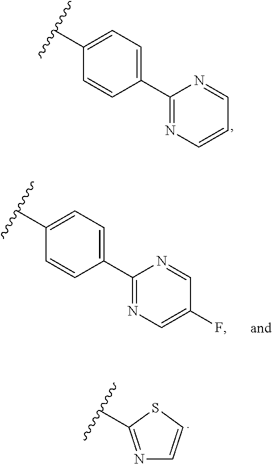

Embodiment No. 67 is directed to a compound of any one of Embodiment Nos. 1 to 43 wherein R⁵ is

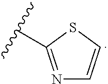

Embodiment No. 68 is directed to a compound of any one of Embodiment Nos. 1 to 43 wherein R⁵ is

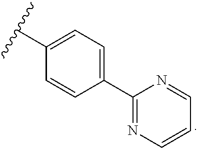

Embodiment No. 69 is directed to a compound of any one of Embodiment Nos. 1 to 43 wherein R⁵ is

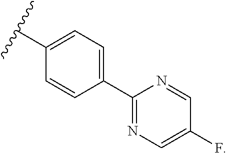

Embodiment No. 70 is directed to a compound of any one of Embodiment Nos. 1 to 43 wherein R⁵ is

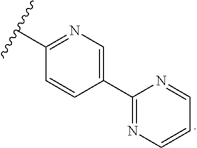

Embodiment No. 71 is directed to a compound of any one of Embodiment Nos. 1 to 43 wherein R⁵ is

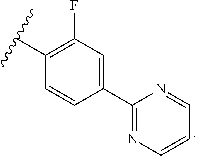

Embodiment No. 72 is directed to a compound of any one of Embodiment Nos. 1 to 43 wherein R⁵ is

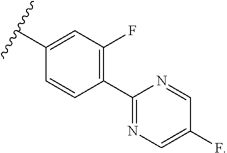

Embodiment No. 73 is directed to a compound of any one of Embodiment Nos. 1 to 103 wherein R¹ is selected from the group consisting of the R¹ groups of any one of Embodiment Nos. 54, 60, 61 or 62, and wherein R⁵ is selected from the group consisting of the R⁵ groups in any one of Embodiment Nos. 67, 68, or 69.

Embodiment No. 74 is directed to a compound of any one of Embodiment Nos. 1 to 73 wherein R² is selected from the group consisting of H, —CH₂OH and —CH₂F.

Embodiment No. 75 is directed to a compound of any one of Embodiment Nos. 1 to 73 wherein R² is H.

Embodiment No. 76 is directed to a compound of any one of Embodiment Nos. 1 to 73 wherein R² is —OR²³ wherein R²³ is alkyl.

Embodiment No. 77 is directed to a compound of any one of Embodiment Nos. 1 to 73 wherein R² is —OCH₃.

Embodiment No. 78 is directed to a compound of any one of Embodiment Nos. 1 to 73 wherein R² is —CN.

Embodiment No. 79 is directed to a compound of any one of Embodiment Nos. 1 to 73 wherein R² is —OCHF₂.

Embodiment No. 80 is directed to a compound selected from the group consisting of the final compounds of Examples 1 to 12.

Embodiment No. 81 is directed to the final compound of Example 1.

Embodiment No. 82 is directed to the final compound of Example 2.

Embodiment No. 83 is directed to the final compound of Example 3.

Embodiment No. 84 is directed to the final compound of Example 4.

Embodiment No. 85 is directed to the final compound of Example 5.

Embodiment No. 86 is directed to the final compound of Example 6.

Embodiment No. 87 is directed to the final compound of Example 7.

Embodiment No. 88 is directed to the final compound of Example 8.

Embodiment No. 89 is directed to the final compound of Example 9.

Embodiment No. 90 is directed to the final compound of Example 10.

Embodiment No. 91 is directed to the final compound of Example 11.

Embodiment No. 92 is directed to the final compound of Example 12.

Embodiment No. 93 is directed to a compound of any one of Embodiment Nos. 1 to 92 in pure and isolated form.

Embodiment No. 94 is directed to a pharmaceutical composition comprising an effective amount of at least one compound (e.g., 1, 2 or 3, or 1 or 2, or 1, and usually 1) of formula 1.0, preferably a compound of formula 1.0C1, and pharmaceutically acceptable carrier.

Embodiment No. 95 is directed to a pharmaceutical composition comprising an effective amount of a compound of formula 1.0, preferably a compound of formula 1.0C1, and a pharmaceutically acceptable carrier.

Embodiment No. 96 is directed to a pharmaceutical composition comprising an effective amount of at least one compound (e.g., 1, 2 or 3, or 1 or 2, or 1, and usually 1) of any one of Embodiment Nos. 1 to 93 and a pharmaceutically acceptable carrier.

Embodiment No. 97 is directed to a pharmaceutical composition comprising an effective amount of a compound of any one of Embodiment Nos. 1 to 93 and a pharmaceutically acceptable carrier.

Embodiment No. 98 is directed to a pharmaceutical composition of any one of Embodiment Nos. 94 to 97 further comprising an effective amount of at least one (e.g., 1, 2 or 3, or 1 or 2, or 1, and usually 1) other active pharmaceutically active ingredient.

Embodiment No. 99 is directed to a pharmaceutical composition of any one of Embodiment Nos. 94 to 97 further comprising an effective amount of another (i.e., one other) pharmaceutically active ingredient.

Embodiment No. 100 is directed to a pharmaceutical composition of any one of Embodiment Nos. 94 to 97 further comprising an effective amount of at least one (e.g., 1, 2 or 3, or 1 or 2, or 1, and usually 1) chemotherapeutic agent.

Embodiment No. 101 is directed to a pharmaceutical composition of any one of Embodiment Nos. 94 to 97 further comprising an effective amount of a chemotherapeutic agent.

Embodiment No. 102 is directed to a method of treating cancer in a patient in need of such treatment, said method comprising administering to said patient an effective amount of at least one (1, 2 or 3, or 1 or 2, or 1, and usually 1) compound of formula 1.0 (preferably formula 1.0C1).

Embodiment No. 103 is directed to a method of treating cancer in a patient in need of such treatment, said method comprising administering to said patient an effective amount of one compound of formula 1.0 (preferably formula 1.0C1).

Embodiment No. 104 is directed to a method of treating cancer in a patient in need of such treatment, said method comprising administering to said patient an effective amount of at least one (1, 2 or 3, or 1 or 2, or 1, and usually 1) compound of any one of Embodiment Nos. 1 to 93.

Embodiment No. 105 is directed to a method of treating cancer in a patient ins need of such treatment, said method comprising administering to said patient an effective amount of a compound of any one of Embodiment Nos. 1 to 93.

Embodiment No. 106 is directed to a method of treating cancer in any one of Embodiment Nos. 102 to 105 further comprising the administration of an effective amount of at least one (1, 2 or 3, or 1 or 2, or 1, and usually 1) chemotherapeutic agent.

Embodiment No. 107 is directed to a method of treating cancer in any one of Embodiment Nos. 102 to 105 further comprising the administration of an effective amount of a chemotherapeutic agent.

Embodiment No. 108 is directed to a method of treating cancer in a patient in need of such treatment comprising administering to said patient an effective amount of a pharmaceutical composition of any one of Embodiment Nos. 94 to 101.

Embodiment No. 109 is directed to a method of treating cancer of any one of Embodiment Nos. 106, 107 and 108 (wherein the pharmaceutical composition is a composition of any one of Embodiment Nos. 100 and 101) wherein the chemotherapeutic agent is selected from the group consisting of: paclitaxel, docetaxel, carboplatin, cisplatin, gemcitabine, tamoxifen, Herceptin, Cetuximab, Tarceva, Iressa, bevacizumab, navelbine, IMC-1C11, SU5416 and SU6688.

Embodiment No. 110 is directed to a method of treating cancer of any one of Embodiment Nos. 106, 107 and 108 (wherein the pharmaceutical composition is a composition of any one of Embodiment Nos. 100 and 101) wherein the chemotherapeutic agent is selected from the group consisting of: paclitaxel, docetaxel, carboplatin, cisplatin, navelbine, gemcitabine, and Herceptin.

Embodiment No. 111 is directed to a method of treating cancer of any one of Embodiment Nos. 106, 107 and 108 (wherein the pharmaceutical composition is a composition of any one of Embodiment Nos. 100 and 101) wherein the chemotherapeutic agent is selected from the group consisting of: Cyclophasphamide, 5-Fluorouracil, Temozolomide, Vincristine, Cisplatin, Carboplatin, and Gemcitabine.

Embodiment No. 112 is directed to a method of treating cancer of any one of Embodiment Nos. 106, 107 and 108 (wherein the pharmaceutical composition is a composition of any one of Embodiment Nos. 100 and 101) wherein the chemotherapeutic agent is selected from the group consisting of: Gemcitabine, Cisplatin and Carboplatin.

This invention also provides a method of treating cancer in a patient in need of such treatment, said treatment comprising administering to said patient a therapeutically effective amount at least one (e.g., 1, 2 or 3, or 1 or 2, or 1, and usually 1) compound of formula 1.0 (for example, as described in any one of Embodiment Nos. 1 to 93), and therapeutically effective amounts of at least one (e.g., 1, 2 or 3, or 1 or 2, or 2, or 1) chemotherapeutic agent selected from the group consisting of: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of aV133 integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

This invention also provides a method of treating cancer in a patient in need of such treatment, said treatment comprising administering to said patient a therapeutically effective amount at least one (e.g., 1, 2 or 3, or 1 or 2, or 1, and usually 1) compound of formula 1.0 (for example, as described in any one of Embodiment Nos. 1 to 93), and therapeutically effective amounts of at least two (e.g., 2 or 3, or 2, and usually 2) different antineoplastic agents selected from the group consisting of: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins; (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and IGF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors (also know as FPT inhibitors or FTI (i.e., farnesyl transfer inhibitors)).

This invention also provides a method of treating cancer in a patient in need of such treatment, said method comprising administering to said patient therapeutically effective amounts at least one (e.g., 1, 2 or 3, or 1 or 2, or 1, and usually 1) compound of formula 1.0 (for example, as described in any one of Embodiment Nos. 1 to 93), and an antineoplastic agent selected from the group consisting of: (1) EGF inhibitors that are antibodies, (2) EGF inhibitors that are small molecules, (3) VEGF inhibitors that are antibodies, and (4) VEGF inhibitors that are small molecules. Radiation therapy can also be used in conjunction with this above combination therapy, i.e., the above method using a combination of compounds of the invention and antineoplastic agent can also comprise the administration of a therapeutically effect amount of radiation.

This invention also provides a method of treating leukemias (e.g., acute myeloid leukemia (AML), and chronic myeloid leukemia (CML)) in a patient in need of such treatment, said method comprising administering to said patient therapeutically effective amounts at least one (e.g., 1, 2 or 3, or 1 or 2, or 1, and usually 1) compound of formula 1.0 (for example, as described in any one of Embodiment Nos. 1 to 93), and: (1) Gleevec and interferon to treat CML; (2) Gleevec and pegylated interferon to treat CML; (3) Gleevec to treat CML; (4) an anti-tumor nucleoside derivative (e.g., Ara-C) to treat AML; or (5) an anti-tumor nucleoside derivative (e.g., Ara-C) in combination with an anthracycline to treat AML.

This invention also provides a method of treating non-Hodgkin's lymphoma in a patient in need of such treatment, said method comprising administering therapeutically effective amounts at least one (e.g., 1, 2 or 3, or 1 or 2, or 1, and usually 1) compound of formula 1.0 (for example, as described in any one of Embodiment Nos. 1 to 93) and: (1) a biologic (e.g., Rituxan); (2) a biologic (e.g., Rituxan) and an anti-tumor nUcledside derivative Fludarabine); or (3) Genasense (antisense to BCL-2).

This invention also provides a method of treating multiple myeloma in a patient in need of such treatment, said method comprising administering to said patient therapeutically effective amounts of at least one (e.g., 1, 2 or 3, or 1 or 2, or 1, and usually 1) compound of formula 1.0 (for example, as described in any one of Embodiment Nos. 1 to 93) and: (1) a proteosome inhibitor (e.g., PS-341 from Millenium); or (2) Thalidomide (or related imid).

This invention also provides a method of treating cancer in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) at least one (e.g., 1, 2 or 3, or 1 or 2, or 2, or 1) antineoplastic agent selected from the group consisting of: (1) taxanes, (2) platinum coordinator compounds, (3) EGF inhibitors that are antibodies, (4) EGF inhibitors that are small molecules, (5) VEGF inhibitors that are antibodies, (6) VEGF kinase inhibitors that are small molecules, (7) estrogen receptor antagonists or selective estrogen receptor modulators, (8) anti-tumor nucleoside derivatives, (9) epothilones, (10) topoisomerase inhibitors, (11) vinca alkaloids, and (12) antibodies that are inhibitors of αVβ3 integrins.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) at least one (e.g., 1, 2 or 3, or 1 or 2, or 2, or 1) antineoplastic agent selected from the group consisting of: (1) taxanes, (2) platinum coordinator compounds, (3) EGF inhibitors that are antibodies, (4) EGF inhibitors that are small molecules, (5) VEGF inhibitors that are antibodies, (6) VEGF kinase inhibitors that are small molecules, (7) estrogen receptor antagonists or selective estrogen receptor modulators, (8) anti-tumor nucleoside derivatives, (9) epothilones, (10) topoisomerase inhibitors, (11) vinca alkaloids, and (12) antibodies that are inhibitors of αVβ3 integrins.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) at least one (e.g., 1, 2 or 3, or 1 or 2, or 2, or 1) antineoplastic agent selected from the group consisting of: (1) taxanes, (2) platinum coordinator compounds, (3) anti-tumor nucleoside derivatives, (4) topoisomerase inhibitors, and (5) vinca alkaloids.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93), (b) carboplatin, and (c) paclitaxel.

This invention also provides a method of treating non small cell lung cancer in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), (b) cisplatin, and (c) gemcitabine.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93), (b) carboplatin, and (c) gemcitabine.

This invention also provides a method of treating non small cell lung cancer in a patient in need of such treatment, said method comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93), (b) Carboplatin, and (c) Docetaxel.

This invention also provides a method of treating cancer in a patient in need of such treatment, said method comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) an antineoplastic agent selected from the group consisting of: (1) EGF inhibitors that are antibodies, (2) EGF inhibitors that are small molecules, (3) VEGF inhibitors that are antibodies, (4) VEGF kinase inhibitors that are small molecules.

This invention also provides a method of treating squamous cell cancer of the head and neck, in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) at least one (e.g., 1, 2 or 3, or 1 or 2, or 2, or 1) antineoplastic agent selected from the group consisting of: (1) taxanes, and (2) platinum coordinator compounds.

This invention also provides a method of treating squamous cell cancer of the head and neck, in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) at least one (e.g., 1, 2 or 3, or 1 or 2, or 2, or 1) antineoplastic agent selected from the group consisting of: (1) taxanes, (2) platinum coordinator compounds, and (3) anti-tumor nucleoside derivatives (e.g., 5-Fluorouracil).

This invention also provides a method of treating CML in a patient in need of such treatment, said method comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93), (b) Gleevec, and (c) interferon (e.g., Intron-A).

This invention also provides a method of treating CML in a patient in need of such treatment comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93), (b) Gleevec; and (c) pegylated interferon (e.g., Peg-Intron, and Pegasys).

This invention also provides a method of treating CML in a patient in need of such treatment comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93) and (b) Gleevec.

This invention also provides a method of treating CMML in a patient in need of such treatment, said method comprising administering to said patient therapeutically effective amounts of at least one (e.g., 1, 2 or 3, or 1 or 2, or 1, and usually 1) compound of formula 1.0 (for example, as described in any one of Embodiment Nos. 1 to 93).

This invention also provides a method of treating AML in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) an anti-tumor nucleoside derivative (e.g., Cytarabine (i.e., Ara-C)).

This invention also provides a method of treating AML in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), (b) an anti-tumor nucleoside derivative (e.g., Cytarabine (i.e., Ara-C)), and (c) an anthracycline.

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) Rituximab (Rituxan).

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), (b) Rituximab (Rituxan), and (c) an anti-tumor nucleoside derivative (e.g., Fludarabine (i.e., F-ara-A).

This invention also provides a method of treating non-Hodgkin's lymphoma in a patient in need of such treatment, said method comprising administering to said patient therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) Genasense (antisense to BCL-2).

This invention also provides a method of treating multiple myeloma in a patient in need of such treatment, said method comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) a proteosome inhibitor (e.g., PS-341 (Millenium)).

This invention also provides a method of treating multiple myeloma in a patient in need of such treatment, said method comprising administering to said patient therapeutically effective amounts of: (a) at least one (e.g., 1, 2 or 3, or 1 or 2, or -1, and usually 1) compound of formula 1.0 (for example, as described in any one of Embodiment Nos. 1 to 93), and (b) Thalidomide or related imid.

This invention also provides a method of treating multiple myeloma in a patient in need of such treatment, said method comprising administering therapeutically effective amounts of: (a) at least one (e.g., 1, 2 or 3, or 1 or 2, or 1, and usually 1) compound of formula 1.0 (for example, as described in any one of Embodiment Nos. 1 to 93), and (b) Thalidomide.

This invention is also directed to the methods of treating cancer described herein, particularly those described above, wherein in addition to the administration of the compound of formula 1.0 (for example, as described in any one of Embodiment Nos. 1 to 93) and antineoplastic agents, radiation therapy is also administered prior to, during, or after the treatment cycle.

This invention also provides a method for treating cancer (e.g., lung cancer, prostate cancer and myeloid leukemias) in a patient in need of such treatment, said method comprising administering to said patient (1) an effective amount of at least one (e.g., 1, 2 or 3, or 1 or 2, or 1, and usually 1) compound of formula 1.0 (for example, as described in any one of Embodiment Nos. 1 to 93), in combination with (2) at least one (e.g., 1, 2 or 3, or 1 or 2, or 2, or 1) antineoplastic agent, microtubule affecting agent and/or radiation therapy.

This invention also provides a method of treating cancer in a patient in need of such treatment, said method comprising administering to said patient an effective amount of at least one (e.g., 1, 2 or 3, or 1 or 2, or 1, and usually 1) compound of formula 1.0 (for example, as described in any one of Embodiment Nos. 1 to 93) in combination with an effective amount of at least one (e.g., 1, 2 or 3, or 1 or 2, or 1, and usually 1) signal transduction inhibitor.

Thus, in one example (e.g., treating non small cell lung cancer): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once per week in an amount of about 50 to about 100 mg/m², and in another example about 60 to about 80 mg/m², and (3) Carboplatin is administered once per week in an amount to provide an AUC of about 2 to about 3.

In another example (e.g., treating non small cell lung cancer): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and yet in another example about 100 mg administered twice a day, (2) Paclitaxel (e.g., Taxol® is administered once per week in an amount of about 50 to about 100 mg/m², and in another example about 60 to about 80 mg/m², and (3) Cisplatin is administered once per week in an amount of about 20 to about 40 mg/m².

In another example (e.g., treating non small cell lung cancer): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, (2) Docetaxel (e.g., Taxotere®) is administered once per week in an amount of about 10 to about 45 mg/m², and (3) Carboplatin is administered once per week in an amount to provide an AUC of about 2 to about 3.

In another example (e.g., treating non small cell lung cancer): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, (2) Docetaxel (e.g., Taxotere®) is administered once per week in an amount of about 10 to about 45 mg/m², and (3) Cisplatin is administered once per week in an amount of about 20 to about 40 mg/m².

In another example (e.g., treating non small cell lung cancer): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once every three weeks in an amount of about 150 to about 250 mg/m², and in another example about 175 to about 225 mg/m², and in yet another example 175 mg/m², and (3) Carboplatin is administered once every three weeks in an amount to provide an AUC of about 5 to about 8, and in another example 6.

In another example of treating non small cell lung cancer: (1) the compound of formula 1.0 (for example, as described in any one of Embodiment Nos. 1 to 93) is administered in an amount of 100 mg administered twice a day, (2) Paclitaxel (e.g., Taxol® is administered once every three weeks in an amount of 175 mg/m², and (3) Carboplatin is administered once every three weeks in an amount to provide an AUC of 6.

In another example (e.g., treating non small cell lung cancer): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, (2) Paclitaxel (e.g., Taxol® is administered once every three weeks in an amount of about 150 to about 250 mg/m², and in another example about 175 to about 225 mg/m², and (3) Cisplatin is administered once every three weeks in an amount of about 60 to about 100 mg/m².

In another example (e.g., treating non small cell lung cancer): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, (2) Docetaxel (e.g., Taxotere® is administered once every three weeks in an amount of about 50 to about 100 mg/m², and (3) Carboplatin is administered once every three weeks in an amount to provide an AUC of about 5 to about 8.

In another example (e.g., treating non small cell lung cancer): (1) the compound of formula 1.0 (for example, as described in any one of Embodiment Nos. 1 to 93) is administered in an amount of about 50 mg to about 200 mg twice a day, in another example about 75 mg to about 125 mg administered twice a day, and in yet another example about 100 mg administered twice a day, (2) Docetaxel (e.g., Taxotere® is administered once every three weeks in an amount of about 50 to about 100 mg/m², and (3) Cisplatin is administered once every three weeks in an amount of about 60 to about 100 mg/m².

In another example for treating non small cell lung cancer using the compounds of formula 1.0, Docetaxel and Carboplatin: (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, (2) Docetaxel (e.g., Taxotere® is administered once every three weeks in an amount of about 75 mg/m², and (3) Carboplatin is administered once every three weeks in an amount to provide an AUC of about 6.

In another example of the treatments of non-small cell lung cancer described above the Docetaxel (e.g., Taxotere®) and Cisplatin, the Docetaxel (e.g., Taxotere®) and Carboplatin, the Paclitaxel (e.g., Taxol®) and Carboplatin, or the Paclitaxel (e.g., Taxol®) and Cisplatin are administered on the same day.

In another example (e.g., CML): (1) the compound of formula 1.0 (for example, as described in any one of Embodiment Nos. 1 to 93) is administered in an amount of about 100 mg to about 200 mg administered twice a day, (2) Gleevec is administered in an amount of about 400 to about 800 mg/day orally, and (3) interferon (Intron-A) is administered in an amount of about 5 to about 20 million IU three times per week.

In another example (e.g., CML): (1) the compound of formula 1.0 (for example, as described in any one of Embodiment Nos. 1 to 93) is administered in an amount of about 100 mg to about 200 mg administered twice a day, (2) Gleevec is administered in an amount of about 400 to about 800 mg/day orally, and (3) pegylated interferon (Peg-Intron or Pegasys) is administered in an amount of about 3 to about 6 micrograms/kg/day.

In another example (e.g., non-Hodgkin's lymphoma): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, and (2) Genasense (antisense to BCL-2) is administered as a continuous IV infusion at a dose of about 2 to about 5 mg/kg/day (e.g., 3 mg/kg/day) for 5 to 7 days every 3 to 4 weeks.

In another example (e.g., multiple myeloma): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day; and in yet another example about 100 mg administered twice a day, and (2) the proteosome inhibitor (e.g., PS-341—Millenium) is administered in an amount of about 1.5 mg/m² twice weekly for two consecutive weeks with a one week rest period.

In another example (e.g., multiple myeloma): (1) the compound of formula 1.0 (for example, as described in any one of Embodiment Nos. 1 to 93) is administered in an amount of about 50 mg to about 200 mg twice a day, and in another example about 75 mg to about 125 mg administered twice a day, and in yet another example about 100 mg administered twice a day, and (2) the Thalidomide (or related imid) is administered orally in an amount of about 200 to about 800 mg/day, with dosing being continuous until relapse or toxicity.

In one embodiment of the methods of treating cancer of this invention, the chemotherapeutic agents are selected from the group consisting of: paclitaxel, docetaxel, carboplatin, cisplatin, gemcitabine, tamoxifen, Herceptin, Cetuximab, Tarceva, Iressa, bevacizumab, navelbine, IMC-1C11, SU5416 and SU6688.

In another embodiment of the methods of treating cancer of this invention, the chemotherapeutic agents are selected from the group consisting of: paclitaxel, docetaxel, carboplatin, cisplatin, navelbine, gemcitabine, and Herceptin.

Thus, one embodiment of this invention is directed to a method of treating cancer comprising administering to a patient in need of such treatment therapeutically effective amounts of the compound of formula 1.0 (for example, as described in any one of Embodiment Nos. 1 to 93), a taxane, and a platinum coordination compound.

Another embodiment of this invention is directed to a method of treating cancer comprising administering to a patient in need of such treatment therapeutically effective amounts of the compound of formula 1.0 (for example, as described in any one of Embodiment Nos. 1 to 93), a taxane, and a platinum coordination compound, wherein said compound of formula 1.0 is administered every day, said taxane is administered once per week per cycle, and said platinum coordinator compound is administered once per week per cycle. In another embodiment the treatment is for one to four weeks per cycle.

Another embodiment of this invention is directed to a method of treating cancer comprising administering to a patient in need of such treatment therapeutically effective amounts of the compound of formula 1.0 (for example, as described in any one of Embodiment Nos. 1 to 93), a taxane, and a platinum coordination compound, wherein said compound of formula 1.0 is administered every day, said taxane is administered once every three weeks per cycle, and said platinum coordinator compound is administered once every three weeks per cycle. In another embodiment the treatment is for one to three weeks per cycle.

Another embodiment of this invention is directed to a method of treating cancer comprising administering to a patient in need of such treatment therapeutically effective amounts of the compound of formula 1.0 (for example, as described in any one of Embodiment Nos. 1 to 93), paclitaxel, and carboplatin. In another embodiment, said compound of formula 1.0 is administered every day, said paclitaxel is administered once per week per cycle, and said carboplatin is administered once per week per cycle. In another embodiment the treatment is for one to four weeks per cycle.

Another embodiment of this invention is directed to a method of treating cancer comprising administering to a patient in need of such treatment therapeutically effective amounts of the compound of formula 1.0 (for example, as described in any one of Embodiment Nos. 1 to 93), paclitaxel, and carboplatin. In another embodiment, said compound of formula 1.0 is administered every day, said paclitaxel is administered once every three weeks per cycle, and said carboplatin is administered once every three weeks per cycle. In another embodiment the treatment is for one to three weeks per cycle.

Another embodiment of this invention is directed to a method for treating non small cell lung cancer in a patient in need of such treatment comprising administering daily a therapeutically effective amount of the compound of formula 1.0 (for example, as described in any one of Embodiment Nos. 1 to 93), administering a therapeutically effective amount of carboplatin once a week per cycle, and administering a therapeutically effective amount of paclitaxel once a week per cycle, wherein the treatment is given for one to four weeks per cycle. In another embodiment said compound of formula 1.0 is administered twice per day. In another embodiment said carboplatin and said paclitaxel are administered on the same day, and in another embodiment said carboplatin and said paclitaxel are administered consecutively, and in another embodiment said carboplatin is administered after said paclitaxel.

Another embodiment of this invention is directed to a method for treating non small cell lung cancer in a patient in need of such treatment comprising administering daily a therapeutically effective amount of a compound of formula 1.0 (for example: as described in any one of Embodiment Nos. 1 to 93), administrating a therapeutically effective amount of carboplatin once every three weeks per cycle, and administering a therapeutically effective amount of paclitaxel once every three weeks per cycle, wherein the treatment is given for one to three weeks. In another embodiment compound of formula 1.0 is administered twice per day. In another embodiment said carboplatin and said paclitaxel are administered on the same day, and in another embodiment said carboplatin and said paclitaxel are administered consecutively, and in another embodiment said carboplatin is administered after said paclitaxel.

Another embodiment of this invention is directed to a method for treating non small cell lung cancer in a patient in need of such treatment comprising administering about 50 to about 200 mg of a compound of formula 1.0 (for example, as described in any one of Embodiment Nos. 1 to 93) twice a day, administering carboplatin once per week per cycle in an amount to provide an AUC of about 2 to about 8 (and in another embodiment about 2 to about 3), and administering once per week per cycle about 60 to about 300 mg/m² (and in another embodiment about 50 to 100 mg/m², and in yet another embodiment about 60 to about 80 mg/m²) of paclitaxel, wherein the treatment is given for one to four weeks per cycle. In another embodiment said compound of formula 1.0 is administered in amount of about 75 to about 125 mg twice a day, and in another embodiment about 100 mg twice a day. In another embodiment said carboplatin and said paclitaxel are administered on the same day, and in another embodiment said carboplatin and said paclitaxel are administered consecutively, and in another embodiment said carboplatin is administered after said paclitaxel.

In another embodiment, this invention is directed to a method for treating non small cell lung cancer in a patient in need of such treatment comprising administering about 50 to about 200 mg of a compound of formula 1.0 (for example, as described in any one of Embodiment Nos. 1 to 93) twice a day, administering carboplatin once every three weeks per cycle in an amount to provide an AUC of about 2 to about 8 (in another embodiment about 5 to about 8, and in another embodiment 6), and administering once every three weeks per cycle about 150 to about 250 mg/m² (and in another embodiment about 175 to about 225 mg/m², and in another embodiment 175 mg/m²) of paclitaxel, wherein the treatment is given for one to three weeks. In another embodiment said compound of formula 1.0 is administered in an amount of about 75 to about 125 mg twice a day, and in another embodiment about 100 mg twice a day. In another embodiment said carboplatin and said paclitaxel are administered on the same day, and in another embodiment said carboplatin and said paclitaxel are administered consecutively, and in another embodiment said carboplatin is administered after said paclitaxel.

Other embodiments of this invention are directed to methods of treating cancer as described in the above embodiments (i.e., the embodiments directed to treating cancer and to treating non small cell lung cancer with a taxane and platinum coordinator compound) except that in place of paclitaxel and carboplatin the taxanes and platinum coordinator compounds used together in the methods are: (1) docetaxel (Taxotere®) and cisplatin; (2) paclitaxel and cisplatin; and (3) docetaxel and carboplatin. In another embodiment of the methods of this invention cisplatin is used in amounts of about 30 to about 100 mg/m². In another embodiment of the methods of this invention docetaxel is used in amounts of about 30 to about 100 mg/m².

In another embodiment this invention is directed to a method of treating cancer comprising administering to a patient in need of such treatment therapeutically effective amounts of a compound of formula 1.0 (for example, as described in any one of Embodiment Nos. 1 to 93), a taxane, and an EGF inhibitor that is an antibody. In another embodiment the taxane used is paclitaxel, and the EGF inhibitor is a HER2 antibody (in one embodiment Herceptin) or Cetuximab, and in another embodiment Herceptin is used. The length of treatment, and the amounts and administration of said compound of formula 1.0 and the taxane are as described in the embodiments above. The EGF inhibitor that is an antibody is administered once a week per cycle, and in another embodiment is administered on the same day as the taxane, and in another embodiment is administered consecutively with the taxane. For example, Herceptin is administered in a loading dose of about 3 to about 5 mg/m² (in another embodiment about 4 mg/m²), and then is administered in a maintenance dose of about 2 mg/m² once per week per cycle for the remainder of the treatment cycle (usually the cycle is 1 to 4 weeks). In one embodiment the cancer treated is breast cancer.

In another embodiment this invention is directed to a method of treating cancer comprising administering to a patient in need of such treatment therapeutically effective amounts of: (1) a compound of formula 1.0 (for example, as described in any one of Embodiment Nos. 1 to 93), (2) a taxane, and (3) an antineoplastic agent selected from the group consisting of: (a) an EGF inhibitor that is a small molecule, (b) a VEGF inhibitor that is an antibody, and (c) a VEGF kinase inhibitor that is a small molecule. In another embodiment, the taxane paclitaxel or docetaxel is used. In another embodiment the antineoplastic agent is selected from the group consisting of: tarceva, Iressa, bevacizumab, SU5416, SU6688 and BAY 43-9006. The length of treatment, and the amounts and administration of said compound of formula 1.0 and the taxane are as described in the embodiments above. The VEGF kinase inhibitor that is an antibody is usually given once per week per cycle. The EGF and VEGF inhibitors that are small molecules are usually given daily per cycle. In another embodiment, the VEGF inhibitor that is an antibody is given on the same day as the taxane, and in another embodiment is administered concurrently with the taxane. In another embodiment, when the EGF inhibitor that is a small molecule or the VEGF inhibitor that is a small molecule is administered on the same day as the taxane, the administration is concurrently with the taxane. The EGF or VEGF kinase inhibitor is generally administered in an amount of about 10 to about 500 mg/m².

In another embodiment this invention is directed to a method of treating cancer comprising administering to a patient in need of such treatment therapeutically effective amounts of a compound of formula 1.0 (for example, as described in any one of Embodiment Nos. 1 to 93), an anti-tumor nucleoside derivative, and a platinum coordination compound.

Another embodiment of this invention is directed to a method of treating cancer comprising administering to a patient in need of such treatment therapeutically effective amounts of a compound of formula 1.0 (for example, as described in any one of Embodiment Nos. 1 to 93), an anti-tumor nucleoside derivative, and a platinum coordination compound, wherein said compound of formula 1.0 is administered every day, said anti-tumor nucleoside derivative is administered once per week per cycle, and said platinum coordinator compound is administered once per week per cycle. Although the treatment can be for one to four weeks per cycle, in one embodiment the treatment is for one to seven weeks per cycle.

Another embodiment of this invention is directed to a method of treating cancer comprising administering to a patient in need of such treatment therapeutically effective amounts of a compound of formula 1.0 (for example, as described in any one of Embodiment Nos. 1 to 93), an anti-tumor nucleoside derivative, and a platinum coordination compound, wherein said compound of formula 1.0 is administered every day, said an anti-tumor nucleoside derivative is administered once per week per cycle, and said platinum coordinator compound is administered once every three weeks per cycle. Although the treatment can be for one to four weeks per cycle, in one embodiment the treatment is for one to seven weeks per cycle.

Another embodiment of this invention is directed to a method of treating cancer comprising administering to a patient in need of such treatment therapeutically effective amounts of a compound of formula 1.0 (for example, as described in any one of Embodiment Nos. 1 to 93), gemcitabine, and cisplatin. In another embodiment, said compound of formula 1.0 is administered every day, said gemcitabine is administered once per week per cycle, and said cisplatin is administered once per week per cycle. In one embodiment the treatment is for one to seven weeks per cycle.

Another embodiment of this invention is directed to a method of treating cancer comprising administering to a patient in need of such treatment therapeutically effective amounts of a compound of formula 1.0 (for example, as described in any one of Embodiment Nos. 1 to 93), gemcitabine, and cisplatin. In another embodiment, said compound of formula 1.0 is administered every day, said gemcitabine is administered once per week per cycle, and said cisplatin is administered once every three weeks per cycle. In another embodiment the treatment is for one to seven weeks.

Another embodiment of this invention is directed to a method of treating cancer comprising administering to a patient in need of such treatment therapeutically effective amounts of a compound of formula 1.0 (for example, as described in any one of Embodiment Nos. 1 to 93), gemcitabine, and carboplatin. In another embodiment said compound of formula 1.0 is administered every day, said gemcitabine is administered once per week per cycle, and said carboplatin is administered once per week per cycle. In another embodiment the treatment is for one to seven weeks per cycle.

Another embodiment of this invention is directed to a method of treating cancer comprising administering to a patient in need of such treatment therapeutically effective amounts of a compound of formula 1.0 (for example, as described in any one of Embodiment Nos. 1 to 93), gemcitabine, and carboplatin. In another embodiment said compound of formula 1.0 is administered every day, said gemcitabine is administered once per week per cycle, and said carboplatin is administered once every three weeks per cycle. In another embodiment the treatment is for one to seven weeks per cycle.

In the above embodiments using gemcitabine, the compound of formula 1.0 (for example, as described in any one of Embodiment Nos. 1 to 93) and the platinum coordinator compound are administered as described above for the embodiments using taxanes. Gemcitabine is administered in an amount of about 500 to about 1250 mg/m². In one embodiment the gemcitabine is administered on the same day as the platinum coordinator compound, and in another embodiment consecutively with the platinum coordinator compound, and in another embodiment the gemcitabine is administered after the platinum coordinator compound.

Another embodiment of this invention is directed to a method of treating cancer in a patient in need of such treatment comprising administering to said patient a compound of formula 1.0 (for example, as described in any one of Embodiment Nos. 1 to 93) and an antineoplastic agent selected from: (1) EGF inhibitors that are antibodies, (2) EGF inhibitors that are small molecules, (3) VEGF inhibitors that are antibodies, and (4) VEGF kinase inhibitors that are small molecules all as described above. The treatment is for one to seven weeks per cycle, and generally for one to four weeks per cycle. The compound of formula 1.0 is administered in the same manner as described above for the other embodiments of this invention. The small molecule antineoplastic agents are usually administered daily, and the antibody antineoplastic agents are usually administered once per week per cycle. In one embodiment the antineoplastic agents are selected from the group consisting of: Herceptin, Cetuximab, Tarceva, Iressa, bevacizumab, IMC-1C11, SU5416, SU6688 and BAY 43-9006.

In the embodiments of this invention wherein a platinum coordinator compound is used as well as at least one other antineoplastic agent, and these drugs are administered consecutively, the platinum coordinator compound is generally administered after the other antineoplastic agents have been administered.

Other embodiments of this invention include the administration of a therapeutically effective amount of radiation to the patient in addition to the administration of a compound of formula 1.0 (for example, as described in any one of Embodiment Nos. 1 to 93) and antineoplastic agents in the embodiments described above. Radiation is administered according to techniques and protocols well know to those skilled in the art.

Another embodiment of this invention is directed to a pharmaceutical composition comprising at least two different chemotherapeutic agents and a pharmaceutically acceptable carrier for intravenous administration. Preferably the pharmaceutically acceptable carrier is an isotonic saline solution (0.9% NaCl) or a dextrose solution (e.g., 5% dextrose).

Another embodiment of this invention is directed to a pharmaceutical composition comprising a compound of formula 1.0 (for example, as described in any one of Embodiment Nos. 1 to 93) and at least two different antineoplastic agents and a pharmaceutically acceptable carrier for intravenous administration. Preferably the pharmaceutically acceptable carrier is an isotonic saline solution (0.9% NaCl) or a dextrose solution (e.g., 5% dextrose).

Another embodiment of this invention is directed to a pharmaceutical composition comprising a compound of formula 1.0 (for example, as described in any one of Embodiment Nos. 1 to 93) and at least one antineoplastic agent and a pharmaceutically acceptable carrier for intravenous administration. Preferably the pharmaceutically acceptable carrier is an isotonic saline solution (0.9% NaCl) or a dextrose solution (e.g., 5% dextrose).

Other embodiments of this invention are directed to the use of a combination of at least one (e.g., one) compound of formula 1.0 (for example, as described in any one of Embodiment Nos. 1 to 93) and drugs for the treatment of breast cancer, i.e., this invention is directed to a combination therapy for the treatment of breast cancer. Those skilled in the art will appreciate that the compounds of formula 1.0 and drugs are generally administered as individual pharmaceutical compositions. The use of a pharmaceutical composition comprising more than one drug is within the scope of this invention.

Thus, another embodiment of this invention is directed to a method of treating (or preventing) breast cancer (i.e., postmenopausal and premenopausal breast cancer, e.g., hormone-dependent breast cancer) in a patient in need of such treatment comprising administering to said patient a therapeutically effective amount of at least one (e.g., one) compound of formula 1.0 (for example, as described in any one of Embodiment Nos. 1 to 93) and a therapeutically effective amount of at least one antihormonal agent selected from the group consisting of: (a) aromatase inhibitors, (b) antiestrogens, and (c) LHRH analogues; and said treatment optionally including the administration of at least one chemotherapeutic agent.

The compound of formula 1.0 is preferably administered orally, and in one embodiment is administered in capsule form.

Examples of aromatase inhibitors include but are not limited to Anastrozole (e.g., Arimidex), Letrozole (e.g., Femara), Exemestane (Aromasin), Fadrozole and Formestane (e.g., Lentaron).

Examples of antiestrogens include but are not limited to: Tamoxifen (e.g., Nolvadex), Fulvestrant (e.g., Faslodex), Raloxifene (e.g., Evista), and Acolbifene.

Examples of LHRH analogues include but are not limited to: Goserelin (e.g., Zoladex) and Leuprolide (e.g., Leuprolide Acetate, such as Lupron or Lupron Depot).

Examples of chemotherapeutic agents include but are not limited to: Trastuzumab (e.g., Herceptin), Gefitinib (e.g., Iressa), Erlotinib (e.g., Erlotinib HCl, such as Tarceva), Bevacizumab (e.g., Avastin), Cetuximab (e.g., Erbitux), and Bortezomib (e.g., Velcade).

Preferably, when more than one antihormonal agent is used, each agent is selected from a different category of agent. For example, one agent is an aromatase inhibitor (e.g., Anastrozole, Letrozole, or Exemestane) and one agent is an antiestrogen (e.g., Tamoxifen or Fulvestrant).

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and at least one antihormonal agent selected from the group consisting of: (a) aromatase inhibitors, (b) antiestrogens, and (c) LHRH analogues; and administering an effective amount of at least one chemotherapeutic agent.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and at least one antihormonal agent selected from the group consisting of: (a) aromatase inhibitors, (b) antiestrogens, and (c) LHRH analogues.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and at least one antihormonal agent selected from the group consisting of: (a) aromatase inhibitors, and (b) antiestrogens.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), at least one antihormonal agent selected from the group consisting of: (a) aromatase inhibitors and (b) antiestrogens; and at least one chemotherapeutic agent.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and at least one aromatase inhibitor.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), at least one aromatase inhibitor, and at least one chemotherapeutic agent.

Another embodiment of this invention is directed to a method of treating or preventing breast cancer in a patient in need of such treatment wherein said treatment comprises administering a therapeutically effective amount of: (1) at least one (e.g., one) compound of formula 1.0 (for example, as described in any one of Embodiment Nos. 1 to 93); and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene, and (c) LHRH analogues that are selected from the group consisting of: Goserelin and Leuprolide; and administering an effective amount of at least one chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0 (for example, as described in any one of Embodiment Nos. 1 to 93); and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene, and (c) LHRH analogues that are selected from the group consisting of: Goserelin and Leuprolide.

Another embodiment of this invention is directed to a method of treating or preventing breast cancer in a patient in need of such treatment wherein said treatment comprises administering a therapeutically effective amount of: (1) at least one (e.g., one) compound of formula 1.0 (for example, as described in any one of Embodiment Nos. 1 to 93); and (2) at least one antihormonal agent selected from the group consisting of: (a) aromatase inhibitors that are selected from the group consisting of Anastrozole, Letrozole, Exemestane, Fadrozole and Formestane, and (b) antiestrogens that are selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of: (1) at least one (e.g., one) compound of formula 1.0 (for example, as described in any one of Embodiment Nos. 1 to 93); and (2) at least one antihormonal agent selected from the group consisting of: (a) aromatase inhibitors that are selected from the group consisting of Anastrozole, Letrozole, Exemestane, Fadrozole and Formestane, (b) antiestrogens that are selected from the group consisting of: Tamoxifen, Fulvestrant, Raloxifene, and Acolbifene; and administering an effective amount of at least one chemotherapeutic agents are selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0 (for example, as described in any one of Embodiment Nos. 1 to 93); and (2) at least one aromatase inhibitor selected from the group consisting of Anastrozole, Letrozole, Exemestane, Fadrozole and Formestane.

Another embodiment of this invention is directed to a method of treating or preventing breast cancer in a patient in need of such treatment wherein said treatment comprises administering a therapeutically effective amount of: (1) at least one (e.g., one) compound of formula 1.0 (for example, as described in any one of Embodiment Nos. 1 to 93); (2) at least one aromatase inhibitor that is selected from the group consisting of Anastrozole, Letrozole, Exemestane, Fadrozole and Formestane; and (3) administering an effective amount of at least one chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of: (1) at least one (e.g., one) compound of formula 1.0 (for example, as described in any one of Embodiment Nos. 1 to 93); (2) at least one aromatase inhibitor; and (3) at least one LHRH analogue.

Another embodiment of this invention is directed to a method of treating or preventing breast cancer in a patient in need of such treatment wherein said treatment comprises administering a therapeutically effective amount of:(1) at least one (e.g., one) compound of formula 1.0 (for example, as described in any one of Embodiment Nos. 1 to 93); (2) at least one antiestrogen; and (3) at least one LHRH analogue.

Another embodiment of this invention is directed to a method of treating or preventing breast cancer in a patient in need of such treatment wherein said treatment comprises administering a therapeutically effective amount of: (1) at least one (e.g., one) compound of formula 1.0 (for example, as described in any one of Embodiment Nos. 1 to 93); (2) at least one aromatase inhibitor that is selected from the group consisting of Anastrozole, Letrozole, Exemestane, Fadrozole and Formestane; and (3) at least one LHRH analogue that is selected from the group consisting of: Goserelin and Leuprolide.

Another embodiment of this invention is directed to a method of treating or preventing breast cancer in a patient in need of such treatment wherein said treatment comprises administering a therapeutically effective amount of: (1) at least one (e.g.; one) compound of formula 1.0 (for example, as described in any one of Embodiment Nos. 1 to 93); (2) at least one antiestrogen that is selected from the group consisting of: Tamoxifen, Fulvestrant, Raloxifene, and Acolbifene; and (3) at least one LHRH analogue that is selected from the group consisting of: Goserelin and Leuprolide.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Letrazole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Exemestane.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Formestane.

Another embodiment of this invention is directed to a method of treating or preventing breast cancer in a patient in need of such treatment wherein said treatment comprises administering a therapeutically effective amount of at least one (e.g., one) compound of formula 1.0 (for example; as described in any one of Embodiment Nos. 1 to 93) and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Goserelin.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93) and Leuprolide.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Anastrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Letrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Exemestane, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Fadrozole, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Formestane, and an antiestrogen selected from the group consisting of: Tamoxifen, Fulvestrant, Raloxifene, and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Anastrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Letrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Exemestane, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Fadrozole, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Formestane, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Anastrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Letrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Exemestane, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Fadrozole, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Formestane, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Anastrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Letrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Exemestane, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Fadrozole, and a chemotherapeutic agent selected from the group consisting of: Trastuzumab, Gefitinib, Erlotinib, Bevacizumab, Cetuximab, and Bortezomib.

Another embodiment of this invention is directed to a method of treating or breast cancer in a patient in need of such treatment wherein said treatment comprises administering a therapeutically effective amount of at least one (e.g., one) compound of formula 1.0 (for example, as described in any one of Embodiment Nos. 1 to 93), Formestane, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Raloxifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Acolbifene, and a chemotherapeutic'agent selected from the group consisting of: Trastuzumab, Gefitinib; Erlotinib, Bevacizumab , Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Goserelin, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolein, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Anastrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Letrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Exemestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Fadrozole, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Formestane, an antiestrogen selected from the group consisting of: Tamoxifen, Fulvestrant, Raloxifene, and Acolbifene, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Anastrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Letrozole, Tamoxifen, and a chemotherapeutic agent selected from the group consisting of: Trastuzumab, Gefitinib, Erlotinib, Bevacizumab, Cetuximab; and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Exemestane, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Fadrozole, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Formestane, Tamoxifen, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Anastrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos: 1 to 93), Letrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Exemestane, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Fadrozole, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Formestane, Fulvestrant, and a chemotherapeutic agent selected from the group consisting of: Trastuzumab, Gefitinib, Erlotinib, Bevacizumab, Cetuximab, and Bortezomib.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Goserelin and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93); Goserelin, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Goserelin, and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Goserelin and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide, and Tamoxifen.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide, and Fulvestrant.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide, and Raloxifene.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide and Acolbifene.

Another embodiment of this invention is directed to a method of treating or preventing breast cancer in a patient in need of such treatment wherein said treatment comprises administering a therapeutically effective amount of at least one (e.g., one) compound of formula 1.0 (for example; as described in any one of Embodiment Nos. 1 to 93), Goserelin and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Goserelin and Letrozole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Goserelin and Exemestane.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Goserelin and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Goserelin and Formestane.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide and Anastrozole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide and Letrozole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide and Exemestane.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide and Fadrozole.

Another embodiment of this invention is directed to a method of treating or preventing breast cancer in a patient in need of such treatment wherein said treatment comprises administering a therapeutically effective amount of at least one (e.g., one) compound of formula 1.0 (for example, as described in any one of Embodiment Nos. 1 to 93), Leuprolide and Formestane.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93) and Anastrozole.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93) and Letrozole.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93) and Exemestane.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93) and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93) and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93), Anastrozole, and Fulvestrant.

Another embodiment of this invention is directed to the treatment or prevention of breast cancer in a patient in need of such treatment, said treatment comprising the administration of a therapeutically effective amount of at least one compound of formula I (e.g., one), Letrozole, and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93), Exemestane, and Fulvestrant.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93), Anastrozole, and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93), Letrozole, and Tamoxifen.

Another embodiment of this invention is directed to the treatment or prevention of breast cancer in a patient in need of such treatment, said treatment comprising the administration of a therapeutically effective amount of at least one (e.g., one) compound of formula 1.0 (for example, as described in any one of Embodiment Nos. 1 to 93), Exemestane, and Tamoxifen.

Other embodiments of this invention are directed to any of the above described embodiments for the treatment of Breast Cancer wherein the chemotherapeutic agent is Trastuzumab.

Other embodiments of this invention are directed to any of the above described embodiments for the treatment or prevention of Breast Cancer wherein the method is directed to the treatment of breast cancer.

The compound of formula 1.0 (for example, as described in any one of Embodiment Nos. 1 to 93), antihormonal agents and chemotherapeutic agents can be administered concurrently or sequentially.

The antihormonal agents and optional chemotherapeutic agents are administered according to their protocols, dosage amounts, and dosage forms that are well know to those skilled in the art (e.g., the Physician's Desk Reference or published literature). For example, for Tamoxifen, Fulvestrant, Raloxifene, Anastrozole, Letrozole, Exemestane, Leuprolide and Goserelin, see the Physician's Desk Reference, 57^{th} Edition, 2003, published by Thomas PDR at Montvale, N.J. 07645-1742, the disclosure of which is incorporated herein by reference thereto.

In general, in the embodiments directed to the methods of treating Breast Cancer: (1) the compound of formula 1.0 (for example, as described in any one of Embodiment Nos. 1 to 93) can be administered daily (e.g., once per day, and in one embodiment twice a day), (2) the aromatase inhibitors can be administered in accordance with the known protocol for the aromatase inhibitor used (e.g., once per day), (3) the antiestrogens can be administered in accordance with the known protocol for the antiestrogen used (e.g., from once a day to once a month), (4) the LHRH analogue can be administered in accordance with the known protocol for the LHRH analogue used (e.g., once a month to once every three months), and (5) the chemotherapeutic agent can be administered in accordance with the known protocol for the chemotherapeutic agent used (e.g., from once a day to once a week).

Radiation therapy, if administered in the above treatments for breast cancer, is generally administered according to known protocols before administration of the compound of formula 1.0 (for example, as described in any one of Embodiment Nos. 1 to 93), antihormonal agents and optional chemotherapeutic agents.

Treatment according to the methods of treating breast cancer is continuous (i.e., a continuous dosing schedule is followed). The treatment is continued until there is a complete response, or until the skilled clinician determines that the patient is not benefiting from the treatment (for example, when there is disease progression).

The continuous treatment protocol for breast cancer can be changed to a discontinuous treatment schedule if, in the judgment of the skilled clinician, the patient would benefit from a discontinuous treatment schedule with one or more of the administered drugs. For example, the compound of formula 1.0 (for example, as described in any one of Embodiment Nos. 1 to 93) can be given using a discontinous treatment schedule while the remaining drugs used in the treatment are given as described herein. An example of a discontinuous treatment protocol for the compound of formula 1.0 (for example, as described in any one of Embodiment Nos. 1 to 93) is a repeating cycle of three weeks with the compound of formula 1.0 followed by one week without the compound of formula 1.0.

After a complete response is achieved with the breast cancer treatment, maintenance therapy with the compound of formula 1.0 (for example, as described in any one of Embodiment Nos. 1 to 93) can be continued using the dosing described in the methods of this invention. Maintenance therapy can also include administration of the antihormonal agents using the dosing described in the methods of this invention. Maintenance therapy can just be with the antihormonal agents. For example, after a complete response is achieved, an aromatase inhibitor (e.g., Anastrozole, Letrozole or Exemestane) can be continued for up to five years. Or, for example, an antiestrogen, e.g., Tamoxifen, may be used for up to five years after a complete response is achieved. Or, for example, an antiestrogen (e.g., Tamoxifen) can be used for up to five years after a complete response is achieved followed by the use of an aromatase inhibitor (e.g., Anastrozole, Letrozole or Exemestane) for up to five years.

In the embodiments directed to the treatment of breast cancer described above, the compound of formula 1.0 (for example, as described in any one of Embodiment Nos. 1 to 93) is administered continuously in a total daily dose of about 100 mg to about 600 mg. Usually this amount is administered in divided doses, and in one embodiment this amount is administered twice a day. In one embodiment the compound of formula 1.0 (for example, as described in any one of Embodiment Nos. 1 to 93) is dosed twice a day in an amount of about 50 mg to about 300 mg per dose. In another embodiment the compound of formula 1.0 (for example, as described in any one of Embodiment Nos. 1 to 93) is dosed twice a day in an amount of about 100 mg to about 200 mg per dose. Examples include the compound of formula 1.0 (for example, as described in any one of Embodiment Nos. 1 to 93) being dosed twice a day at 100 mg per dose. Examples also include the compound of formula 1.0 (for example, as described in any one of Embodiment Nos. 1 to 93) being dosed twice a day at 200 mg per dose.

Anastrozole is administered p.o. and is dosed once a day in amounts of about 0.5 to about 10 mg per dose, and in one embodiment in an amount of about 1.0 mg per dose.

Letrozole is administered p.o. and is dosed once a day in amounts of about 1.0 to about 10 mg per dose, and in one embodiment in an amount of about 2.5 mg per dose.

Exemestane is administered p.o. and is dosed once a day in amounts of about 10 to about 50 mg per dose, and in one embodiment in an amount of about 25 mg per dose.

Fadrozole is administered p.o. and is dosed twice a day in amounts of about 0.5 to about 10 mg per dose, and in one embodiment in an amount of about 2.0 mg per dose.

Formestane is administered i.m. and is dosed once every two weeks in amounts of about 100 to about 500 mg per dose, and in one embodiment in an amount of about 250 mg per dose.

Tamoxifen is administered p.o. and is dosed once a day in amounts of about 10 to about 100 mg per dose, and in one embodiment in an amount of about 20 mg per dose.

Fulvestrant is administered i.m. and is dosed once a month in amounts of about 100 to about 1000 mg per dose, and in one embodiment in an amount of about 250 mg per dose.

Raloxifene is administered p.o. and is dosed once a day in amounts of about 10 to about 120 mg per dose, and in one embodiment in an amount of about 60 mg per dose.

Acolbifene is administered p.o. and is dosed once a day in amounts of about 5 to about 20 mg per dose, and in one embodiment in an amount of about 20 mg per dose.

Goserelin is administered s.c. and is dosed once a month, or once every three months, in amounts of about 2 to about 20 mg per dose, and in one embodiment in an amount of about 3.6 mg per dose when administered once a month, and in another embodiment in an amount of about 10.8 mg per dose when administered once every three months.

Leuprolide is administered s.c. and is dosed once a month, or once every three months, in amounts of about 2 to about 20 mg per dose, and in one embodiment in an amount of about 3.75 mg per dose when administered once a month, and in another embodiment in an amount of about 11.25 mg per dose when administered once every three months.

Trastuzumab is administered by i.v. and is dosed once a week in amounts of about 2 to about 20 mpk per dose, and in one embodiment in an amount of about 2 mpk per dose. Trastuzumab is generally initially administered in a loading dose that is generally twice the dose of the weekly dose. Thus, for example, a 4 mpk loading dose is administered and then dosing is 2 mpk per dose per week.

Gefitinib is administered p.o. and is dosed once a day in amounts of about 100 to about 1000 mg per dose, and in one embodiment in an amount of about 250 mg per dose.

Erlotinib is administered p.o. and is dosed once a day in amounts of about 100 to about 500 mg per dose, and in one embodiment in an amount of about 150 mg per dose.

Bevacizumab is administered i.v. and is dosed once every two weeks in amounts of about 2.5 to about 15 mg per kilogram of body weight per dose, and in one embodiment in an amount of about 10 mg per kilogram per dose.

Cetuximab is administered i.v. and is dosed once a week in amounts of about 200 to about 500 mg per meter squared dose, and in one embodiment in an amount of about 250 mg per meter squared per dose.

Bortezomib is administered i.v. and is dosed twice a week for 2 weeks followed by a 10 day rest period (21 day treatment cycle) for a maximum of 8 treatment cycles in amounts of about 1.0 to about 2.5 mg per meter squared per dose, and in one embodiment in an amount of about 1.3 mg per meter squared per dose.

Thus in one embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) orally in an amount of about 50 mg to about 300 mg per dose wherein each dose administered twice a day, and (2) Anastrozole p.o. in an amount of about 0.5 to about 10 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) orally in an amount of about 100 to 200 mg per dose, wherein each dose is administered twice a day, and (2) Anastrozole in an amount of about 1.0 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) orally in an amount of about 50 mg to about 300 mg per dose wherein each dose is administered twice a day, and (2) Letrozole p.o. in an amount of about 1.0 to about 10 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) orally in an amount of about 100 to 200 mg per dose, wherein each dose is administered twice a day, and (2) Letrozole p.o. in an amount of about 2.5 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) orally in an amount of about 50 mg to about 300 mg per dose wherein each dose is administered twice a day, and (2) Exemestane p.o. in an amount of about 10 to about 50 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) orally in an amount of about 100 to 200 mg per dose, wherein each dose is administered twice a day, and (2) Exemestane in an amount of about 25 mg pier dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) orally in an amount of about 50 mg to about 300 mg per dose wherein each dose is administered twice a day, and (2) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) orally in an amount of about 100 to 200 mg per dose, wherein each dose is administered twice a day, and (2) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 50 mg to about 300 mg per dose wherein each dose is administered twice a day, and (2) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 100 to 200 mg per dose, wherein each dose is administered twice a day, and (2) Tamoxifen p.o. in an amount of about 20 mg per dose wherein each dose is given once a day.

In other embodiments of the invention breast cancer is treated in a patient in need of such treatment wherein said treatment comprises the administration of the compound of formula 1.0 (for example, as described in any one of Embodiment Nos. 1 to 93), one of the aromatase inhibitors (e.g., Anastrozole, Letrozole, or Exemestane, and in one embodiment Anastrozole), and one of the antiestrogens (e.g., Fulvestrant or Tamoxifen), wherein the compound of formula 1.0, aromatase inhibitor and antiestrogen are administered in the dosages described above.

Thus, for example in another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 50 mg to about 300 mg per dose wherein each dose is administered twice a day, (2) Anastrozole p.o. in an amount of about 0.5 to about 10 mg per dose wherein each dose is given once a day, and (3) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o in an amount of about 100 to 200 mg per dose, wherein each dose is administered twice a day, (2) Anastrozole p.o. in an amount of about 1.0 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 50 mg to about 300 mg per dose wherein each dose is administered twice a day, (2) Letrozole p.o in an amount of about 1.0 to about 10 mg per dose wherein each dose is given once a day, and (3) Fulvestrant in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 100 to 200 mg per dose, wherein each dose is administered twice a day, (2) Letrozole p.o. in an amount of about 2.5 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 50 mg to about 300 mg per dose wherein each dose is administered twice a day, (2) Exemestane p.o. in an amount of about 10 to about 50 mg per dose wherein each dose is given once a day, and (3) Fulvestrant i.m. in an amount of about 100 to about 1000 mg per dose wherein each dose is given once a month.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 100 to 200 mg per dose, wherein each dose is administered twice a day, (2) Exemestane p.o. in an amount of about 25 mg per dose wherein each dose is given once a day, and (3) Fulvestrant i.m. in an amount of about 250 mg per dose wherein each dose is given once a month.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 50 mg to about 300 mg per dose wherein each dose is administered twice a day, (2) Anastrozole p.o. in an amount of about 0.5 to about 10 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 100 to 200 mg per dose, wherein each dose is administered twice a day, (2) Anastrozole p.o. in an amount of about 1.0 mg per dose wherein each dose is given once a day, and (3) Tamoxifen p.o. in an amount of about 20 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 50 mg to about 300 mg per dose wherein each dose is administered twice a day, (2) Letrozole p.o. in an amount of about 1.0 to about 10 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 100 to 200 mg per dose, wherein each dose is administered twice a day, (2) Letrozole p.o. in an amount of about 2.5 mg per dose wherein each dose is given once a day, and (3) Tamoxifen p.o. in an amount of about 20 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 50 mg to about 300 mg per dose wherein each dose is administered twice a day, (2) Exemestane p.o. in an amount of about 10 to about 50 mg per dose wherein each dose is given once a day, and (3) Tamoxifen p.o. in an amount of about 10 to about 100 mg per dose wherein each dose is given once a day.

In another embodiment of this invention breast cancer is treated (or prevented) in a patient in need of such treatment wherein said treatment comprises administering to said patient: (1) the compound of formula 1.0 (for example, as described in any one of Embodiment Nos. 1 to 93) p.o. in an amount of about 100 to 200 mg per dose, wherein each dose is administered twice a day, (2) Exemestane p.o. in an amount of about 25 mg per dose wherein each dose is given once a day, and (3) Tamoxifen p.o. in an amount of about 20 mg per dose wherein each dose is given once a day.

Those skilled in the art will appreciate that when other combinations of antihormonal agents are used, the individual antihormonal agent is used in the amounts specified above for that individual antihormonal agent.

Other embodiments of the treatment of Breast Cancer are directed to the methods of treating Breast Cancer described above wherein the compound of formula 1.0 (for example, as described in any one of Embodiment Nos. 1 to 93) is dosed twice a day in an amount of about 100 mg per dose.

Other embodiments of the treatment of Breast Cancer are directed to the methods of treating Breast Cancer described above wherein the compound of formula 1.0 (for example, as described in any one of Embodiment Nos. 1 to 93) is dosed twice a day in an amount of about 200 mg per dose.

Other embodiments of the treatment of Breast Cancer are directed to the methods of treating Breast Cancer described above wherein a chemotherapeutic agent is administered in addition to the compound of formula 1.0 (for example, as described in any one of Embodiment Nos. 1 to 93) and antihormonal agent (or antihormonal agents). In these embodiments the dosage ranges of the compound of formula 1.0 and antihormonal agents are as those described above in the combination therapies, or those described above for the individual compound of formula I and antihormonal agents, and the dosages of the chemotherapeutic agents are those described above for the individual chemotherapeutic agent. The dosages for the chemotherapeutic agents are well known in the art.

Other embodiments of this invention are directed to pharmaceutical compositions comprising the compound of formula 1.0 (for example, as described in any one of Embodiment Nos. 1 to 93) and at least one antihormonal agent and a pharmaceutically acceptable carrier.

Other embodiments of this invention are directed to pharmaceutical compositions comprising the compound of formula 1.0 (for example, as described in any one of Embodiment Nos. 1 to 93), at least one antihormonal agent, at least one chemotherapeutic agent, and a pharmaceutically acceptable carrier.

Other embodiments of this invention are directed to pharmaceutical compositions comprising the compound of formula 1.0 (for example, as described in any one of Embodiment Nos. 1 to 93), at least one chemotherapeutic agent, and a pharmaceutically acceptable carrier.

Those skilled in the art will appreciate that the compounds (drugs) used in the methods of this invention are available to the skilled clinician in pharmaceutical compositions (dosage forms) from the manufacturer and are used in those compositions. So, the recitation of the compound or class of compounds in the above described methods can be replaced with a recitation of a pharmaceutical composition comprising the particular compound or class of compounds. For example, the embodiment directed to a method of treating cancer comprising administering to a patient in need of such treatment therapeutically effective amounts of the compound of formula 1.0, a taxane, and a platinum coordination compound, includes within its scope a method of treating cancer comprising administering to a patient in need of such treatment therapeutically effective amounts of a pharmaceutical composition comprising the compound of formula a pharmaceutical composition comprising a taxane, and a pharmaceutical composition comprising a platinum coordination compound.

Those skilled in the art will recognize that the actual dosages and protocols for administration employed in the methods of this invention may be varied according to the judgment of the skilled clinician. The actual dosage employed may be varied depending upon the requirements of the patient and the severity of the condition being treated. Determination of the proper dosage for a particular situation is within the skill of the art. A determination to vary the dosages and protocols for administration may be made after the skilled clinician takes into account such factors as the patient's age, condition and size, as well as the severity of the cancer being treated and the response of the patient to the treatment.

The amount and frequency of administration of the compound of formula 1.0 and the chemotherapeutic agents will be regulated according to the judgment of the attending clinician (physician) considering such factors as age, condition and size of the patient as well as severity of the cancer being treated.

The chemotherapeutic agent can be administered according to therapeutic protocols well known in the art. It will be apparent to those skilled in the art that the administration of the chemotherapeutic agent can be varied depending on the cancer being treated and the known effects of the chemotherapeutic agent on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the cancer to the administered therapeutic agents.

The initial administration can be made according to established protocols known in the art, and then, based upon the observed effects, the dosage, modes of administration and times of administration can be modified by the skilled clinician.

The particular choice of chemotherapeutic agent will depend upon the diagnosis of the attending physicians and their judgement of the condition of the patient and the appropriate treatment protocol.

The determination of the order of administration, and the number of repetitions of administration of the chemotherapeutic agent during a treatment protocol, is well within the knowledge of the skilled physician after evaluation of the cancer being treated and the condition of the patient.

Thus, in accordance with experience and knowledge, the practicing physician can modify each protocol for the administration of an chemotherapeutic agent according to the individual patient's needs, as the treatment proceeds. All such modifications are within the scope of the present invention.

The particular choice of antihormonal agents, optional chemotherapeutic agents and optional radiation will depend upon the diagnosis of the attending physicians and their judgment of the condition of the patient and the appropriate treatment protocol.

The determination of the order of administration, and the number of repetitions of administration of the antihormonal agents, optional chemotherapeutic agents and optional radiation during a treatment protocol, is well within the knowledge of the skilled physician after evaluation of the breast cancer being treated and the condition of the patient.

Thus, in accordance with experience and knowledge, the practicing physician can modify each protocol for the administration of antihormonal agents, optional chemotherapeutic agents and optional radiation according to the individual patient's needs, as the treatment proceeds. All such modifications are within the scope of the present invention.

The attending clinician, in judging whether treatment is effective at the dosage administered, will consider the general well-being of the patient as well as more definite signs such as relief of cancer-related symptoms (e.g., pain, cough (for lung cancer), and shortness of breath (for lung cancer)), inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

The compounds of the invention can be made according to the processes described below.

The LCMS conditions are: (1) column: C-18 reverse phase, 5 um, 4.6×50 mm, (2) MS:PE Sciex API-150EX, and (3) HPLC: Shimadzu LC-10 ADvp, 1 ml/min, linerar gradient 10% acetonitirle in water to 95% acetonitrile in water, both contain 0.05% TFA

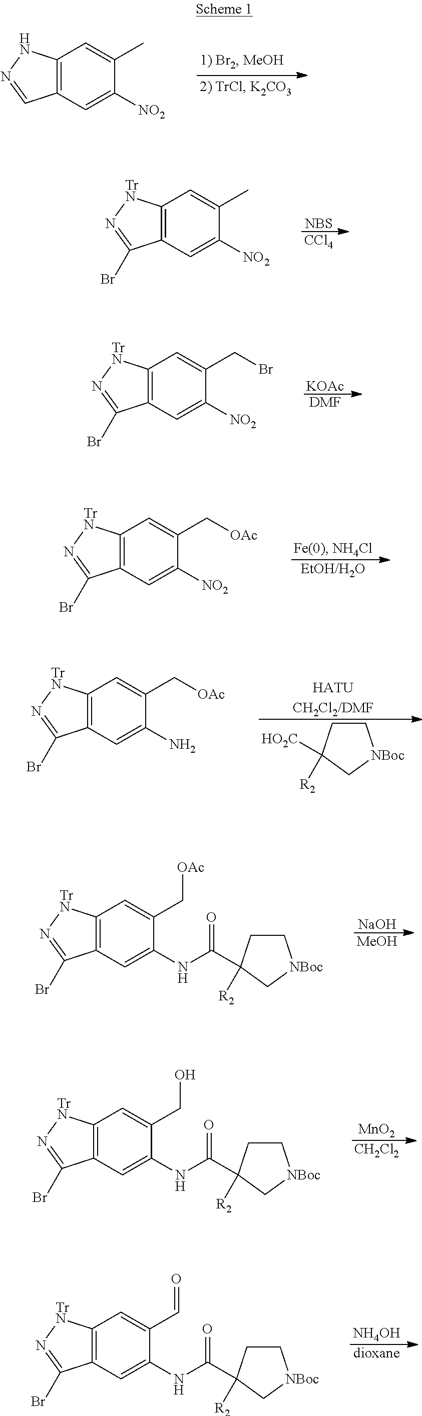

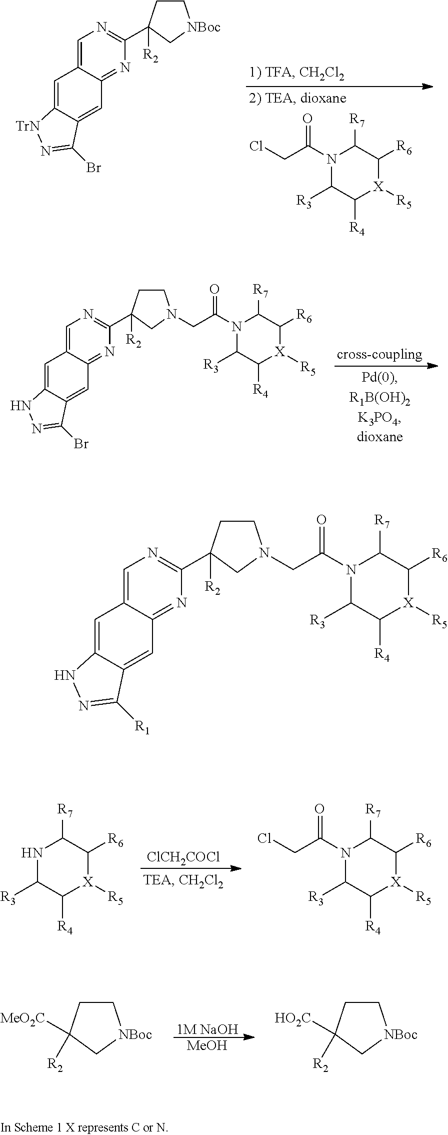

### Preparation 1

### Step 1: Synthesis of 3-Bromo-6-methyl-5-nitro-1H-indazole (2)

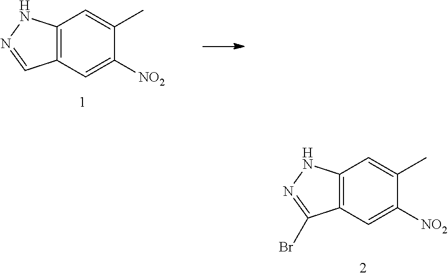

To a refluxing solution of 1 (3.65 g, 20.6 mmol) in MeOH (60 mL) was added Br₂ (1.06 mL, 20.6 mmol) slowly. The reaction was continued with stirring at reflux for 1 hr and was then cooled in an ice bath. The resulting precipitate was collected by filtration and washed with cold CH₂Cl₂/hexanes (1:1). The filtrate was concentrated and washed with cold CH₂Cl₂/hexanes (1:1). The combined solid was dried under high vacuum to yield 2 as a peach solid (3.76 g).

### Step 2: Synthesis of 3-Bromo-6-methyl-5-nitro-1-trityl-1H-indazole (3)

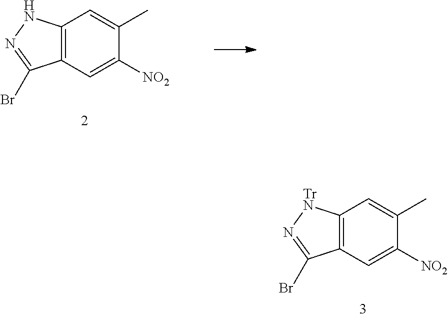

3-Bromo-6-methyl-5-nitro-1H-indazole 2 (1.0 g, 3.9 mmol) and (1.3 g, 9.4 mmol) were dissolved in CH₃CN (22 mL). To this was added TrCl (1.31 g, 4.7 mmol). The resulting mixture was heated at 70° C. for 8 hrs. The reaction was cooled to rt and partitioned between CH2Cl2 and H2O. The aqueous layer was extracted with CH2Cl2 twice. The combined organic layers was dried (MgSO₄), filtered and conc. in vacuo to provide a crude 3 (1.73 g).

### Step 3: Synthesis of 3-Bromo-6-bromomethyl-5-nitro-1-trityl-1H-indazole (4)

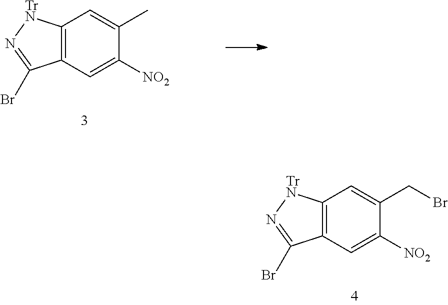

3-Bromo-6-methyl-5-nitro-1-trityl-1H-indazole 3 (3.63 g, 7.28 mmol) and NBS (1.43 g, 8.01 mmol) was dissolved in CCl₄ (45 mL). (PhCO)₂O₂ (353 mg, 1.46 mmol) was added and heated at 85° C. overnight. After cooling, the mixture was diluted with CH₂Cl₂ and treated with Na₂S₂O₃ solution followed by extraction with CH₂Cl₂ (3×). The combined organic layer was dried and conc. in vacuo. The resulting crude was purified via flash column using 3% to 5% ethyl acetate in hexanes to give 4 as an off white solid (790 mg).

### Step 4: Synthesis of Acetic acid 3-bromo-5-nitro-1-trityl-1H-indazol-6-ylmethyl ester (5)

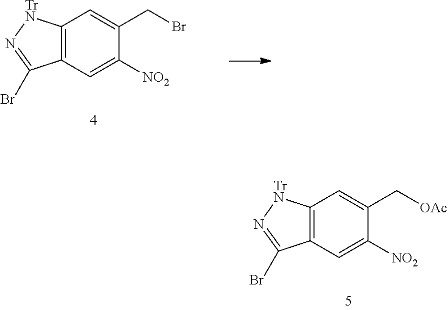

To solution of 4 (555 mg; 0.87 mmol) in DMF (3 mL) was added KOAc (425 mg, 4.33 mmol). The reaction was stirred at it for 20 min before diluted with CH₂Cl₂ and quenched with NH₄Cl solution. The aqueous layer was extracted with CH₂Cl₂ (3×). The combined organic layer was washed with brine, dried and conc. in vacuo. The resulting crude solid was washed with 5% ethyl acetate in hexanes repeatedly and dried under vacuum to afford 5 as a solid (464 mg).

### Step 6: Synthesis of Acetic acid 5-amino-3-bromo-1-trityl-1H-indazol-6-ylmethyl ester (6)

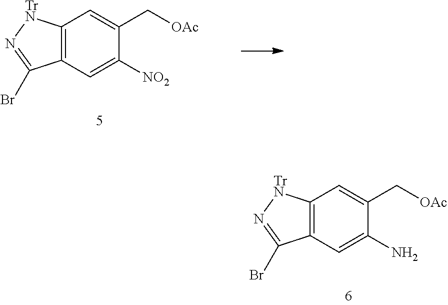

To a suspension of 5 (3.8 g, 6.83 mmol) in EtOH/CH₃Ph/CH₂Cl₂/H₂O (160 mL/160 mL/20 mL/20 mL) was added Fe(0) powder (3.82 g, 68.3 mmol) and several drops of conc. HCl. The reaction was heated at reflux for 2 hrs and then cooled and quenched with sat. NaHCO₃ solution. The mixture was filtered through Celite rinsing with CH₂Cl₂/MeOH. The filtrate was conc. in vacuo and partitioned between CH₂Cl₂/H₂O. The aqueous layer was extracted with ethyl acetate and CH₂Cl₂. The combined organic layer was dried and conc. The crude was purified via flash column eluting with 20% to 50% ethyl acetate/hexanes to yield 6 (1.93 g).

### Step 7: Synthesis of 3-(6-Acetoxymethyl-3-bromo-1-trityl-1H-indazol-5-ylcarbamoyl)-pyrrolidine-1-carboxylic acid tert-butyl ester (7)

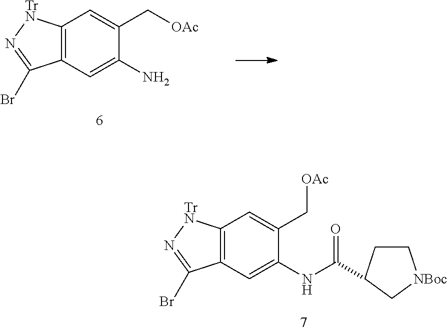

A mixture of 6 (600 mg, 1.14 mmol), pyrrolidine-1,3-dicarboxylic acid 1-tert-butyl ester (736 mg, 3.4 mmol), HATU (1.3 g, 3.4 mmol) and triethyl amine (1.6 mL) in DMF/CH₂Cl₂ (6 mL/6 mL) was stirred at rt overnight. The reaction was partitioned between CH₂Cl₂ and H₂O. The aqueous layer was extracted with CH₂Cl₂ twice and the combined organic layer was washed with brine, dried (MgSO₄) and conc. in vacuo. The crude was purified via flash column using 30% ethyl acetate in hexanes to get 7 (1.34 g).

### Step 8: Synthesis of 3-(3-Bromo-6-hydroxymethyl-1-trityl-1H-indazol-5-ylcarbamoyl)-pyrrolidine-1-carboxylic acid tert-butyl ester (8)

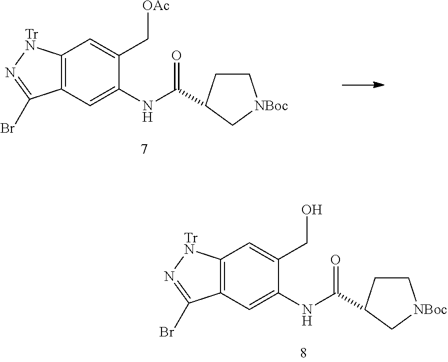

To a solution of 7 (33 mg, 0.06 mmol) in THF/EtOH (3 mL/1 mL) was added 1N NaOH (0.9 mL). The mixture was stirred at rt for 16 hrs before quenched with NH₄Cl solution. The crude was extracted with CH₂Cl₂ (3×) and ethyl acetate (1×). The combined organic layer was dried and conc. in vacuo. The crude was purified via prep TLC plate developing with 20% ethyl acetate in hexanes to afford 8 as a yellow oil (25 mg).

### Step 9: Synthesis of 3-(3-Bromo-6-formyl-1-trityl-1H-indazol-5-ylcarbamoyl)-pyrrolidine-1-carboxylic acid tert-butyl ester (9)

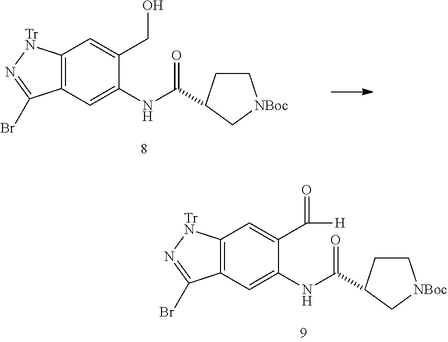

To a solution of 8 (720 mg, 1.06 mmol) in CH₂Cl₂ (10 mL) was added MnO₂ (3.0 g). The mixture was stirred at rt for 24 hrs and then filtered through Celite. The filtrate was conc. in vacuo and the resulting crude was purified via silica gel column using 5% to 3% ethyl acetate in hexanes to yield 9 (582 mg) as a yellow solid.

### Step 10: Synthesis of 3-(3-Bromo-1-trityl-1H-pyrazolo[3,4-g]quinazolin-6-yl)-pyrrolidine-1-carboxylic acid tert-butyl ester (10)

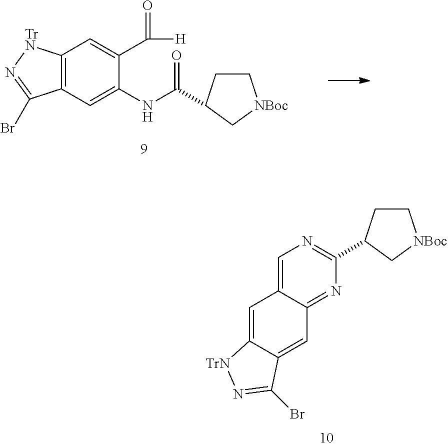

To a solution of 9 (580 mg, 0.85 mmol) in dioxane (45 mL) in a seal tube was added NH₄OH (45 mL, 28% wt in H₂O). The mixture was heated at 130° C. for 90 mins. The reaction was cooled to rt. and diluted with ethyl acetate. The aqueous layer was extracted with ethyl acetate twice and the combined organic layer was washed with brine, dried (MgSO₄) and conc. in vacuo. The crude was purified via flash column using 5% to 3% ethyl acetate in hexanes to get 10 as a yellow solid (414 mg).

### Step 11: Synthesis of 8-Bromo-2-pyrrolidin-3-yl-6H-pyrrolo[3,4-g]quinazoline (11)

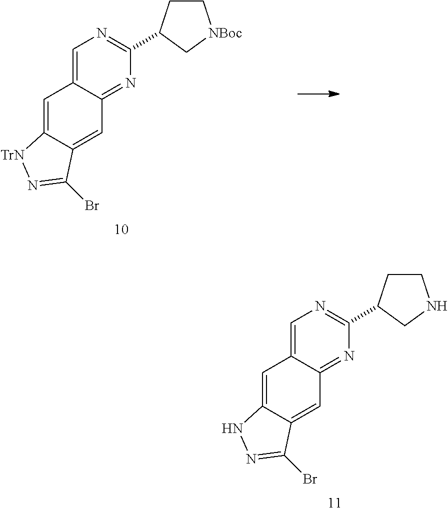

To a solution of 10 (285 mg, 0.43 mmol) in 10 ml of dichloromethane was added 3.24 mL of 4N HCl in dioxane. The crude reaction mixture was stirred at ambient temperature for 18 hrs. The crude was evaporated under vacuum and dissolved in dichloromethane/methanol and neutralized with saturated NaHCO₃. The aqueous extracted with ethyl acetate 2×, dried over sodium sulfate, filtered and evaporated under vacuum. The crude was purified by silica gel chromatography using 5%-20% 2N methanol-ammonia/dichloromethane to obtain 167 mg of title product.

### Preparation 2

### Step 1: Synthesis of 4-(4-Bromo-phenyl)-piperazine-1-carboxylic acid tert-butyl ester (12)

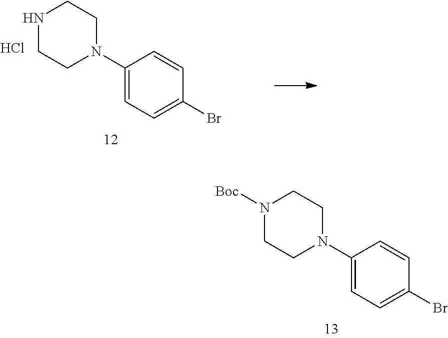

1-(4-Bromo-phenyl)-piperazine hydrochloride (9 gm, 38 mmol) was dissolved in 250 ml of dichloromethane and 9 ml of triethylamine added. Di-tert.butyldicarbonate (8.34 gm, 39 mmol) was added and the reaction mixture stirred for 1 hr. The reaction mixture was washed with a solution of saturated sodium bicarbonate (100 ml), the organic layer separated, dried over magnesium sulfate and evaporated to obtain 10.19 gm of crystalline product.

### Step 2: Synthesis of 4(4-boronic acid-phenyl)-piperazine-1-carboxylic acid tert-butyl ester (14)

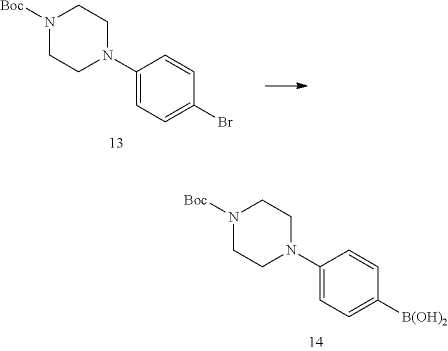

4-(4-Bromo-phenyl)-piperazine-1-carboxylic acid tert-butyl ester (10.19 gm, 30 mmol) was dissolved in 26 ml of tetrahydrofuran. The mixture was cooled to −78 C under a dry nitrogen atmosphere. A 2.5 N solution nButyl lithium in hexanes (26 ml, 65 mmol) was added dropwise and stirred for 30 min. Triisopropylborate (14.68 ml, 63.6 mmol) was added over 10 min. and the reaction mixture let warm to ambient temperature gradually. The reaction mixture was stirred for 18 hrs. A saturated solution of Ammonium chloride (75 ml) was added and the reaction mixture stirred for 5 min. 85% o-Phosphoric acid (7.27 gm) was added and the reaction mixture stirred for 1 hr. The reaction mixture was extracted with ethylacetate three times, dried over magnesium sulfate, filtered and evaporated. The crude product was chromatographed on a silica column to obtain 5.74 gm of title product.

### Step 3: Preparation of 4-[4-(5-Fluoro-pyrimidin-2-yl)-phenyl]-piperazine-1-carboxylic acid tert-butyl ester (15)

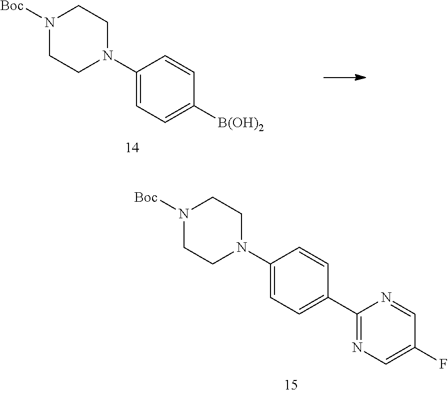

4-[4-(5-Fluoro-pyrimidin-2-yl)-phenyl]-piperazine-1-carboxylic acid tert-butyl ester was prepared similarly as in Preparation 3 Step 1 substituting 4-(4-boronic acid-phenyl)-piperazine-1-carboxylic acid tert-butyl ester with 4-[4-(tert-Butoxycarbonyl)-piperazin-1-yl]phenylboronic acid (C. Chen et. al. J. Org. Chem. 2003, 68, 2633).

### Step 4: Synthesis of 5-Fluoro-2-(4-piperazin-1-yl-phenyl)-pyrimidine (16

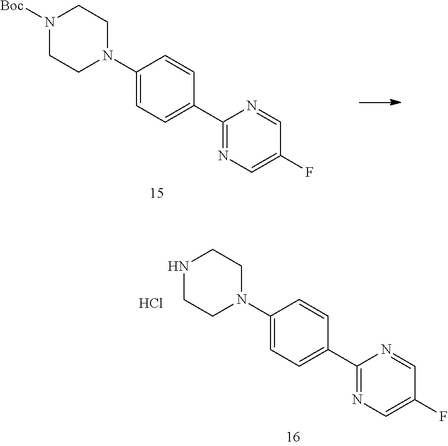

4-fluoro-(4-Pyrimidin-2-yl-phenyl)piperazine-1-carboxylic acid tert-butyl ester 5.03 gm was dissolved in 25 ml dichloromethane and 10 ml of 4N HCl dioxane added. After stirring for 2 hrs, the mixture was then evaporated to obtain the title product.

### Step 5: Synthesis of 2-Chloro-1-{4-[4-(5-fluoro-pyrimidin-2-yl)-phenyl]-piperazin-1-yl}-ethanone (17)

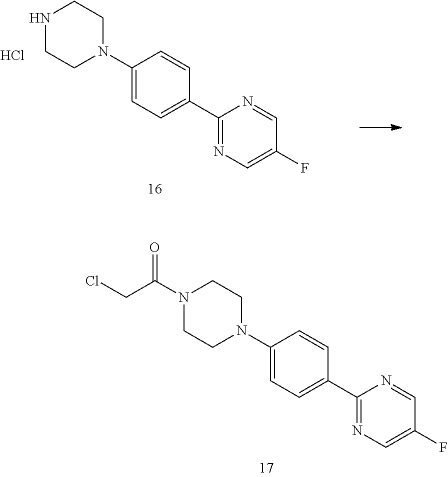

Follow procedure as in the preparation of 2-Chloro-1-[4-(4-pyrimidin-2-yl-phenyl)-3,6-dihydro-2H-pyridin-1-yl]-ethanone (20 in Preparation 3) below substituting 5-Fluoro-2-(4-piperazin-1-yl-phenyl)-pyrimidine.

### Preparation 3

### Step 1: Synthesis of 4-(4-Pyrimidin-2-yl-phenyl)-piperazine-1-carboxylic acid tert-butyl ester (18)

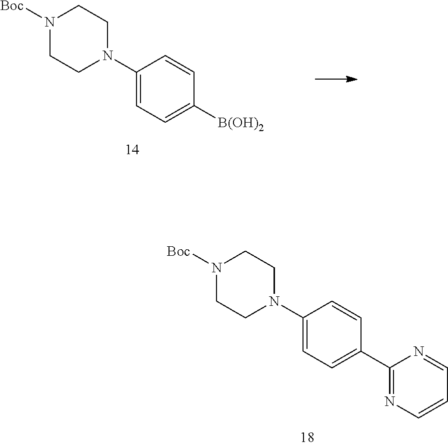

4-(4-boronic acid-phenyl)-piperazine-1-carboxylic acid tert-butyl ester (5.93 gm, 19.3 mmol) was dissolved in 50 ml of a 50% mixture of N,N-dimethylformamide/water. K2CO3 (16 gm) was added and the mixture de-gased and purged with nitrogen. Pd (dppf)2Cl2 (1.57 gm) and 2-chloropyrimidine (2.72 gm) was added and the reaction mixture stirred at 80 C. After 8 hours the product was extracted into ethylacetate, dried over magnesium sulfate, filtered and evaporated. The crude product was chromatographed on silica gel to obtain 5.03 gm (76.6%) of title product.

### Step 2: Synthesis of 2-(4-piperazin-1-yl-phenyl)-pyrimidine (19)

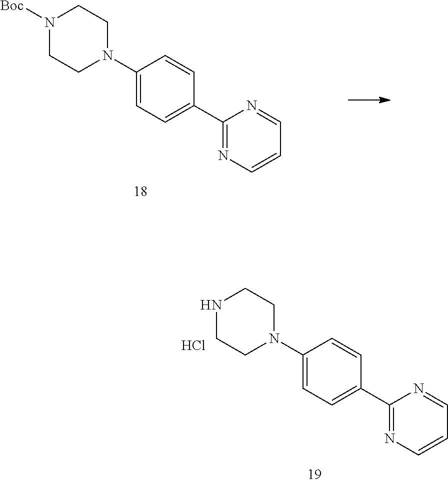

4-(4-Pyrimidin-2-yl-phenyl)piperazine-1-carboxylic acid tert-butyl ester 5.03 gm was dissolved in 25 ml dichloromethane and 10 ml of 4N HCl dioxane added. After stirring for 2 hrs, the mixture was then evaporated to obtain the title product.

### Step 3: Preparation of 2-Chloro-1-[4-(4-pyrimidin-2-yl-phenyl)-3,6-dihydro-2H-pyridin-1-yl]-ethanone (20)

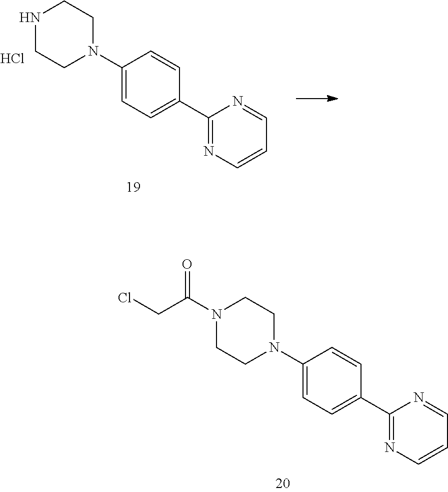

2-[4-(1,2,3,6-Tetrahydro-pyridin-4-yl)-phenyl]-pyrimidine trifluoroacetate (2.3 g, 9.7 mmol) was dissolved in 75 ml of dichloromethane and 4.1 ml of triethylamine added at 0 C. Chloroacetylchloride (0.92 ml, 11.7 mmol) was added and the reaction mixture stirred for 30 min. The reaction mixture was washed with a solution of saturated sodium bicarbonate (80 ml), the organic layer separated, dried over magnesium sulfate and evaporated to obtain 2.41 g of crystalline product.

### Preparation 4

### Preparation of 2-[4-(1,2,3,6-Tetrahydro-pyridin-4-yl)-phenyl]-pyrimidine trifluoroacetic acid salt (23)

### Steps 1 and 2:

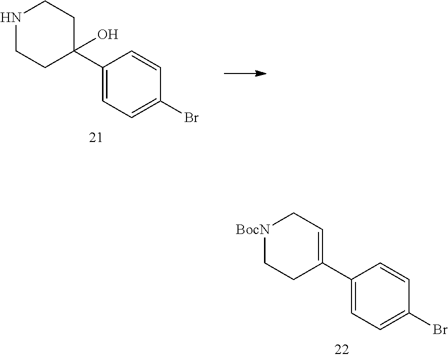

### Step 1:

4-(4-Bromophenyl)-4-piperidinol (68 g, 0.27 mol) was added in small portions to a solution of trifluoroacetic acid (205 ml) at r.t. and the mixture was heated at 90° C. for 2 hr. Solvents were then removed in vacuum to give 4-(4-bromophenyl)-1,2,3,6-tetrahydropyridine as pale yellow oil. The yellow oil was used in the next step without further purification.

### Step 2:

4-(4-Bromophenyl)-1,2,3,6-tetrahydropyridine (crude from step 1) was stirred in dichloromethane (500 ml) at r.t. Triethylamine (148 ml, 1.06 mol) followed by (Boc)₂O (87 g, 0.40 mol) were added. The suspension slowly dissolved and the yellow solution was stirred at r.t. for 2 hr. The mixture was washed with water (×2), dried (MgSO₄) and chromatograph through a short pad of silica. The fractions with the product 4-(4-Bromophenyl)-3,6-dihydro-2H-pyridine-1-carboxylic acid tert-butyl ester were combined and solvents were removed in vacuum to give pale yellow oil which solidified on standing at r.t. to become white solid (91 g, quant.)

### Steps 3 and 4:

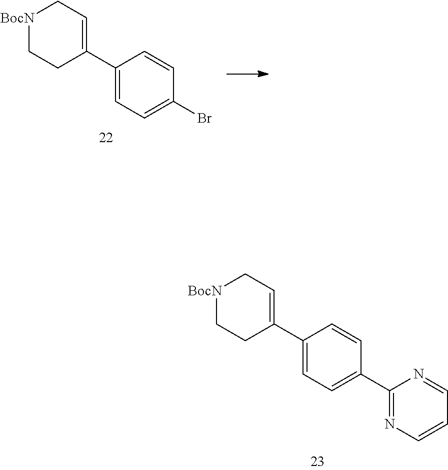

### Step 3:

4-(4-Bromophenyl)-3,6-dihydro-2H-pyridine-1-carboxylic acid tert-butyl ester (19.5 g, 0.058 mol), bis(pinacolate)diboron (22.0 g, 0.086 mol), PdCl₂(dppf).CH₂Cl₂ (4.74 g, 0.0058 mol), potassium acetate (17.0 g, 0.17 mol) were weighted into a 1 L 2-necked round bottomed flask equipped with a reflux condenser. Methyl sulfoxide (400 ml) was added and the mixture was purged with nitrogen for 20 min before it was heated at 100° C. for 2 hr under nitrogen. The mixture was cooled to r.t. Potassium carbonate (40 g, 0.29 mol), 2-bromopyrimidine (11.0 g, 0.070 mol) and water (200 ml) were added. The mixture was again purged with nitrogen for 20 min. Palladium tetrakistriphenylphosphine (2.4 g, 0.0029 mol) was added and the final mixture was stirred at 100° C. for a further 2 hr. After being cooled to r.t., ethyl acetate and water were added. The mixture was filtered through a pad of Celite. Layers were separated and the organic layer was washed with water (×2). The combined aqueous layers were extracted with ethyl acetate (×1). The combined organic layers were stirred with enough charcoal to give a yellow solution. The mixture was filtered through a pad of Celite and the solvents in the filtrate were removed in vacuum to give 4-(4-pyrimidin-2-yl-phenyl)-3,6-dihydro-2H-pyridine-1-carboxylic acid tert-butyl ester as dark brown oil.

### Step 4:

4-(4-pyrimidin-2-yl-phenyl)-3,6-dihydro-2H-pyridine-1-carboxylic acid tert-butyl ester (crude from step 3) was dissolved in dichloromethane (200 ml) and trifluoroacetic acid (22 ml, 0.29 mol) was added at r.t. The mixture was stirred at r.t. for 5 hr and solvents were removed in vacuum. Diethyl ether was added and off-white solid was formed. The solid was filtered and washed with diethyl ether to give a salt (14.4 g, 71%).

### Preparation 5

### Synthesis of 3-Bromo-6-pyrrolidin-3-yl-1-trityl-1H-pyrazolo[3,4-g]quinazoline (10a)

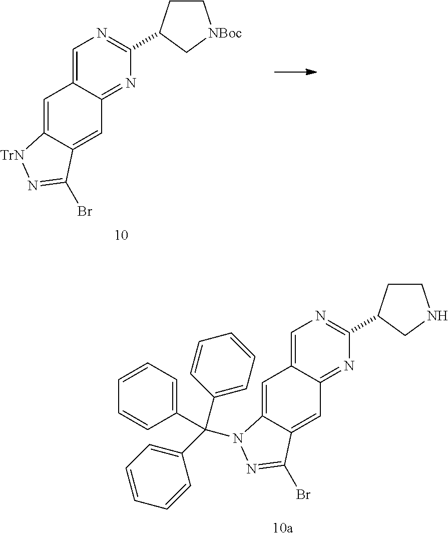

To a solution of Synthesis of 3-(3-Bromo-1-trityl-1H-pyrazolo[3,4-g]quinazolin-6-yl)-pyrrolidine-1-carboxylic acid tert-butyl ester (285 mg, 0.43 mmol) was added 3.24 mL of 4 N HCl in dioxane and the reaction mixture stirred overnite at room temperature. The crude was evaporated under vacuum, dissolved in 75% dichloromethane/methanol and quenched with saturated, NaHCO3. The organic-layer was separated and the water layer washed 3× with dichloromethane. The organic layers were dried over Na₂SO₄, filtered and evaporated to obtain a solid. The crude solid was chromatographed on silica gel using 5% methanol/dichoromethane as eluent to obtain 167 mg of title product. MS (561, M+1)

### Example 1

### Step 1: Synthesis of 2-[3-(3-Bromo-1-trityl-1H-pyrazolo[3,4-g]quinazolin-6-yl)-pyrrolidin-1-yl]-1-{4-[4-(5-fluoro-pyrimidin-2-yl)-phenyl]-piperazin-1-yl}-ethanone (24)

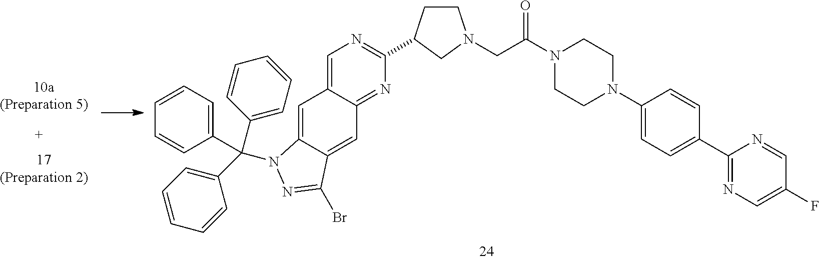

3-Bromo-6-pyrrolidin-3-yl-1-trityl-1H-pyrazolo[3,4-g]quinazoline (167 mg, 0.30 mmol) was dissolved in 10 ml of dry dioxane. 2-Chloro-1-{4-[4-(5-fluoro-pyrimidin-2-yl)-phenyl]-piperazin-1-yl}-ethanone (120 mg, 0.36 mmol) was added followed by 0.17 ml of triethylamine. The reaction mixture was stirred overnight. The crude was diluted with dichloromethane, washed with water and dried over Na2SO4. The crude was chromatographed on silica gel using 3-5% methanol (2N NH3)/dichloromethane to obtain 167 mg of title product. MS (858, M+1).

### Step 2: Synthesis of 1-{4-[4-(5-Fluoro-pyrimidin-2-yl)-phenyl]-piperazin-1-yl}-2-[3-(3-pyridin-4-yl-1-trityl-1H-pyrazolo[3,4-g]quinazolin-6-yl)-pyrrolidin-1-yl]-ethanone (25)

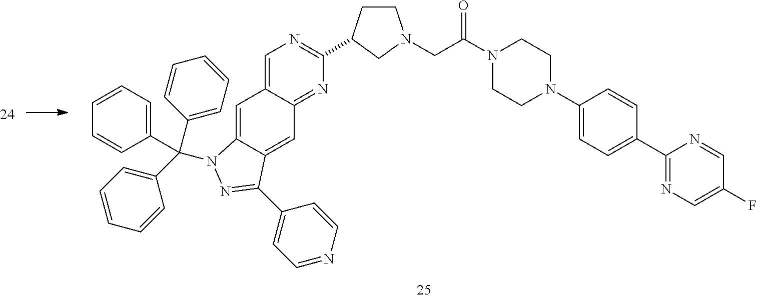

2-[3-(3-Bromo-1-trityl-2H-pyrazolo[3,4-g]quinazolin-6-yl)-pyrrolidin-1-yl]-1-{4-[4-(5-fluoro-pyrimidin-2-yl)-phenyl]piperazin-1-yl}-ethanone (40 mg, 0.05 mmol) was dissolved in 3.2 ml dioxane & 0.8 ml water. Pyridine-4-boronic acid (8.6 mg:0.07 mmol), Pd(dppf)Cl2 (8.2 mg, 0.01 mmol) and K3PO4 (27 mg, 0.13 mmol) were added and the reaction mixture flushed with nitrogen. The reaction mixture was stirred at 90° C. for 4 hrs. The crude was cooled, diluted with dichloromethane and the aqueous layer extracted with dichloromethane 2× and ethylacetate 1×. The combined organic layers were dried over Na2SO4, filtered and concentrated under vacuum. The crude was purified by prep. plate chromatography using 5% methanol (2N NH3)/dichloromethane to obtain 16 mg of final product.

### Step 3: 1-{4-[4-(5-Fluoro-pyrimidin-2-yl)-phenyl]-piperazin-1-yl}-2-[3-(3-pyridin-4-yl-1H-pyrazolo[3,4-g]quinazolin-6-yl)-pyrrolidin-1-yl]-ethanone (26)

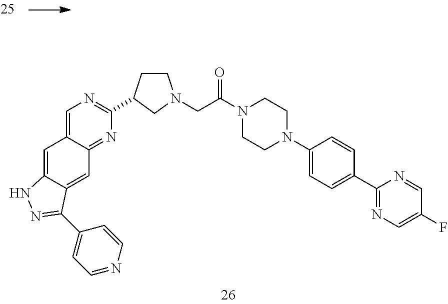

1-{4-[4-(5-Fluoro-pyrimidin-2-yl)-phenyl]-piperazin-1-yl}-2-[3-(3-pyridin-4-yl-1-trityl-1H-pyrazolo[3,4-g]quinazolin-6-yl)-pyrrolidin-1-yl]-ethanone (16 mg, 0.019 mmol) was dissolved in 5 ml of dichloromethane and 1 ml of trifluoroacetic acid added. After stirring for 18 hrs, the crude was evaporated and evaporated from dichloromethane 2×. The crude was dissolved in 75% dichloromethane/methanol and quenched with 2N NH₃ in methanol. The crude was then concentrated to dryness and chromatographed on a silica flash column to obtain 5.5 mg of title compound. MS (615.3, M+1 observed, Retention Time: 2.28 minutes).

Following essentially the same procedures as described above the compounds in Table 1 were prepared.

TABLE 1

| | LCMS | |
|---|---|---|
| | Retention | |
| | time | M + 1 |
| Compound | (Minutes) | Observed |
| 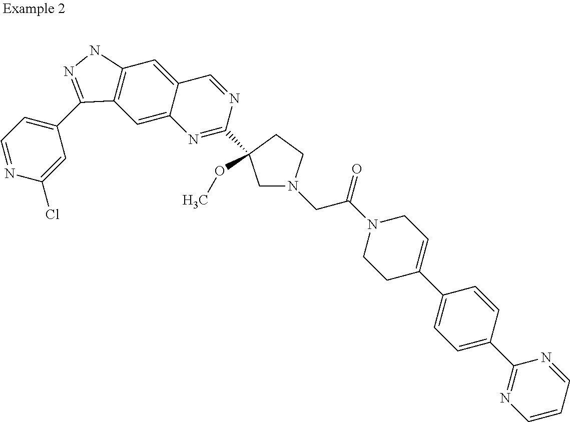 | 2.73 | 658.4 |
| 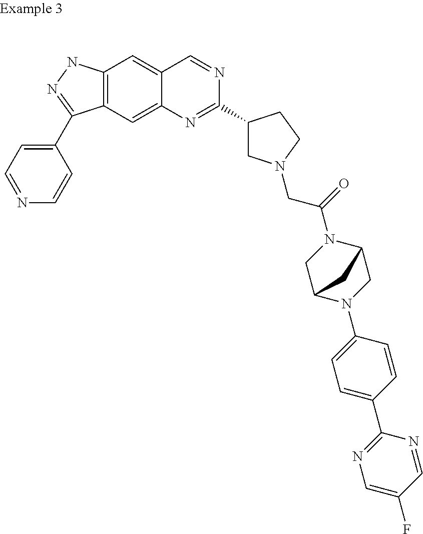 | 2.05 | 627.3 |
| 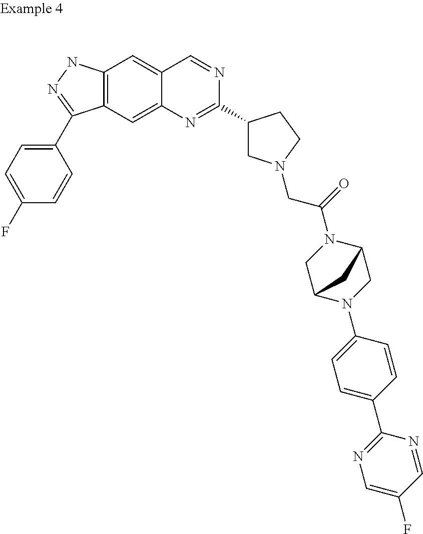 | 2.96 | 629.3 |
| 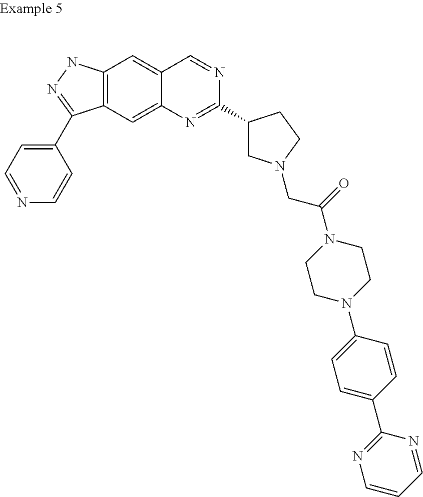 | 1.83 | 597.3 |
| 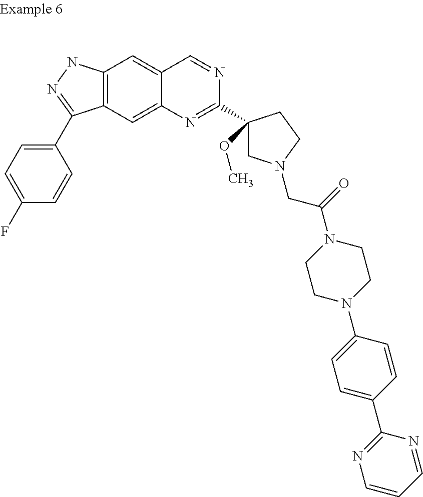 | 2.72 | 644.4 |
| 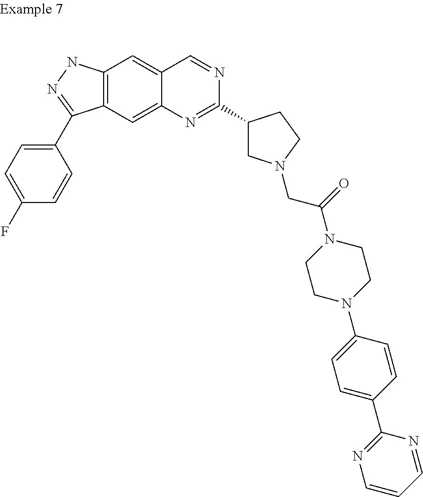 | 3.15 | 614.3 |
| 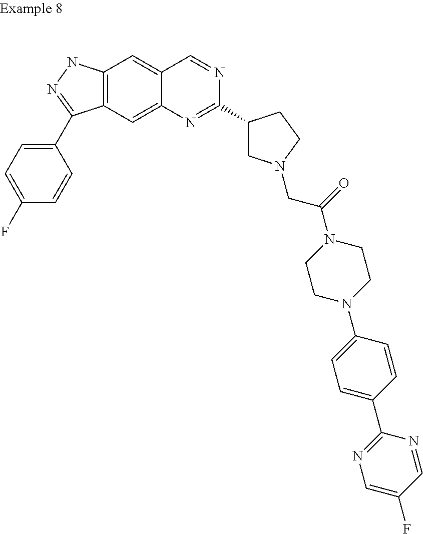 | 3.26 | 632.3 |
| 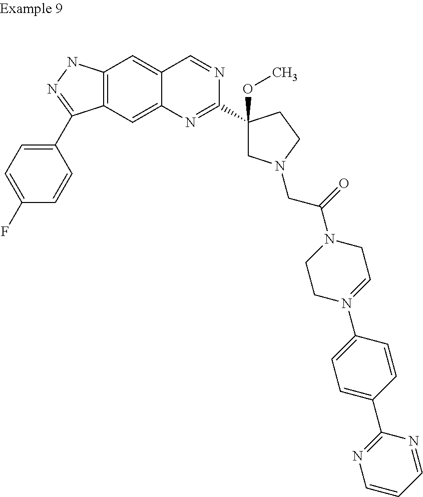 | 3.33 | 641.4 |
| 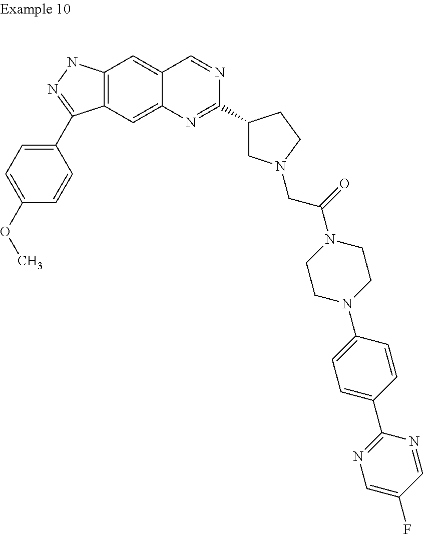 | 2.92 | 644.4 |
| 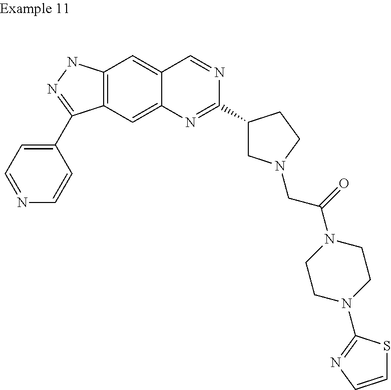 | 1.39 | 526.3 |
| 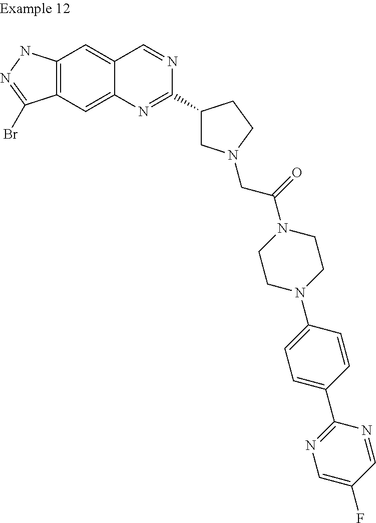 | 2.69 | 616.3 |

Following the above procedures and those given below, additional compounds of the invention can be made.

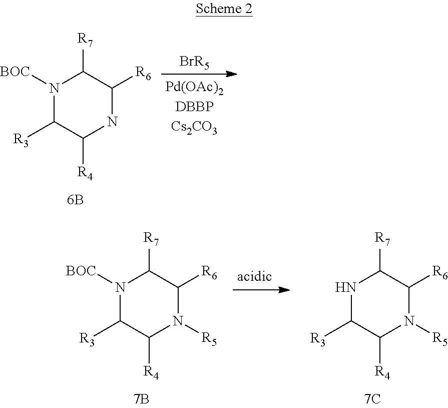

The R⁵ substituted piperazine is prepared by Buchwald type coupling of the piperazine 6B with an aryl bromide in the presence of palladium to obtain the piperazine 7B. The BOC group is removed using acidic conditions (e.g., TFA) to give piperazine 7C.

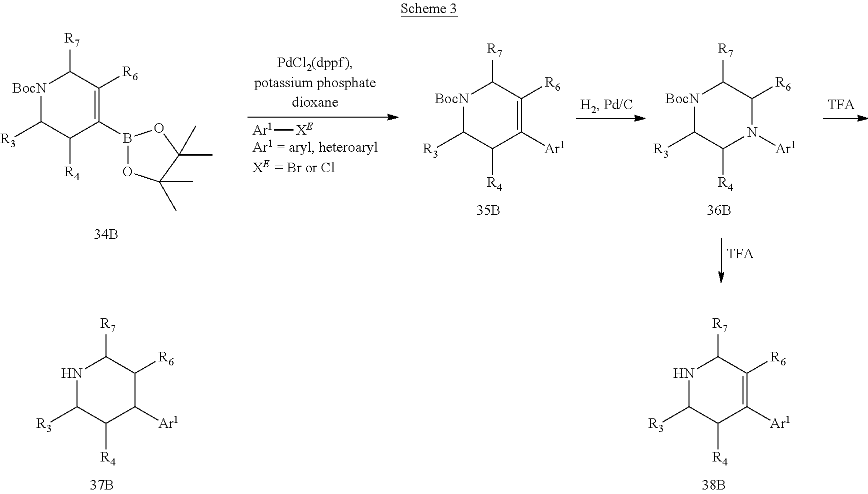

Aryl or heteroaryl substituted piperidines can be prepared by Suzuki coupling of an aryl or heteroaryl halide with the pinicolboronate 34B to obtain 35B. The ring double bond can then be hydrogenated to obtain 36B followed by removal of the Boc protecting group under trifluoroacetic acid conditions. Alternatively the double bond can be retained and the Boc group removed to give 38B.

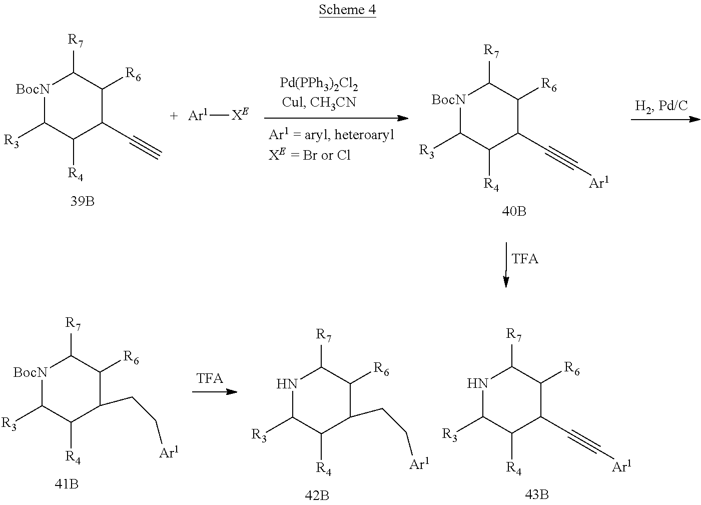

Similarly aryl or heteroaryl substituted piperizines with a 2 carbon spacer can be prepared as shown in Scheme 4 by coupling an aryl or heteroaryl halide with an acetylene derivative 39B that can be prepared according to procedures known in the art to obtain 40B. 40B can then be reduced to 41B followed by removal of the Boc protecting group under trifluoroacetic acid conditions. Alternatively the Boc protecting group from 40B can be removed under trifluoroacetic acid conditions to give 43B.

### Preparation 6

### Step 1: Preparation of 2-(6-Bromo-pyridin-3-yl)-pyrimidine

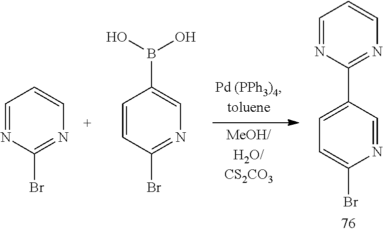

A mixture of 2-bromopyrimidine (0.43 g, 2.70 mmol), 2-bromopyridine-5-boronic acid (0.55 g, 2.72 mmol), tetrakis(triphenylphosphine)palladium(0) (300 mg, 0.259 mmol), cesium carbonate (1.15 g, 3.03 mmol) was stirred in MeOH/toluene/water (15 ml, 1/1/1) at reflux temperature overnight. The reaction was cooled to room temperature and diluted with EtOAc (200 ml) and water (50 ml). The organic layer was separated, dried over MgSO₄, filtered and solvent evaporated yielding a residue which was purified on silica gel eluting with 25% v/vEtOAc/hexanes yielding product 76 as white solid. (0.55 g, 85%) ESMS (MH, 236).

### Step 2: Preparation of 2-(6-piperazin-1-yl-pyridin-3-yl)-pyrimidine

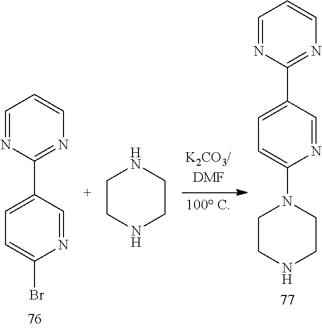

A mixture of 2-(6-Bromo-pyridin-3-yl)-pyrimidine 76 (100 mg, 0.425 mmol), potassium carbonate (100 mg, 0.724 mmol), and piperazine (100 mg, 1.16 mmol) in DME (5 ml) were stirred at 100° C. for 1 hour. The reaction was cooled, solvent evaporated under reduced pressure, and the residue dissolved in MeCl₂ (150 ml), washed with H₂O (50 ml), dried over MgSO₄, filtered and evaporated solvent yielding title product 77 as a white solid (100 mg, 98%). ESMS (MH, 242).

### Preparation 7

### Step 1: Preparation of 5-Methyl-2-[4-(3-(S)-methyl-piperazin-1-yl)-phenyl]-pyrimidine

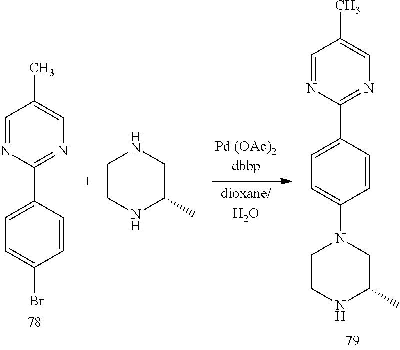

A mixture of 2-(4-bromophenyl)-5-methylpyrimidine 78 (250 mg, 1.008 mmol), palladium acetate (50 mg), cesium carbonate (400 mg, 1.23 mmol), (S)-2-methyl piperazine (200 mg, 2 mmol) and 2-Di-t-butylphosphino)-biphenyl (50 mg, 0.167 mmol) was stirred in dioxane:water (10 ml, v/v 5:1) at reflux temperature for 4 hours. The reaction was cooled, diluted with MeCl₂ (100 ml) and H₂O (50 ml). The organic layer was separated, dried (MgSO₄), filtered and solvent evaporated. The residue was purified by chromatography eluting with 100% EtOAc then with 10% v/v MeOH/EtOAc/NH₄OH yielding product 79 as a white solid. (220 mg.81%) ESMS (MH, 269).

### Preparation 8

### Step 1

### Preparation of 5-(4-Bromo-phenyl)-pyrimidin-2-ylamine

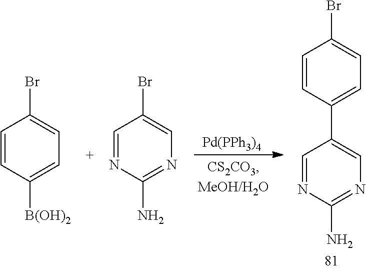

A mixture of 5-bromo-pyrimidin-2-ylamine (0.8 g, 4.59 mmol), 4-bromophenyl boronic acid (1 g, 4.97 mmol), tetrakis(triphenylphosphine)palladium(0) (300 mg, 0.259 mmol), cesium carbonate (1.15 g, 3.03 mmol) was stirred in MeOH/H₂O (20 ml, 1/1) at reflux temperature overnight. The reaction was cooled to room temperature and diluted with EtOAc (200 ml) and water (50 ml). The organic layer was separated, dried over MgSO₄, filtered and solvent evaporated yielding a residue which was purified on silica gel eluting with 85% v/vEtOAc/hexanes yielding product 81 as white solid. (0.7 g, 63%). ESMS (MH, 250).

### Step 2: Preparation of 5-(4-piperazin-1-yl-phenyl)-pyrimidin-2-ylamine

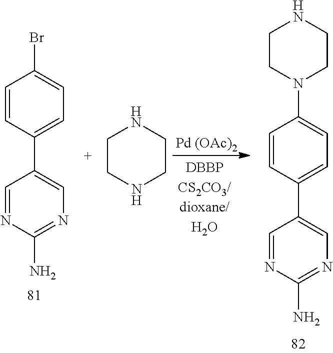

A mixture of 5-(4-bromo-phenyl)-pyrimidin-2-ylamine (100 mg, 0.401 mmol), palladium acetate (20 mg, 0.089 mmol), cesium carbonate (200 mg, 0.62 mmol), piperazine (100 mg, 1.16 mmol) and 2-di-t-butylphosphino)-biphenyl (50 mg, 0.167 mmol) was stirred in dioxane:water (10 ml, v/v 5:1) at reflux temperature for 4 hours. The reaction was cooled, diluted with MeCl₂ (100 ml) and H₂O (50 ml). The organic layer was separated, dried (MgSO₄), filtered and solvent evaporated. The residue was purified by chromatography eluting with 100% EtOAc then with 10% v/v MeOH/EtOAc/NH₄OH yielding product 82 as a white solid. (70 mg.68%) ESMS (MH, 256).

### Preparation 9

### Step 1: Preparation of (S,S)-5-[4-(4,4,5,5-Tetramethyl-[1,3,2]dioxaborolan-2-yl)-phenyl]-2,5-diaza-bicyclo[2.2.1]heptane-2-carboxylic acid tert-butyl ester

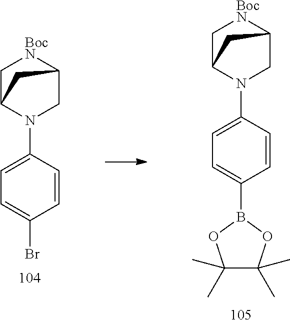

A mixture of (S,S)-5-(4-Bromo-phenyl)-2,5-diaza-bicyclo[2.2.1]heptane-2-carboxylic acid tert-butyl ester (4.0 g, 11.3 mmol), Bis(pinacolato)diboron (4.0 g, 15.7 mmol), KOAc (3.2 g) and Cl₂Pd(dppf)CH₂Cl₂ (800 mg) in 40 mL dioxane was evacuated and recharged with N₂ several times. The reaction mixture was then heated to 85° C. overnight. After cooling down to rt, 150 mL ethyl acetate and 30 mL water was added. The mixture was filtered through a pad of Celite and washed with additional ethyl acetate. The separated organic layer was dried (MgSO4) and concentrated. The crude was purified on silica gel column eluting with 30% to 50% ethyl acetate/hexanes to yield the title compound as a white solid (3.3 g). MS (401, MH)

### Step 2: Preparation of (S,S)-5-[4-(5-Fluoro-pyrimidin-2-yl)-phenyl]-2,5-diaza-bicyclo[2.2.1]-heptane-2-carboxylic acid tert-butyl ester

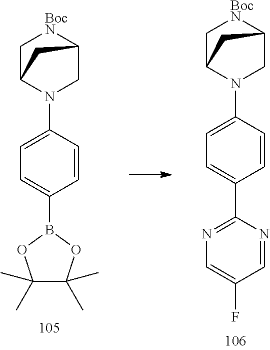

A mixed DMF/H2O (5 mL/5 mL) solution of (S,S)-5-[4-(4,4,5,5-Tetramethyl-[1,3,2]dioxaborolan-2-yl)-phenyl]-2,5-diaza-bicyclo[2.2.1] heptane-2-carboxylic acid tert-butyl ester (800 mg, 2 mmol), 2-chloro-5-fluoro-pyrimidine (340 mg, 2.6 mmol), K₂CO₃ (552 mg, 4 mmol) and Cl₂Pd(dppOCH₂Cl₂ (160 mg) was evacuated and recharged with N₂ several times. The reaction was heated at 70° C. over 18 hrs. After cooling down to rt, 40 mL ethyl acetate and 10 mL water was added. The mixture was filtered through a pad of Celite and washed with additional ethyl acetate. The separated organic layer was dried (MgSO₄) and concentrated. The crude was purified on silica gel column eluting with 50% ethyl acetate/hexanes to yield the title compound (420 mg) as a light yellow solid.

In a similar manner, 106a:

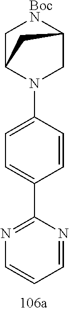

was prepared by substituting 2-chloropyrimidine for 2-chloro-5-fluoro-pyrimidine.

### Preparation 10

### Preparation of (S,S)-5-(5-Vinyl-pyrimidin-2-yl)-2,5-diaza-bicyclo[2.2.1]heptane-2-carboxylic acid tert-butyl ester

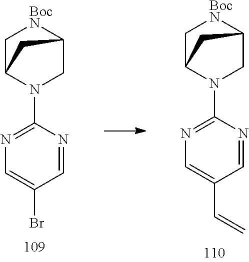

(S,S)-5-(5-Bromo-pyrimidin-2-yl)-2,5-diaza-bicyclo[2.2.1]heptane-2-carboxylic acid tert-butyl ester (177 mg, 0.5 mmol), tributyl vinyl tin (634 mg, 2 mmol) and Cl₂Pd(dppf)CH₂Cl₂ (60 mg) was mixed in DMF (3 mL). The mixture was heated at 90° C. over 3 days. The cooled down reaction was participate between ethyl acetate (50 mL) and H₂O (10 mL). The organic layer was washed with H₂O (10 mL), brine (10 mL), dried (MgSO₄) and filtered. The conc. filtrate was purified on silica gel column eluting with 33% to 50% ethyl acetate/hexanes to yield the title compound as a white solid (54 mg). MS (303, MH).

### Preparation 11

### Preparation of 4-Hydroxy-4-thiazol-2-yl-piperidine-1-carboxylic acid tert-butyl ester

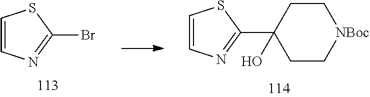

2-Bromo-thiazole (0.27 mL, 2.99 mmol) was dissolved in Et₂O (8 mL) and cooled down to −78° C. BuLi (1.3 mL, 2.5 M) was added dropwise. The resulting yellow solution was stirred at −78° C. for 45 min. 4-Oxo-piperidine-1-carboxylic acid tert-butyl ester (720 mg, 3.61 mmol) in Et₂O (5 mL) was then added dropwise. The reaction temperature rose to rt naturally overnight. H₂O (10 mL) was added to quench the reaction and extracted with ethyl acetate. The combined organic layer was dried (MgSO₄), filtered and concentrated. The residue was purified on silica gel column eluting with 33% to 50% ethyl acetate/hexanes to give 4-Hydroxy-4-thiazol-2-yl-piperidine-1-carboxylic acid tert-butyl ester (800 mg) as a colorless oil.

### Preparation 12

### Step 1: Preparation of 4-Thiazol-2-yl-piperazine-1-carboxylic acid tert-butyl ester

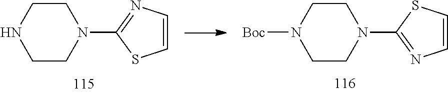

To a solution of 1-thiazol-2-yl-piperazine (2 g, 12 mmol), triethylamine (2.4 g, 24 mmol) and DMAP (150 mg, 1.2 mmol) in acetonitrile (15 ml) was added di-tert-butyl dicarbonate. The resulted reaction mixture was stirred at RT for 3 hours. Then water (20 mL) was added and the formed slurry was stirred for 30 min. The formed product was collected by filtration and washed with water. After dry in air, 2.8 g product was obtained (90% yield)

### Step 2: Preparation of 4-(5-Bromo-thiazol-2-yl)-piperazine-1-carboxylic acid tert-butyl ester

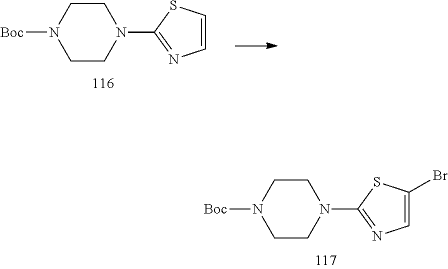

To a mixture containing 4-thiazol-2-yl-piperazine-1-carboxylic acid tert-butyl ester (0.5 g, 1.9 mmol) and cesium carbonate (0.62 mmol) in chloroform (5 mL) at 0° C., bromine (110 mL) was added through a syringe. After the addition, the reaction mixture was stirred at room temperature for 1 hour. Water was added and the organic layer was collected and dried over sodium sulfate. After removal of solvent, 0.6 g of product was obtained (95% yield).

### Preparation 13

### Preparation of 5-Thiazol-2-yl-2,5-diaza-bicyclo[2.2.1]heptane-2-carboxylic acid tert-butyl ester

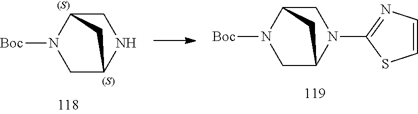

A mixture of 2-bromothiazole (200 mg, 1.22 mmol), palladium acetate (15 mg, 0.06 mmol), sodium tert-butoxide (217 mg, 2.26 mmol), (S,S) 2,5-diaza-bicyclo[2.2.1]heptane-2-carboxylic acid tert-butyl ester (280 mg, 1.4 mmol) and 2-Di-t-butylphosphino)-biphenyl (37 mg, 0.118 mmol) was stirred in dioxane (10 ml) at 80° C. for overnight. The reaction was cooled, diluted with ethyl acetate (40 ml) and H₂O (50 ml). The organic layer was separated, dried (Na₂SO₄), filtered and solvent evaporated. The residue was purified by chromatography eluting with 5% MeOH/DCM yielding product as a white solid. (180 mg, 52% yield)

### Preparation 14

### Step 1: Preparation of 4-(5-Pyrimidin-2-yl-thiazol-2-yl)-piperazine-1-carboxylic acid tert-butyl ester

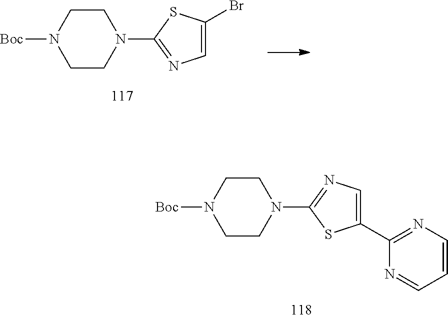

A round bottom flask containing 4-(5-bromo-thiazol-2-yl)-piperazine-1-carboxylic acid tert-butyl ester (100 mg, 0.29 mmol), 2-tributylstannanyl-pyrimidine (130 mg, 0.36 mmol), cesium fluoride (85 mg, 0.56 mmol) and palladium di-tert-butylphosphine was degassed three times with Ar. Dioxane was added and the formed reaction mixture was stirred at 90° C. overnight under Ar. Then the reaction mixture was filter through celite and the solvent was removed under vacuum and crude product was used directly in the next step.

### Step 2: Preparation of 2-(2-piperazin-1-yl-thiazol-5-yl)-pyrimidine

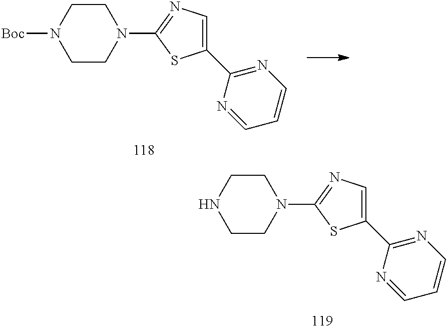

To the crude product obtained in the previous step, was added 90% TFA (1 mL) and the reaction mixture was stirred at ambient temperature for 1 hour. The excess TFA was removed under vacuum and the residue was purified using prep-HPLC to give desired product (45 mg, 44% yield for two steps) as TFA salt.

### Preparation 15

### Step 1: Preparation of 4-(4-Bromo-phenyl)-piperidine-1-carboxylic acid tert-butyl, ester

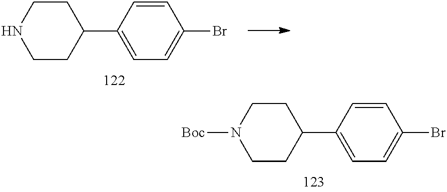

To a solution of 4-(4-bromo-phenyl)-piperidine (2.8 g, 12 mmol), triethylamine (2.4 g, 24 mmol) and DMAP (150 mg, 1.2 mmol) in acetonitrile (15 ml) was added di-tert-butyl dicarbonate. The resulted reaction mixture was stirred at RT for 3 hours. Then water (20 mL) was added and the formed slurry was stirred for 30 min. The formed product was collected by filtration and washed with water. After dry in air, 3.8 g product was obtained (95% yield).

### Step 2: Preparation of 4-(4-Pyrimidin-2-yl-phenyl)-piperidine-1-carboxylic acid tert-butyl ester

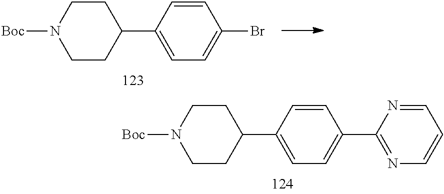

A mixture containing 4-(4-bromo-phenyl)-piperidine-1-carboxylic acid tert-butyl ester (100 mg, 0.29 mmol), 2-tributylstannanyl-pyrimidine (130 mg, 0.36 mmol), cesium fluoride (85 mg, 0.56 mmol) and palladium di-tert-butylphosphine was degassed three times with Ar. Dioxane was added and the formed reaction mixture was stirred at 90° C. overnight under Ar. Then the reaction mixture was filter through celite and the solvent was removed under vacuum and crude product was used directly in the next step.

### Step 3: Preparation of 2-(4-Piperidin-4-yl-phenyl)-pyrimidine

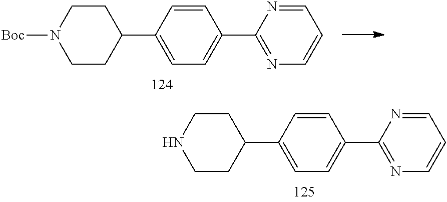

To the crude product obtained in the previous step, was added 90% TFA (1 mL) and the reaction mixture was stirred at ambient temperature for 1 hour. The excess TFA was removed under vacuum and the residue was purified using prep-HPLC to give desired product (38 mg, 37% yield for two steps) as TFA salt.

### Preparation 16

### Step 1: Preparation of 4-Thiazol-2-yl-3,6-dihydro-2H-pyridine-1-carboxylic acid tert-butyl ester

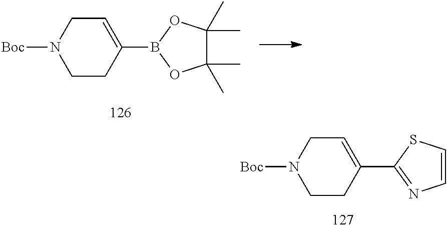

To a mixture of (N-tert-butoxycarbonyl)-1,2,3,6-tetrahydropryidine-2-boronic acid pinacol ester (100 mg, 0.32 mmol), 2-bromothiazole (64 mg, 0.39 mmol), PdCl₂(dppf) (24 mg, 0.03 mmol) and potassium phosphate (213 mg, 1 mmol) was degassed three times with Ar, was added dixoane. The formed reaction mixture was then heated at 80° C. overnight under Ar. After the reaction was complete, the mixture was filter through celite and was chromatographed on a silica column (10% ethyl acetate/DCM) to obtain desired product (30 mg, 35% yield).

### Step 2

### Preparation of 4-Thiazol-2-yl-1,2,3,6-tetrahydro-pyridine

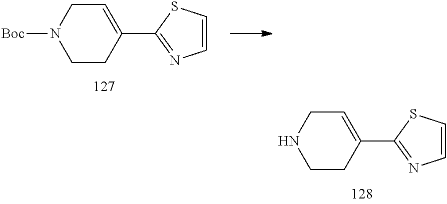

To the product obtained in the previous step, was added 90% TFA (1 mL) and the reaction mixture was stirred at ambient temperature for 1 hour. The excess TFA was removed under vacuum and the residue was purified using prep-HPLC to give desired product (15 mg, 50° A) yield) as TFA salt.

### Preparation 17

### Step 1: Preparation of 4-(methoxy-methyl-carbamoyl)-piperidine-1-carboxylic acid tert-butyl ester

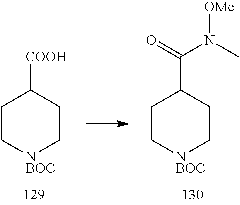

N,O-dimethylhydroxylamine hydrochloride (851 mg, 8.72 mmols) was suspended in dichloromethane (6 ml) and cooled to 0 C. N,N′-diisopropylethylamine (1.66 ml, 9.53 mmols) was added and the mixture was stirred at 0 C until a clear solution was obtained. The resulting solution was kept at 0 C for further use. Boc-isonipecotic acid (2 g, 8.72 mmol), 1-hydroxybenzotriazole (1.2 g, 8.88 mmols) and 1-(3-dimethylaminopropyl)-3-ethylcarbodiimide hydrochloride (EDCI) (1.83 g, 9.58 mmols) were dissolved in DMF (15 ml) and cooled to 0 C. The solution of N,O-dimethylhydroxylamine in dichloromethane was added with stirring, and the resulting reaction mixture was allowed to stir overnight at room temperature. DMF was removed under reduced pressure and residue was partitioned between ethyl acetate and 10% citric acid. Organic layer was isolated, washed with water, saturated NaHCO₃, water and brine and dried over MgSO₄. Solvent was removed under reduced pressure and the residue was purified on silica gel eluting with ethyl acetate in hexanes (2:1) to provide the title compound (1.88 g, 79%). LCMS m/e (295, M+Na).

### Step 2: Preparation of 4-formyl-piperidine-1-carboxylic acid tert-butyl ester

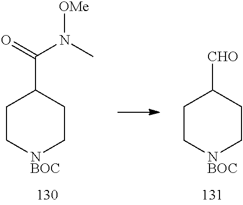

To a mixture of lithium aluminum hydride (1 M THF solution, 4.4 ml) in ether (4 ml) was added dropwise at −60 C 4-(methoxy-methyl-carbamoyl)-piperidine-1-carboxylic acid tent-butyl ester (1 g, 3.67 mmols) in ether (6 ml). The reaction mixture was allowed to warm to 0-5 C and then re-cooled to −60 C. Celite was added and reaction was quenched with a solution of KHSO₄ (1 g) in water (3 ml), filtered through Celite. The filtrate was washed with cold 1N HCl, saturated NaHCO₃, brine and dried (MgSO₄) and concentrated. The residue was purified by column chromatography on silica geleluting with ethyl acetate in hexanes (1:1) to provide title compound (656 mg, 84%). (Org. Prep. Proced. Int., 2000, 32, 96.)

### Preparation 18

### Step 1: Preparation of 4-methyl-benzenesulfonyl azide

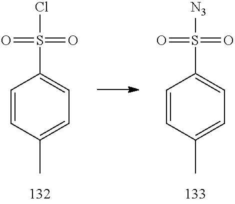

To a solution of tosyl chloride (4 g, 21 mmols) in acetone (60 ml) was added at 0-5 C a solution of sodium azide (1.37 g, 21 mmols) and the resulting solution was stirred at that temperature for 2 hours. Acetone was removed and the aqueous mixture was extracted with ether three times. The combined extracts were dried over MgSO₄. Evaporation of solvents provided tosyl azide (4 g, 97%). (Eur. J. Org. Chem. 2003, 821-832.)

### Step 2: Preparation of (1-diazo-2-oxo-propyl)-phosphonic acid dimethyl ester

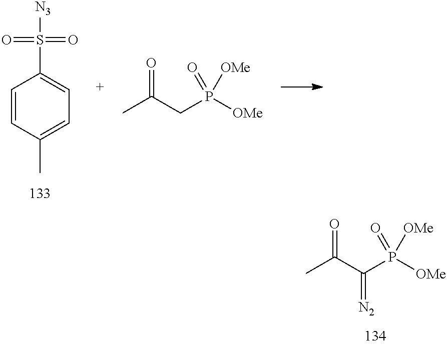

To a suspension of NaH (60% in mineral oil, 0.83 g, 20.8 mmols) in THF (50 ml) was added dropwise at 0 C (2-oxo-propyl)-phosphonic acid dimethyl ester (3.1 g, 18.7 mmols) in THF (50 ml), and the solution was stirred at 0 C for one hour. Tosyl azide (4 g, 20 mmols) was added in one portion, stirred at 0 C for 10 minutes, filtered through Celite and concentrated. The residue was purified by column chromatography on silica gel using ethyl acetate to yield the title compound (2.9 g, 81%) as oil. (Eur. J. Org. Chem. 2003, 821-832.)

### Step 3: Preparation of 4-ethynyl-piperidine-1-carboxylic acid tert-butyl ester

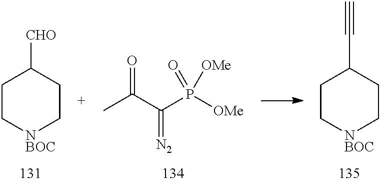

At 0 C, to a stirred mixture of 4-formyl-piperidine-1-carboxylic acid tert-butyl ester (358 mg, 1.68 mmols) and potassium carbonate (464 mg, 3.36 mmols) in methanol (16 ml) was added dropwise a solution of (1-diazo-2-oxo-propyl)-phosphonic acid dimethyl ester (323 mg, 1.68 mmols) in methanol (2 ml). The resulting mixture was stirred at room temperature overnight, filtered and concentrated. The residue was chromatographed on silica gel using a solution of ethyl acetate in hexanes (1:5) to provide the title compound (308 mg, 88%) as colorless crystals. LCMS m/e (154, M-t-Bu+2H). (J. Am. Chem. Soc. 2003, 125, 3714.)

### Step 4: Preparation of 4-phenylethynyl-piperidine-1-carboxylic acid tert-butyl ester

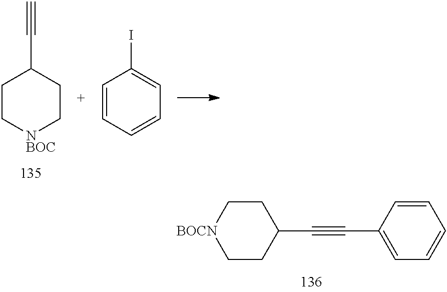

Iodobenzene (135 μl, 1.2 mmols), 4-ethynyl-piperidine-1-carboxylic acid tert-butyl ester (209 mg, 1 mmols) and triethylamine (167 μl, 1.2 mmols) were dissolved in acetonitrile (6 ml). Dichlorobis(triphenylphosphine)palladium(II) (35 mg, 0.05 mmols) and CuI (10 mg, 0.05 mmols) were added, and reaction mixture was stirred at room temperature overnight and continued to stir at 50 C for two more hours before partitioning between ethyl acetate and water. Organic layer was isolated, washed with 1 N HCl, brine and dried (MgSO₄). Solvents were removed and residue was purified by column chromatography on silica gel using solutions of ethyl acetate in hexanes (1:4; 1:2) to yield the title compound (74 mg). LCMS m/e (230, M-t-Bu+2H)

### Step 5: Preparation of 4-phenylethynyl-piperidine

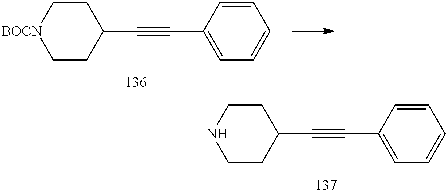

4-Phenylethynyl-piperidine-1-carboxylic acid tert-butyl ester was treated with TFA for 10 minutes and concentrated, lyophilized to provide the title product.

### Preparation 19

### Step 1: Preparation of 4-pyrimidin-2-ylethynyl-piperidine-1-carboxylic acid tert-butyl ester

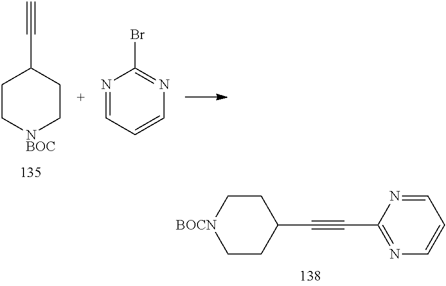

To a suspension of 2-bromopyrimidine (175 mg, 1.1 mmols), dichlorobis(triphenylphosphine)palladium(II) (35 mg, 0.05 mmols) and CuI (10 mg, 0.05 mmols) was added a solution of 4-ethynyl-piperidine-1-carboxylic acid tert-butyl ester (209 mg, 1 mmol). The mixture was stirred overnight, filtered through Celite, concentrated. The residue was partitioned between ethyl acetate and water, organic layer was isolated, dried (MgSO₄), and concentrated. The residue was chromatographed on silica gel eluting with ethyl acetate in hexanes (1:1) to give un-reacted 2-bromopyrimidine (130 mg), then the title compound (23 mg). LCMS m/e (288, M+H).

### Step 2: Preparation of 2-piperidin-4-ylethynyl-pyrimidine

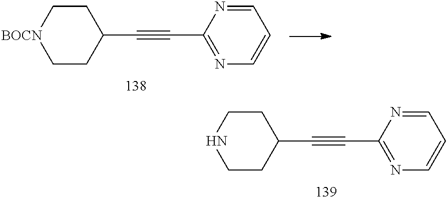

4-Pyrimidin-2-ylethynyl-piperidine-1-carboxylic acid tert-butyl ester was treated with TFA for 10 minutes and concentrated, lyophilized to provide the title product.

### Preparation 20

### Step 1: Preparation of 3-methyl-1-thiazol-2-yl-piperazine

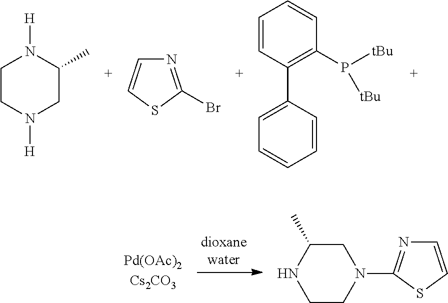

A mixture of 2(R)-methyl piperazine (300 mg, 3 mmol), 2-bromo thiazole (0.27 ml, 3 mmol), (2-biphenylyldi-tert-butylphosphine (134 mg, 0.449 mmol), palladium acetate (101 mg, 0.45 mml), and cesium carbonate (1.46 g, 4.49 mmol) in dioxane 25 ml (v/v 5/1) was kept at reflux temperature for 2 hours, then cooled to room temperature, then filtered through celite, then concentrated and then purified by chromatography eluting with 12% MeOH/MeCl₂/NH₄OH to yield the product as a white solid (145 mg, 26%).

### Preparation 21

### Preparation of 2-Chloro-1-[4-(4-pyrimidin-2-yl-phenyl)-3,6-dihydro-2H-pyridin-1-yl]-ethanone

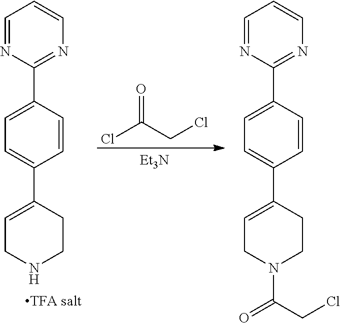

2-[4-(1,2,3,6-Tetrahydro-pyridin-4-yl)-phenyl]-pyrimidine trifluoroacetate (2.3 g, 9.7 mmol) was dissolved in 75 ml of dichloromethane and 4.1 ml of triethylamine added 0 C. Chloroacetylchloride (0.92 ml, 11.7 mmol) was added and the reaction mixture stirred for 30 min. The reaction mixture was washed with a solution of saturated sodium bicarbonate (80 ml), the organic layer separated, dried over magnesium sulfate and evaporated to obtain 2.41 g of crystalline product.

### Preparation 22

### Preparation of 4-fluoro-4-thiazol-2-yl-piperidine-1-carboxylic acid tert-butyl ester

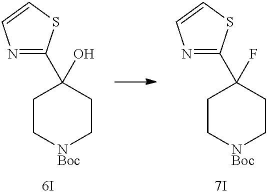

4-Hydroxy-4-thiazol-2-yl-piperidine-1-carboxylic acid tert-butyl ester 61 (500 mg, 1.76 mmol) was dissolved in CH₂Cl₂ (20 mL) and cooled to 0° C. DAST (0.46 mL, 3.52 mmol) was then added. The mixture was stirred at 0° C. for 1 hr and then quenched with sat. NaHCO₃. The separated organic layer was dried and concentrated in vacuo. The crude was purified with silica gel column (eluting with 12.5% ethyl acetate in hexanes) to yield an off-white solid (443 mg) as the title compound.

### Preparation 22A

### Step 1

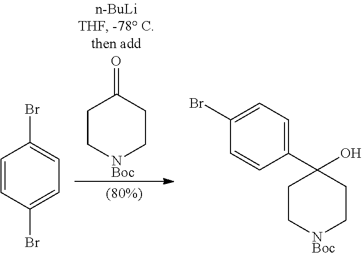

To a solution of 1,4-dibromobenzene (1.0 g, 4.24 mmol) in tetrahydrofuran (10 ml) at −78° C. under nitrogen, a solution of n-butyl lithium (1.7 ml, 4.24 mmol, 1.6M in hexane) was added slowly. The mixture was allowed to warm from −78° C. to −20° C. in 1 hr. A solution of piperidone (703 mg, 3.53 mmol) in tetrahydrofuran (5 ml) was added at −78° C. and the mixture was stirred at the same temperature for 1 hr. Saturated ammonium chloride solution was added and the mixture was allowed to warm to r.t. Water and ethyl acetate were added and layers were separated. The aqueous layer was extracted with ethyl acetate (×2). The combined organic layers were dried (MgSO₄) and filtered. Solvents were removed in vacuum and column chromatography [ethyl acetate-hexane, 5:1 (v/v)] gave 4-(4-bromophenyl)-4-hydroxypiperidine-1-carboxylic acid tert-butyl ester (1.0 g, 80%) as colorless oil.

### Step 2

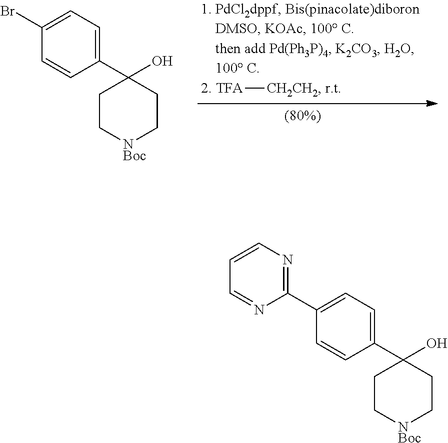

4-(4-bromophenyl)-4-hydroxypiperidine-1-carboxylic acid tert-butyl ester (800 mg, 2.25 mmol), bis(pinacolate)diboron (856 mg, 3.37 mmol), PdCl₂(dppf).CH₂Cl₂ (184 mg, 0.23 mmol), potassium acetate (660 mg, 6.74 mmol) were weighted into a sealed-tube. Methyl sulfoxide (20 ml) was added and the mixture was purged with nitrogen for 20 min before it was heated at 100° C. for 2 hr under nitrogen. The mixture was cooled to r.t. Potassium carbonate (1.55, 11.2 mmol), 2-bromopyrimidine (429 mg, 2.70 mmol) and water (10 ml) were added. The mixture was again purged with nitrogen for 20 min. Palladium tetrakistriphenylphosphine (260 mg, 0.23 mmol) was added and the final mixture was stirred at 100° C. for a further 2 hr. After being cooled to r.t., ethyl acetate and water were added. The mixture was filtered through a pad of Celite. Layers were separated and the organic layer was washed with water (×2). The combined aqueous layers were extracted with ethyl acetate (×1). The combined organic layers were dried (MgSO₄) and filtered. Solvents were removed in vacuum and column chromatography [ethyl acetate-hexane, 1:1 (v/v)] gave 4-hydroxy-4-(4-pyrimidin-2-ylphenyl)-piperidine-1-carboxylic acid tert-butyl ester (639 mg, 80%) as colorless oil.

### Preparation 23

### Preparation of 4-methoxy-4-(4-pyrimidin-2-yl-phenyl)-piperidine-1-carboxylic acid tert-butyl ester 9I

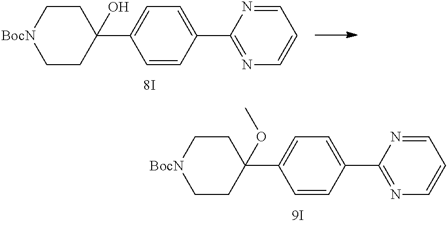

4-Hydroxy-4-(4-pyrimidin-2-yl-phenyl)-piperidine-1-carboxylic acid tert-butyl ester 81 (138 mg, 0.39 mmol) was dissolved in DMF (2 mL) and cooled to 0° C. MeI (0.1 mL) was added followed by the addition of NaH (26 mg, 60% suspension in mineral oil). After 30 min at 0° C., the reaction was quenched with sat. NH₄Cl and extracted with ethyl acetate. The combined organic layers was washed with brine, dried and concentrated in vacuo. The residue was purified with prep TLC plates (developing with 50% ethyl acetate/hexanes) to yield a colorless film (80 mg) as the title compound.

### Preparation 24

### Preparation of 4-bromo-2,6-dimethyl-pyridine (11I)

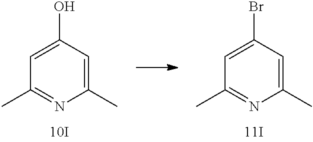

2,6-Dimethyl-pyridin-4-ol 10I (6.16 g, 50 mmol), PBr₅ (11.9 g, 27.65 mmol) and POBr₃ (2.5 mL, 24.6 mmol) was combined and CHCl₃ (2.5 mL) was added. The reaction was heated at 100° C. for 5 hrs and then cooled in an ice bath. Solid KOH was added till PH reached 7-8 followed by extraction with Et₂O (3×75 mL). The combined ether layer was dried and evaporated in vacuo to give a thick clear crude oil (10.1 g) as the title compound.

### Preparation 25

### Preparation of 2,6-dimethyl-4-pyridine boronic acid (12I)

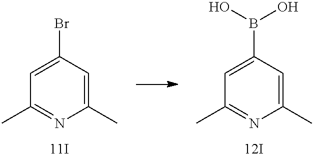

4-Bromo-2,6-dimethyl-pyridine (910 mg, 4.9 mmol) and triisopropyl borate (2.3 mL, 10 mmol) in THF (10 mL) were cooled in a −78° C. bath. BuLi (2.7 M, 7 mL) was added in drop wise. After 3 hrs, the bath was removed. The reaction was acidified with 1N HCl till pH=1. The separated aqueous layer was neutralized with NaOH and subsequently extracted with ethyl acetate. A crude white solid was obtained (800 mg) as the title compound.

### Preparation 26

### Preparation of 2-trifluoromethyl-4-pyridine boronic acid (14I)

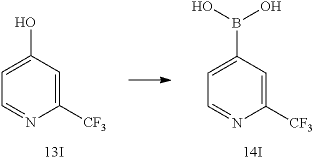

The title compound was prepared from 2-trifluoromethyl-pyridin-4-ol (13I) by a procedure essentially similar to that described in Chem. Het. Cpds, 1997, p. 995, the disclosure of which is incorporated herein by reference thereto.

### Preparation 27

### Step 1: Synthesis of 2-morpholin-4-ylmethyl-acrylic acid methyl ester

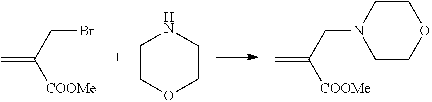

To a mixture of methyl 2-(bromomethyl)acrylate (119 μl, 1 mmol) and K₂CO₃ (138 mg, 1 eq) in acetonitrile (2 ml) was added morpholine (96 μl, 1.1 mmols). The mixture was stirred overnight, filtered and concentrated. The residue was partitioned between ether and water, and organic layer was isolated, washed with brine and dried (MgSO₄). Solvent was removed and residue was purified by column chromatography. Ethyl acetate eluted out the title compound as clear oil (110 mg, 59%).

### Preparation 28

### Preparation of 2-[6-(3-R-Methyl-piperazin-1-yl)-pyridin-3-yl]-pyrimidine

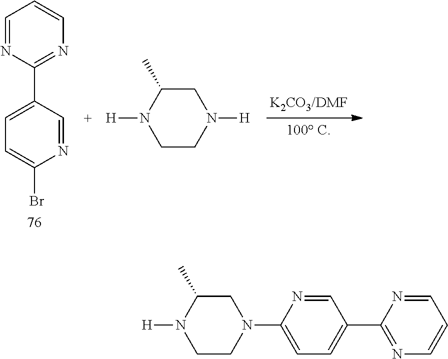

Following essentially the same procedure described in Preparation 8, except substituting an equivalent quantity of 2-R-Methyl piperazine for piperazine the title compound is obtained as a white solid (ESMS MH, 256) 95% Yield.

### Preparation 29

### Step 1: Preparation of 5-Pyrimidin-2-yl-3′,6′-dihydro-2′H-[2,4′]bipyridinyl-1′-carboxylic acid tert-butyl ester (2Q)

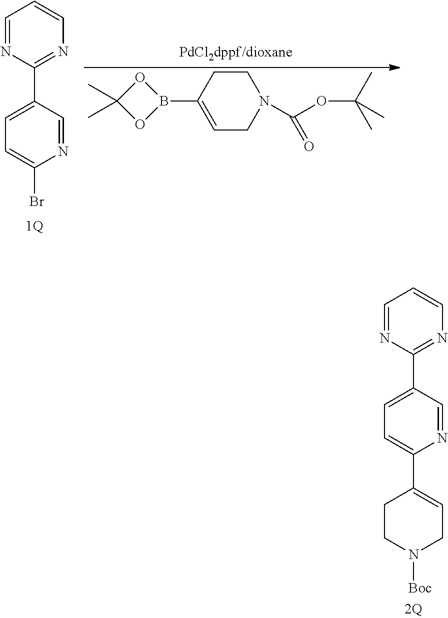

Refluxed mixture of 2-(6-Bromo-pyridin-3-yl)-pyrimidine (1Q) (200 mg, 0.85 mmol), N-tert-butoxycarbonyl-1,2,3,6-tetrahydropyridine-4-boronic acid, pinacol ester (290 mg, 0.93 mmol); Cesium Carbonate (500 mg, 1.538 mmol); PdCl₂dppf (30 mg) in dioxane/H₂O (10 ml v/v 4/1) for 4 hours. Cooled reaction, then evaporated solvent. Extracted with EtOAc (200 ml) washed with H₂O (50 ml), dried over MgSO₄, filtered and solvent evaporated yielding a solid which chromatographed on silica gel eluting with 30% v/v acetone/hexanes yielding 2Q as a white solid (110 mg, 38%) ESMS (MH,339).

### Step 2: Preparation of 5-Pyrimidin-2-yl-1′,2′,3′,6′-tetrahydro-[2,4′]bipyridinyl (3Q)

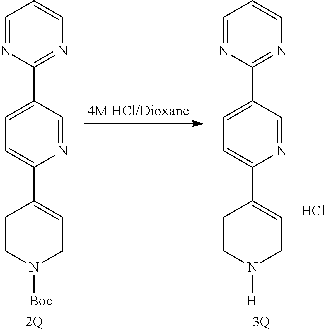

Added 4M HCl/dioxane (5 ml) to solution of 5-Pyrimidin-2-yl-3′,6′-dihydro-2′H-[2,4]bipyridinyl-1′-carboxylic acid tert-butyl ester (2Q) (110 mg, 0.325 mmol) in MeCl₂ (5 ml) at room temperature, then stirred 4 hours. Evaporated solvent. Added MeCl₂ (100 ml), H₂O (50 ml) and 10% NaOH (3 ml). The organic layer was separated, dried over MgSO₄, filtered and solvent evaporated yielding 3Q as a white solid (90 mg, 100%) ESMS (MH, 239) LCMS (MH, 239) Retention time=1.53 minutes.

### Step 3: Preparation of 2-Chloro-1-(5-pyrimidin-2-yl-3′,6′-dihydro-2′H-[2,4′]bipyridinyl-1′-yl)-ethanone (4Q)

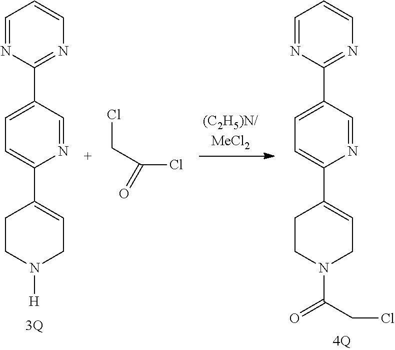

Added chloroacetyl chloride (0.35 g, 4.39 mmol) in MeCl₂ (15 ml) to a solution of 5-Pyrimidin-2-yl-1′,2′,3′,6′-tetrahydro-[2,4]bipyridinyl (3Q) (0.4 g, 1.68 mmol) and triethylamine (0.4 g, 2.87 mmol) in MeCl₂ (10 ml) at 0° C., then stirred 2 hours at 0° C. Added saturated NaHCO₃ solution and stirred an additional hour at 0° C. MeCl₂ (100 ml) was added, organic layer separated, dried over Na₂SO₄, filtered and solvent evaporated yielding 4Q as a pale yellow solid (0.53 g, 100%) ESMS (MH 315).

### Preparation 30

### Step 1: Preparation of 4-(4-bromo-2-fluoro-phenyl)-3,6-dihydro-2H-pyridine-1-carboxylic acid tert-butyl ester

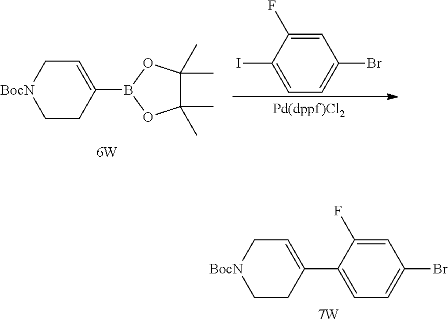

A mixture of compound 6W (1 g, 3.23 mmol), 4-bromo-2-fluoro-1-iodo-benzene (1.46 g, 4.85 mmol), potassium carbonate (1.4 g, 9.69 mmol), Pd(dppf)Cl₂ (0.264 g, 0.323 mmol) and 4/1/dioxane/water (10 ml) was degassed for 15 minutes. Then it was heated at 80° C. for overnight. Cooled to room temperature and diluted with EtOAc (200 ml). The organic layer was washed with water (100 ml), dried over Na₂SO₄, filtered and concentrated. The residue was purified on silica gel eluting with 1/10 EtOAc/hexane to give the desired product 7W (0.9 g, 78%).

### Step 2: Preparation of 4-(2-fluoro-4-pyrimid-2-yl-phenyl)-3,6-dihydro-2H pyridine-1-carboxylic acid 1-tert-butyl ester

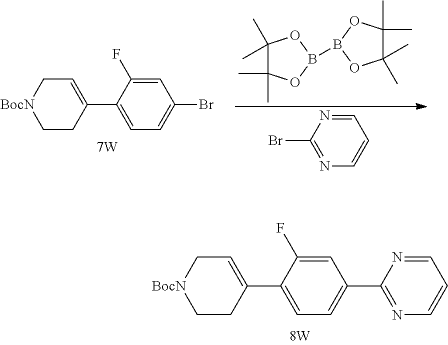

A mixture of compound 7W (0.9 g, 2.53 mmol), bis(pinacolato)diboron (0.96 g, 3.79 mmol), potassium acetate (0.74 g, 7.6 mmol), Pd(dppf)Cl₂ (0.21 g, 0.25 mmol) and dimethyl sulfoxide (10 ml) was degassed for 10 minutes. Then it was heated at 100° C. for overnight. The reaction mixture was cooled to room temperature and potassium carbonate (1.75 g, 12.63 mmol), 2-bromopyrimidine (0.48 g, 3.03 mmol) and water (10 ml) were added. The mixture was again purged with nitrogen for 20 min. Palladium tetrakistriphenylphosphine (0.29 g, 0.25 mmol) was added and the reaction mixture was stirred at 100° C. for a further 2 hr. Cooled to room temperature, filtered through a pad of celite and washed with ethyl acetate. Diluted with water (50 ml) and the organic layer was separated. The organic layer was dried over Na₂SO₄, filtered and concentrated. The residue was purified on silica gel eluting with 1/5 EtOAc/hexane to give the desired product 8W.

### Preparation of 31

### Step 1: Preparation of 4-(4-bromo-3-fluoro-phenyl)-3,6-dihydro-2H-pyridine-1-carboxylic acid tert-butyl ester

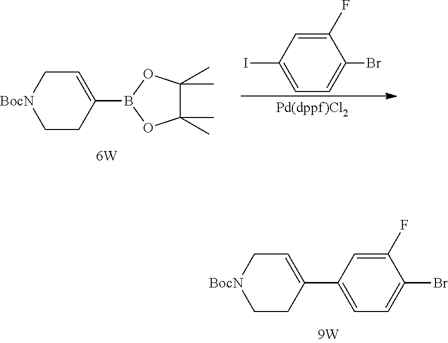

The compound 9W was prepared from compound 6W using essentially the same procedure as described for the preparation of compound 7W from compound 6W.

### Step 2: Preparation of 4-(3-fluoro-4-pyrimid-2-yl-phenyl)-3,6-dihydro-2H pyridine-1-carboxylic acid 1-tert-butyl ester

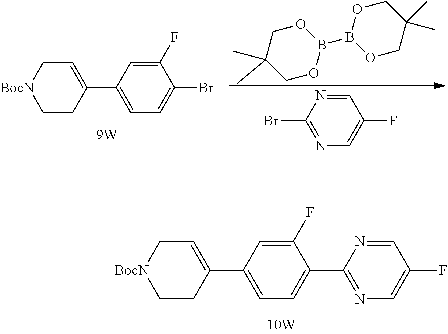

The compound 10W was prepared from compound 9W using essentially the same procedure as described for the preparation of compound 8W from compound 7W but using bis(neopentylglycolato)diboron and 2-bromo-6-fluoro-pyrimidine in place of bis(pinacolato)diboron and 2-bromo-pyrimidine.

### Preparation 32

### Step 1: Synthesis of 2-(2-Fluoro-4-piperazin-1-yl-phenyl)-pyrimidine (5AB)

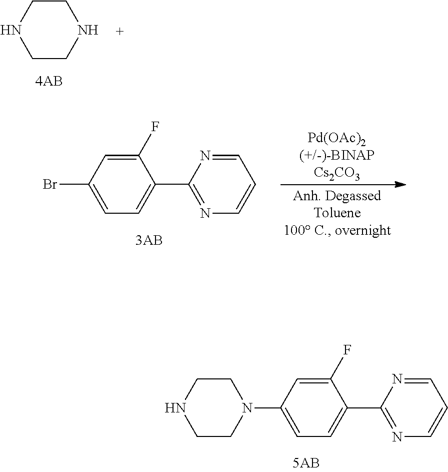

2-(4-Bromo-2-fluoro-phenyl)-pyrimidine (3AB) (2.0 g, 7.9 mmol, 1 equiv), piperazine (4AB) (2.72 g, 31.6 mmol, 4 equiv), cesium carbonate (20.6 g, 63.2 mmol, 8 equiv), racemic (+/−) BINAP (492 mg, 0.79 mmol, 0.1 equiv), and palladium (II) acetate (89 mg, 0.395 mmol, 0.05 equiv) were all weighed out in a flamed dried pressure vessel and the vessel was sealed with a rubber septa and the content of the reaction vessel was kept under vacuum for 2 hours. Anhydrous degassed toluene (100 mL) was added to the reaction vessel using a cannula. The rubber septa was replaced with a Teflon cap and the vessel was tightly sealed and placed in an oil bath at 100° C. to stir the content overnight.

The reaction vessel was cooled down to room temperature and the content was transferred into a flask. Some water was added to solubilize the excess inorganic base along with some ethyl acetate. The organic layer was then washed with water and brine twice, and separated and dried over magnesium sulfate. The crude product was then filtered into a flask and the solvent was removed on rotovap. The residue was taken up in as little dichloromethane as possible and purified by column chromatography using Analogix purification system with the following conditions: Solvent A: Dichloromethane; Solvent B: 40% 7N NH₃ in Methanol. Flow Rate. 65 mL/min. Gradient: 0% Solvent B to 30% Solvent B in 52 minutes and stayed at 30% Solvent B for 10 minutes.

Yield=889 mg (44%).

### Step 2: Synthesis of 2-Chloro-1-[4-(3-fluoro-4-pyrimidin-2-yl-phenyl)-piperazin-1-y]-ethanone (7AB)

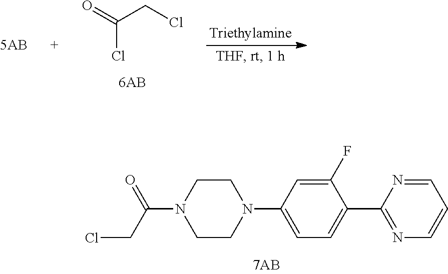

2-(2-Fluoro-4-piperazin-1-yl-phenyl)-pyrimidine (5AB) (889 mg, 3.442 mmol, 1 equiv) was dissolved in anhydrous tetrahydrofuran (5 mL) and triethylamine (697 mg, 959 uL, 6.884 mmol, 2 equiv) was added, followed by slow addition of a solution of chloroacetyl chloride (6AB) (466.5 mg, 330 uL, 4.13 mmol, 1.2 equiv) in tetrahydrofuran at room temperature. The mixture was then stirred for about 1 hour at room temperature.

Upon the completion of the reaction, the solvent was removed on rotovap and the residue was taken up in dichloromethane and washed with a (1v:1v) mixture of brine and water in a seperatory funnel. The organic layer was separated, concentrated down, and dried on pump. The crude residue was then taken up in dichloromethane and purified by column chromatography using Analogix purification system with the following conditions: Solvent A: Dichloromethane; Solvent B: 40% 7N NH₃ in Methanol. Flow Rate: 65 mL/min. Gradient: 0% Solvent B to 30% Solvent B in 52 minutes and stayed at 30% Solvent B for 10 minutes.

Yield=1.05 g (91.1%)

### Preparation 33

### Step 1: Synthesis of 4-(4-Bromo-3-fluoro-phenyl)-3,6-dihydro-2H-pyridine-1-carboxylic acid tert-butyl ester (16AB)

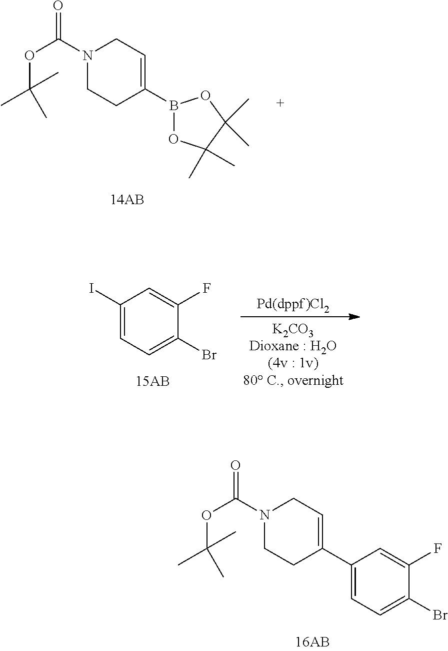

4-(4,4,5,5-Tetramethyl-[1,3,2]dioxaborolan-2-yl)-3,6-dihydro-2H-pyridine-1-carboxylic acid tert-butyl ester (14AB) (4.0 g, 12.9 mmol, 1 equiv), 1-Bromo-2-fluoro-4-iodo-benzene (15AB) (5.84 g, 19.4 mmol, 1.5 equiv), potassium carbonate (5.4 g, 38.8 mmol, 3 equiv), and a (4v:1 v) mixture of 1,4-dioxane and water (120 mL:30 mL) were all added in a pressure vessel (350 mL) and the mixture was bubbled with nitrogen gas for about 10 minutes. To this mixture was added dichloro[1,1′-bis(diphenylphosphino)ferrocene] palladium (II)/dichloromethane adduct (1.05 g, 1.29 mmol, 0.1 equiv), and the reaction vessel was tightly capped, placed in an oil bath at 80° C., and stirred overnight.

The reaction mixture was cooled down to room temperature and the content was transferred into a flask and concentrated down on rotovap. The residue was then taken up in ethyl acetate and, in a seperatory funnel; the crude mixture was washed with water, 10% sodium carbonate and brine. The organic layer was dried on magnesium sulfate and passed through a Celite plug. The filtrate was then treated with activated carbon at 65° C. in an Erlenmeyer in a water bath for about 10 minutes to decolorize the solution. The charcoal was separated by a Celite plug. The solvent was removed on rotovap and the residue was dried on pump overnight. The residue was taken up in as little dichloromethane as possible and purified by column chromatography using Analogix purification system with the following conditions: Solvent A: Hexanes; Solvent B: Ethylacetate. Flow Rate: 65 mL/min. Gradient: 0% Solvent B to 50% Solvent B in 60 minutes.

Yield=3.12 g (68%)

### Step 2: Synthesis of 4-[4-(5,5-Dimethyl-[1,3,2]dioxaborinan-2-yl)-3-fluoro-phenyl]-3,6-dihydro-2H-pyridine-1-carboxylic acid tert-butyl ester (18AB)

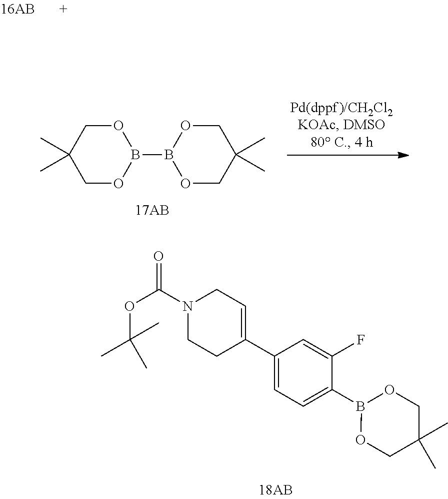

4-(4-Bromo-3-fluoro-phenyl)-3,6-dihydro-2H-pyridine-1-carboxylic acid tert-butyl ester (16AB)(2.3 g, 6.59 mmol, 1 equiv), bis(neopentylglycolato)diboron (17AB)(1.79 g, 7.91 mmol, 1.2 equiv), and potassium acetate (1.94 g, 19.77 mmol, 3 equiv) were all weighed out in a dry pressure vessel and dissolved in dimethylsulfoxide (50 mL). The mixture was bubbled with nitrogen gas for 10 minutes. Dichloro[1,1′-bis(diphenylphosphino) ferrocene]palladium (II)/dichloromethane adduct (540 mg, 0.66 mmol, 0.1 equiv) was added and the reaction vessel was sealed tightly with a cap and placed in on oil bath at 80° C. for 4 hours.

Upon the completion of 4 hours, the reaction vessel was cooled down to room temperature and the content was transferred into a flask. Some water was added to solubilize the excess inorganic base along with some ethyl acetate. The organic layer was then washed with water and brine twice, and separated and dried over magnesium sulfate. The organic layer was concentrated down on rotovap and taken up with dichloromethane. In an Erlenmeyer the crude compound was treated with activated carbon at 65° C. in a water bath for about 10 minutes to decolorize the solution. The charcoal was separated by a Celite plug. The solvent was removed on rotovap and the residue was dried on pump overnight. The residue was taken up in as little dichloromethane as possible and purified by column chromatography using Analogix purification system with the following conditions: Solvent A: Hexanes; Solvent B: Ethylacetate. Flow Rate: 65 mL/min. Gradient: 0% Solvent B to 50% Solvent B in 60 minutes. Relatively clean fractions were combined and the solvent was concentrated down. The product spot on TLC was streaking; that is probably because during the purification of this compound on a silica gel column some of the boronic acid ester was getting hydrolyzed to boronic acid. Therefore, even though the separation was not as desirable, the compound was used as-is in the next reaction after the purification step.

### Step 3: Synthesis 4-[3-Fluoro-4-(5-fluoro-pyrimidin-2-yl)-phenyl]-3,6-dihydro-2H-pyridine-1-carboxylic acid tert-butyl ester (21AB)

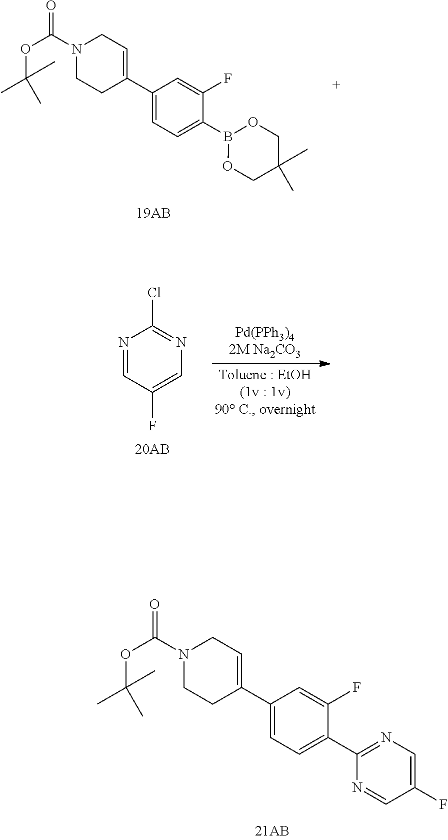

4-[4-(5,5-Dimethyl-[1,3,2]dioxaborinan-2-yl)-3-fluoro-phenyl]-3,6-dihydro-2H-pyridine-1-carboxylic acid tert-butyl ester (19AB)(1.55 g, 3.98 mmol, 1 equiv), 2-Chloro-5-fluoro-pyrimidine (20AB)(634 mg, 591 uL, 4.78 mmol, 1.2 equiv), and 2M sodium carbonate (9.95 mL) were added in a pressure vessel (350 mL) and a (1v:1v) mixture of toluene and ethanol (25 mL:25 mL) was added. The mixture was then bubbled with nitrogen gas for about 10 minutes. Tetrakis(triphenylphosphine) palladium (0) (462 mg, 0.4 mmol, 0.1 equiv) was added to the mixture. The reaction vessel was tightly capped, placed in an oil bath at 90° C., and stirred overnight.

The reaction mixture was cooled down to room temperature and diluted with ethyl acetate. The crude mixture was transferred into a seperatory funnel and washed with a (1v:1v) brine and water mixture. The organic layer was separated and combined and dried over magnesium sulfate. The crude product was then filtered into a flask and the solvent was removed on rotovap. The residue was taken up in as little dichloromethane as possible and purified by column chromatography using Analogix purification system with the following conditions: Solvent A: Dichloromethane; Solvent B: Methanol. Flow Rate: 45 mL/min. Gradient: 0% Solvent B to 10% Solvent B in 60 minutes.

Yield=677 mg (46%)

### Step 4: Synthesis of 5-Fluoro-2-[2-fluoro-4-(1,2,3,6-tetrahydro-pyridin-4-yl)-phenyl]-pyrimidine (22AB)

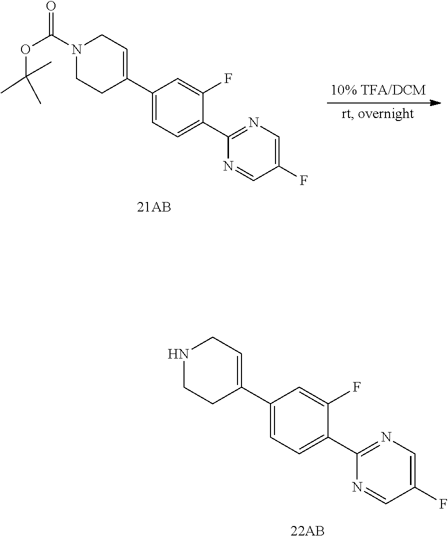

4-[3-Fluoro-4-(5-fluoro-pyrimidin-2-yl)-phenyl]-3,6-dihydro-2H-pyridine-1-carboxylic acid tert-butyl ester (21AB) (717 mg, 1.92 mmol, 1 equiv) was treated with 10% solution of trifluoroacetic acid in dichloromethane at room temperature overnight.

The solvent was concentrated down and the residue was taken up in ethyl acetate and washed with 10% aqueous sodium carbonate twice. Water layers were combined and saturated with sodium chloride and the remaining product in water layer thus extracted with ethyl acetate. The organic layers were combined and evaporated to dryness on a rotovap. The residue was taken up in as little dichloromethane as possible and purified by column chromatography using Analogix purification system with the following conditions: Solvent A: Dichloromethane; Solvent B: 40% 7N NH₃ in Methanol. Flow Rate: 65 mL/min. Gradient: 0% Solvent B to 30% Solvent B in 60 minutes and stayed at 30% Solvent B for 10 minutes. LCMS [M+H⁺]=274.2.

### Step 5: Synthesis of 2-Chloro-1-{4-[3-fluoro-4-(5-fluoro-pyrimidin-2-yl)-phenyl]-3,6-dihydro-2H-pyridin-1-yl}-ethanone (23)

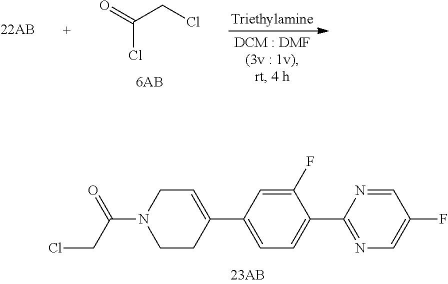

5-Fluoro-2-[2-fluoro-4-(1,2,3,6-tetrahydro-pyridin-4-yl)-phenyl]-pyrimidine (22AB) (1.94 g, 7.1 mmol, 1 equiv) was dissolved in a (3v:1v) mixture of dichloromethane (30 mL) and N,N-dimethyl formamide (10 mL) and triethylamine (862 mg, 1.19 mL, 8.52 mmol, 1.2 equiv) was added, followed by slow addition of chloroacetyl chloride (962 mg, 678 uL, 8.52 mmol, 1.2 equiv) at room temperature. The mixture was then stirred for about 4 hours at room temperature.

Upon the completion of the reaction, the solvent mixture was removed on rotovap and the residue was taken up in dichloromethane and washed with saturated solution of sodium bicarbonate and a (1v:1v) mixture of brine and water in a seperatory funnel. The organic layer was separated, concentrated down, and dried on pump. The crude residue was then taken up in dichloromethane and purified by column chromatography using Analogix purification system with the following conditions: Solvent A: Dichloromethane; Solvent B: 40% 7N NH₃ in Methanol. Flow Rate: 65 mL/min. Gradient: 0% Solvent B to 30% Solvent B in 52 minutes and stayed at 30% Solvent B for 10 minutes.

Yield=851 mg (34%)

### Preparation 34

### Step 1: Synthesis of 2-(4-Bromo-3-fluoro-phenyl)-pyrimidine (29AB)

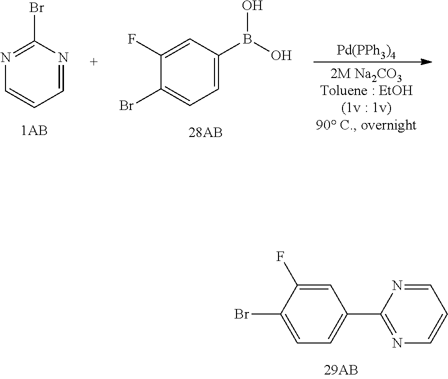

4-Bromo-3-fluorophenyl boronic acid (28AB) (1.0 g, 4.57 mmol, 1 equiv), 2-bromopyrimidine (1AB) (2.18 g, 13.7 mmol, 3 equiv), and 2M sodium carbonate (12 mL) were added in a pressure vessel (150 mL) and a (1v:1v) mixture of toluene and ethanol (25 mL:25 mL) was added. The mixture was then bubbled with nitrogen gas for about 10 minutes. Tetrakistriphenylphosphine palladium (0) (266 mg, 0.23 mmol, 0.05 equiv) was added to the mixture. The reaction vessel was tightly capped, placed in an oil bath at 90° C., and stirred overnight.

The reaction mixture was cooled down to room temperature and the content was filtered into a flask and the solvent mixture was evaporated off on the rotovap. The residue was then taken up in one to one mixture of toluene and ethyl acetate and washed with (3v:1v) mixture of brine: DI water twice. The organic layer was separated and combined and dried over magnesium sulfate. The crude product was then filtered into a flask and the solvent was removed on rotovap. The residue was taken up in as little dichloromethane as possible and purified by column chromatography using Analogix purification system with the following conditions: Solvent A: Hexanes; Solvent B: Ethylacetate. Flow Rate: 65 mL/min. Gradient: 0% Solvent B to 50% Solvent B in 60 minutes.

Yield=1.08 g (94%)

### Step 2: Synthesis of 2-(3-Fluoro-4-piperazin-1-yl-phenyl)-pyrimidine (30AB)

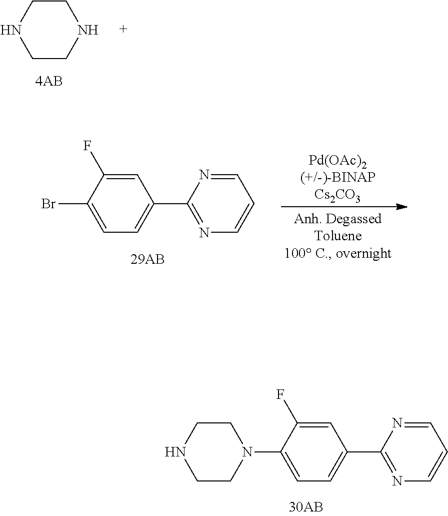

2-(4-Bromo-3-fluoro-phenyl)-pyrimidine (29AB) (874 mg, 3.45 mmol, 1 equiv), piperazine (1.19 g, 13.8 mmol, 4 equiv), cesium carbonate (9.0 g, 27.6 mmol, 8 equiv), racemic (+/−) BINAP (215 mg, 0.345 mmol, 0.1 equiv), and palladium (II) acetate (38.8 mg, 0.173 mmol, 0.05 equiv) were all weighed out in a flamed dried pressure vessel and the vessel was sealed with a rubber septa and the all-solid mixture was kept under vacuum for 2 hours. Anhydrous degassed toluene (30 mL) was added to the reaction vessel using a cannula. The rubber septa was replaced with a Teflon cap and the vessel was tightly sealed and placed in an oil bath at 100° C. to stir the content overnight.

The reaction vessel was cooled down to room temperature and the content was transferred into a flask. Some water was added to solubilize the excess inorganic base along with some ethyl acetate. The organic layer was then washed with water and brine twice, and separated and dried over magnesium sulfate. The crude product was then filtered into a flask and the solvent was removed on rotovap. The residue was taken up in as little dichloromethane as possible and purified by column chromatography using Analogix purification system with the following conditions: Solvent A: Dichloromethane; Solvent B: 40% 7N NH₃ in Methanol. Flow Rate: 40 mL/min. Gradient: 0% Solvent B to 30% Solvent B in 52 minutes and stayed at 30% Solvent B for 10 minutes.

Yield=675 mg (76%)

### Step 3: Synthesis of 2-Chloro-1-[4-(2-fluoro-4-pyrimidin-2-yl-phenyl)-piperazin-1-yl]-ethanone (31AB)

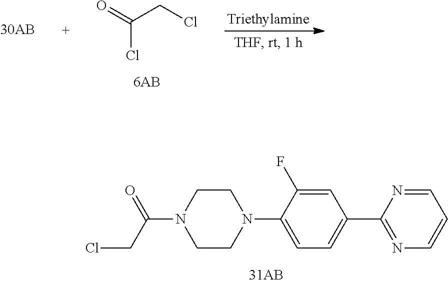

2-(3-Fluoro-4-piperazin-1-yl-phenyl)-pyrimidine (29AB) (675 mg, 2.61 mmol, 1 equiv) was dissolved in anhydrous tetrahydrofuran (5 mL), and triethylamine (1.32 mg, 1.82 mL, 13.05 mmol, 5 equiv) was added, followed by slow addition of a solution of chloroacetyl chloride (591 mg, 417 uL, 5.23 mmol, 2 equiv) in tetrahydrofuran at room temperature. The mixture was then stirred for about 1 hour at room temperature.

Upon the completion of the reaction, the solvent was removed on rotovap and the residue was taken up in dichloromethane and washed with a (1v:1v) mixture of brine and water in a seperatory funnel. The organic layer was separated, concentrated down, and dried on pump. The crude residue was then taken up in dichloromethane and purified by column chromatography using Analogix purification system with the following conditions: Solvent A: Dichloromethane; Solvent B: 40% 7N NH₃ in Methanol. Flow Rate: 40 mL/min. Gradient: 0% Solvent B to 30% Solvent B in 52 minutes and stayed at 30% Solvent B for 10 minutes.

Yield=821 mg (94%)

### Preparation 35

### Preparation of 2-Chloro-1-(5,8-dichloro-3,4-dihydro-1H-isoquinolin-2-yl)-ethanone

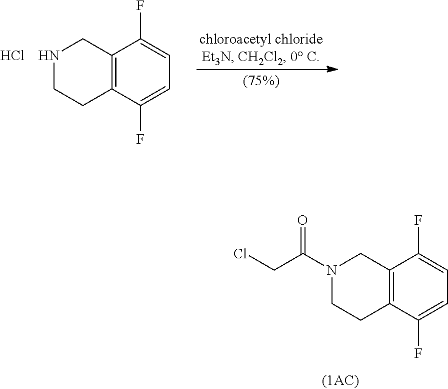

To a stirred suspension of 5,8-di-fluoro-1,2,3,4-tetrahydroisoquinoline hydrochloride (0.69 g, 3.36 mmol) in dichloromethane (20 ml) at 0° C. under nitrogen, diisopropylethylamine (1.40 ml, 8.05 mmol) followed by chloroacetyl chloride (0.32 ml, 4.03 mmol) were added. The mixture was stirred at 0° C. for 2 hr. After being quenched with saturated sodium carbonate solution, water and dichloromethane were added. Layers were separated and the separated aqueous layer was extracted with dichloromethane. The combined organic layers were dried (MgSO₄), filtered and solvents were removed in vacuum. Column purification [Hexanes-Ethyl acetate, 4:1 (v/v)] gave chloride 1AC (619 mg, 75%) as colourless oil.

### Preparation 36

### Step 1: Preparation of 2-(4-Bromo-2,3-difluoro-phenyl)-pyrimidine

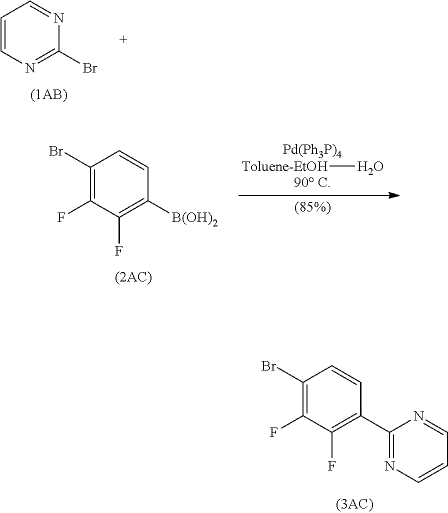

A mixture of 2-bromopyrimidine (2.0 g, 12.7 mmol), 4-bromo-2,3-difluorobenzeneboronic acid (1.0 g, 4.22 mmol), potassium carbonate (2.93 g, 21.1 mmol) in a mixture of toluene (30 ml)/ethanol (30 ml)/water (15 ml) were purged with nitrogen for 15 min. Tetrakis(triphenylphosphine)palladium(0) (488 mg, 0.42 mmol) was added and the mixture was stirred at 90° C. in a sealed-tube for overnight. The mixture was cooled to r.t. and was diluted with water and ethyl acetate. Layers were separated. The separated organic layer was dried (MgSO₄), filtered and solvents were removed in vacuum. Column purification [Hexanes-Ethyl acetate, 2:1 (v/v)] gave bromide 3AC (0.97 g, 85%) as white solid.

### Step 2: Preparation of 2-(2,3-Difluoro-4-piperazin-1-yl-phenyl)-pyrimidine

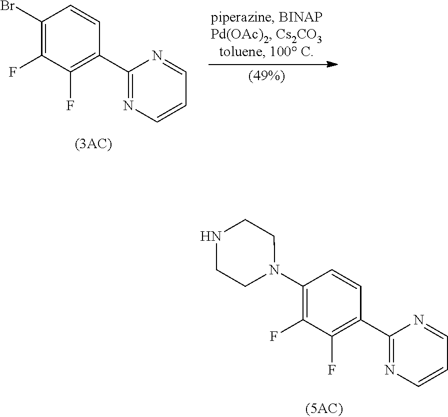

A mixture of bromide 3AC (300 mg, 1.11 mmol); piperazine (286 mg, 3.32 mol), BINAP (69 mg, 0.11 mmol), cesium carbonate (721 mg, 2.21 mmol) in toluene (10 ml) were purged with nitrogen for 15 min. Palladium (II) acetate (13 mg, 0.055 mmol) was added and the mixture was stirred at 100° C. in a sealed-tube for overnight. The mixture was cooled to r.t. and was diluted with water and ethyl acetate. Layers were separated. The separated organic layer was dried (MgSO₄), filtered and solvents were removed in vacuum. Column purification [Methanol-Ethyl acetate, 1:1 (v/v)] gave piperazine 5AC (150 mg, 49%) as white solid.

### Step 3: Preparation of 2-Chloro-1-[4-(2,3-Difluoro-4-pyrimidin-2-yl-phenyl)-piperazin-1-yl]-ethanone

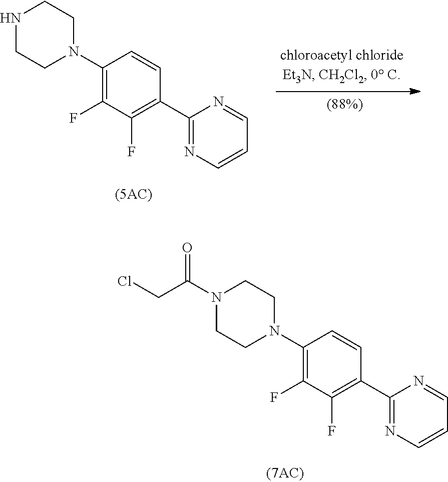

To a stirred solution of piperazine 5AC (150 mg, 0.54 mmol) in dichloromethane (5 ml) at 0° C. under nitrogen, triethylamine (0.076 ml, 0.54 mmol) followed by chloroacetyl chloride (0.043 ml, 0.54 mmol) were added. The mixture was stirred at 0° C. for 2 hr. After being quenched with saturated sodium carbonate solution, water and dichloromethane were added. Layers were separated and the separated aqueous layer was extracted with dichloromethane. The combined organic layers were dried (MgSO₄), filtered and solvents were removed in vacuum. Column purification [Hexanes-Ethyl acetate, 1:1 (v/v)] gave chloride 7AC (169 mg, 88%) white solid.

### Preparation 37

### Step 1: Preparation of 2-(4-Bromo-2,5-difluoro-phenyl)-pyrimidine

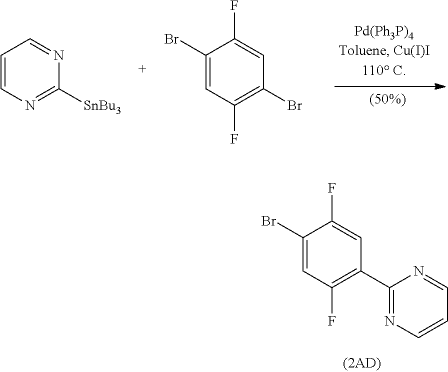

A mixture of 1,4-dibromobenzene (4.4 g, 16.3 mmol), 2-(tributylstannyl)pyrimidine (3.0 g, 8.13 mmol), copper (I) iodide (154 mg, 0.81 mmol) in toluene (50 ml) were purged with nitrogen for 15 min. Tetrakis(triphenylphosphine)palladium(0) (939 mg, 0.81 mmol) was added and the mixture was stirred at 110° C. in a sealed-tube for 1 day. The mixture was cooled to r.t. and was diluted with water and ethyl acetate. Layers were separated. The separated organic layer was dried (MgSO₄), filtered and solvents were removed in vacuum. Column purification [Hexanes-Ethyl acetate, 2:1 (v/v)] gave bromide 2AD (1.09 g, 50%) as white solid.

### Step 2: Preparation of 2-(2,5-Difluoro-4-piperazin-1-yl-phenyl)-pyrimidine

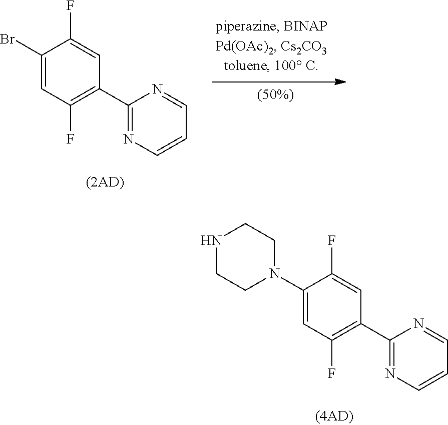

A mixture of bromide 2AD (850 mg, 3.14 mmol), piperazine (810 mg, 9.41 mmol), BINAP (196 mg, 0.31 mmol), cesium carbonate (2.0 g, 6.27 mmol) in toluene (30 ml) were purged with nitrogen for 15 min. Palladium (II) acetate (35 mg, 0.16 mmol) was added and the mixture was stirred at 100° C. in a sealed-tube for overnight. The mixture was cooled to r.t. and was diluted with water and ethyl acetate. Layers were separated. The separated organic layer was dried (MgSO₄), filtered and solvents were removed in vacuum. Column purification [Methanol-Ethyl acetate, 1:1 (v/v)] gave piperazine 4AD (393 mg, 43%) as white solid.

### Step 3: Preparation of 2-Chloro-1-[4-(2,5-Difluoro-4-pyrimidin-2-yl-phenyl)-piperazin-1-yl]-ethanone

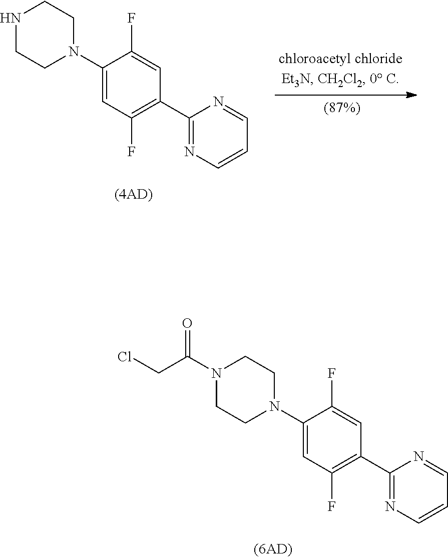

To a stirred solution of piperazine 4AD (255 mg, 0.92 mmol) in dichloromethane (5 ml) at 0° C. under nitrogen, triethylamine (0.13 ml, 0.92 mmol) followed by chloroacetyl chloride (0.074 ml, 0.92 mmol) were added. The mixture was stirred at 0° C. for 2 hr. After being quenched with saturated sodium carbonate solution, water and dichloromethane were added. Layers were separated and the separated aqueous layer was extracted with dichloromethane. The combined organic layers were dried (MgSO₄), filtered and solvents were removed in vacuum. Column purification [Hexanes-Ethyl acetate, 1:1 (v/v)] gave chloride 6AD (283 mg, 87%) white solid.

### Preparation 38

### Preparation of 4-(3-Methoxy-4-pyrimidin-2-yl-phenyl)-3,6-dihydro-2H-pyridine-1-carboxylic acid tert-butyl ester

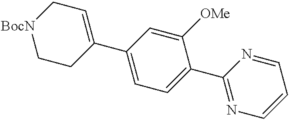

4-(3-Methoxy-4-pyrimidin-2-yl-phenyl)-3,6-dihydro-2H-pyridine-1-carboxylic acid tert-butyl ester was prepared using essentially the same scheme for Preparation 39 starting from 1-bromo-4-iodo-2-methoxy-benzene.

### Preparation 39

### Preparation of 4-Fluoro-4-(4-pyrimidin-2-yl-phenyl)-piperidine-1-carboxylic acid tert-butyl ester

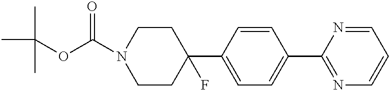

The above compound was prepared using a procedure similar to that of Preparation 40 Step 4 by using 4-(4-Bromo-phenyl)-4-fluoro-piperidine-1-carboxylic acid tert-butyl ester in place of 3AE.

### Preparation 40

### Step 1

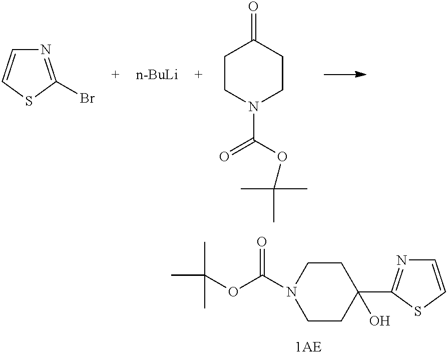

Compound 1AE was prepared following a procedure similar to that of Preparation 11

### Step 2

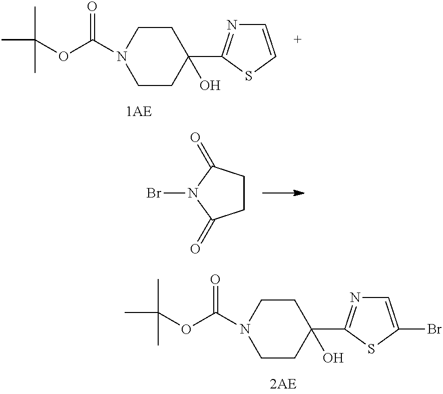

4.03 g (14.18 m.mole) 1AE was dissolved in 50 ml anhy. CH₃CN at r.t. under dry N2 this 2.524 g (14.18 m.mole) of N-Bromosuccinimde was added at r.t. and mixture was heated 50° C. under dry N2 gas for 3 hrs. The mixture was evaporated to dryness. The residue was partitioned between 100 ml EtOAc and 100 ml saturated NaHCO₃ solution. The organic phase dried over MgSO₄ and evaporated to dryness. The resulting brown gum was purified on silica (Hexane-30% EtOAc/Hexane) gave 1.3 g (25%) yellow solid.

### Step 3

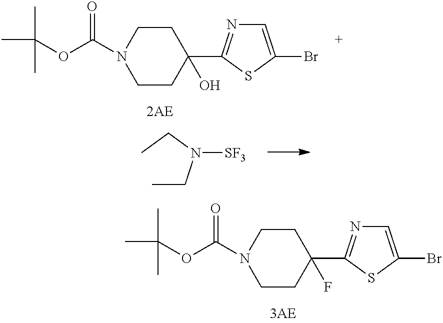

2.38 g (6.55 m.mole) of 3 was dissolved in 30 ml anhy. Dichloromethane at r.t. under dry N₂ gas. The mixture was cooled to 0° C. in ice-bath and 2.112 g (13.1 m. mole) of DAST was added dropwise at 0° C. under dry N₂. The mixture was stirred at 0° C. for 1 hr. The reaction mixture was cautiously (CO₂ gas evolution) basified with saturated NaHCO₃ solution at 0° C. The mixture was tranfered to separatory funnel and shaken well. The organic phase was removed and aqueous phase was extracted with 2×50 ml Dichloromethane. The combine organic phases were dried over MgSO₄ and evaporated to dryness, gave 2.368 (99%) off-white low melting solid.

### Step 4

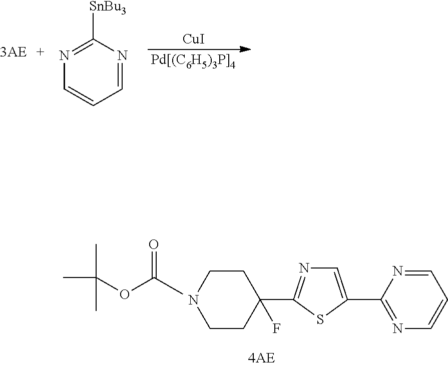

1.7 g (4.65 m. mole) of 4AE was dissolved in 15 ml anhy DMF at r.t. under dry N₂ gas. To this mixture CuI 0.93 g (4.88 m.mole), Tetrakis(triphenylphosphine) palladium 0.537 g (0.465 m.mole) and 2-Tributylstannylpyrimidine 1.762 g (5.2 m.mole) were added and the mixture was stirred at 60° C. under dry N₂ gas for 24 hrs. The mixture was concentrated to small volume, diluted with 50 ml EtOAc and filtered through pad of celite. The filtrate was washed with brine and dried over MgSO₄. The evaporation of the solvent gave dark brown gum which was purified on silica gel (Hexane-25% EtOAc/Hexane) gave 0.300 g (17%) of brown solid.

### Preparation 41

### Step 1

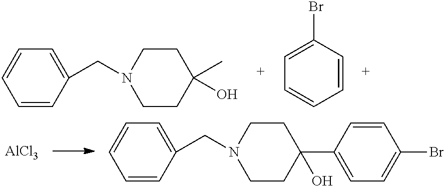

1-Benzyl-4-hydroxy-4-methyl piperidine (4.927 g, 24 m.mole) was dissolved in Bromobenzene (12 ml, 114 m.mole) at r.t.under dry N₂ gas. AlCl₃ (4.81 g, 36 m.mole) as solid was added to the above mixture at r.t. under dry N₂ gas. There was slightly exothermic reaction. The resulting dark brown solution was heated at 100° C. over the week-end. The reaction was allowed to cool to r.t and was poured into ice-water. Saturated aqueous NaHCO₃ was added till pH 7. The mixture was extracted with 3×100 ml EtOAc. The combined organic extract dried over MgSO₄ and evaporated to dryness. The resulting dark brown gum was purified on silica and was eluted with (Hexane-25% EtOAc/Hexane), gave 4.43 g (53%) as violet clear thick oil.

### Step 2

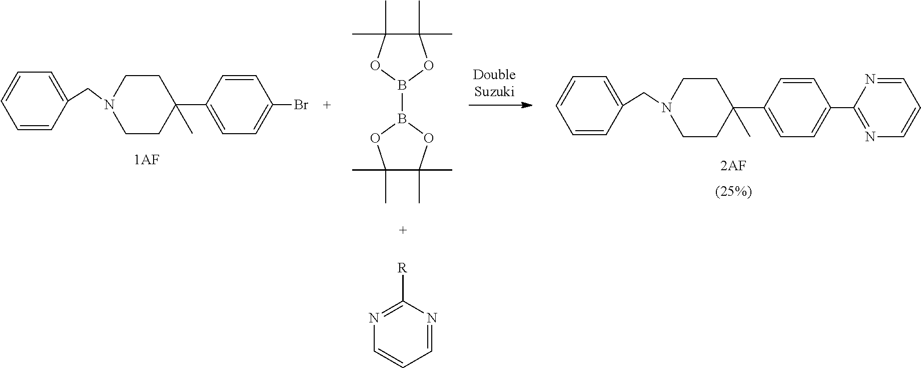

To a solution of 4.43 g (12.87 m.mole) of 1AF in 100 ml anhy. DMSO were added 4.903 g (19.31 m.mole) of Bis(pinacolato)diboron, 3.784 g (38.61 m.mole) of Potassium acetate and 1.051 g (1.287 m.mole) of Pd(dppf)Cl₂ at r.t. under dry N₂ gas. The contents were degassed couple of times with N₂ gas and stirred, at 100° C. for 2 hrs. The mixture was allowed to cool to r.t. and 50 ml of water was added followed by 2.455 g (15.44 m.mole) of 2-Bromopyrimidine, 8.894 g (64.35 m.mole) of Potassium carbonate and 1.49 g (1.29 m.mole) of Tetrakis(triphenylphosphine)palladium. The contents were degassed couple of times with N2 gas and stirred at 100 C for 2 hrs. The mixture was allowed to cool to r.t. 100 ml of water and 100 ml of EtOAc were added to the reaction mixture and filtered through pad of celite and washed with EtOAc. The contents were transfered to separatory funnel and the organic phase was separated and the aqueous phase was extracted with EtOAC. The organic phases were combined and washed with water and dried over MgSO₄. The solvent was evaporated to dryness and dark brown gum was purified on silica gel (Hexane-25% EtOAC/Hexane), gave 1.00 off white solid.

### Step 3

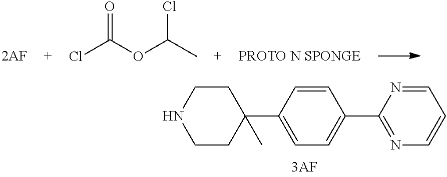

1.00 g (2.9 m.mole) of 2AF was in 20 ml anhy. dichloromethane at r.t. under dry N₂ gas. To this solution 0.178 g (0.83 m.mole) Proton Sponge was added at r.t. followed by dropwise addition of 0.713 g (4.99 m.mole) of 2-Chloroethyl chloroformate. The reaction mixture was stirred at r.t. under dry N₂ gas for 4 hrs. The mixture was evaporated to dryness and dried under high vacuum for 15 minutes. The resulting residue was dissolved in 20 ml anhy. MeOH under dry N₂ gas and was stirred under reflux under dry N₂ gas for 4 hrs. The mixture was allowed to cool to r.t. and evaporated to dryness. The crude was purified on silica gel (CH₂Cl₂-25% MeOH/CH₂Cl₂) and gave 0.606 g (82%) white solid.

### Step 4

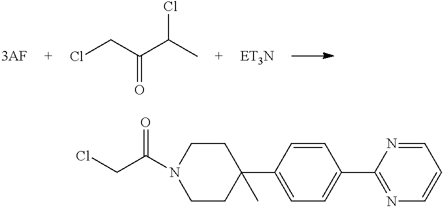

To a stirred solution of 0.60 g (2.37 m.mole) of 3AF in 15 ml anhy. Dichloromethane (11.85 m.mole) of Triethylamine was added at r.t. under dry N₂ gas. The mixture was cooled ice-water bath and 0.321 g (2.84 m.mole) of Chloroacetylchloride was added dropwise at 0 dry N₂ gas. the mixture was stirred at 0° C. for half an hr. 25 ml of CH₂Cl₂ and aqueous saturate NaHCO₃ solution were added at 0° C. The contents were transferred to separatory funnel are well. The oranic phase was separated, dried over MgSO₄ and evaporated to dryness, gave solid. This solid was used without purification for subsequent reaction.

### Preparation 42

### Step 1: 5′-Iodo-3-methyl-3,4,5,6-tetrahydro-2H-[1,2′]bipyrazinyl (1)

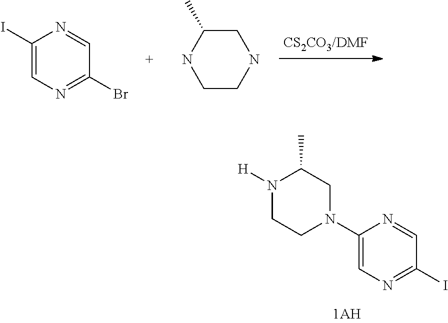

A mixture of 2-bromo-5-iodopyrazine (200 mg, 0.704 mmol), cesium carbonate (400 mg, 1.23 mmol) and 2R methyl piperazine (85 mg, 0.85 mmol) in DMF (10 ml) was stirred at 100° C. overnight. The reaction was cooled and solvent evaporated. Water (100 ml) was added and insoluble solid was filtered, then dissolved in MeCl₂ (100 ml), dried over Na₂SO₄, filtered and solvent evaporated yielding product (205 mg, 95%)

Mass Spec (MH, 305)

### Step 2: 3-Methyl-5′-pyrimidin-2-yl-3,4,5,6-tetrahydro-2H-[1,2′]bipyrazinyl (2)

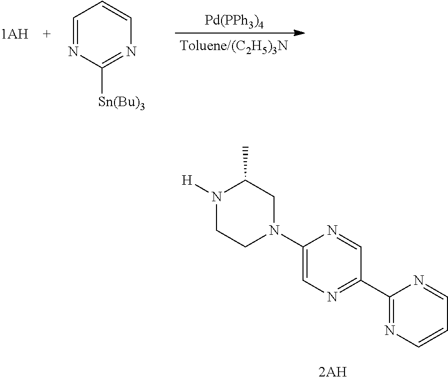

Added Pd(PPh₃)₄ (30 mg, 0.025 mmol) to a mixture of 5′-Iodo-3-methyl-3,4,5,6-tetrahydro-2H-[1,2′]bipyrazinyl (1AH) (50 mg, 0.164 mmol), 2-tributyl stannyl pyrimidine (0.2 ml), triethylamine (0.2 ml, 1.43 mmol) in toluene (3 ml) at room temperature then stirred at 100° C. for 5 hours. The reaction was cooled, diluted with EtOAC (50 ml) and water (20 ml). The organic layer was separated, dried (Na₂SO₄) filtered and solvent evaporated. The residue was purified on Prep TLC eluting with 10% MeOH:MeCl₂:NH₄OH yielding product (10 mg, 24%) Mass Spec MH 256

### Preparation 43

### 2-[6-(3-R-Methyl-piperazin-1-yl)-pyridin-3-yl]-pyrimidine

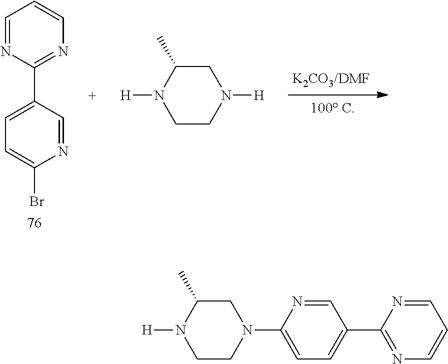

Following the procedure described in Preparation 6, but substituting an equivalent quantity of 2-R-Methyl piperazine for piperazine, the title compound is obtained as a white solid (ESMS MH,256) 95% Yield.

### Preparation 44

### Step 1: 2-Pyrazol-1-yl-pyrimidine

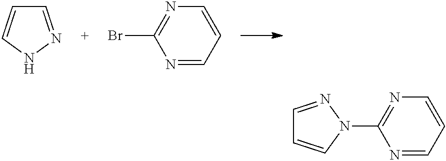

A reaction mixture containing pyrazole (2 g, 29 mmol), 2-bromopyrimidine (3.8 g, 24 mmol), copper (I) iodide (0.91 g, 4.8 mmol) and 1,10-phenanthroline (1.7 g, 9.6 mmol) in DMA was heated at 140° C. in a sealed tube for 6 hours. After the reaction, ethyl acetate (30 mL) was added, followed by water. The aqueous layer was extract three times (20 mL) and the organic layer was collected, dry over sodium sulfate. After concentration under vacuum, the crude product was purified using column chromatography (10% ethyl acetate in dichloromethane) to give 0.55 g of pure product. 15% yield. MS (ESMS, M+H 146).

### Step 2: 2-(4-Bromo-pyrazol-1-yl)-pyrimidine

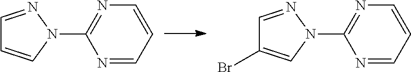

To a solution of 2-pyrazol-1-yl-pyrimidine (0.55 g, 3.7 mmol) in acetic acid (5 mL) was added bromine (1.2 g, 7.5 mmol) in acetic acid (3 mL) dropwisely. After addition, the reaction mixture was stirred at room temperature overnight. After removed the acetic acid, the crude product was purified using column chromatography (2% methanol in dichloromethane) to give 0.7 g of pure product in 85% yield. MS (ESMS, M+H 225).

### Step 3: 2-[4-(1,2,3,6-Tetrahydro-pyridin-4-yl)-pyrazol-1-yl]pyrimidine

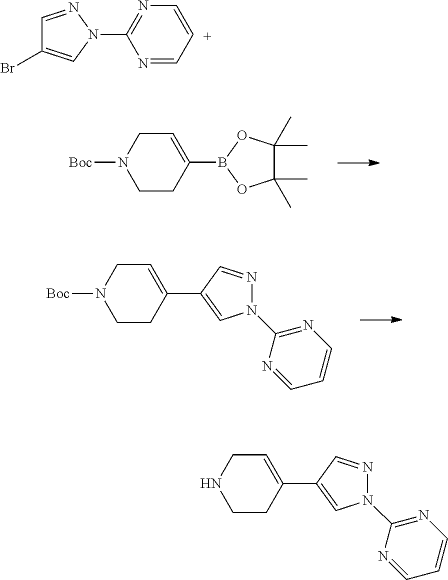

A solution containing 2-(4-bromo-pyrazol-1-yl)-pyrimidine (300 mg, 1.34 mmol), 4-(4,4,5,5-tetramethyl-[1,3,2]dioxaborolan-2-yl)-3,6-dihydro-2H-pyridine-1-carboxylic acid tert-butyl ester (280 mg, 1.34 mmol), PdCl₂(dppf) (95 mg, 0.13 mmol) and potassium phosphate (800 mg, 4 mmol) in dioxane was heated at 80° C. under argon for overnight. After removed the solvent, ethylacetate was added and the mixture was filtered, washed with water. After concentration under vacuum, it was found the product was hard to separated from impurity and the crude product was treated with 90% of TFA for 20 min and TFA was removed under vacuum. The crude product was then purified using prep HPLC to give desired product as TFA salt (120 mg, 0.37 mmol) in 27% overall yield. MS (ESMS, M+H 228)

### Preparation 45

### Preparation of 4-(2,5-Difluoro-4-pyrimidin-2-yl-phenyl)-3,6-dihydro-2H-pyridine-1-carboxylic acid tert-butyl ester (Compound 12AQ)

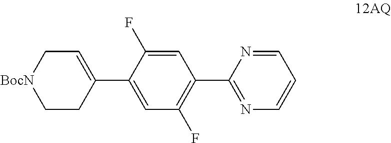

The Compound 12AQ was prepared from 4-(4,4,5,5-tetramethyl-[1,3,2]dioxaborolan-2-yl)-3,6-dihydro-2H-pyridine-1-carboxylic acid tert-butyl ester using the procedure as described for the preparation of Compound 4-(2-fluoro-4-pyrimidin-2-yl-phenyl)-3,6-dihydro-2H-pyridine-1-carboxylic acid tert-butyl ester but using 1,4-dibromo-2,5-difluoro-benzene in place of 4-bromo-2-fluoro-1-iodobenzene.

### Preparation 46

### Step 1: Preparation of 4-Bromo-3-fluoro-benzoic acid hydrazide

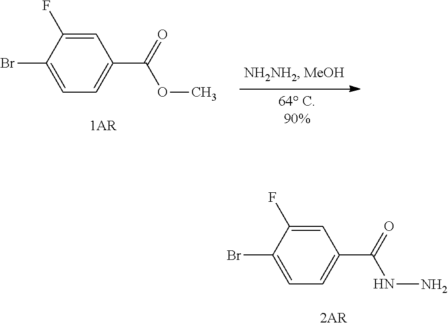

A mixture of compound 4-bromo-3-fluoro-benzoic acid methyl ester (1 g, 4.29 mmol), hydrazine hydrate (2.2 mL, 42.9 mmol) and MeOH (20 mL) was heated at 70° C. for overnight. Concentrated, diluted with EtOAc (300 mL) and washed with water (100 mL). The organic layer was dried over Na₂SO₄, filtered and concentrated give the desired product 2AR (0.9 g, 90%).

### Step 2: Preparation of [5-(4-Bromo-3-fluoro-phenyl)-[1,3,4]oxadiazol-2-yl]-ethyl-amine

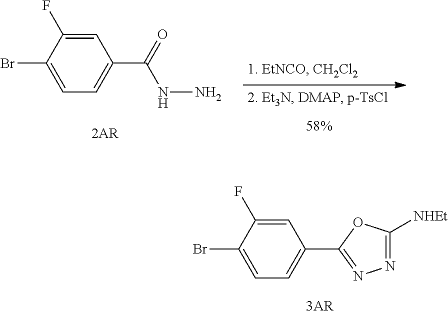

A mixture of Compound 2AR (0.9 g, 2.53 mmol), CH₂Cl₂ (5 mL) and ethyl isocyanate (0.34 mL, 4.35 mmol) was stirred at room temperature for 3 hours. To the reaction mixture was added triethylamine (0.94 mL, 6.7 mmol), DMAP (0.205 g, 1.675 mmol) and a solution of p-toluenesulfonyl chloride (0.83 g, 4.36 mmol) in CH₂Cl₂ (10 mL). Reaction mixture was stirred at room temperature for 18 hours. Diluted with CH₂Cl₂ (200 mL) and washed with water (100 mL). The organic layer was dried over Na₂SO₄, filtered and concentrated. The residue was purified on silica gel eluting with 3% MeOH/CH₂Cl₂ to give the desired product 3AR (0.56 g, 58%).

### Step 3: Preparation of 4-[4-(5-Ethylamino-1,3,4]oxadiazol-2-yl)-2-fluoro-phenyl]-3,6-dihydro-2H-pyridine-1-carboxylic acid tert-butyl ester

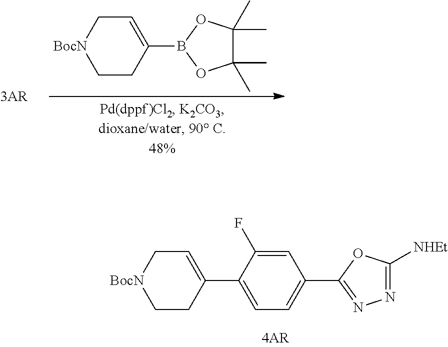

A mixture of Compound 3AR (0.56 g, 1.96 mmol), 4-(4,4,5,5-tetramethyl-[1,3,2]dioxaborolan-2-yl)-3,6-dihydro-2H-pyridine-1-carboxylic acid tert-butyl ester (0.73 g, 2.35 mmol), potassium carbonate (0.81 g, 5.88 mmol), Pd(dppf)Cl₂ (0.192 g, 0.235 mmol) and 4/1/dioxane/water (10 ml) was degassed for 15 minutes. Then it was heated at 80° C. for overnight. Cooled to room temperature and diluted with EtOAc (200 ml). The organic layer was washed with water (100 ml), dried over Na₂SO₄, filtered and concentrated. The residue was purified on silica gel eluting with 5% MeOH/CH₂Cl₂ to give the desired product 4AR (0.44 g, 48%).

### Step 4: Preparation of Ethyl-{5-[3-fluoro-4-(1,2,3,6-tetrahydro-pyridin-4-yl]-[1,3,4]oxadiazol-2yl}-amine

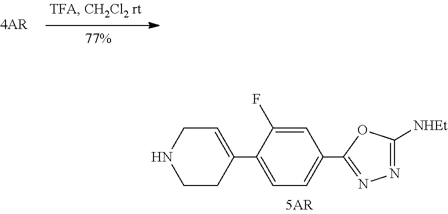

A mixture of Compound 4AR (0.44 g, 1.13 mmol), CH₂Cl₂ (20 mL) and TFA (2 mL) was stirred at room temperature for 18 hours. Concentrated and purified on silica gel eluting with 5% MeOH(NH₃)/CH₂Cl₂ to give the desired product 5AR (0.25 g, 77%).

### Step 5: Preparation of 2-Chloro-1-{4-[4-(5-ethylamino-[1,3,4]oxadiazol-2-yl)-2-fluoro-phenyl]-3,6-dihydro-2H-pyridin-1-yl}-ethanone

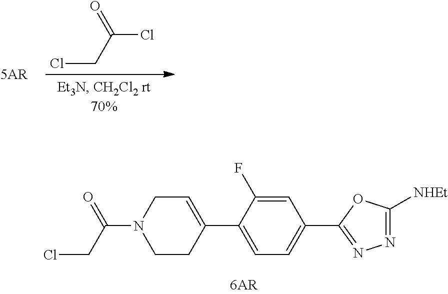

To a mixture of Compound 5AR (0.1 g, 0.35 mmol), CH₂Cl₂ (5 mL), MeOH (1 mL) and triethyl amine (0.041 mL, 0.29 mmol) at −78° C. was added chloroacetyl chloride (0.021 mL, 0.264 mmol). Reaction mixture was stirred at −78° C. for 10 minutes then warm to 0° C. and stirred for 1 hour. Diluted with CH₂Cl₂ (100 mL) and washed with saturated aq. NaHCO₃ (100 mL). The organic layer was dried over Na₂SO₄, filtered and concentrated. The residue was purified on silica gel eluting with 2% MeOH/CH₂Cl₂ to give the desired product 6AR (0.09 g, 70%).

### Preparation 47

### Step 1

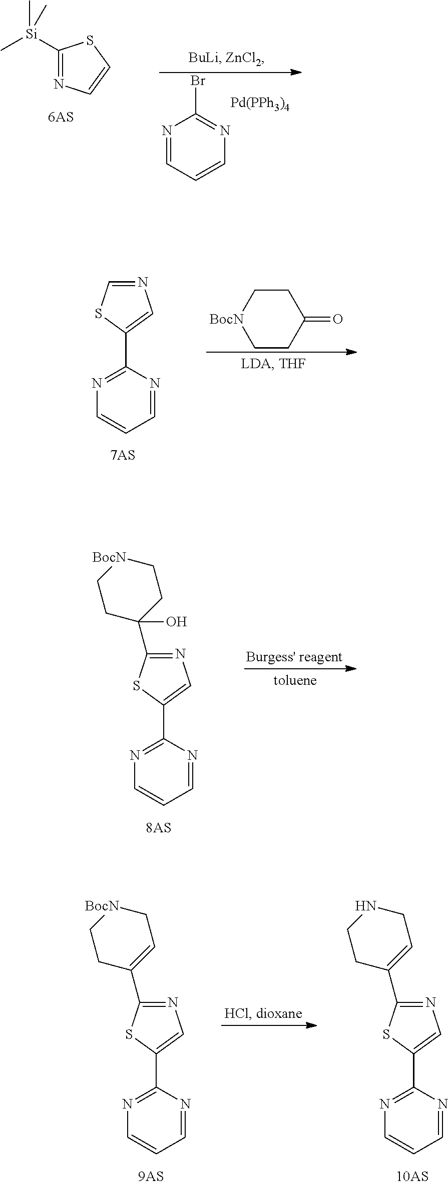

In a 250 round bottom flask was placed butyl lithium (6.1 mL, 2.5 M in hexanes, 15.2 mmol) in THF at −78° C. under Ar. To this was added 6 (2.0 g, 12.7 mmol), stirred for 15 min and added Zinc chloride (38.1 mL, 0.5 M in THF, 19.1 mmol). The mixture was warmed up to room temperature and stirred for 1 hr. To this was added 2-bromopyrimidine (2.4 g, 15.2 mmol) and Pd(PPh₃)₄ (293 mg, 0.252 mmol). The reaction was heated to reflux overnight, cooled to room temperature and filtered. The filtrate was partitioned between brine and ethyl acetate. The organic layer was dried over sodium sulfate and concentrated. The resulting mixture was purified by biotage column chromatography to afford 7AS (936 mg, 54.2%)

To a solution of 7AS (710 mg, 4.35 mmol) in THF at −78° C. was added LDA (2.61 mL, 2.0 M, 5.22 mmol), and then Boc-4-piperidone (1.04 g, 5.22 mmol). The reaction was stirred at −78° C. for 1 hr, warmed up to room temperature and quenched with ammonium chloride solution. The mixture was extracted with ethyl acetate. The combined organic layer was dried over sodium sulfate and concentrated. The resulting oil was purified by biotage column chromatography to afford 8AS (1.08 g, 68%)

To a solution of 8AS (800 mg, 2.21 mmol) in toluene was added Burgess reagent (1.09 g, 4.58 mmol). The mixture was heated to 100° C., stirred for 2 hrs, cooled to room temperature and concentrated. The residue was purified biotage column chromatography to afford 9AS (562 mg, 74%)

### Step 2

To 9AS (560 mg, 1.63 mmol) in a 20 mL vial was added 4 mL of HCl in dioxane (4 M). The reaction was stirred at room temperature for 4 hrs and the precipitate was filtered. The resulting solid was pump dried to afford 10 (350 mg, 88%)

### Step 3

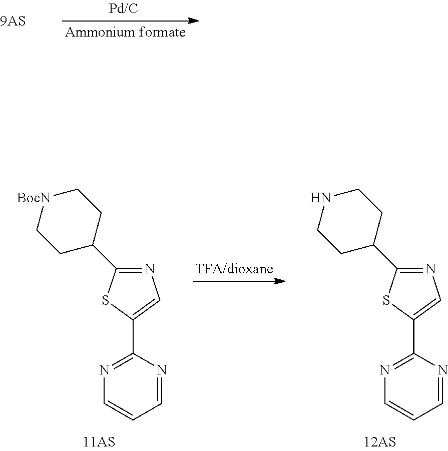

To a solution of 9AS (100 mg, 0.291 mmol) and ammonium formate (183 mg, 2.91 mmol) in methanol was added catalytic amount of 10% Palladium on carbon. The mixture was heated to reflux overnight, cooled to room temperature and filtrated. The filtrate was concentrated and the residue was purified biotage column chromatography to afford 11AS (52 mg, 52%) and recovered 9 (18 mg, 18%)

To 11AS (52 mg, 0.15 mmol) in 1 mL of DCM was added 1 mL of TFA. The reaction was stirred at room temperature for 2 hrs and concentrated to afford crude 12AS.

### Preparation 48

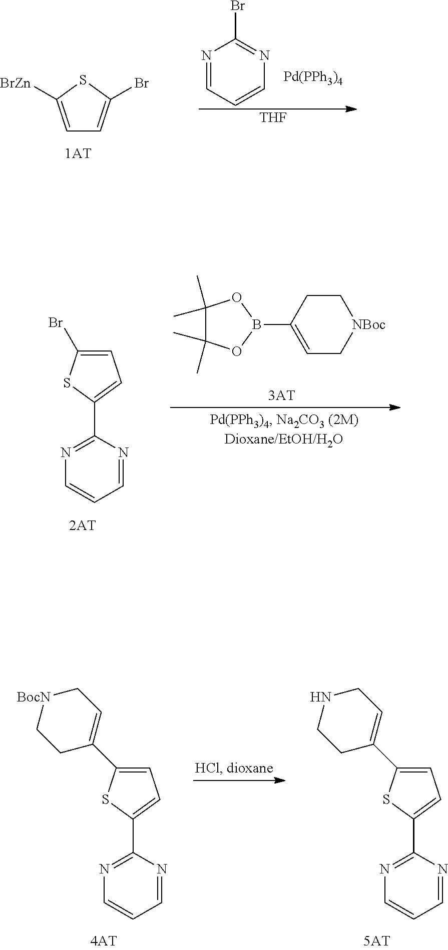

In a 250 mL of round bottom flask was placed 1 AT (0.5 M in THF, 20.0 mL, 10.0 mol). To this was added 2-bromopyrimidine (2.00 g, 12.6 mol) and Pd(PPh₃)₄ (346 mg, 0.3 mmol). The mixture was heated to reflux under Ar overnight and cooled down to room temperature. The reaction was quenched with ammonium chloride solution and extracted with ethyl acetate. The combined organic layer was dried over sodium sulfate and concentrated. The resulting oil was purified by biotage column chromatography to afford 2AT (1.62 g, 69%)

In a 5 mL of biotage microwave vessel was placed 2AT (136 mg, 0.568 mmol), pinacol ester 3AT (193 mg, 0.625 mmol), Pd(PPh₃)₄ (32.8 mg, 0.0284 mmol) and sodium carbonate solution (0.85 mL, 2 M) in dioxane/EtOH/H₂O (7:3:2, 2.5 mL) under Ar. The vessel was sealed and heated in microwave reactor at 150° C. for 10 minutes. The reaction was partitioned between ethyl acetate and water. The organic layer was dried over sodium sulfate and concentrated. The resulting oil was purified by biotage column chromatography to afford 4AT (149 mg, 76%).

To 4AT (144 mg, 0.420 mmol) in a 20 mL vial was added 2 mL of HCl in dioxane (4 M). The reaction was stirred at room temperature for 4 hrs and the precipitate was filtered. The resulting solid was pump dried to afford 5AT (83 mg, 82%)

### Preparation 49

### Synthesis of 6-Pyrimidin-2-yl-1,2,3,4-tetrahydro-isoquinoline

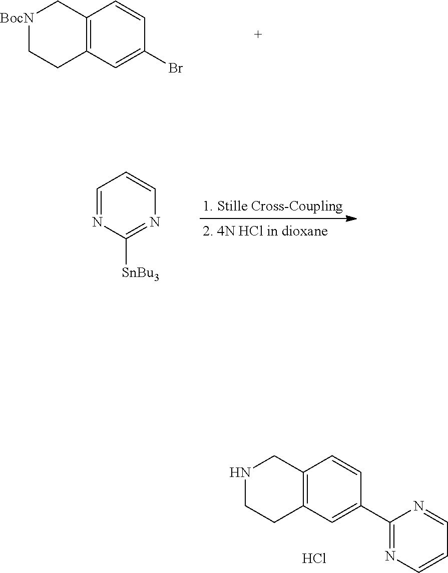

To a Schlenk tube were charged Pd₂(dba)₃ (10 mg, 0.01 mmol), bis(tri-tert-butylphosphine)palladium (20 mg, 0.04 mmol), CuI (16 mg, 0.08 mmol) and CsF (334 mg, 2.2 mmol). The tube was evacuated under high vacuum and back-filled with nitrogen for three cycles. DMF (2 ml) was introduced, followed by 2-tributylstannylpyrimidine (537 mg, 1.4 mmol). The tube was sealed with a Teflon cap and the reaction mixture was heated with stirring at 120° C. for 2 hours. After cooling, the mixture was filtered through Celite, washed with ethyl acetate. Filtrate was washed with water three times, brine and dried (MgSO₄). After concentration the residue was purified on silica gel eluting with ethyl acetate in hexanes (0-100%) to give 6-pyrimidin-2-yl-3,4-dihydro-1H-isoquinoline-2-carboxylic acid tert-butyl ester (28 mg). The compound was treated with 4N HCl in dioxane for 30 minutes. After concentration title compound was obtained as hydrochloride salt.

### Preparation 50

### Synthesis of 6-Pyrimidin-2-yl-1,2,3,4-tetrahydro-isoquinoline

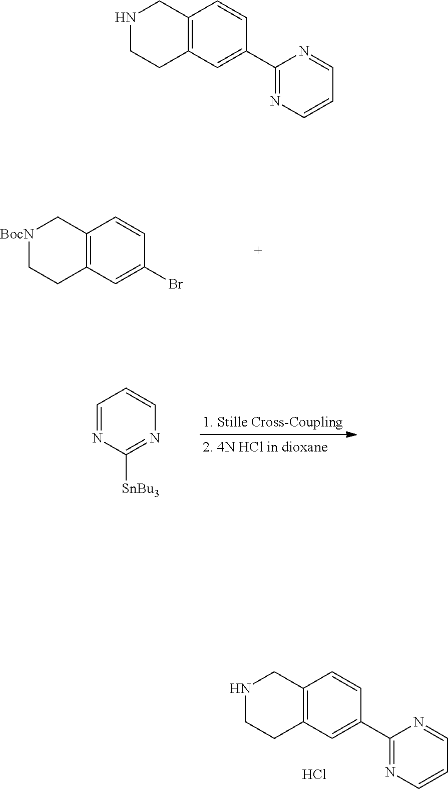

To a Schlenk tube were charged Pd₂(dba)₃ (10 mg, 0.01 mmol), bis(tri-tert-butylphosphine)palladium (20 mg, 0.04 mmol), CuI (16 mg, 0.08 mmol) and CsF (334 mg, 2.2 mmol). The tube was evacuated under high vacuum and back-filled with nitrogen for three cycles. DMF (2 ml) was introduced, followed by 2-tributylstannylpyrimidine (537 mg, 1.4 mmol). The tube was sealed with a Teflon cap and the reaction mixture was heated with stirring at 120° C. for 2 hours. After cooling, the mixture was filtered through Celite, washed with ethyl acetate. Filtrate was washed with water three times, brine and dried (MgSO₄). After concentration the residue was purified on silica gel eluting with ethyl acetate in hexanes (0-100%) to give 6-pyrimidin-2-yl-3,4-dihydro-1H-isoquinoline-2-carboxylic acid tert-butyl ester (28 mg). The compound was treated with 4N HCl in dioxane for 30 minutes. After concentration title compound was obtained as hydrochloride salt.

### Preparation 51

### Preparation of 4-Benzothiazol-2-yl-3,6-dihydro-2H-pyridine-1-carboxylic acid tert-butyl ester (Compound 21 BB)

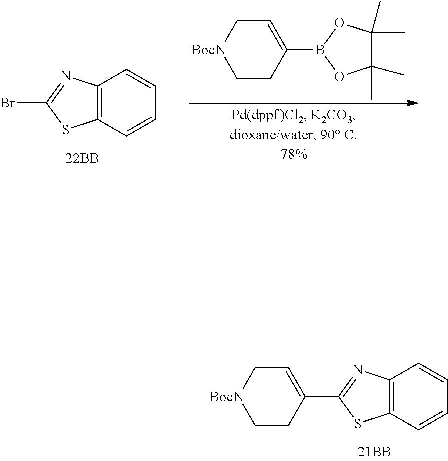

A mixture of 2-bromo-benzothiazole (0.38 g, 1.1 mmol), 4-(4,4,5,5-tetramethyl-[1,3,2]dioxaborolan-2-yl)-3,6-dihydro-2H-pyridine-1-carboxylic acid tent-butyl ester (0.5 g, 1.62 mmol), potassium carbonate (0.67 g, 4.85 mmol), Pd(dppf)Cl₂ (0.132 g, 0.16 mmol) and 4/1/dioxane/water (10 ml) was degassed for 15 minutes. Then it was heated at 90° C. for overnight. Cooled to room temperature and diluted with EtOAc (200 mL). The organic layer was washed with water (100 ml), dried over Na₂SO₄, filtered and concentrated. The residue was purified on silica gel to give the desired product 21 BB (0.4 g, 78%).

### Preparation 52

### Step 1: Preparation of 4-(4-Methoxycarbonyl-phenyl)-piperidine-1-carboxylic acid tert-butyl ester

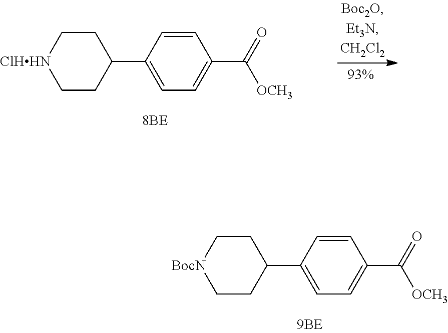

A mixture of Compound 8BE (3 g, 11.73 mmol), CH₂Cl₂ (30 mL), triethyl amine (4.9 mL, 35.19 mmol) and di-tert-butyl dicarbonate (3.83 g, 17.55 mmol) was stirred at room temperature for 3 hours. Diluted with CH₂Cl₂ (100 mL) and washed with water (100 mL). The organic layer was dried over Na₂SO₄, filtered and concentrated. The residue was purified on silica gel eluting with 100% EtOAc to give the desired product 9BE (3.5 g, 93%).

### Step 2: Preparation of 4-(4-Hydrazinocarbonyl-phenyl)-piperidine-1-carboxylic acid tert-butyl ester

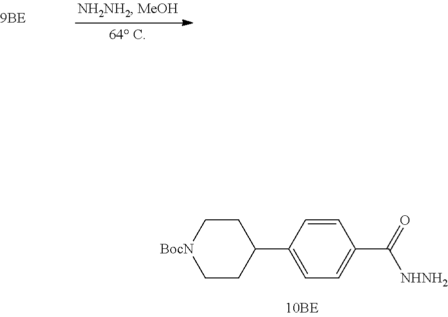

The Compound 9BE was converted to Compound 10BE using the procedure as described for the preparation of Compound 2AR from Compound 1AR (Preparation 46 Step 1).

### Step 3: Preparation of 4-[4-(5-Ethylamino-[1,3,4]oxadiazol-2-yl)-phenyl]-piperidine-1-carboxylic acid tert-butyl ester (Compound 11BE)

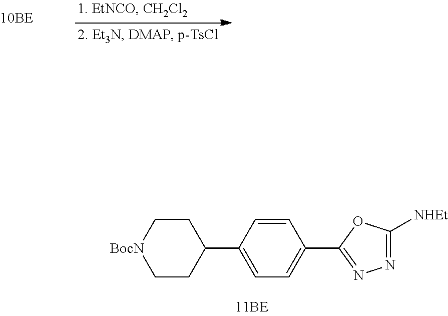

The Compound 10BE was converted to Compound 11 BE using the procedure as described for the preparation of Compound 3AR from Compound 2AR (Preparation 46 Step 2).

### Preparation 53

### Preparation of 2-Chloro-1-{4-[2-Fluoro-4-(5-methyl-[1,3,4]oxadiazol-2-yl)-phenyl]-3,6-dihydro-2H-pyridin-1-yl}-ethanone (Compound 20BF)

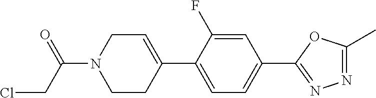

### Compound 20BF

### Step 1: Preparation of 2-(4-Bromo-3-fluoro-phenyl)-5-methyl-[1,3,4]oxadiazole

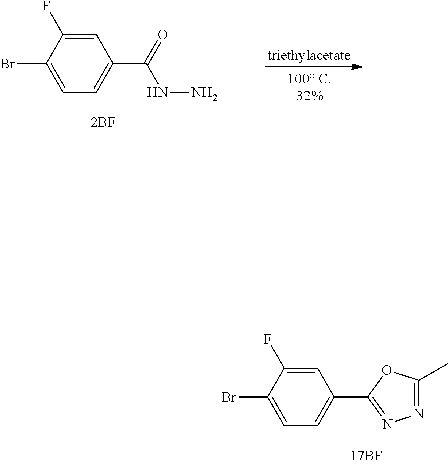

A mixture of Compound 2BF (0.9 g, 2.53 mmol) and triethylacetate (5 mL) was heated at 100° C. for 18 hours. Cooled to room temperature and poured into water (100 mL). Extracted with EtOAc (100 mL). The organic layer was dried (Na₂SO₄), filtered and concentrated. The residue was purified on silica gel eluting with 20% EtOAc/hexane to give the desired product 17BF (0.36 g, 32%).

### Step 2: Preparation of 4-[2-Fluoro-4-(5-methyl-[1,3,4]oxadiazol-2-yl)-phenyl]-3,6-dihydro-2H-pyridine-1-carboxylic acid tert-butyl

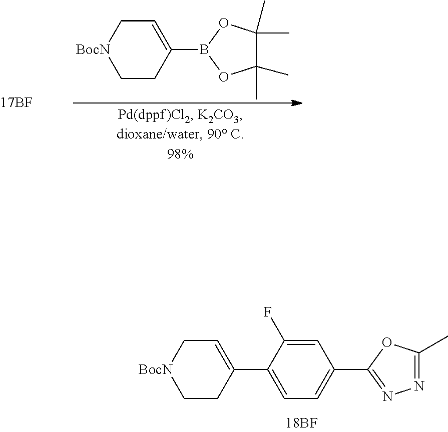

A mixture of Compound 17BF (0.34 g, 0.99 mmol), 4-(4,4,5,5-tetramethyl-[1,3,2]dioxaborolan-2-yl)-3,6-dihydro-2H-pyridine-1-carboxylic acid tert-butyl ester (0.37 g, 1.19 mmol), potassium carbonate (0.41 g, 2.97 mmol), Pd(dppf)Cl₂ (0.081 g, 0.099 mmol) and 4/1/dioxane/water (10 ml) was degassed for 15 minutes. Then it was heated at 90° C. for overnight. Cooled to room temperature and diluted with EtOAc (200 ml). The organic layer was washed with water (100 ml), dried over Na₂SO₄, filtered and concentrated. The residue was purified on silica gel to give the desired product 18BF (0.35 g, 98%).

### Step 3: Preparation of 4-[2-Fluoro-4-(5-methyl-[1,3,4]oxadiazol-2-yl)-phenyl]-1,2,3,6-tetrahydro-pyridine

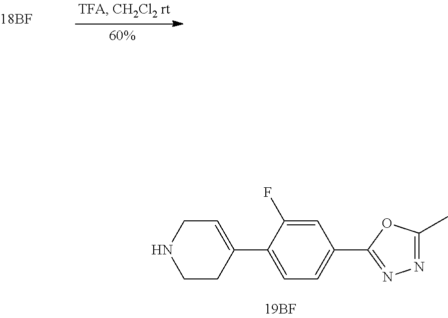

The Compound 18BF (0.44 g, 1.13 mmol) was converted to Compound 19BF using the procedure as described for the preparation of Compound 5AR from Compound 4AR (preparation 45 Step 4).

### Step 4

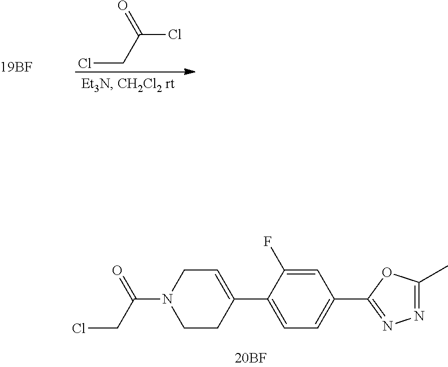

The Compound 19BF (0.44 g, 1.13 mmol) was converted to Compound 20BF using the procedure as described for the preparation of Compound 6AR from Compound 5AR (Preparation 45 Step 5).

### Preparation 54

### 4-Quinoxalin-6-yl-3,6-dihydro-2H-pyridine-1-carboxylic acid tert-butyl ester

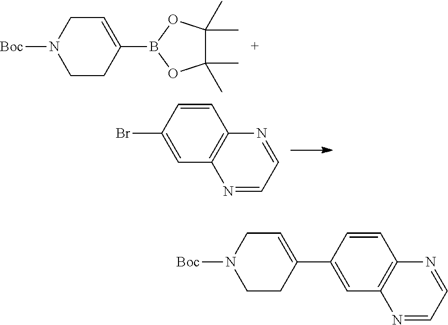

A solution containing 6-bromo-quinoxaline (417 mg, 2.0 mmol), 4-(4,4,5,5-tetramethyl-[1,3,2]dioxaborolan-2-yl)-3,6-dihydro-2H-pyridine-1-carboxylic acid tert-butyl ester (600 mg, 1.94 mmol), tetrakis [triphenylphosphine] palladium (108 mg, 0.1 mmol) and sodium carbonate (2 M solution, 3 mL) in 5 mL of dioxane/ethanol/water (7:3:1) was heated at 160° C. using microwave reactor for 15 minutes. After the reaction, ethylacetate was added and the mixture was filtered, washed with water. After concentration under vacuum, the product was purified using column chromatography (5% methanol in dichloromethane)

### Assays

### Coupled ERK2 Assay:

Activity of compounds against inactive ERK2 can be tested in a coupled MEK1/ERK2 IMAP assay as follows: Compounds can be diluted to 25× final test concentration in 100% DMSO. 14 μl of kinase buffer (10 mM Tris.HCl pH 7.2, 10 mM MgCl₂, 0.01% Tween-20, 1 mM DTT) containing 0.4 ng unphosphorylated Mouse ERK2 protein can be added to each well of a black 384-well assay plate. 1 μl of 25× compound can be added to each well and incubated at room temperature for 30 minutes to allow an opportunity for the compound to bind to the inactive enzyme. DMSO concentration during initial incubation can be 6.7%. ERK2 activity can be determined to be insensitive to DMSO concentrations up to 20%. ERK2 can then be activated and it's kinase activity measured by the addition of 10 μl kinase buffer with the following components (final concentration per reaction): 2 ng active (phosphorylated) human MEK1 protein and 4 μM (total) ERK2 IMAP substrate peptides (3.9 μM unlabeled IPTTPITTTYFFFK-CONH₂ and 100 nM IPTTPITTTYFFFK(5-carboxyfluorescein)-CONH₂) and 30 μM ATP. DMSO concentration during ERK activation can be 4%. After one hour, reactions can be terminated by addition of 60 μl IMAP detections beads in binding buffer (Molecular Devices). Binding can be allowed to equilibrate for 30 minutes before reading the plate on an LJL Analyst Fluorescence Polarization plate reader. Compound inhibition can be calculated relative to DMSO and fully inhibited standards. Active compounds can be reconfirmed in an independent assay.

### Active ERK2 Assay:

Activated ERK2 activity was also determined in the IMAP assay format using the procedure outlined above. 1 μl of 25× compound was added to 14 μl of kinase buffer containing 0.25 ng fully phosphorylated, active Mouse ERK2 protein. Following a 30 minute incubation, the reactions were initiated by addition of 10 μl of kinase buffer containing 1 μM ERK2 IMAP substrate peptide (0.9 μM unlabeled IPTTPITTTYFFFK-CONH₂ and 100 nM IPTTPITTTYFFFK(5-carboxyfluorescein)-CONH₂) and 30 μM ATP. Reactions proceeded for 30 minutes before termination by addition of 60 μl IMAP detection beads in binding buffer. Plates were read as above after 30 minute binding equilibration. Active compounds were reconfirmed in an independent assay.

### Soft Agar Assay:

Anchorage-independent growth is a characteristic of tumorigenic cell lines. Human tumor cells can be suspended in growth medium containing 0.3% agarose and an indicated concentration of a farnesyl transferase inhibitor. The solution can be overlayed onto growth medium solidified with 0.6% agarose containing the same concentration of ERK1 and ERK2 inhibitor as the top layer. After the top layer is solidified, plates can be incubated for 10-16 days at 37° C. under 5% CO₂ to allow colony outgrowth. After incubation, the colonies can be stained by overlaying the agar with a solution of MTT (3-[4,5-dimethyl-thiazol-2-yl]-2,5-diphenyltetrazolium bromide, Thiazolyl blue) (1 mg/mL in PBS). Colonies can be counted and the IC₅₀'s can be determined.

### The AUC (Area Under the Concentration-time Curve During the First 6 Hours (AUC₆ₕᵣ) can be Determined Using the Protocol of Cassette Accelerating Rapid Rat Screen (CARRS)

### Animal Dosing and Sample Collection

Male Sprague-Dawley rats (Charles River, Co.) can be pre-cannulated (femoral artery) in order to facilitate precise blood sampling times, and to reduce the stress on the animals caused by serial bleedings. Following an overnight fast, two rats can be dosed orally with one compound at a dose of 10 mg/kg in a 5-mL/kg dose volume. Blood can be collected into heparin-containing tubes serially from each animal at 0.5, 1, 2, 3, 4 and 6 h post-dosing and can be centrifuged to generate plasma. Approximately 100 μL of plasma can be collected at the individual time points. The plasma samples can be stored at −20° C. until analysis.

### Plasma Sample and Standard Curve Preparation

A set of 12 rat plasma samples can be generated for each NCE (i.e. 6 timepoints and n=2 rats). These 12 samples can be pooled across the two rats at each timepoint to provide 6 pooled samples (one sample per time point) for each NCE. The pooled samples can be assayed as cassettes of six (36 samples total) to provide data on the six compounds. The 50-μL aliquots of the 36 plasma samples can be placed into individual wells of a 96-well plate. An additional compound (often a structural analog of the test compounds) can be selected as the internal standard. A mini-calibration curve can be prepared (three points plus a zero) for each compound assayed. Drug-free rat plasma can be measured into 1-mL aliquots and each aliquot can be spiked with known concentrations of the compounds to generate standards of the desired concentrations. The concentrations of the standards can be chosen to bracket the expected concentration of the pooled samples based on historical data from previous studies on other compounds. For this work, the standards can be set to contain concentrations of 25, 250 and 2500 ng NCE/mL plasma. The plasma standards can be precipitated in duplicate along with the samples. Protein precipitation can occurr after addition of 150 μL of acetonitrile containing the internal standard at a concentration of 1 ng/mL into each sample well using the Tomtec Quadra 96 system. The precipitated samples and standards can be vortexed and centrifuged in the 96-well plate. Approximately 50-100 μL of the supernatant can be removed and placed into a fresh 96-well plate using the Tomtec Quadra 96 system. A volume of 5-10 μL of the supernatant can be used for analysis by HPLC-MS/MS. The mini-standard curve can be run in duplicate, once before and once after the samples. Thus, a total of 14 study samples plus standards can be analyzed per compound. In addition, solvent blanks can be injected before and after each set of 14 and after the highest calibration standard for each compound; therefore, a total of 103 injections can be made into each HPLC system for each set of six compounds. Multiple solvent blank injections can be made from a single well. Twelve solvent blank wells can be designated in each 96-well plate. Thus, one batch (cassette) of six NCEs can be prepared and assayed using one 96-well plate format.

### HPLC-MS/MS Analysis

All the compounds can be analyzed using selected reaction monitoring (SRM) methods with LC/MS/MS instruments. Once the method development is completed, the assay can be quickly set up using a standard injection sequence template for the CARRS assay.

The final compounds of Examples 1 to 12 had an AERK2 IC50 in the range of 9 to 3001 nM.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington: The Science and Practice of Pharmacy, 20^{th} Edition, (2000), Lippincott Williams & Wilkins, Baltimore, Md.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparations subdivided into suitably sized unit doses containing appropriate quantities of the active component, e.g., an effective amount to achieve the desired purpose.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg according to the particular application.

The actual dosage employed may be varied depending upon the requirements of the patient and the severity of the condition being treated. Determination of the proper dosage regimen for a particular situation is within the skill in the art. For convenience, the total daily dosage may be divided and administered in portions during the day as required.

The amount and frequency of administration of the compounds of the invention and/or, the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in two to four divided doses.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

1. A compound of formula 1.0: 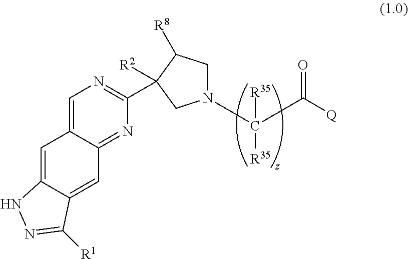
or the pharmaceutically acceptable salts, esters or solvates thereof, wherein:
z is 1 to 3;
Q is a substituent selected from the group consisting of: 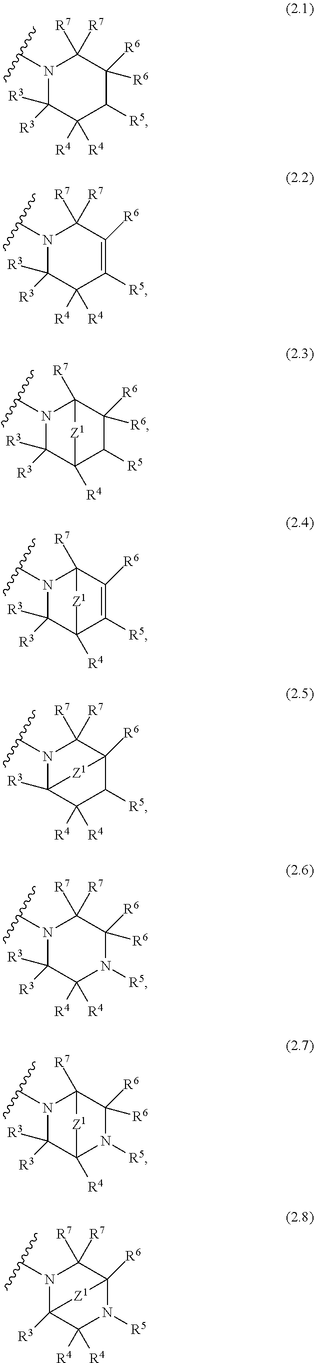 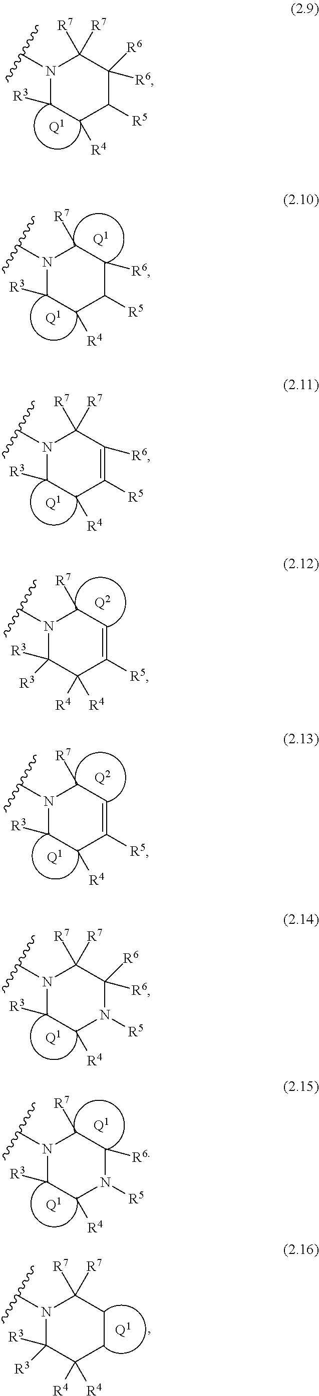 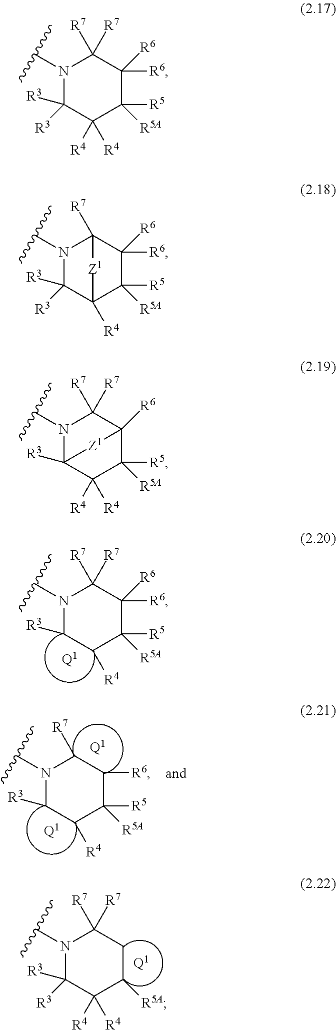
Each Q¹ represents a ring independently selected from the group consisting of: cycloalkyl, substituted cycloalkyl, heterocycloalkyl, substituted heterocycloalkyl, aryl, substituted aryl, heteroaryl, and substituted heteroaryl, wherein said substituted rings are substituted with 1 to 3 substituents independently selected from the group consisting of: halo and the R¹⁰ moieties; provided that when Q¹ is aryl, heteroaryl, substituted aryl or substituted heteroaryl then the carbon atoms at the ring junction are not substituted;
Q² represents a ring selected from the group consisting of: cycloalkyl, substituted cycloalkyl, heterocycloalkyl, and substituted heterocycloalkyl, wherein said substituted rings are substituted with 1 to 3 substituents independently selected from the group consisting of: the R¹⁰ moieties;
Z¹ represents —(C(R²⁴)₂)_{w}— wherein each R²⁴ is independently selected from the group consisting of: H, alkyl and F, and wherein w is 1, 2 or 3;
Z² is selected from the group consisting of: —N(R⁴⁴)—, —O— and —C(R⁴⁶)₂—;
m is 1 to 6;
n is 1 to 6;
p is 0 to 6;
t is 0, 1, or 2;
R¹ is selected from the group consisting of:
(1) —CN,
(2) —NO₂,
(3) —OR¹⁰,
(4) —SR¹⁰,
(5) —N(R¹⁰)₂,
(6) R¹⁰,
(7) —C(O)R¹⁰,
(8) —(C(R³⁰)₂)ₙ—NR³²—C(O)—R¹⁰, wherein in one example n is 1, each R³⁰ is H, R³² is H, and R¹⁰ is selected from the group consisting of: cycloalkyl and alkyl,
(9) —(C(R³⁰)₂)ₙ—NR³²—S(O)ₜR¹⁰,
(10) —(C(R³⁰)₂)ₙ—NR³²—C(O)—N(R³²)—R¹⁰,
(11) 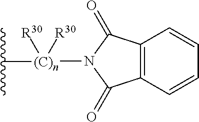
(12) —CF₃,
(13) —C(O)OR¹⁰,
(14) —(C(R³⁰)₂)ₙR¹³,
(15) alkenyl (e.g., —CH═CHCH₃),
(16) —NR³²—C(O)—R¹⁴,
(17) 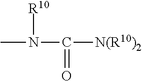
wherein each R¹⁰ is independently selected,
(18) 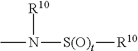
wherein each R¹⁰ is independently selected,
(19) 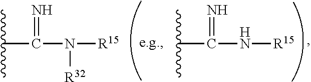
(20) —C(O)—NR³²—(C(R³⁰)₂)ₚ—OR¹⁰,
(21) —C(O)N(R¹⁰)₂ wherein each R¹⁰ is independently selected,
(22) —C(O)—NR³²—C(R¹⁸)₃,
(23)) —C(O)—NR³²—(C(R³)₂)ₙ—C(O)—N(R¹⁰)₂,
(24) heterocycloalkenyl, such as, for example: 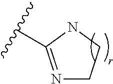
wherein r is 1 to 3,
(25) 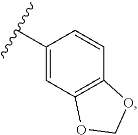
(26) arylalkenyl-, and
(27) halo;
R² is selected from the group consisting of:
(1) H,
(2) —CN,
(3) halo,
(4) alkyl,
(5) substituted alkyl wherein said substituted alkyl is substituted with 1 to 3 substitutents selected from the group consisting of: (a) —OH, (b) —O-alkyl, (c) —O-alkyl substituted with 1 to 3 F atoms, and (d) —N(R⁴⁰)₂ wherein each R⁴⁰ is independently selected from the group consisting of: (i) H, (ii) C₁-C₃ alkyl, (iii) —CF₃, and (e) halo,
(6) alkynyl,
(7) alkenyl,
(8) —(CH₂)ₘR¹¹,
(9) —N(R²⁶)₂,
(10) —OR²³,
(11) —N(R²⁶)C(O)R⁴²,
(12) cycloalkyl,
(13) cycloalkylalkyl,
(14) 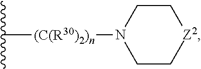
(15) —O-(substituted alkyl) wherein said substituted alkyl is substituted with 1 to 3 F atoms,
(16) —S(O)ₜ-alkyl,
(17) —C(O)-alkyl,
(18) 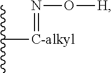
(19) 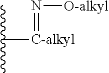
wherein each alkyl is independently selected,
(20) 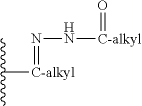
wherein each alkyl is independently selected,
(21) 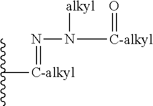
wherein each alkyl is independently selected,
(22) —N(R⁴⁸)—C(O)—R⁴⁸ wherein each R⁴⁵ is independently selected from the group consisting of: H and alkyl, and
(23) —C(O)-alkyl;
each R³, R⁴, R⁵, R⁶ and R⁷ is independently selected from the group consisting of:
(1) H,
(2) alkenyl,
(3) substituted alkenyl,
(4) alkyl,
(5) substituted alkyl,
(6) cycloalkyl,
(7) substituted cycloalkyl,
(8) cycloalkylalkyl-,
(9) substituted cycloalkylalkyl-,
(10) heterocycloalkyl,
(11) substituted heterocycloalkyl,
(12) heterocycloalkylalkyl-,
(13) substituted heterocycloalkylalkyl-,
(14) —C(O)R¹⁶,
(15) arylheteroaryl-,
(16) substituted arylheteroaryl-,
(17) heteroarylaryl-,
(18) substituted heteroarylaryl-,
(19) aryl,
(20) substituted aryl,
(21) heteroaryl,
(22) substituted heteroaryl,
(23) heteroarylheteroaryl-,
(24) substituted heteroarylheteroaryl-,
(25) arylaminoheteroaryl-,
(26) substituted arylaminoheteroaryl-,
(27) arylalkynyl-,
(28) substituted arylalkynyl-,
(29) heteroarylalkynyl-,
(30) substituted heteroarylalkynyl-,
(31) benzoheteroaryl;
wherein said R³, R⁴, R⁵, R⁶ and R⁷ substituted groups (7), (9), (11), (13), (16), (18), (20), (22), (24), (26), (28) and (30) are substituted with 1 to 3 substituents independently selected from the group consisting of: —NH₂, —NHR²⁰, —N(R²⁰)₂ wherein each R²⁰ is independently selected, alkyl, alkenyl, halo, —C(O)—NH—R²⁸, —C(O)OR²⁸—C(O)R²⁸, and —OR²⁰,
wherein said R³, R⁴, R⁵, R⁶ and R⁷ substituted groups (3) and (5) are substituted with 1 to 3 substituents independently selected from the group consisting of: —NH₂, halo, —C(O)—NH—R²⁸, —C(O)OR²⁸, and —C(O)R²⁸;
R^{5A} is selected from the group consisting of: halo, —OH, alkyl, —O-alkyl;
R⁸ is selected from the group consisting of: H, —OH, —N(R¹⁰)₂, —NR¹⁹C(O)R¹²;
each R⁹ is independently selected from the group consisting of:halogen, —CN, —NO₂, —OR¹⁹, —SR¹⁰, —N(R¹⁰)₂, and R¹⁰;
each R¹⁹ is independently selected from the group consisting of: H, alkyl, aryl, arylalkyl, heteroaryl, heteroarylalkyl, cycloalkyl, cycloalkylalkyl, heterocycloalkyl, heterocycloalkylalkyl, alkylheteroaryl-, alkylaryl-, substituted alkyl, substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl-, substituted alkylaryl-, heterocycloalkenyl 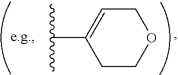
and substituted heterocycloalkenyl, and wherein:
said R¹⁹ substituted alkyl is substituted with 1 to 3 substituents independently selected from the group consisting of: —NH₂, —NHR²⁰, —NO₂, —CN, —OR²⁶, halo, —C(O)—NH—R²⁶, —C(O)OR²⁶, and —C(O)R²⁶, and said R¹⁹ substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl- are substituted with 1 to 3 substituents independently selected from the group consisting of: (1) —NH₂, (2) —NO₂, (3) —CN, (4) —OH, (5) —OR²⁰, (6) —OCF₃, (7) alkyl substituted with 1 to 3 independently selected halo atoms, (8) —C(O)R³⁸, (9) alkyl, (10) alkenyl, (11) halo, (12) —C(O)—NH—R²⁶, (13) —C(O)OR³⁸, (14) —C(O)—NR³²—(C(R³⁰)₂)ₙ—N(R³⁸)₂, (15) —S(O)R³⁸, (16) —C(O)—NR³²—R³⁸, (17) —NR³²—C(O)—R³⁸, (18) 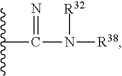
(19) —NHR²⁰, (20) cycloalkyl, (21) —O-alkyl-O—R²⁰, (22) hydroxyalkyl, (23) —N(R²⁰)₂ wherein each R²⁰ is independently selected, (24) -alkyl-OR²⁰, (25) —O-alkyl-OH, (26) —NH(hydroxyalkyl), and (27) oxazolidinone;
R¹¹ is selected from the group consisting of: F, —OH, —ON, —OR¹⁰, —NHNR¹R¹⁰, —SR¹⁰ and heteroaryl;
R¹² is selected from the group consisting of: alkyl, aryl, heteroaryl, cycloalkyl, cycloalkylalkyl, heterocycloalkyl and heterocycloalkylalkyl;
R¹⁴ is selected from the group consisting of: alkyl, aryl, heteroaryl, cycloalkyl, cycloalkylalkyl-, heterocycloalkyl, alkylheterocycloalkyl, heterocycloalkylalkyl-, alkylheteroaryl- and alkylaryl-;
R¹⁵ is selected from the group consisting of: H, —OH, alkyl, aryl, heteroaryl, cycloalkyl, cycloalkylalkyl-, heterocycloalkyl and heterocycloalkylalkyl-, alkylheteroaryl- and alkylaryl-;
R²⁰ represents alkyl;
R²³ is selected from the group consisting of: H, alkyl, aryl, cycloalkyl, and cycloalkylalkyl-;
each R²⁶ is independently selected from the group consisting of: H and alkyl;
R²⁸ is alkyl;
each R³⁰ is independently selected from the group consisting of: H, alkyl, and F;
each R³² is independently selected from the group consisting of: H and alkyl;
each R³⁵ is independently selected from the group consisting of: H and C₁ to C₆ alkyl;
each R³⁸ is independently selected from the group consisting of: H, alkyl, aryl, arylalkyl, heteroaryl, heteroarylalkyl, cycloalkyl, cycloalkylalkyl, heterocycloalkyl, heterocycloalkylalkyl, alkylheteroaryl-, alkylaryl-, substituted alkyl, substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl-, and wherein:
said R³⁸ substituted alkyl is substituted with 1 to 3 substituents independently selected from the group consisting of: —NH₂, —NO₂, —CN, —OR²⁶, halo, —C(O)—NH—R²⁸, —C(O)OR²⁸, and
said R³⁸ substituted aryl, substituted arylalkyl, substituted heteroaryl, substituted heteroarylalkyl, substituted cycloalkyl, substituted cycloalkylalkyl, substituted heterocycloalkyl, substituted heterocycloalkylalkyl, substituted alkylheteroaryl- and substituted alkylaryl- are substituted with 1 to 3 substituents independently selected from the group consisting of: (1) —NH₂, (2) —NO₂, (3) —CN, (4) —OH, (5) —OR²⁶, (6) —OCF₃, (7) —CF₃, (8) —C(O)R²⁶, (9) alkyl, (10) alkenyl, (11) halo, (12) —C(O)—NH—R²⁶, (13) —C(O)OR²⁶, (14))-C(O)—NR³²—(C(R³⁰)₂)ₙ—N(R²⁶)₂, (15) —S(O)R²⁶, (16) —C(O)N(R³²)(R²⁶), (17) —NR³²C(O)R²⁶, (18) 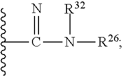
and (19) —NHR²⁰;
R⁴² is selected from the group consisting of: alkyl, aryl (e.g., phenyl), heteroaryl, and cycloalkyl;
R⁴⁴ is selected from the group consisting of: H, alkyl, cycloalkyl, and cycloalkylalkyl; and
Each R⁴⁶ is independently selected from the group consisting of: H, alkyl, cycloalkyl, and cycloalkylalkyl.

2. The compound of claim 1 having the formula:

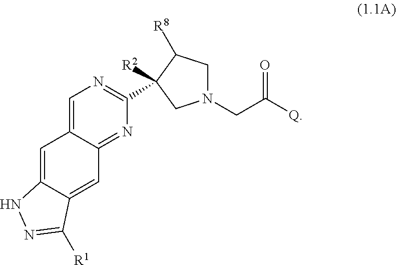

3. The compound of claim 1 wherein Q is selected from the group consisting of: 2.1, 2.2, 2.3, 2.4, 2.5, 2.6, 2.7, and 2.8.

4. The compound of claim 1 wherein Q is selected from the group consisting of: 2.17, 2.18, 2.19, 2.20, 2.21, and 2.22.

5. The compound of claim 1 wherein Z¹ is —CH₂—.

6. The compound of claim 1 wherein each R³, R⁴, R⁶, and R⁷ is independently selected from the group consisting of: H and alkyl.

7. The compound of claim 6 wherein each R³, R⁴, R⁶, and R⁷ is independently selected from the group consisting of: H and methyl.

8. The compound of claim 3 wherein Q is selected from the group consisting of: moieties 2.1, 2.2, and 2.3.

9. The compound of claim 3 wherein:
(A) 0 is selected from the group consisting of: moieties 2.1, 2.2, and 2.3, and 2.3 is selected from the group consisting of: 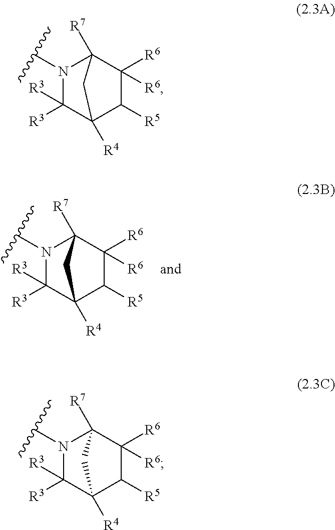
or
(B) Q is selected from the group consisting of: moieties 2.6 and 2.7, and 2.7 is selected from the group consisting of:

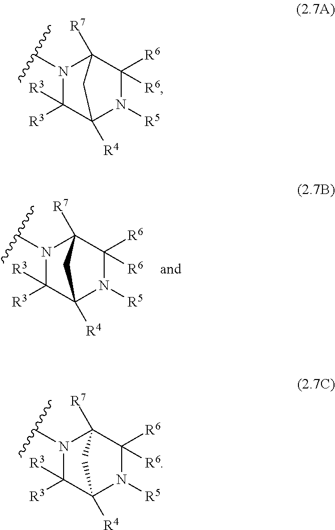

10. The compound of claim 9 wherein each R³, R⁴, R⁶, and R⁷ is independently selected from the group consisting of: H and alkyl.

11. The compound of claim 10 wherein each R³, R⁴, R⁶, and R⁷ is independently selected from the group consisting of: H and methyl.

12. The compound of claim 11 wherein each R³, R⁴, R⁶, and R⁷ is H.

13. The compound of claim 12 wherein Q is 2.1.

14. The compound of claim 12 wherein Q is 2.3B.

15. The compound of claim 3 wherein Q is selected from the group consisting of: moieties 2.6 and 2.7.

16-19. (canceled)

20. The compound of claim 12 wherein Q is 2.6.

21. The compound of claim 12 wherein Q is 2.7A.

22. The compound of claim 12 wherein Q is 2.7B.

23. The compound of claim 1 wherein Q is 2.17.

24. The compound of claim 1 wherein Q is 2.17 wherein each R³, R⁴, R⁶, and R⁷ is independently selected from the group consisting of: H and methyl.

25. The compound of claim 1 wherein Q is 2.17 wherein each R³, R⁴, R⁶, and R⁷ is H.

26. The compound of claim 1 wherein Q is selected from the group consisting of:

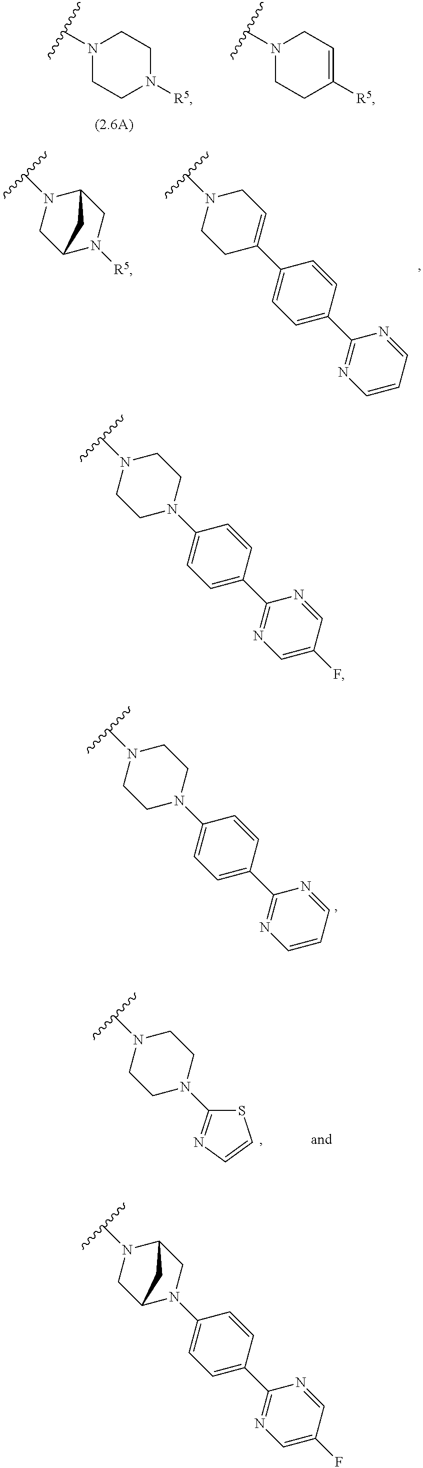

27-33. (canceled)

34. The compound of claim 1 wherein R¹ is selected from the group consisting of:

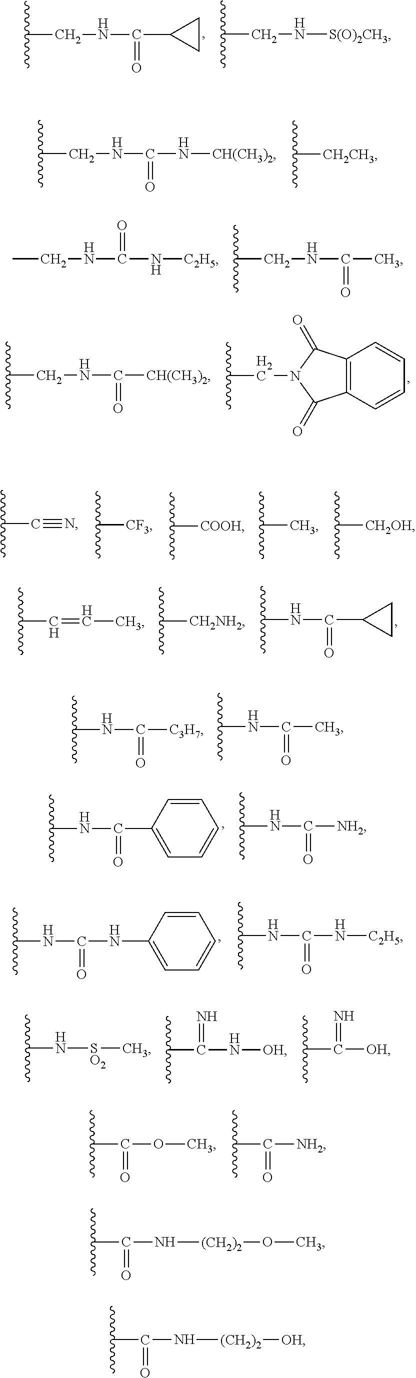

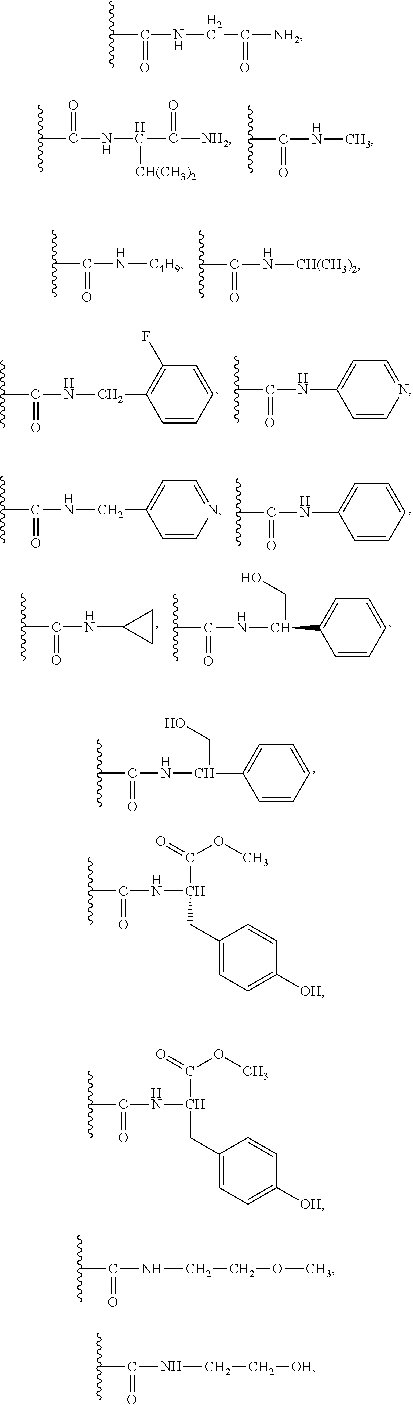

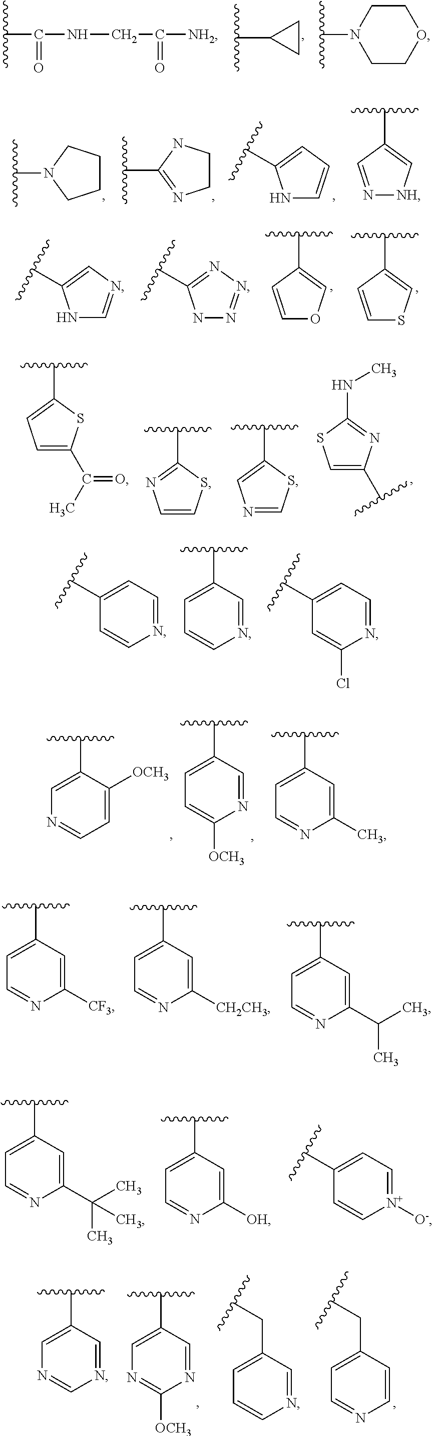

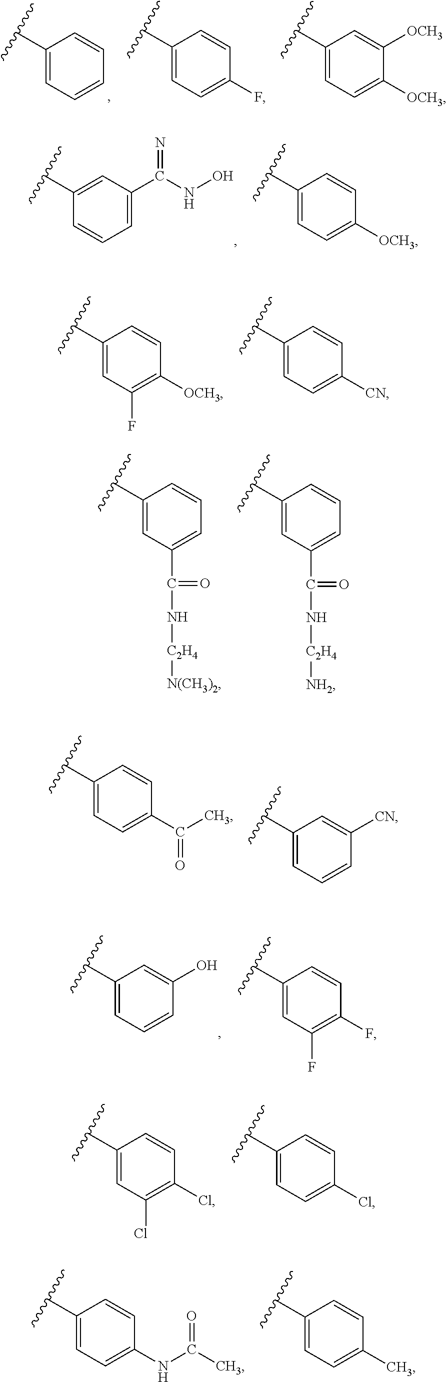

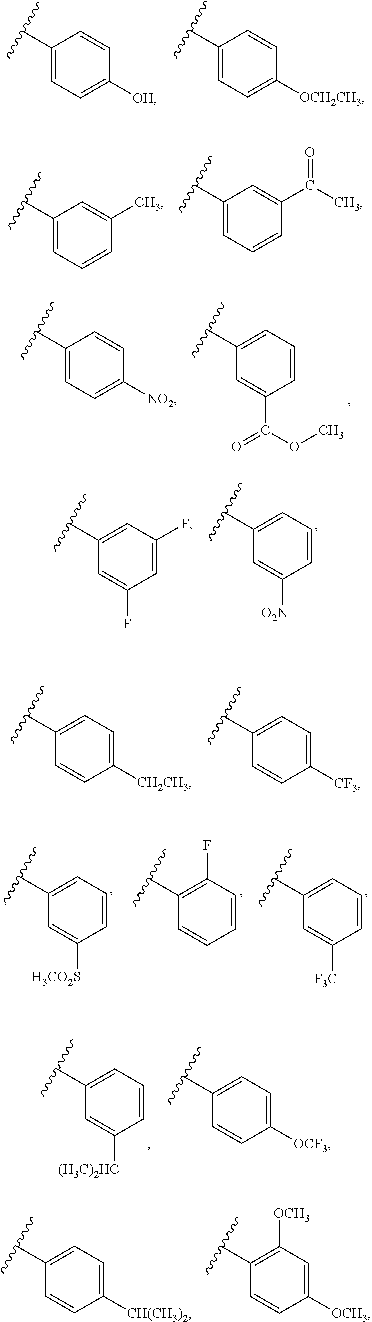

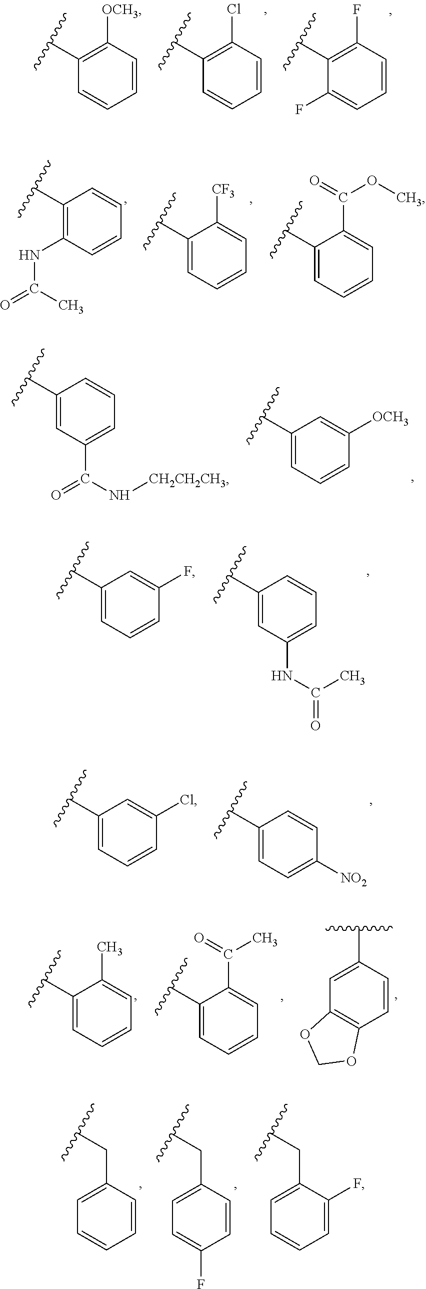

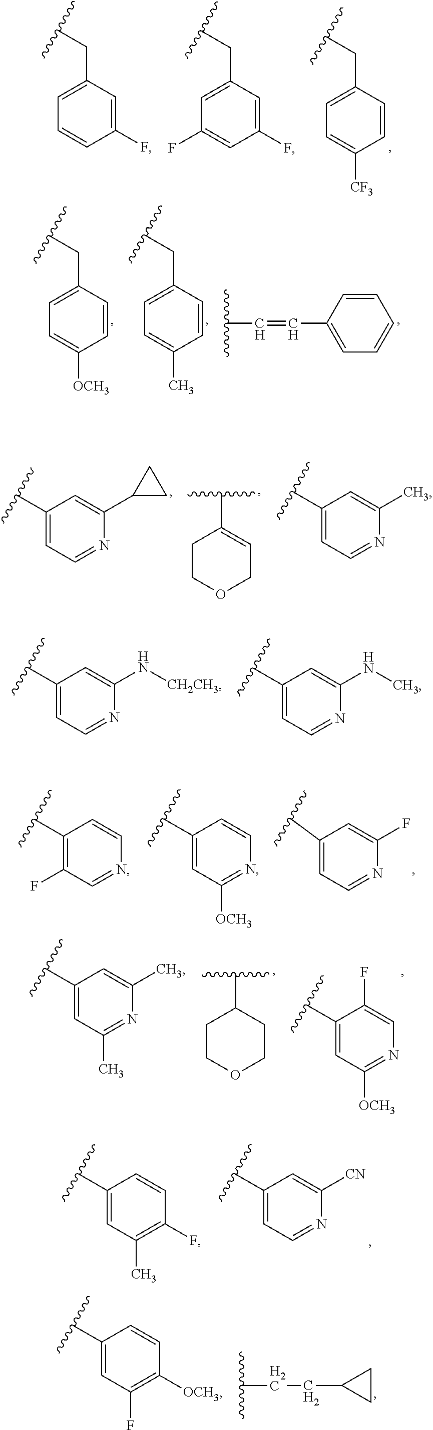

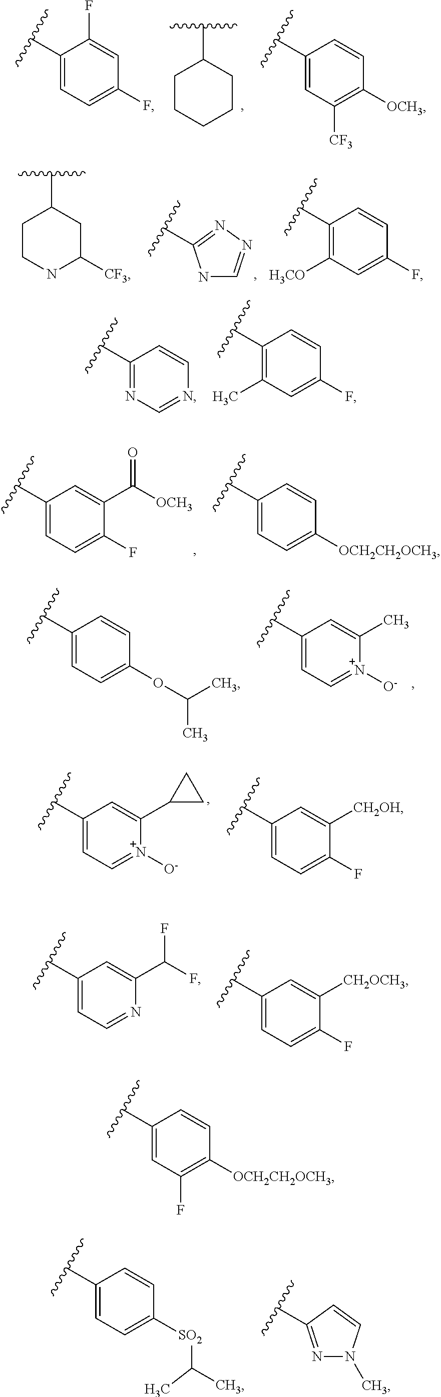

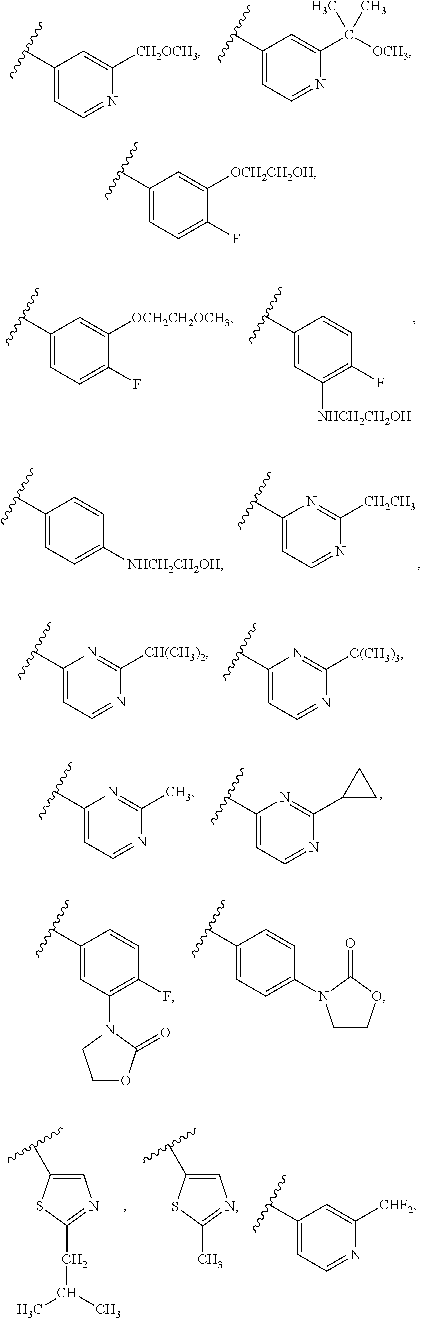

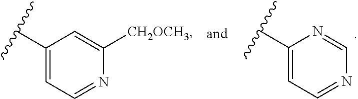

35. The compound of claim 1 wherein R¹ is selected from the group consisting of: 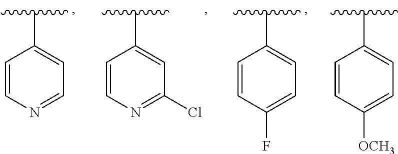
and Br.

36. The compound of claim 1 wherein R¹ is selected from the group consisting of:

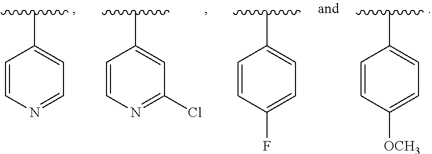

37. The compound of claim 1 wherein R¹ is selected from the group consisting of: aryl and substituted aryl.

38. (canceled)

39. The compound of claim 1 wherein R¹ is heteroaryl or substituted heteroaryl.

40. The compound of claim 1 wherein R⁵ is selected from the group consisting of:

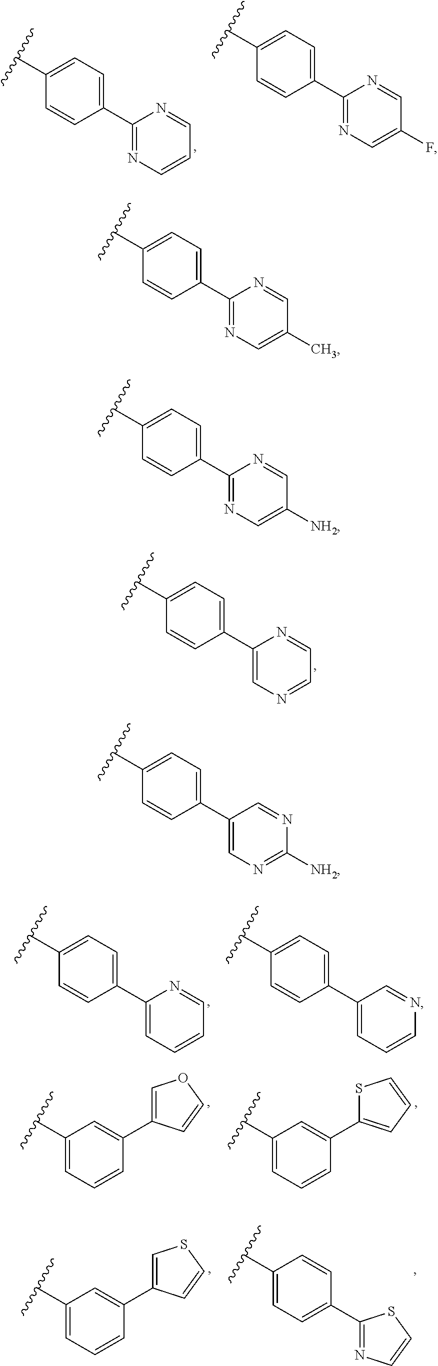

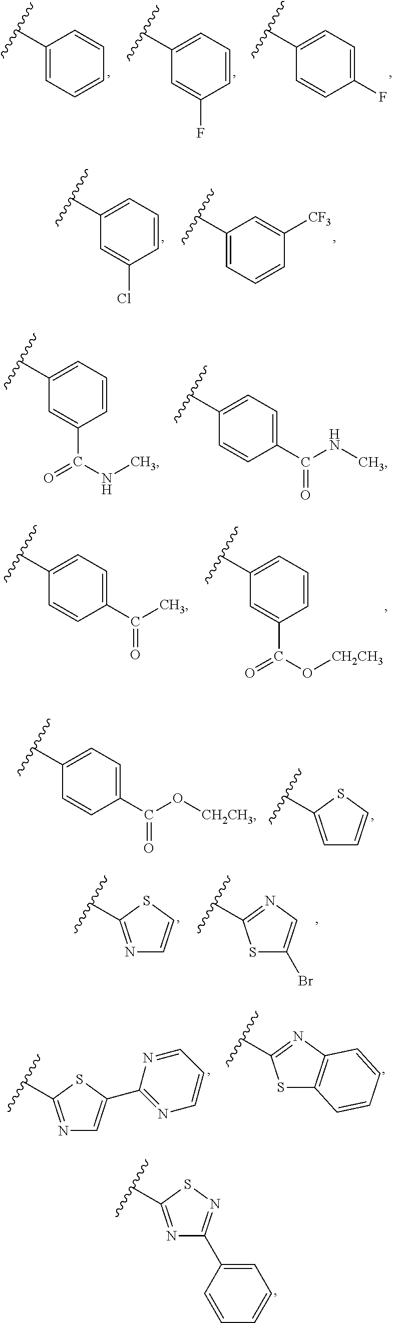

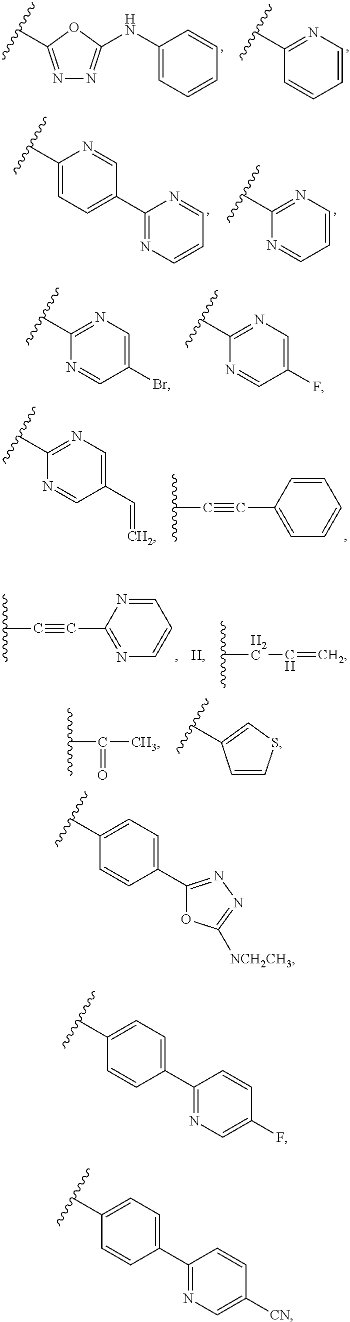

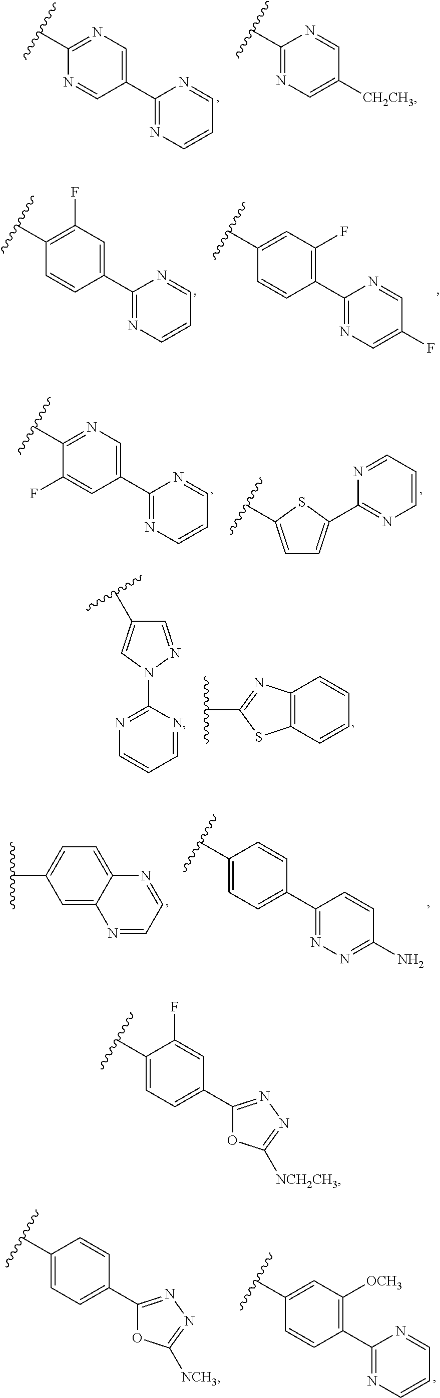

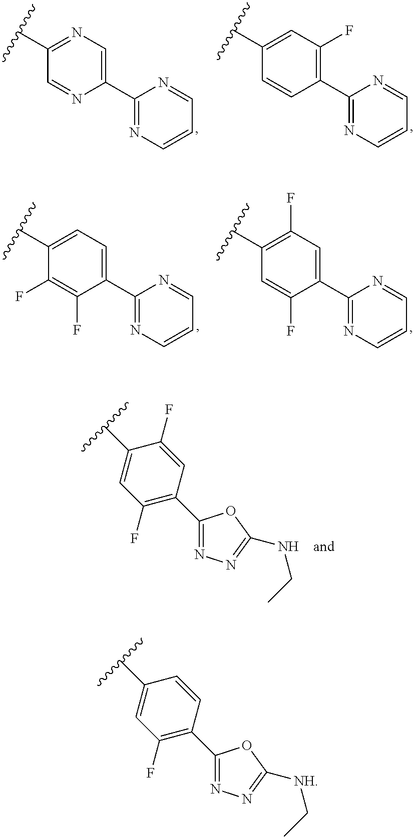

41. The compound of claim 1 wherein R⁵ is selected from the group consisting of:

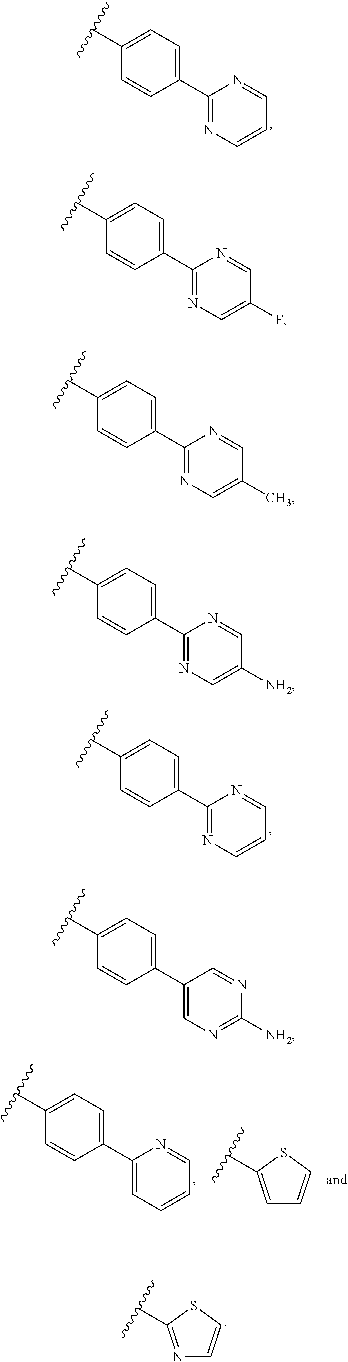

42. The compound of claim 1 wherein R⁵ is selected from the group consisting of:

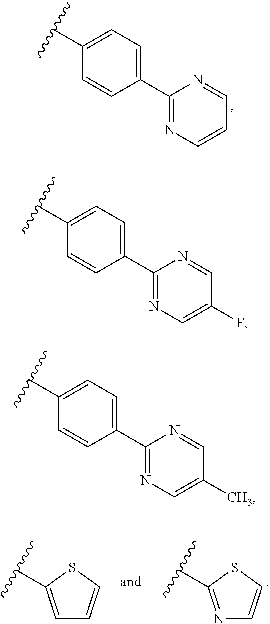

43. The compound of claim 1 wherein R⁵ is selected from the group consisting of:

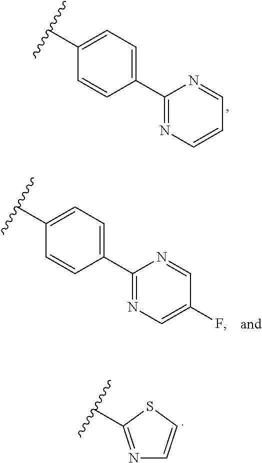

44. The compound of claim 34 wherein R⁵ is selected from the group consisting of:

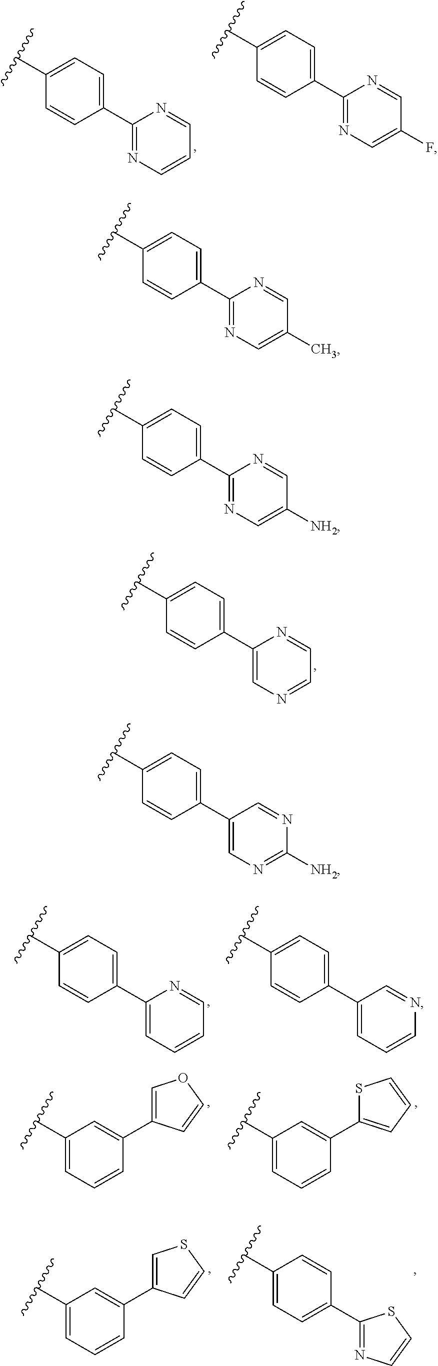

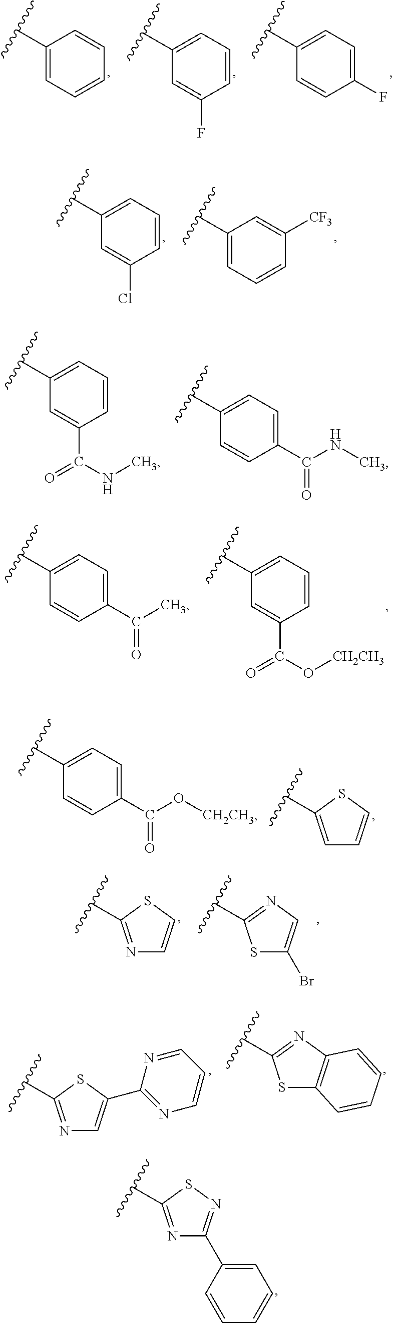

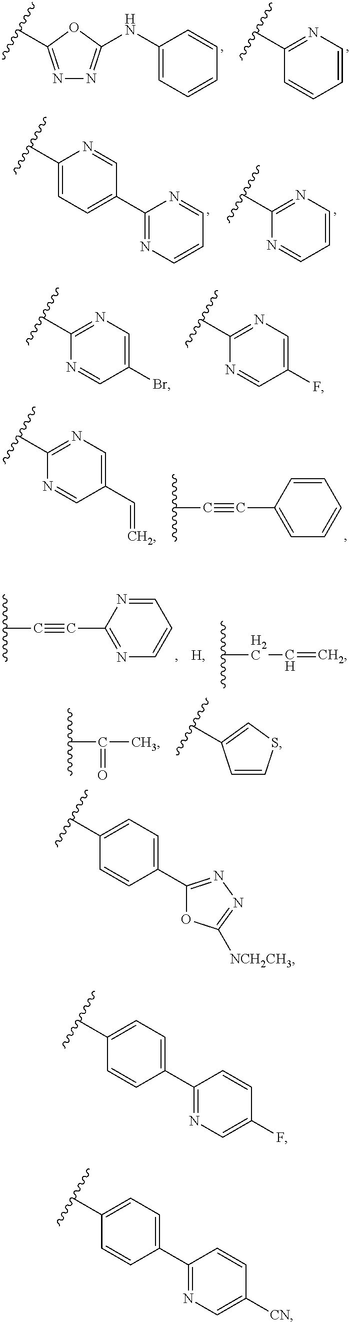

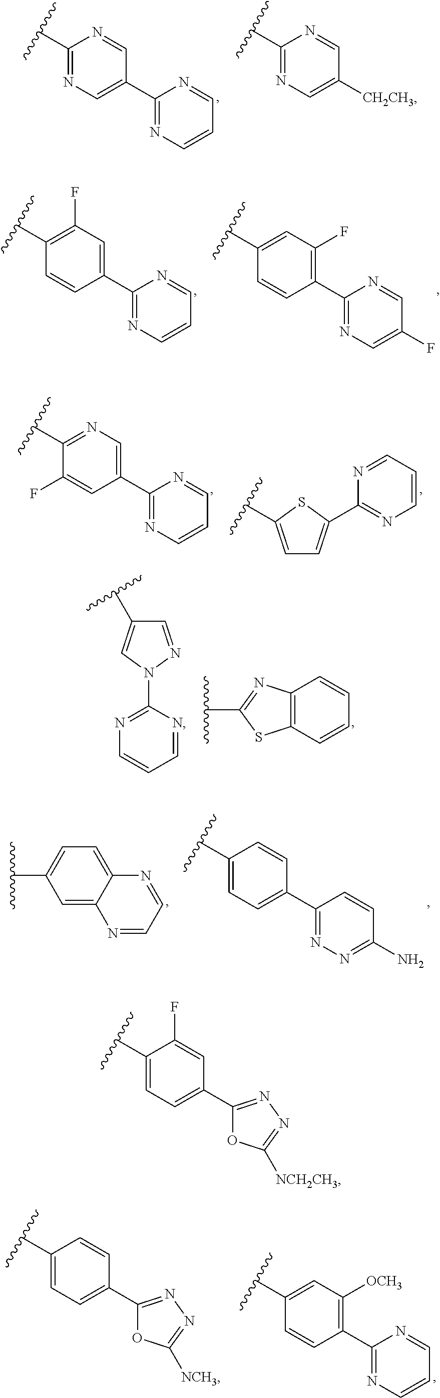

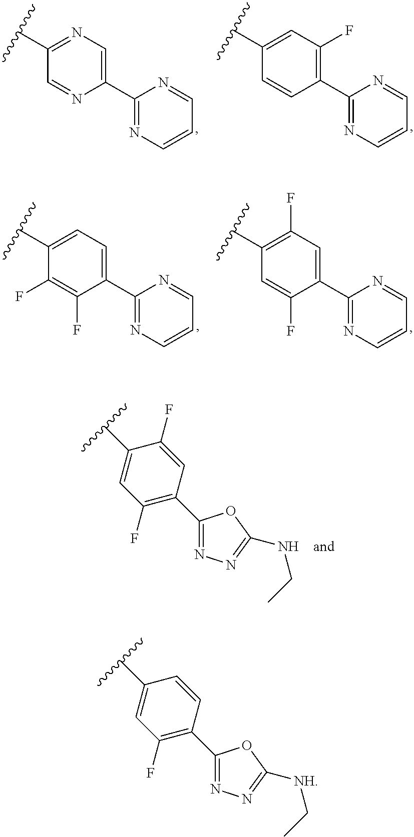

45. The compound of claim 44 wherein R¹ is selected from the group consisting of:

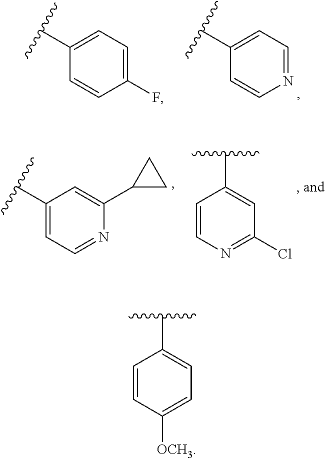

46-49. (canceled)

50. The compound of claim 1 wherein R² is selected from the group consisting of:

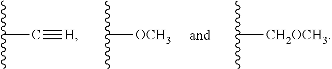

51. The compound of claim 44 wherein R² is selected from the group consisting of:

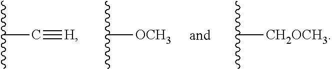

52. The compound of claim 44 wherein R² is selected from the group consisting of: —OCH₃ and H.

53. (canceled)

54. The compound of claim 44 wherein Q is selected from the group consisting of:

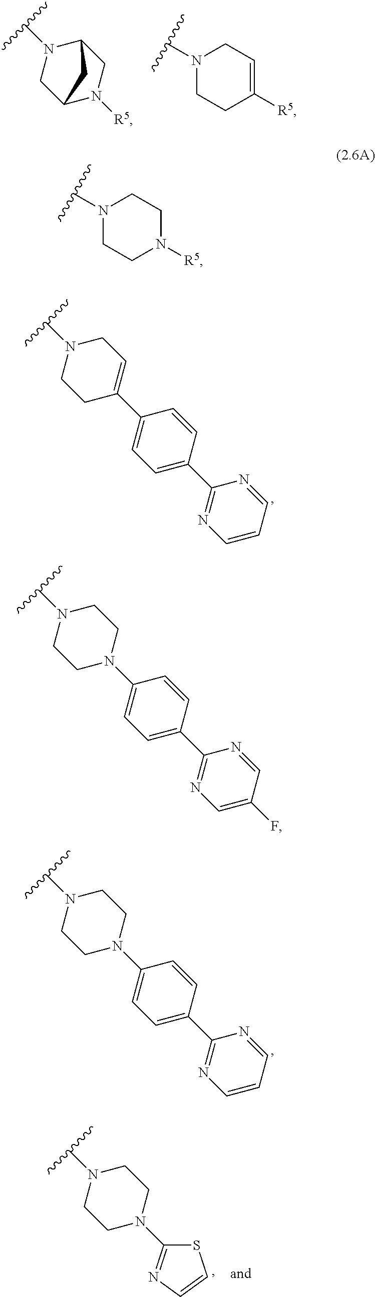

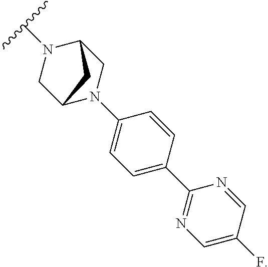

55. (canceled)

56. The compound of claim 54 wherein R² is —OCH₃ and Q is selected from the group consisting of:

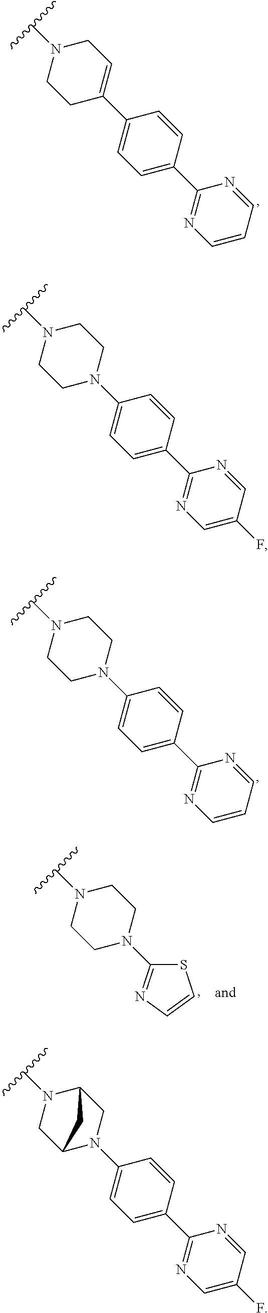

57. The compound of claim 54 wherein R² is H and Q is selected from the group consisting of:

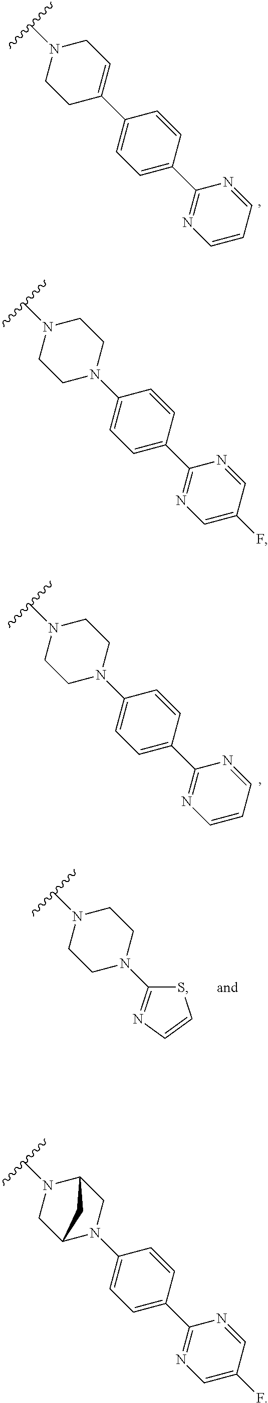

58. The compound of claim 56 wherein R¹ is selected from the group consisting of:

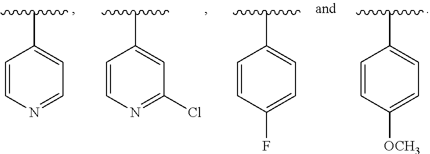

59. (canceled)

60. The compound of claim 1 wherein said compound is a compound of formula 1.0.

61. The compound of claim 1 wherein said compound is a salt of the compound of formula 1.0.

62. The compound of claim 1 wherein said compound is an ester of the compound of formula 1.0, or wherein said compound is a solvate of the compound of formula 1.0.

63. (canceled)

64. The compound of claim 1 selected from the group consisting of:

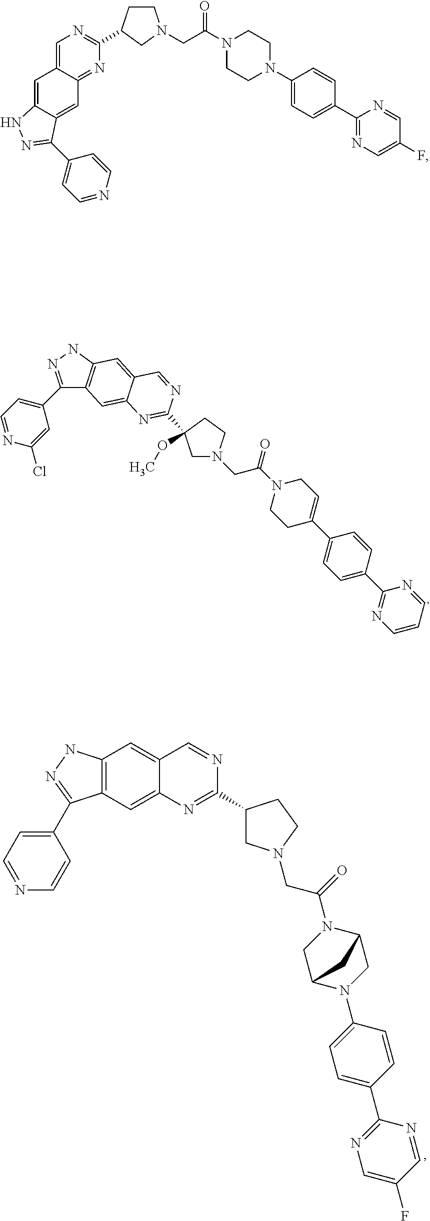

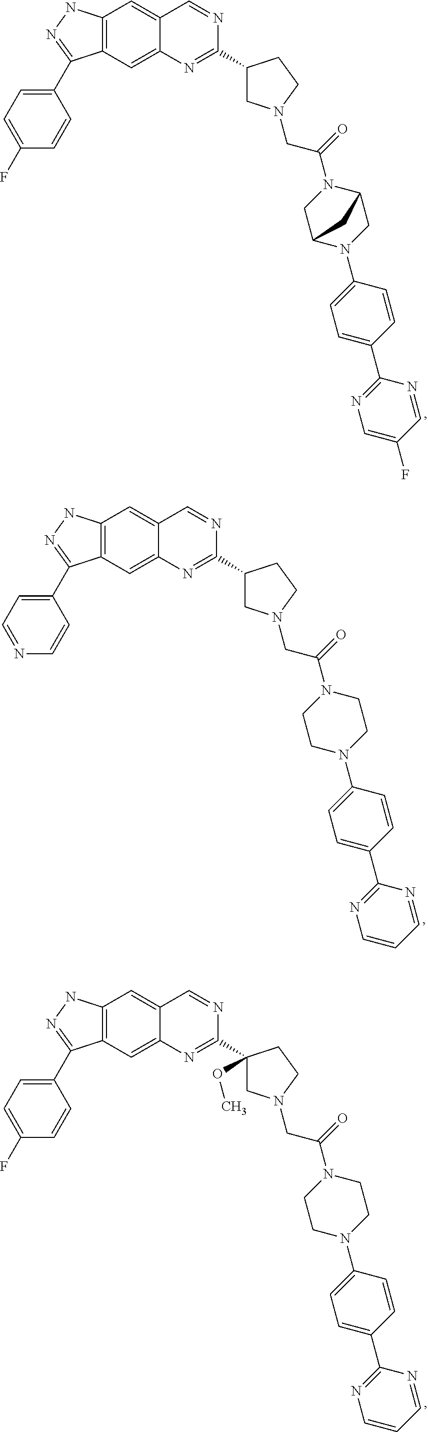

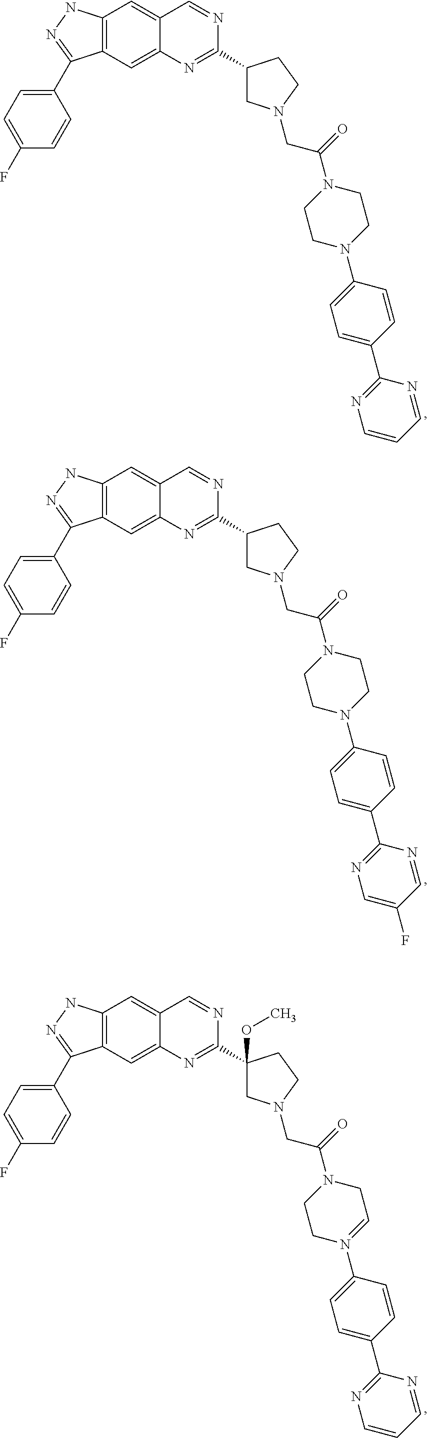

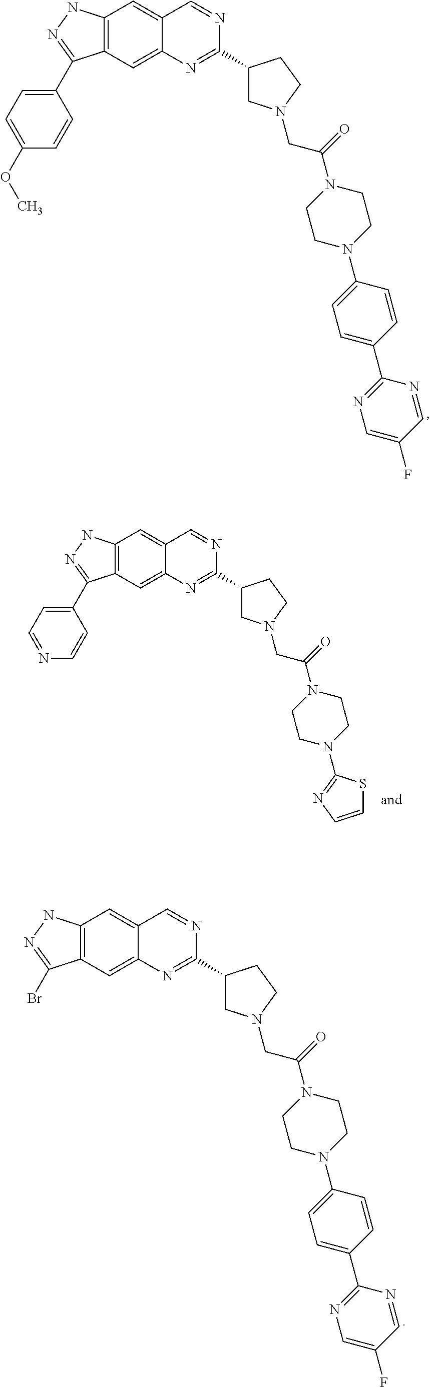

65. A pharmaceutical composition comprising at least one compound of claim 1 and a pharmaceutically acceptable carrier.

66-90. (canceled)

91. A method of preventing hormone-dependent breast cancer in a patient in need of such treatment, said treatment comprising the administration of an effective amount of at least one compound of claim 1 in combination with antihormonal agents, and in combination with an effective amount of at least one chemotherapeutic agent.

92-112. (canceled)

113. A method of treating cancer in a patient in need of such treatment, said method comprising:
(A) administering to said patient an effective amount of at least one compound of claim 1; or
(B) administering to said patient an effective amount of at least one compound of claim 1 in combination with an effective amount of at least one chemotherapeutic agent; or
(C) administering to said patient an effective amount of a compound of claim 1 in combination with an effective amount of at least one chemotherapeutic agent, and an effective amount of radiation therapy; or
(D) administering to said patient an effective amount of at least one compound of claim 1, and therapeutically effective amounts of at least one chemotherapeutic agent selected from the group consisting of: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins, (13) folate antagonists, (14) ribonucleotide reductase inhibitors, (15) anthracyclines, (16) biologics; (17) inhibitors of angiogenesis and/or suppressors of tumor necrosis factor alpha (TNF-alpha) such as thalidomide (or related imid), (18) Bcr/abl kinase inhibitors, (19) MEK1 and/or MEK 2 inhibitors that are small molecules, (20) IGF-1 and 1GF-2 inhibitors that are small molecules, (21) small molecule inhibitors of RAF and BRAF kinases, (22) small molecule inhibitors of cell cycle dependent kinases such as CDK1, CDK2, CDK4 and CDK6, (23) alkylating agents, and (24) farnesyl protein transferase inhibitors; or
(E) administering to said patient an effective amount of at least one compound of claim 1 in combination with at least one signal transduction inhibitor; or
(F) administering to said patient an effective amount of at least one compound of claim 1, said cancer being selected from the group consisting of: lung cancer, pancreatic cancer, colon cancer, myeloid leukemias, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, nuroblastomas, kidney carcinomas, hepatomas, non-Hodgkin's lymphoma, multiple myeloma, and anaplastic thyroid carcinoma; or
(G) administering to said patient an effective amount of at least one compound of claim 1, wherein said cancer is selected from the group consisting of: melanoma, pancreatic cancer, thyroid cancer, colorectal cancer, lung cancer, breast cancer, and ovarian cancer; or
(H) administering to said patient an effective amount of at least one compound of claim 1, in combination with an effective amount of at least one chemotherapeutic agent, wherein said cancer is selected from the group consisting of: melanoma, pancreatic cancer, thyroid cancer, colorectal cancer, lung cancer, breast cancer, and ovarian cancer.

114. A method for treating:
(1) melanoma in a patient in need of such treatment, said method comprising administering to said patient an effective amount of at least one compound of claim 1; or
(2) melanoma in a patient in need of such treatment, said method comprising administering to said patient an effective amount of at least one compound of claim 1, in combination with an effective amount of at least one chemotherapeutic agent; or
(3) pancreatic cancer in a patient in need of such treatment, said method comprising administering to said patient an effective amount of at least one compound of claim 1; or
(4) pancreatic cancer in a patient in need of such treatment, said method comprising administering to said patient an effective amount of at least one compound of claim 1, in combination with an effective amount of at least one chemotherapeutic agent; or
(5) thyroid cancer in a patient in need of such treatment, said method comprising administering to said patient an effective amount of at least one compound of claim 1; or
(6) treating thyroid cancer in a patient in need of such treatment, said method comprising administering to said patient an effective amount of at least one compound of claim 1, in combination with an effective amount of at least one chemotherapeutic agent; or
(7) colorectal cancer in a patient in need of such treatment, said method comprising administering to said patient an effective amount of at least one compound of claim 1; or
(8) colorectal cancer in a patient in need of such treatment, said method comprising administering to said patient an effective amount of at least one compound of claim 1, in combination with an effective amount of at least one chemotherapeutic agent; or
(9) lung cancer in a patient in need of such treatment, said method comprising administering to said patient an effective amount of at least one compound of claim 1; or
(10) lung cancer in a patient in need of such treatment, said method comprising administering to said patient an effective amount of at least one compound of claim 1, in combination with an effective amount of at least one chemotherapeutic agent; or
(11) treating breast cancer in a patient in need of such treatment, said method comprising administering to said patient an effective amount of at least one compound of claim 1; or
(12) treating breast cancer in a patient in need of such treatment, said method comprising administering to said patient an effective amount of at least one compound of claim 1, in combination with an effective amount of at least one chemotherapeutic agent; or
(13) ovarian cancer in a patient in need of such treatment, said method comprising administering to said patient an effective amount of at least one compound of claim 1; or
(14) treating ovarian cancer in a patient in need of such treatment, said method comprising administering to said patient an effective amount of at least one compound of claim 1, in combination with an effective amount of at least one chemotherapeutic agent; or
(15) hormone-dependent breast cancer in a patient in need of such treatment, said treatment comprising the administration of an effective amount of at least one compound of claim 1 in combination with antihormonal agents; or
(16) hormone-dependent breast cancer in a patient in need of such treatment, said treatment comprising the administration of an effective amount of at least one compound of claim 1 in combination with antihormonal agents, and in combination with an effective amount of at least one chemotherapeutic agent; or
(17) hormone-dependent breast cancer in a patient in need of such treatment, said treatment comprising the administration of an effective amount of at least one compound of claim 1 in combination with antihormonal agents; or
(18) brain cancer in a patient in need of such treatment, said method comprising administering to said patient an effective amount of at least one compound of claim 1; or
(19) brain cancer in a patient in need of such treatment, said method comprising administering to said patient an effective amount of at least one compound of claim 1, in combination with an effective amount of at least one chemotherapeutic agent: or
(20) brain cancer in a patient in need of such treatment, said method comprising administering to said patient an effective amount of at least one compound of claim 1, in combination with an effective amount of a chemotherapeutic agent wherein said chemotherapeutic agent is temozolomide; or
(21) prostate cancer in a patient in need of such treatment, said method comprising administering to said patient an effective amount of at least one compound of claim 1; or
(22) prostate cancer in a patient in need of such treatment, said method comprising administering to said patient an effective amount of at least one compound of claim 1, in combination with an effective amount of at least one chemotherapeutic agent; or
(23) myelodysplastic syndrome in a patient in need of such treatment, said method comprising administering to said patient an effective amount of at least one compound of claim 1; or
(24) treating myelodysplastic syndrome in a patient in need of such treatment, said method comprising administering to said patient an effective amount of at least one compound of claim 1, in combination with an effective amount of at least one chemotherapeutic agent; or
(25) myeloid leukemias in a patient in need of such treatment, said method comprising administering to said patient an effective amount of at least one compound of claim 1; or
(26) myeloid leukemias in a patient in need of such treatment, said method comprising administering to said patient an effective amount of at least one compound of claim 1, in combination with an effective amount of at least one chemotherapeutic agent; or
(27) acute myelogenous leukemia in a patient in need of such treatment, said method comprising administering to said patient an effective amount of at least one compound of claim 1; or
(28) acute myelogenous leukemia in a patient in need of such treatment, said method comprising administering to said patient an effective amount of at least one compound of claim 1, in combination with an effective amount of at least one chemotherapeutic agent; or
(29) treating chronic myelomonocytic leukemia in a patient in need of such treatment, said method comprising administering to said patient an effective amount of at least one compound of claim 1; or
(30) chronic myelomonocytic leukemia in a patient in need of such treatment, said method comprising administering to said patient an effective amount of at least one compound of claim 1, in combination with an effective amount of at least one chemotherapeutic agent; or
(31) chronic myelogenous leukemia in a patient in need of such treatment, said method comprising administering to said patient an effective amount of at least one compound of claim 1; or
(32) chronic myelogenous leukemia in a patient in need of such treatment, said method comprising administering to said patient an effective amount of at least one compound of claim 1, in combination with an effective amount of at least one chemotherapeutic agent; or
(33) bladder cancer in a patient in need of such treatment, said method comprising administering to said patient an effective amount of at least one compound of claim 1; or
(34) bladder cancer in a patient in need of such treatment, said method comprising administering to said patient an effective amount of at least one compound of claim 1, in combination with an effective amount of at least one chemotherapeutic agent; or
(35) non-Hodgkin's lymphoma in a patient in need of such treatment, said method comprising administering to said patient an effective amount of at least one compound of claim 1;
(36) non-Hodgkin's lymphoma in a patient in need of such treatment, said method comprising administering to said patient an effective amount of at least one compound of claim 1, in combination with an effective amount of at least one chemotherapeutic agent; or
(37) multiple myeloma in a patient in need of such treatment, said method comprising administering to said patient an effective amount of at least one compound of claim 1; or
(38) multiple myeloma in a patient in need of such treatment, said method comprising administering to said patient an effective amount of at least one compound of claim 1, in combination with an effective amount of at least one chemotherapeutic agent.
